(19) United States
(12) Patent Application Publication
(10) Pub. No.: US 2018/0100026 A1
(43) Pub. Date: Apr. 12, 2018

(54) OPTIMIZED CHIMERIC RECEPTOR T CELL SWITCHES AND USES THEREOF

(71) Applicant: California Institute for Biomedical Research, La Jolla, CA (US)

(72) Inventors: Chanhyuk KIM, San Diego, CA (US); Travis YOUNG, La Jolla, CA (US); Jennifer MA, La Jolla, CA (US); Ji Young KIM, San Diego, CA (US); Xinxin WANG, San Diego, CA (US); Eduardo LABORDA, San Diego, CA (US); Peter G. SCHULTZ, La Jolla, CA (US)

(21) Appl. No.: 15/566,685

(22) PCT Filed: Apr. 15, 2016
(86) PCT No.: PCT/US16/27990

§ 371 Date: Oct. 13, 2017

Related U.S. Application Data
(60) Provisional application No. 62/148,070, filed on Apr. 15, 2015.
(60) Provisional application No. 62/253,465, filed on Nov. 10, 2015.

(51) Int. Cl. C07K 16/44; C07K 14/725; C07D 311/84; C07K 16/28; C07K 16/32; A61K 9/00; A61P 35/02; A61P 35/00; G01N 33/50; A61K 49/00; A61K 47/68; A61K 39/395

(52) U.S. Cl. CPC C07K 16/44; A61K 2039/5156; C07D 311/84; C07K 16/2851; C07K 16/2803; C07K 16/32; C07K 16/2866; A61K 9/0019; A61P 35/02; A61P 35/00; G01N 33/5011; A61K 49/0004; A61K 47/6855; A61K 39/39558; C07K 2319/03; C07K 2319/02; C07K 2317/55; C07K 2317/56; C07K 2317/522; C07K 2317/524; C07K 2317/526; C07K 2317/53; C07K 2319/74; C07K 14/7051

(57) ABSTRACT

Disclosed herein are switches for regulating the activity of a chimeric antigen receptor effector cells (CAR-ECs). The switches generally comprise a chimeric antigen receptor-interacting domain (CAR-ID) and a target interacting domain (TID). The switch may further comprise a linker. Further disclosed herein are methods of using the switches for the treatment of one or more conditions or diseases in a subject in need thereof.

### CROSS-REFERENCE

This application claims priority to U.S. Provisional Application No. 62/148,070, filed, Apr. 15, 2015, and U.S. Provisional Application No. 62/253,465, Nov. 10, 2015, each of which application is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is CIBR_009_02WO_ST25.txt. The text file is 171 KB, was created on Apr. 15, 2016, and is being submitted electronically via EFS-Web.

### BACKGROUND OF THE INVENTION

Immunotherapies are becoming attractive alternatives to chemotherapies, including immunotherapies that use adoptive transfer of genetically modified T cells to “reteach” the immune system to recognize and eliminate malignant tumor cells. Genetically modified T cells express chimeric antigen receptors (CARs), which generally consist of an intracellular signaling domain, a CD3-zeta (ζ) transmembrane domain, and an extracellular single-chain variable fragment (scFv) derived from a monoclonal antibody which gives the receptor specificity for a tumor-associated antigen on a target malignant cell. Upon binding the tumor-associated antigen via the chimeric antigen receptor, the chimeric antigen receptor expressing T cell (CAR T-cell) mounts an immune response that is cytotoxic to the malignant cell. Such therapies can circumvent chemotherapy resistance and are be active against relapsed/refractory disease, resulting in sustained remission for some chronic lymphocytic leukemia (CLL), acute lymphoblastic leukemia (ALL), and acute myeloid leukemia (AML) patients. However, these therapies require further investigation and optimization, as they can cause undesirable effects such as toxic cytopenias and chronic hypogammaglobulinemia for hematological targets, fatal off-target cytolysis for solid tumor targets, persistent B cell aplasia with the use of anti-CD19 antibody expressing CAR T-cells, and, in some cases, death.

### SUMMARY OF THE INVENTION

Disclosed herein are CARs comprising an extracellular domain, a transmembrane domain, and an intracellular signaling domain, wherein the extracellular domain comprises: a region that interacts with a CAR switch; and a hinge domain. The hinge domain may be about 10 amino acids long. The hinge domain may be about 45 amino acids long. The hinge domain may be flexible. The hinge domain may be rigid. A first cysteine of a first CAR and a second cysteine of a second CAR may form a disulfide bond, resulting in multimerization of the first CAR and the second CAR. The hinge domain may have a sequence selected from SEQ ID NOS: 34-37. The hinge domain may have a sequence that is at least about 50% homologous to a sequence selected from SEQ ID NOS: 34-37. The extracellular domain may comprise an antibody or antibody fragment that binds a hapten of the CAR switch. The hapten may be fluorescein isothiocyanate (FITC) or a derivative thereof. The hinge domain may comprise a peptide derived from a protein selected from a CD8, an IgG, portions thereof, and combinations thereof.

Further disclosed herein are soluble T cell receptor (sTCR) switches comprising: a CAR interacting domain (CAR-ID); and a soluble T cell receptor or portion thereof. The CAR-ID may be linked or conjugated to a terminus of a domain of the sTCR. The CAR-ID may be linked or conjugated into an internal site of a domain of the soluble T cell receptor. The domain of the sTCR may be selected from an alpha (α) chain, a beta (β) chain, a gamma (γ) chain, a delta (δ) chain, an epsilon (ϵ) chain and a zeta (ζ) chain. The sTCR switch may further comprise a linker, wherein the linker links the CAR-ID to the sTCR or portion thereof. The linker may be selected from a linker depicted in FIGS. 19-22 and 51, 52, 54 and 55. The CAR-ID may comprise a hapten. The hapten may be FITC or a derivative thereof. The CAR-ID may not comprise a peptide. The sTCR may comprise an unnatural amino acid. The CAR-ID may be linked or conjugated to the unnatural amino acid.

Disclosed herein are CAR switches comprising: a CAR-ID; and a target interacting domain (TID), wherein the CAR-ID is connected to the TID. The TID may be an antibody or an antibody fragment, wherein the CAR-ID is connected to a chain of the targeting antibody or antibody fragment is selected from a light chain, a heavy chain, or a portion thereof. The targeting antibody or antibody fragment may be selected from an anti-CS1 antibody, an anti-Her2 antibody, a B cell maturation antigen (BCMA) antibody, an anti-CD19 antibody, an anti-CD22 antibody, an anti-CLL1 antibody, an anti-CD33 antibody, an anti-CD123 antibody, an anti-EGFRVIII antibody, an anti-CD20 antibody, and an anti-CEA antibody and fragments thereof. The antibody fragment may be a Fab. The antibody fragment may be a variable region of the targeting antibody. The heavy chain may have a sequence selected from SEQ ID NOS: 11, 13, 15, 17, 19, 21, 23, 25, 27, 29 and 31. The heavy chain may have a sequence that is at least about 50% homologous to a sequence selected from SEQ ID NOS: 11, 13, 15, 17, 19, 21, 23, 25, 27, 29 and 31. The light chain may have a sequence selected from SEQ ID NOS: 10, 12, 14, 16, 18, 20, 22, 24, 26, 28 and 30. The light chain may have a sequence that is at least about 50% homologous to a sequence selected from SEQ ID NOS: 10, 12, 14, 16, 18, 20, 22, 24, 26, 28 and 30. The CAR-ID may be a small molecule. The CAR-ID may be a hapten. The CAR-ID may be selected from FITC, biotin, and dinitrophenol. The CAR switch may further comprise a linker, wherein the linker connects the CAR-ID and the TID. The TID may comprise an unnatural amino acid. The CAR-ID and the TID may be connected or linked by the unnatural amino acid. The TID may be an anti-CLL1 antibody or antibody fragment comprising a variable light chain of SEQ ID NO: 18 and optionally SEQ ID NO: 53 and a variable heavy chain of SEQ ID NO: 19 and optionally SEQ ID NO: 52, and the unnatural amino acid may be located at a site selected from Glycine 69, Alanine 110, and Serine 203 of a light chain of the anti-CLL1 antibody or antibody fragment, and Serine 75, Alanine 124, Lysine 139 of a heavy chain of the anti-CLL1 antibody or antibody fragment. The targeting antibody or antibody fragment may be an anti-CD33 antibody or antibody fragment comprising a variable light chain of SEQ ID NO: 20 and optionally SEQ ID NO: 53and a variable heavy chain of SEQ ID NO: 21 and optionally SEQ ID NO: 52, and the unnatural amino acid may be located at a site selected from Glycine 72, Threonine 113 and Serine 206 of a light chain of the anti-CD33 antibody or antibody fragment, and Proline 75, Alanine 117 and Lysine 132 of a heavy chain of the anti-CD33 antibody or antibody fragment. The targeting antibody or antibody fragment may be an anti-CD33 antibody or antibody fragment comprising a variable light chain of SEQ ID NO: 22 and optionally SEQ ID NO: 53 and a variable heavy chain of SEQ ID NO: 23 and optionally SEQ ID NO: 52, and the unnatural amino acid may be located at a site selected from Glycine 72, Threonine 113 and Serine 206 of a light chain of the anti-CD33 antibody or antibody fragment, and Serine 75, Alanine 117 and Lysine 132 of a heavy chain of the anti-CD33 antibody or antibody fragment. The targeting antibody or antibody fragment may be an anti-CD19 antibody or antibody fragment comprising a variable light chain of SEQ ID NO: 16 and optionally SEQ ID NO: 53 and a variable heavy chain of SEQ ID NO: 17 and optionally SEQ ID NO: 52, and the unnatural amino acid may be located at a site selected from Glycine 68, Threonine 109 and Serine 202 of a light chain of the anti-CD19 antibody or antibody fragment, and Serine 74, Alanine 121, Lysine 136 of a heavy chain of the anti-CD19 antibody or antibody fragment. The targeting antibody or antibody fragment may be an anti-CD22 antibody or antibody fragment comprising a variable light chain of SEQ ID NO: 30 and optionally SEQ ID NO: 53 and a variable heavy chain of SEQ ID NO: 31 and optionally SEQ ID NO: 52, and the unnatural amino acid may be located at a site selected from Glycine 74, Threonine 114 and Serine 207 of a light chain of the anti-CD22 antibody or antibody fragment, and Serine 75, Alanine 117, Lysine 132 of a heavy chain of the anti-CD22 antibody or antibody fragment. The targeting antibody or antibody fragment may be an anti-CD22 antibody or antibody fragment comprising a variable light chain of SEQ ID NO: 28 and optionally SEQ ID NO: 53 and a variable heavy chain of SEQ ID NO: 29 and optionally SEQ ID NO: 52, and the unnatural amino acid may be located at a site selected from Glycine 68, Threonine 109 and Serine 202 of a light chain of the anti-CD22 antibody or antibody fragment, and Serine 78, Alanine 125, Lysine 140 of a heavy chain of the anti-CD22 antibody or antibody fragment. The targeting antibody or antibody fragment may be an anti-Her2 antibody or antibody fragment comprising a variable light chain of SEQ ID NO: 12 and optionally SEQ ID NO: 53 and a variable heavy chain of SEQ ID NO: 13 and optionally SEQ ID NO: 52, and the unnatural amino acid may be located at a site selected from Glycine 68 (as in SEQ ID NO: 42), Threonine 109 and Serine 202 (as in SEQ ID NO: 43) of a light chain of the anti-Her2 antibody or antibody fragment, and Serine 75 (as in SEQ ID NO: 44), Alanine 121, Lysine 136 (as in SEQ ID NO: 45) of a heavy chain of the anti-Her2 antibody or antibody fragment. The targeting antibody or antibody fragment may be an anti-CD123 antibody or antibody fragment comprising a variable light chain of SEQ ID NO: 26 and optionally SEQ ID NO: 53 and a variable heavy chain of SEQ ID NO: 27 and optionally SEQ ID NO: 52, and the unnatural amino acid may be located at a site selected from Glycine 68, Threonine 109 and Serine 202 of a light chain of the anti-CD123 antibody or antibody fragment, and Serine 75, Alanine 116, Lysine 131 of a heavy chain of the anti-CD123 antibody or antibody fragment. The targeting antibody or antibody fragment may be an anti-CD123 antibody or antibody fragment comprising a variable light chain of SEQ ID NO: 24 and optionally SEQ ID NO: 53 and a variable heavy chain of SEQ ID NO: 25 and optionally SEQ ID NO: 52, and the unnatural amino acid may be located at a site selected from Arginine 72, Threonine 113 and Serine 206 of a light chain of the anti-CD123 antibody or antibody fragment, and Serine 75, Alanine 119, Lysine 134 of a heavy chain of the anti-CD123 antibody or antibody fragment. Further disclosed herein are pharmaceutical compositions comprising these CAR switches.

Disclosed herein are methods of treating a disease or condition in a subject in need thereof, comprising administering a CAR switch disclosed herein, wherein the CAR switch is administered by a method selected from intraperitoneal injection and intravenous injection. The method may comprise administering the CAR switch and/or a CAR effector cell (CAR-EC) multiple times. The disease or condition may be acute myeloid leukemia (AML).The method may comprise administering a first CAR switch comprising a first targeting antibody or antibody fragment and a second CAR switch comprising a second targeting antibody or antibody fragment, wherein the first targeting antibody or antibody fragment binds a first antigen and the second targeting antibody or antibody fragment binds a second antigen, wherein the first antigen and the second antigen are different. The first and/or second antigen may be selected from CD19, CD22, Her2, CLL1, CD33, CD123, BCMA, CS1, EGFR, EGFRVIII, CD20, and CEA.

Further disclosed herein are methods of selecting an optimal switchable CAR (sCAR) platform, comprising: attaching a first CAR-ID to a first site of a target interacting domain (TID) that binds a first cell surface molecule on a first target cell to produce a first switch; attaching a second CAR-ID to a second site of a second TID that binds a second cell surface molecule on a second target cell to produce a second switch; contacting the first target cell with the first switch and a first CAR-EC expressing a first CAR; contacting the second target cell with the second switch and a second CAR-EC expressing a second CAR; and comparing a first cytotoxic effect of the first switch and the first CAR-EC on the first target cell to a second cytotoxic effect of the second switch and the second CAR-EC on the second target cell; and selecting the first switch and first CAR-EC or the second switch and the second CAR-EC as the optimal sCAR platform based on comparing the first cytotoxic effect to the second cytotoxic effect. The first CAR-ID and the second CAR-ID may be the same. The first TID and the second TID may be the same. The first site and the second site may be different. The first site and the second site may be the same. The first and/or second targeting moiety may comprise a peptide or protein. The first site and/or second site may be selected from an N terminus of the peptide or protein, a C terminus of the peptide or protein, and an internal site of the peptide or protein. The first and/or second targeting moiety may comprise an antibody or antibody fragment. The first site and/or second site may be selected from an N terminus of the antibody or antibody fragment, a C terminus of the antibody or antibody fragment, and an internal site of the antibody or antibody fragment. The first site and/or second site may be selected from a light chain of the antibody or antibody fragment and a heavy chain of the antibody or antibody fragment. The first site and/or second site may be selected from a variable region of the antibody or antibody fragment and a constant region of the antibody or antibody fragment. The first site and/or second site may be selected from a VL domain, a CL domain, a VH domain, a CH1 domain, a CH2 domain, a CH3 domain, and a hinge domain of the antibody or antibody fragment. Attaching the first/second CAR-ID may comprise a method selected from fusing, grafting, conjugating and linking. The method may further comprise attaching a first linker to the first site, wherein the first linker links the first CAR-ID to the first TID. The method may further comprise attaching a second linker to the second site wherein the second linker links the second CAR-ID to the second TID. The first linker and the second linker may be the same. The first linker and the second linker may be different. The first linker and the second linker may differ by a feature selected from flexibility, length, chemistry, and combinations thereof. The first CAR and the second CAR may be the same. The first CAR and the second CAR may be different. The first CAR and the second CAR may differ by a domain selected from an extracellular domain, a transmembrane domain, an intracellular domain and a hinge domain. The first hinge domain of the first CAR and a second hinge domain of the second CAR may differ by a feature selected from flexibility, length, amino acid sequence and combinations thereof. The method may further comprise incorporating one or more additional CAR-IDs to the first and/or second TID to produce a first multivalent switch and/or a second multivalent switch. The method may further comprise incorporating a cysteine residue into the first CAR and/or the second CAR in order to multimerize the first CAR and/or the second CAR through a disulfide bond. Contacting the first target cell and/or contacting the second target cell may occur in vitro. Contacting the first target cell and/or contacting the second target cell may occur in vivo. Comparing the first cytotoxic effect to the second cytotoxic effect may comprise comparing a feature selected from viability of target cells, viability of off-target cells, tumor burden, and health of a subject in an in vivo model.

Disclosed herein are optimized CAR-EC platforms, comprising: a CAR switch comprising a CAR-ID and a TID; and a CAR-EC that expresses a CAR, wherein the CAR-EC platform is derived by a method comprising: attaching a first CAR-ID to a first site of a TID that binds a first cell surface molecule on a first target cell to produce a first switch; attaching a second CAR-ID to a second site of a second TID that binds a second cell surface molecule on a second target cell to produce a second switch; contacting the first target cell with the first switch and a first CAR-EC expressing a first CAR; contacting the second target cell with the second switch and a second CAR-EC expressing a second CAR; and comparing a first cytotoxic effect of the first switch and the first CAR-EC on the first target cell to a second cytotoxic effect of the second switch and the second CAR-EC on the second target cell; and selecting the first switch and first CAR-EC or the second switch and the second CAR-EC as the optimal sCAR platform based on comparing the first cytotoxic effect to the second cytotoxic effect. The CAR-EC may be derived from a T cell. The TID may be selected from a protein, a peptide, an antibody, an antibody fragment, a small molecule, and a soluble T cell receptor or portion thereof. The TID may comprise an antibody or antibody fragment that binds a cell surface molecule selected from CD19, CD22, Her2, CLL1, CD33, CD123, BCMA, CS1, EGFR, EGFRVIII, CD20, and CEA. The TID may comprise a sequence selected from SEQ ID NOS: 10-31 and optionally SEQ ID NOs: 52 and 53. The TID may comprise a sequence at least about 50% homologous to a sequence selected from SEQ ID NOS: 10-31 and optionally SEQ ID NOs: 52 and 53. The CAR-ID may comprise a small molecule. The CAR-ID may comprise a hapten. The CAR-ID may be selected from FITC, biotin, and dinitrophenol.

### BRIEF DESCRIPTION OF THE DRAWINGS

FIG. 1A illustrates a general overview of chimeric antigen receptor-T cell (CAR T-cell) and CAR T-cell switch therapy with switches disclosed herein. Lymphocytes are isolated from a subject and an expression vector encoding a chimeric antigen receptor is subsequently introduced to the lymphocytes to produce chimeric antigen receptor expressing cells. Resulting engineered lymphocytes are administered to the subject, along with a CAR T-cell switch.

FIG. 1B illustrates a CAR T-cell switch, comprising FITC that is bound by the CAR of the CAR T-cell and a targeting antibody that is selective for a target cell. Binding of the CAR T-cell switch to the CAR T-cell induces an immune response that would be cytotoxic to the malignant cell also bound to the CAR T-cell switch.

FIG. 2 shows unnatural amino acid incorporation sites in a human IgG1 Fab crystal structure. Amino acid residues and numbers are based on anti-human CD33 Fab (clone hM195) sequence (SEQ ID NOS: 22 and 23).

FIG. 3 shows various potential FITC conjugation sites on the anti-CD19 Fab (FITC conjugation sites: A=light chain glycine 68, B=heavy chain serine 74, C=light chain threonine 109, D=heavy chain alanine 121, E=light chain serine 202, and F=heavy chain lysine 136 of SEQ ID NOS: 16 (light chain) and 17 (heavy chain)).

FIG. 4 shows the effect of the conjugation site on the length and geometry of the immunological synapse between the CAR-T cell and the CD19⁺ target cell (FITC conjugation sites: B=heavy chain serine 74, F=heavy chain lysine 136 of SEQ ID NO: 16 (light chain) and SEQ ID NO:17 (heavy chain)).

FIG. 5 shows the effect of varying the sites of conjugation on the cytotoxic effect of FITC-anti-CD19 switches in NALM 6 (CD19⁺) cells (FITC conjugation sites: A=light chain glycine 68, B=heavy chain serine 74, C=light chain threonine 109, D=heavy chain alanine 121, E=light chain serine 202, and F=heavy chain lysine 136 of SEQ ID NO:16 (light chain) and SEQ ID NO:17 (heavy chain)).

FIG. 6 shows the effect of valency on the cytotoxic effect of FITC-anti-CD19 switches in NALM 6 (CD19⁺) cells (FITC conjugation sites: A=light chain glycine 68, B=heavy chain serine 74, E=light chain serine 202, and F=heavy chain lysine 136, AB=light chain glycine 68 and heavy chain serine 74, EF=light chain serine 202 and heavy chain lysine 136 of SEQ ID NO:16 (light chain) and SEQ ID NO:17 (heavy chain)).

FIG. 7 shows the difference in cytotoxicity between randomly conjugated FITC-anti-CD19 switches and site specifically conjugated FITC-anti-CD19 switches in NALM 6 (CD19⁺) cell (FITC conjugation sites: A=light chain glycine 68, AB=light chain glycine 68 and heavy chain serine 74 of SEQ ID NO:16 (light chain) and SEQ ID NO:17 (heavy chain).

FIG. 8 depicts the effect of conjugation site on the length and geometry of the immunological synapse between the CAR-T cell and the CLL1⁺ target (FITC conjugation sites: B=heavy chain serine 75, F=heavy chain lysine 139 of SEQ ID NO:19).

FIG. 9 shows the effect of conjugation site on the cytotoxic effect of anti-CLL1 switches (FITC conjugation sites: A=light chain glycine 69, B=heavy chain serine 75, C=light chain alanine 110, D=heavy chain alanine 124, E=light chain serine 203, F=heavy chain lysine 139 of SEQ ID NO:18 (light chain) and SEQ ID NO:19 (heavy chain)).

FIG. 10 shows the effect of valency on the cytotoxic effect of anti-CLL1 switches (FITC conjugation sites: A=light chain glycine 69, B=heavy chain serine 75, AB=light chain glycine 69 and heavy chain serine 75 of SEQ ID NO:18 (light chain) and SEQ ID NO:19 (heavy chain)).

FIG. 11 depicts an anti-Her2 Fab switch with various FITC conjugation sites (FITC conjugation sites: A=light chain glycine 68, B=heavy chain serine 75, E=light chain serine 202, F=heavy chain lysine 136 of SEQ ID NO:12 (light chain) and SEQ ID NO:13 (heavy chain)).

FIG. 12 shows the effect of the conjugation site on the length and geometry of the immunological synapse between the CAR-T cell and the Her2⁺ target cell (FITC conjugation sites: B=heavy chain serine 75, E=light chain serine 202 of SEQ ID NO:12 (light chain) and SEQ ID NO:13 (heavy chain)).

FIG. 13 shows cytotoxicity data for anti-Her2 switches in SKBR3 Her2 3+ cells (FITC conjugation sites: B=heavy chain serine 75, E=light chain serine 202, AB=light chain glycine 68 and heavy chain serine 75, EF=light chain serine 202 and heavy chain lysine 136 of SEQ ID NO:12 (light chain) and SEQ ID NO:13 (heavy chain)).

FIG. 14 shows cytotoxicity data for anti-Her2 switches in MDA MB231 Her2 1+ cells (FITC conjugation sites: B=heavy chain serine 75, E=light chain serine 202, AB=light chain glycine 68 and heavy chain serine 75, EF=light chain serine 202 and heavy chain lysine 136 of SEQ ID NO:12 (light chain) and SEQ ID NO:13 (heavy chain)).

FIG. 15 depicts two anti-CD22 switches that bind membrane different epitopes (membrane distal for clone hLL2 and proximal for clone M971) in the CD22 extracellular domain. A-F represents FITC conjugation sites in each switch (FITC conjugation sites: A=light chain glycine 74, B=heavy chain serine 75, C=light chain threonine 114, D=heavy chain alanine 117, E=light chain serine 207, F=heavy chain lysine 132 of SEQ ID NO:30 (light chain) and SEQ ID NO:31 (heavy chain); A=light chain glycine 68, B=heavy chain serine 78, C=light chain threonine 109, D=heavy chain alanine 125, E=light chain serine 202, F=heavy chain lysine 140 of SEQ ID NO:28 (light chain) and SEQ ID NO:29 (heavy chain)).

FIG. 16 shows cytotoxicity data for M971 switches in Daudi (CD22+) cells (FITC conjugation sites: A=light chain glycine 74, B=heavy chain serine 75, E=light chain serine 207, F=heavy chain lysine 132, AB=light chain serine 74 and heavy chain serine 75, EF=light chain serine 207 and heavy chain lysine 132 of SEQ ID NO:28 (light chain) and SEQ ID NO:29 (heavy chain)).

FIG. 17 shows cytotoxicity data for hLL2 switches in Raji (CD22+) cells (FITC conjugation sites: A=light chain glycine 68, B=heavy chain serine 78, C=light chain threonine 109, D=heavy chain alanine 117, E=light chain serine 202, F=heavy chain lysine 140 of SEQ ID NO:30 (light chain) and SEQ ID NO:31 (heavy chain)).

FIG. 18 shows cytotoxicity comparison for hLL2 (FITC conjugation sites: AB=light chain glycine 74 and heavy chain serine 75, EF=light chain serine 207 and heavy chain lysine 132 of SEQ ID NO:30 (light chain) and SEQ ID NO:31 (heavy chain)) and M971 (FITC conjugation sites: AB=light chain glycine 68 and heavy chain serine 78, EF=light chain serine 202 and heavy chain lysine 140 of SEQ ID NO:28 (light chain) and SEQ ID NO:29 (heavy chain)) switches in NALM 6 (CD19+) cells.

FIG. 19 shows distance between target cells and CAR-T cells as well as the accessibility of anti-FITC scFv to switch molecules can be optimized by adjusting the linker length to 1 polyethylene glycol molecules.

FIG. 20 shows distance between target cells and CAR-T cells as well as the accessibility of anti-FITC scFv to switch molecules can be optimized by adjusting the linker length to 4 polyethylene glycol molecules.

FIG. 21 shows distance between target cells and CAR-T cells as well as the accessibility of anti-FITC scFv to switch molecules can be optimized by adjusting the linker length to 12 polyethylene glycol molecules.

FIG. 22 demonstrates the effect of linker length on the overall distance between target and CAR-T cells. The relative dimension of each component should be noted; the 10 Å difference between 1PEG and 4PEG linkers roughly reaches 20% of Fab length.

FIG. 23 shows expression and conjugation of M971-FITC switches by SDS-PAGE.

FIG. 24 shows characterization of M971-LG68-4PEG-FITC by mass spectrometry. Expected: 49273 Da, Observed: 49273 Da.

FIG. 25 shows characterization of M971-LT109-4PEG-FITC by mass spectrometry. Expected: 49229 Da, Observed: 49229 Da.

FIG. 26 shows characterization of M971-LK169-4PEG-FITC by mass spectrometry. Expected: 49202 Da, Observed: 49202 Da.

FIG. 27 shows characterization of M971-LS202-4PEG-FITC by mass spectrometry. Expected: 49243 Da, Observed: 49243 Da.

FIG. 28 shows characterization of M971-HS78-4PEG-FITC by mass spectrometry. Expected: 49243 Da, Observed: 49244 Da.

FIG. 29 shows characterization of M971-HA125-4PEG-FITC by mass spectrometry. Expected: 49285 Da, Observed: 49260 Da.

FIG. 30 shows characterization of M971-HK140-4PEG-FITC by mass spectrometry. Expected: 49202 Da, Observed: 49202 Da.

FIG. 31 shows characterization of M971-LG68HS78-4PEG-FITC by mass spectrometry. Expected: 50132 Da, Observed: 50132 Da.

FIG. 32 shows characterization of M971-LS202HK140-4PEG-FITC by mass spectrometry. Expected: 50061 Da, Observed: 50061 Da.

FIG. 33 shows characterization of M971-LS202HS78-4PEG-FITC by mass spectrometry. Expected: 50102 Da, Observed: 50102 Da.

FIG. 34 shows expression and conjugation of hLL2-FITC switches by SDS-PAGE.

FIG. 35 shows characterization of hLL2-LG74-4PEG-FITC by mass spectrometry. Expected: 49215 Da, Observed: 49215 Da.

FIG. 36 shows characterization of hLL2-HS75-4PEG-FITC by mass spectrometry. Expected: 49186 Da, Observed: 49186 Da.

FIG. 37 shows characterization of hLL2-LG74HS75-4PEG-FITC by mass spectrometry. Expected: 50074 Da, Observed: 50074 Da.

FIG. 38 shows characterization of hLL2-LS207-4PEG-FITC by mass spectrometry. Expected: 49186 Da, Observed: 49186 Da.

FIG. 39 shows characterization of hLL2-HK132-4PEG-FITC by mass spectrometry. Expected: 49144 Da, Observed: 49145 Da.

FIG. 40 shows characterization of hLL2-LS207HK132-4PEG-FITC by mass spectrometry. Expected: 50004 Da, Observed: 50005 Da.

FIG. 41 shows expression of hM195-LG72HS75-pAzF by SDS-PAGE.

FIG. 42 shows characterization of hM195-LG72HS75-pAzF by mass spectrometry. Expected: 47924 Da, Observed: 47927 Da.

FIG. 43 shows mass spectrometry of FITC conjugated hM195-LG72HS75-1PEG-FITC switch. Expected: 49174 Da, Observed: 49171 Da.

FIG. 44 shows mass spectrometry of FITC conjugated hM195-LG72HS75-4PEG-FITC switch. Expected: 49438 Da, Observed: 49434Da.

FIG. 45 shows an anti-CLL1 light chain with potential FITC conjugation sites.

FIG. 46 shows an anti-CLL1 heavy chain with potential FITC conjugation sites.

FIG. 47 shows dose-dependent cytotoxicity in CLL1⁺ U937 cells treated with the indicated FITC-conjugated switches and anti-FITC sCAR-T cells. Half-maximal killing concentrations (EC50s) for each switch are indicated in the table.

FIG. 48 shows dose-dependent cytotoxicity in CLL1⁺ HL60 cells treated with the indicated FITC-conjugated switches and anti-FITC sCAR-T cells. EC50s for each switch are indicated in the table.

FIG. 49 shows IL-2, IFNγ, and TNFα cytokine measurements (pg/mL) from the cytotoxicity assay shown in FIG. 48 at 2 nM concentrations of the indicated FITC-conjugated switches.

FIG. 50A-FIG. 50C shows in vivo antitumor efficacy of conventional anti-Her2 CAR-T and sCAR-T cell approaches in HCC1954 (A), MDA MB453 (B) and MDA MB231 (C) xenograft models. Each data point represents tumor volume of five mice in each group. Error bars represent SD. Arrows indicate the time of CAR-T cell injection or of treatment with specific antibodies.

FIG. 51 shows a two-step conjugation reaction consisting of an oxime reaction followed by “click” reaction in which a ketone of a p-acetylphenylalanine (pAcF) residue is used as a chemical handle to modify the protein with a heterobifunctional N3-TEG-ONH₂ linker. In this schematic, FITC is modified with a linker ending in a cyclooctyne, which can be clicked to the modified protein.

FIG. 52 shows a schematic of conjugation via p-azidophenylalanine (pAzF). The pAzF unnatural amino acid is incorporated into anti-CD19 to produce a proteinogenic substrate for a single step “click” conjugation to a FITC molecule modified with a cyclooctyne linker.

FIG. 53 shows schematics of CAR-EC regulators and CAR-ECs.

FIG. 54 depicts exemplary linkers.

FIG. 55 depicts exemplary heterobifunctional linkers.

FIG. 56 shows a schematic of exemplary switches.

FIG. 57 shows an exemplary schematic of producing a switch.

FIG. 58 shows an example of switchable CAR-T cell and formation of a bivalent immunological synapse from a bivalent switch and a monovalent CAR.

FIG. 59 shows exemplary site and stoichiometry of FITC conjugation.

FIG. 60A shows a study design of an in vivo B cell depletion study in C57BL/6 mice.

FIG. 60B shows CD3⁺ and CD19⁺ populations in blood with conventional versus switchable CAR-T-CD19 therapy.

FIG. 61 shows an exemplary chimeric antigen receptor expression cassette.

FIG. 62 shows an example of sCAR-T cell and formation of a bivalent immunological synapse from a monovalent switch and a bivalent CAR.

FIG. 63A shows a crystal structure of a mouse anti-CD19 Fab (clone 93f3, Protein Data Bank (PDB) ID: 1T4K) indicating FITC conjugation sites.

FIG. 64A-FIG. 64C shows anti-FITC CAR-T cells and NALM-6 cells co-cultured at a 5:1 ratio, respectively, with different concentrations of anti-CD19 FITC conjugates in cytotoxicity assays. One representative experiment is shown to demonstrate the impact of (FIG. 64A) conjugation site, (FIG. 64B) valency, and (FIG. 64C) conjugation method (site-specific vs random) on CAR-T cell activity.

FIG. 64D-FIG. 64E shows results from cytotoxicity assays comparing conjugation sites and valency of anti-CD22 FITC conjugates against CD22⁺ target cells.

FIG. 64F-FIG. 64G shows results from optimized CD19 and CD22 targeting switches against tumor cell lines with differential antigen expression levels: NALM-6 (CD19^{high},CD22^{low}) and Raji (CD19^{high},CD22^{high}). Each data point represents a mean of duplicate samples, and error bars represent SD. Results shown are a representative of three independent experiments.

FIG. 65A shows in vitro and in vivo comparison of CART-19 and anti-FITC CAR-T cells with optimized anti-CD19 AB-FITC switch. Anti-FITC CAR-T cells and Nalm-6 (CD19+) cells were co-cultured for at indicated E:T ratios with 1 nM of anti-CD19 AB-FITC switch, and target cell lysis was determined by flow cytometry.

FIG. 65B shows measurement of indicated cytokines in supernatant from activation assays using BD Cytometric Bead Array (CBA) Human Th1/Th2 II Kit according to manufacturer's protocol. All results are representative of independent experiments with CAR-T cells generated from three different donors. Data shown are an average of duplicate or triplicate samples, and error bars represent SD. n.s.=p>0.05 and *=p<0.05 were calculated using one-tailed Student's t-test.

FIG. 65C-D shows results from 0.5×10⁶ Nalm-6 cells transfected with luciferase were injected intravenously (IV) into 6-8 weeks-old female NOD.Cg-Prkdc^{scid} Il2rg^{tm1Wjl}/SzJ (NSG) mice. Seven days later, mice were infused with 40×10⁶ CAR-T cells IV and switch treatment was initiated with indicated anti-CD19 FITC switches at 0.5 mg/kg or PBS every other day for a total of six doses (IV). (FIG. 65C) Tumor burden was monitored by weekly bioluminescence imaging (BLI), and (FIG. 65D) quantified by bioluminescent signal intensity. Results are derived from 6 mice/group, and error bars represent SD.

FIG. 66A shows dose titratable in vivo response of anti-FITC CAR-T cells achieved with anti-CD19 AB-FITC switch. BLI of NSG mice inoculated with 0.5×10⁶ NALM-6 cells on day 1, and infused with 40×10⁶ CART-19 or anti-FITC CAR-T cells on day 7. On the same day, switch treatment was initiated at indicated doses at every other day for 6 doses. Tumor bearing mice treated with vehicle (PBS) were included as a negative control.

FIG. 66B shows CD3⁺ peripheral blood lymphocyte (PBL) count from weekly retro-orbital bleeds after treatment with anti-CD19 AB-FITC. As a control for potential non-specific T cell expansion, disease-free mice were injected with anti-FITC CAR-T cells and received six doses of anti-CD19-FITC (0.5 mg/kg). Averages and error bars represent SD derived from 3-4 mice from each group.

FIG. 66C-FIG. 66D shows BLI and body weight of NSG mice inoculated with Nalm-6 cells, and infused anti-FITC CAR-T cells as described in FIG. 65A. On day 7, anti-CD19 AB-FITC treatment was initiated at indicated doses and continued every other day. Parentheses indicate the total number of doses that each group received. Arrow specifies time of increase in switch dose from 0.05 to 0.5 mg/kg. (FIG. 66D) Percentage of body weight change observed from (FIG. 66C). Data points and error bars represent average and SD derived from 6 mice/group, respectively.

FIG. 67A shows in vitro efficacy of mouse anti-FITC CAR-T cells with anti-mouse CD19 (1D3)-FITC switch. Cytotoxicity assay using mouse anti-FITC CAR-T cells and Myc5 CD19⁺ co-cultured at a 10:1 ratio, respectively, in the presence of different concentrations of indicated anti-CD19 (1D3)-FITC switch. Non-transduced mouse T cells and mouse T cells transduced with an irrelevant anti-TNP CAR served as negative controls. Each data point represents a mean of triplicate samples, and error bars represent SEM. Results presented are representative of three independent experiments.

FIG. 67B shows flow cytometry analysis of CD3⁻ or CD19⁺ cells in mice treated with anti-CD19 (1D3) CAR-T or anti-FITC CAR-T with anti-CD19 (1D3)-FITC switch. C57BL/6 mice were preconditioned with cyclophosphamide (150 mg/kg) on day 1, and received 6×10⁶ syngeneic anti-CD19 (ID3) CAR or anti-FITC CAR-T cells by tail vein injections the next day. Daily treatments with anti-mouse CD19 (1D3)-FITC switch at 1 mg/kg were initiated the same day as CAR-T cell infusions for a total of 10 injections (Day 2-11). Weekly retro-orbital bleeds were carried out to assess CD3⁺ and CD19⁺. Dot plots are a representative of 5-6 mice/group.

FIG. 67C shows graphical representation of CD19⁺ cells quantified from FIG. 66. Results are displayed as an average of 5-6 mice, and error bars represent SD. NS=p>0.05, *=p<0.05, and ***=p<0.0005 were calculated using one-tailed Student's t-test.

FIG. 68A-FIG. 68D shows cytotoxicity assays comparing CAR-T cells derived from different anti-FITC scFvs against various CD19⁺ target cell lines (FIG. 68A-C). Anti-FITC-CAR-T cells and CD19⁺ cells were co-cultured at a 5:1 ratio, respectively, with different concentrations of anti-CD19 AB-FITC. A CD19⁻ target cell line (K562) (FIG. 68D) was included as a negative control. Each data point represents a mean of duplicate samples, and error bars represent SD. Results presented are a representative of three independent experiments.

FIG. 68E shows second generation CAR construct consisting of the fully human anti-FITC scFv (FITC-E2) and signaling domains of 41BB and CD3ζ.

FIG. 68F shows cell surface CAR expression levels on transduced human T cells. Transduction efficiency was evaluated weekly by flow cytometry with APC-conjugated anti-human IgG F(ab)′2 antibody or FITC-labeled isotype antibodies.

FIG. 69A shows a general scheme to generate site-specific FITC-antibody conjugates. Mutant antibodies incorporated with pAzF were conjugated with BCN-PEG₄-FITC by “Click” reaction.

FIG. 69B shows mass spectrometry analysis of site-specific anti-CD19-FITC conjugates obtained on an Agilent Quadruple Time-of-Flight (QTOF) mass spectrometer. Deconvoluted masses were obtained using Agilent Qualitative Analysis software.

FIG. 69C shows a table of expected and observed masses of site-specific anti-CD19 FITC switches.

FIG. 70A shows a general scheme to generate random FITC-antibody conjugates with FITC-PEG₄-NHS.

FIG. 70B shows mass spectrometry analysis of random anti-CD19-FITC conjugated with deconvolution profiles of random anti-CD19 FITC conjugate obtained on an Agilent QTOF mass spectrometer.

FIG. 70C-FIG. 70E show butterfly plots of FITC-conjugated and unconjugated peptides from a single CESI-MS IDA peptide mapping run to identify modified sites found on random FITC switches generated by N-hydroxysuccinimide (NHS) chemistry as described in FIG. 69A.

FIG. 71A-FIG. 71D shows binding capacity of anti-CD19 FITC conjugates evaluated using the CD19⁺ cells, Nalm-6 (FIG. 71A), the CD19⁻ cells, K562 (FIG. 71B) and anti-FITC CAR-T cells (FIG. 71C). Cells were incubated with indicated switch antibodies at 4° C. for 30-60 min and washed twice with staining buffer (1% BSA in PBS). Primary antibodies were revealed with Alexa Fluor®647 conjugated anti-human IgG or anti-human κ secondary antibodies. After several washes, samples were acquired on a BD LSR II or BD Accuri C6 and analyzed using FlowJo 7.6.2 software. In each study, cells were incubated with secondary antibody alone and the observed mean fluorescence intensity (MFI) was used to subtract for background and non-specific staining.

FIG. 72A-FIG. 72D shows results of a cytotoxicity assay comparing indicated anti-CD19 FITC against target cells with different CD19 expression levels. Data points depict a mean of duplicate samples, and error bars represent SD.

FIG. 73A shows a table of EC₅₀ values from cytotoxicity assays described in FIG. 71A.

FIG. 73B shows results of surface CD19 expression on indicated cell lines quantified with PE-conjugated CD19 flow cytometry antibodies using Quanti-Brite PE beads.

FIG. 73C-FIG. 73E shows quantification of indicated cytokines in co-cultures containing equal numbers (1×10⁵) of anti-FITC CAR-T cells and NALM-6 (CD19⁺) cells in the presence of 1 nM anti-CD19 AB-FITC switch. The next day, cultured media was harvested and cytokines were quantified using BD Cytometric Bead Array (CBA) Human Th1/Th2 II.

FIG. 73F-FIG. 73G shows results of cytotoxicity assays consisting of 10 pM anti-CD19 AB-FITC with anti-FITC CAR-T cells and NALM-6 (CD19⁺) cells co-cultured at a 5:1 ratio supplemented with excess amounts of anti-CD19 antibody (FMC63 IgG) or isotype control (Millipore) or fluorescein (Sigma).

FIG. 73H shows surface antigen expression quantified with PE-conjugated CD19 and CD22 flow cytometry antibodies using Quanti-Brite PE beads. All results are a representative or summary of independent experiments with CAR-T cells generated from three different donors. Data shown are an average of duplicate or triplicate samples, and error bars represent SD.

FIG. 74A shows a second generation CAR construct consisting of anti-CD19 scFv (clone FMC63) and signaling domains of 41BB and CD3ζ.

FIG. 74B shows cell surface CAR expression levels on enriched transduced human T cells. Transduction efficiency was evaluated with APC-conjugated anti-mouse or anti-human IgG F(ab)′2 antibody.

FIG. 74C-FIG. 74D shows results from cytotoxicity and cytokine release assays comparing CART-19 and anti-FITC CAR-T cell activity against K562 (CD19⁻) target cells.

FIG. 74E shows results of 1×10⁵ anti-FITC CAR-T cells and NALM-6 (CD19⁺) or K562 (CD19⁻) cells cultured in the presence of 1 nM of anti-CD19 AB-FITC switch. The next day, CD3⁺ cells were evaluated for upregulation of activation markers (CD69 and CD25) by flow cytometry.

FIG. 74F shows results from equal number (1×10⁵) of anti-FITC CAR-T cells and target cells were co-cultured in the presence of 1 nM anti-CD19 AB-FITC switch. The next day, cell mixtures were surface labeled with anti-CD3, fixed, and permeabilized prior to anti-Bcl-xl staining. Data shown represent an average of duplicate samples and error bars represent SD. All results are a representative or summary of independent experiments with CAR-T cells generated from three different donors. n.s.=p>0.05 using one-tailed Student's t-test.

FIG. 75A shows tumor burden quantified using bioluminescent signal intensity from switch dose titration study as described in FIG. 66A. No significant difference was found between CART-19 and sCAR-T with 0.5 mg/kg treatment groups; n.s.=p>0.05. However, significance was observed when comparing sCAR-T with 0.05 mg/kg and CART-19 or sCAR-T with 0.5 mg/kg treatment; ***=p<0.0005. Significance was determined using 2-way ANOVA with Turkey's multiple comparisons test (shown for final time-point only).

FIG. 75B shows a graphical representation of percentage body weight change observed after initiation of CART-19 or anti-FITC CAR-T cells with anti-CD19 AB-FITC therapy on day 10 of the study. **=p<0.005 using one-tailed Student's t-test.

FIG. 75C shows a summary of body weight change observed throughout the study. Data points and error bars represent average and SD derived from 6 mice/group, respectively.

FIG. 76A shows a Nalm-6 xenograft model where 0.5×10⁶ luciferized NALM-6 cells were intravenously (IV) injected into 6-8 weeks-old female NSG mice. Seven days later, mice were IV infused with 40×10⁶ CAR-T cells and switch treatment was initiated with anti-CD19 AB-FITC conjugate at indicated concentrations or PBS every other day. Parentheses indicate the total number of doses that each group received.

FIG. 76B shows results of tumor burden monitored by weekly BLI. Results are derived from 6 mice/group and error bars represent SD. *=p<0.05 and ***=p<0.0005 was determined using 2-way ANOVA with Turkey's multiple comparisons test (shown for final time-point only).

FIG. 77A shows tumor burden quantified by bioluminescent signal intensity from FIG. 66C. Parentheses indicate the total number of doses that each group received. Arrow specifies time of increase in switch dose from 0.05 to 0.5 mg/kg. No significant difference was found between 0.5 mg/kg (×6) and dose escalation treatment groups; n.s.=p>0.05. However, significance was observed when comparing 0.05 mg/kg (×6) and 0.5 mg/kg (×6) or dose escalation regimen; ***=p<0.0005.Significance was determined using 2-way ANOVA with Turkey's multiple comparisons test (shown for final time-point only).

FIG. 77B-FIG. 77E shows serum cytokine levels 24 hours after initiation of sCAR-T cell therapy with indicated switch dose. Cytokines were quantified with CBA Human Th1/Th2 Kit II and Mouse Inflammation Kit (BD). Averages and SD values were derived from 6 mice/group. Significance was calculated using 2-way ANOVA with Turkey's multiple comparisons test: n.s.=p>0.05, *=p<0.05, **=p<0.005, ***=p<0.0005.

FIG. 78A shows second generation retroviral construct consisting of anti-mouse CD19 (1D3) or anti-FITC (FITC-E2) scFv, and signaling domains of CD28 and CD3ζ.

FIG. 78B shows cell surface CAR expression levels on transduced mouse T cells. Transduction efficiency of anti-mouse CD19 CAR T cells and anti-FITC CAR-T cells were evaluated by flow cytometry using PE-conjugated anti-rat or APC-conjugated anti-human IgG F(ab)′2 antibody, respectively.

FIG. 79 shows an in vivo efficacy assay where U937 cells were injected into NSG mice to induce tumor growth. Mice were injected with E2 FITC CAR-T after tumors reached 150-200 mm³ and switch treatment was initiated with anti-CLL1 Ab-FITC conjugates (red), E2 FITC CAR-T alone (yellow) or PBS (black) IV injections for 10 doses. Shown are tumor measurements taken every other day. Error bars represent SD, results are derived from 5 mice/group.

FIG. 80 shows dose-dependent cytotoxicity in CD123⁺ KASUMI cells treated with the indicated FITC-conjugated switches and anti-FITC sCAR-T cells. EC50s for each switch are indicated in the table.

FIG. 81 shows dose-dependent cytotoxicity in CD123⁺ MOLM13 cells treated with the indicated FITC-conjugated switches and anti-FITC sCAR-T cells. EC50s for each switch are indicated in the table.

FIG. 82 shows dose-dependent cytotoxicity in CD33⁺ U937 cells treated with the indicated FITC-conjugated switches and anti-FITC sCAR-T cells. EC50s for each switch are indicated in the table.

FIG. 83 shows dose-dependent cytotoxicity in CD33⁺ U937 cells treated with the indicated bivalent FITC-conjugated switches and anti-FITC sCAR-T cells. EC50s for each switch are indicated in the table.

FIG. 84 shows dose-dependent cytotoxicity in CD33⁺ THP-1 cells treated with the indicated bivalent FITC-conjugated switches and anti-FITC sCAR-T cells. EC50s for each switch are indicated in the table.

FIG. 85 shows dose-dependent cytotoxicity in CD33⁺ MOLM14 cells treated with the indicated bivalent FITC-conjugated switches and anti-FITC sCAR-T cells. EC50s for each switch are indicated in the table.

FIG. 86A-FIG. 86B shows the binding capacity of anti-CD33 Fabs evaluated using MOLM14 (CD33⁺, FIG. 86A) and NALM-6 (CD33⁻, FIG. 86B) cells. Binding capacity is measured through MFI. MFI of secondary antibody alone was subtracted to account for background and non-specific staining.

FIG. 87A show the FITC labeling sites superimposed on the crystal structure of the anti-Her2 Fab for the generation of FITC-based switches.

FIG. 87B FIG.87B shows exemplary CAR expression cassettes with CD8 hinges (top) and IgG4m hinges (bottom).

FIG. 88 shows a general scheme to generate site-specific FITC antibody conjugates. Mutant antibodies incorporated with pAzF were conjugated with BCN-PEG4-FITC by “Click” reaction.

FIG. 89 shows SDS-PAGE analysis of switches before and after FITC conjugation.

FIG. 90 shows mass spectrometry analysis of site-specific anti-Her2-FITC conjugates obtained on an Agilent Quadruple Time-of-Flight (QTOF) mass spectrometer. Deconvoluted masses were obtained using Agilent Qualitative Analysis software.

FIG. 91 shows flow cytometry analysis of FITC-conjugated switches and wild type 4D5 Fab bound to breast cancer cells with a range of Her2 expression levels. Bound switches were detected with an Alexa Fluor647 conjugated anti-human IgG (H+L) secondary antibody. The order from top to bottom in the panels corresponds to the order from top to bottom in the key.

FIG. 92A-FIG. 92C shows the evaluation of the binding activity of FITC CD8 (FIG. 92A) IgG4m (FIG. 92B), and unrelated (FIG. 92C) switches on FITC specific CAR-T cells. Cells were incubated with indicated switches and detected with Alexa Fluor647 conjugated anti-human κ-chain antibody. The EC₅₀ (Half-maximal binding concentration) value was calculated by the Graphpad Prism software.

FIG. 93 shows the simultaneous binding of switches to sCAR-T cells and the Her2 extracellular domain (ECD). sCAR-T cells were labeled with different concentration of switches and subsequently stained with APC conjugated soluble Her2 extracellular domain (ECD). Binding was measured by flow cytometry and depicted as mean fluorescence intensity±SD (n=2).

FIG. 94A-FIG. 94B shows sCAR-T activation by combinations of various switches and hinge length sCAR-T cells against different Her2 expressing cancer cells. FITC-based sCAR-T cells were co-cultured with SKBR3, MDA MB453, MDA MB435 and MDA MB468 cells at E:T=10:1 with 100 pM of the corresponding switches for 24 h. T cell activation was evaluated by flow cytometry with staining for CD69 and CD25 (FIG. 94A) redirected sCAR-T cells. IL-2 (FIG. 94B), IFN-γ (FIG. 94C) and TNF-α (FIG. 94D) level from the incubation medium was measured by ELISA kit. ***=p<0.005, **=p<0.05 and NS=p>0.05 were calculated using the Student's t-test, and a p value<0.05 was considered significant.

FIG. 95 shows in vitro cytotoxicity comparisons of sCAR-T cells harboring the CD8 or IgG4m hinges and bivalent switches. FITC-specific sCAR-T cells containing different hinges were incubated with SKBR3 (Her2 3+), MDA MB453 (Her2 2+), MDA MB435 (Her2 1+) or MDA MB468 (Her2 0) cancer cells at an E:T=10:1 ratio with serial dilution of FITC switch LS202X/HK136X. The cytolytic activity was determined after 24 hr by measuring the amount of lactate dehydrogenase (LDH) released into cultured media.

FIG. 96 shows in vitro cytotoxicity comparison of different FITC-based switches with anti-FITC CAR-T (CD8 hinge) cells against Her2 1+ cancer cells. The cytolytic activity was determined by measuring the amount of LDH released into culture medium

FIG. 97 shows the in vitro cytotoxicity comparison of different hinge length sCAR-T cells on Her2 1+ BT20 and MDA MB231 cancer cells. Different concentrations of FITC LS202X/HK136X conjugate were used to induce the corresponding CAR-T activity on target cells. The cytolytic activity was determined by measuring the amount of LDH released into cultured medium.

FIG. 98 shows correlations of conjugation sites and CAR hinge design with FITC-based switches. The location of LS202X/HK136X site and relative distances between bivalent sites were calculated by UCSF chimera 1.10.2. The structures are derived from a reported crystal structure (Protein Data Bank ID 1N8Z)

FIG. 99 shows optical imaging with IRDye800 labeled anti-Her2 Fab in MDA MB435/Her2 tumor-bearing mice. (Top) 5×10⁶ tumor cells were subcutaneous implanted in the right flank of female NSG mice. When tumor reached 500mm³, bioluminescence imaging of tumors was taken after intraperitoneal injection of D-luciferin. (Bottom) Whole-body fluorescence imaging of mice after intravenous administration of IRDye800 labeled Fab at 1 nmol. Images were acquired by IVIS imaging at the indicated time point after injection of Fab. Arrows indicate the location of implanted tumor.

### DETAILED DESCRIPTION OF THE INVENTION

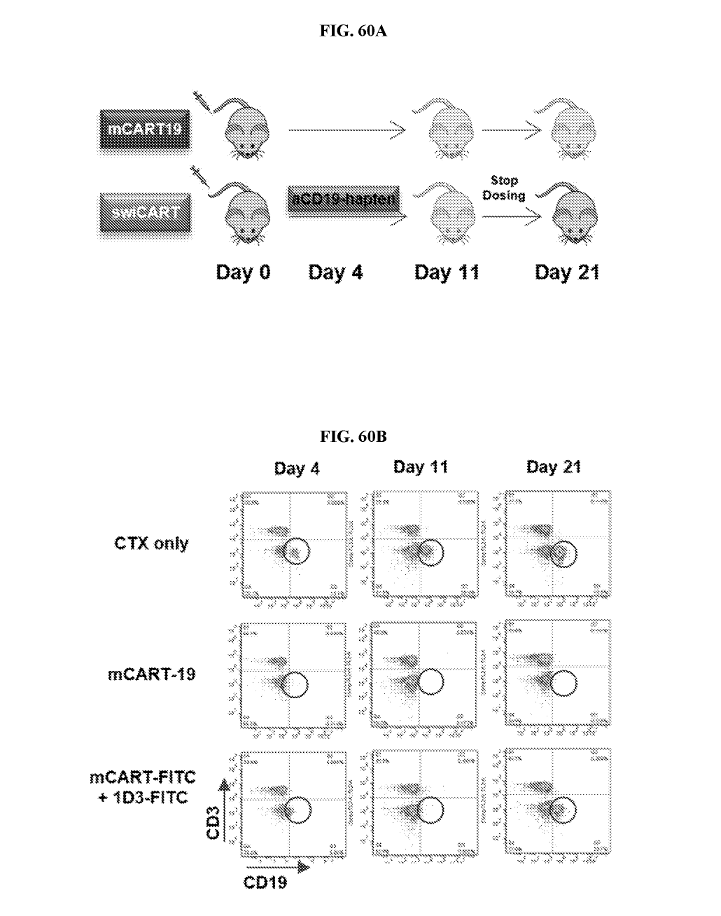

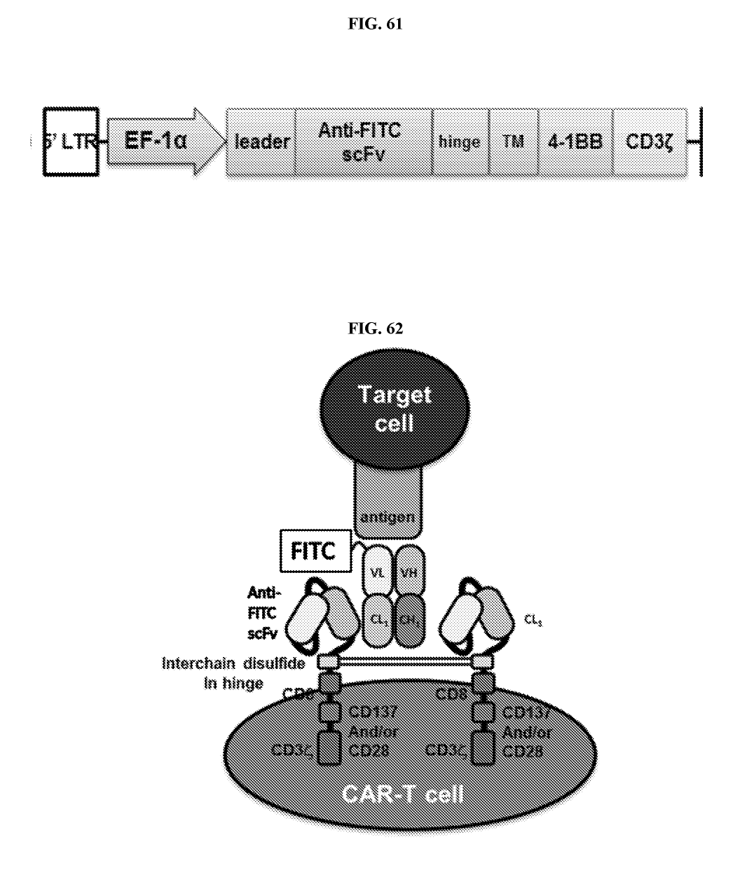

Current chimeric antigen receptor T cell (CAR T-cell) therapies can be unreliable due to lack of means to control CAR T-cell activity. Disclosed herein are compositions and methods for selectively activating and deactivating CAR T-cells, which may provide for safer and more versatile immunotherapies than those currently being tested and administered. Disclosed herein are switchable chimeric antigen receptor effector cells (sCAR-ECs) and chimeric antigen receptor effector cell (CAR-EC) switches, wherein the CAR-EC switches have a first region that is bound by a chimeric antigen receptor on the CAR-EC and a second region that binds a cell surface molecule on target cell, bringing the target cell in proximity of the CAR-EC and stimulating an immune response from the CAR-EC that is cytotoxic to the bound target cell. In general, the CAR-EC is a T cell, and the CAR-EC is referred to as a switchable CAR-T cell (sCAR-T cell). In this way, the sCAR-EC switch may act as an “on-switch” for CAR-EC activity. Activity may be “turned off” by reducing or ceasing administration of the switch or adding a switch component that competes with the switch. These methods and compositions disclosed herein allow for the site-specific incorporation of an unnatural amino acid at one or more desired sites of the target interacting domain (TID) that binds the cell surface molecule and subsequent site-specific modification of antibody with FITC via click chemistry, which enables establishment of the most productive pseudo-immunological synapse between engineered T cells and target cells by precisely adjusting the sites and stoichiometry of FITC conjugation (e.g., FIG. 59).

Major advantages of the switchable CAR platforms disclosed herein include control, safety, titratability, and universality. Switchable CARs can be turned on and off with addition and cessation or competition of the switch. In addition, CAR-EC switches can be titrated to a desired response. For example, solid tumors may be targeted by titration of therapy to achieve a suitable therapeutic index. The response may be titrated “on” to avoid cytokine release syndrome (CRS) and tumor lysis syndrome (TLS) events, providing for personalized therapy. Furthermore, administration of a switch can be terminated in case of an adverse event. The sCAR-EC can be designed to target a non-endogenous antigen which is only active in the presence of a switch that can be reduced at any time. In contrast, a canonical CAR-T cell is always “on” as long as a target exists. This always “on” can lead to T cell anergy as exemplified by functionally exhausted CD8 T cells during chronic viral infection. In contrast, a sCAR-EC can be stimulated and rested. This is more analogous to the natural stimulation of a T cell responding to an infection. Iterative stimulations in this nature, if timed corrected, may be able to better recapitulate the natural stimulation and resting cycles of T cells that would be encountered, for example, with an acute infection. This type of natural life expansion and contraction of T cells may off-set anergy (or T cell dysfunction) and promote the formation of long-lived memory cells. Long-lived memory cells are known to be advantageous in CAR-T cells. Therefore, a switchable approach to CAR-T cells may be advantageous in that it can promote more favorable T cell responses and phenotypes than canonical CAR-T cells.

Another advantage of the sCAR-EC system is that it is easier and faster to design multiple switches for each CAR-EC rather than empirically building and testing CAR hinge designs. This is because the switches are biologics which may be easier, less expensive, and faster to build multiple variants of than the CAR which requires cell engineering and cell handling. Further, a universal sCAR-EC has a significant advantage in design of the optimal immunological synapse, as CAR-EC switches make it possible for a single CAR-EC to be redirected to multiple therapeutic targets. Redirection during therapy, by variation of switches, can combat antigen-loss escape mutants with a single CAR-EC. Treatment of heterologous tumors with multiple switches is more straightforward than with multiple CARs. Switches also enable standardized treatment protocols which may increase safety and lower up front treatment costs.

Each target antigen and epitope requires empirical design of a canonical CAR in order to achieve optimal CAR-T cell activation, but a switch enables more geometric options than a canonical CAR, allowing for optimal geometry of the immunological synapse. Therefore, an advantage of sCAR-ECs is the additional flexibility in geometric orientations that can be provided by a switch that cannot be provided by modifying the CAR hinge alone. The additional geometric orientations may be useful in forming optimal immunological synapses. Switch designs that provide maximal ternary complex formation may correlate with increased sCAR-EC activity. On the other hand, improper balancing of kinetics, as a result of sub-optimal design, can result in auto-inhibition of the ternary complex formation, translating to a decrease in the number of immunological synapses that can form between the sCAR-EC and target cell, which may be detrimental to activity. States of auto-inhibition may result in increased activation induced cell death (AICD) through sub-optimal signaling. Recursive CAR signaling can also result in AICD. Other sCAR-T cell platforms with non-specific labeling of antibodies or constrained site labeling are unable to comprehensively explore these design considerations.

Mathematical models related to binary binding equilibria and concentration (which is related to antigen density) of each component to the formation of the ternary complex as a function of switch concentration are considered in optimizing the sCAR-EC immunological synapse. These models take into account auto-inhibition of the ternary complex by high concentrations of the switch or disproportionately high affinities of the binary interactions, which may reduce the cytotoxic capacity of the sCAR-EC cells. In order to apply these models, sCARs and switches with varied affinities are produced by specific mutations, grafting/fusion sites and quantitative flow cytometry is used to establish sCAR density. The increased avidity of the IgG based switch, relative to a Fab switch, enables a larger range of off-rates to be studied. Target cells with a range of cell surface antigen density are also employed. Candidate designs are tested for cytotoxicity of target cells, cytokine release, AICD and up-regulation of activation markers on sCAR-EC cells. Optimal ternary complex and immunological synapse formation may be achieved when the affinity of the sCAR is relatively low and the concentration of sCAR on the surface of the T cell is relatively high. Alternatively, optimal ternary complex and immunological synapse formation may be achieved when the affinity of the sCAR is relatively high and the concentration of sCAR on the surface of the T cell is relatively low.

The examples disclosed herein demonstrate that changes in the length of the hinge region of the sCAR can be matched with compensatory switch modifications (e.g., geometry) to yields switch-sCAR pairs that exhibit robust cytotoxicity, upregulation of activation markers, and production of cytokines. For example, optimal configurations of switches and sCAR hinges afforded high potency on breast cancer cells expressing high (3+) mid (2+) and low (1+) levels of herceptin (Her2) expression with no activity on Her2 negative (0) cell lines. This is shown with at least two switches that differ by their CAR binding region. These platforms eliminated established Her2 3+, 2+ and 1+ solid and orthotopic tumor xenografts with comparable efficacy to the conventional anti-Her2 CAR-T cells. This controllable nature of sCAR-T cells is expected to provide increased safety in the translation of CAR-T cell strategies for solid tumors, especially in the treatment of patients with Her2-low malignancies for which trastuzumab is not approved.

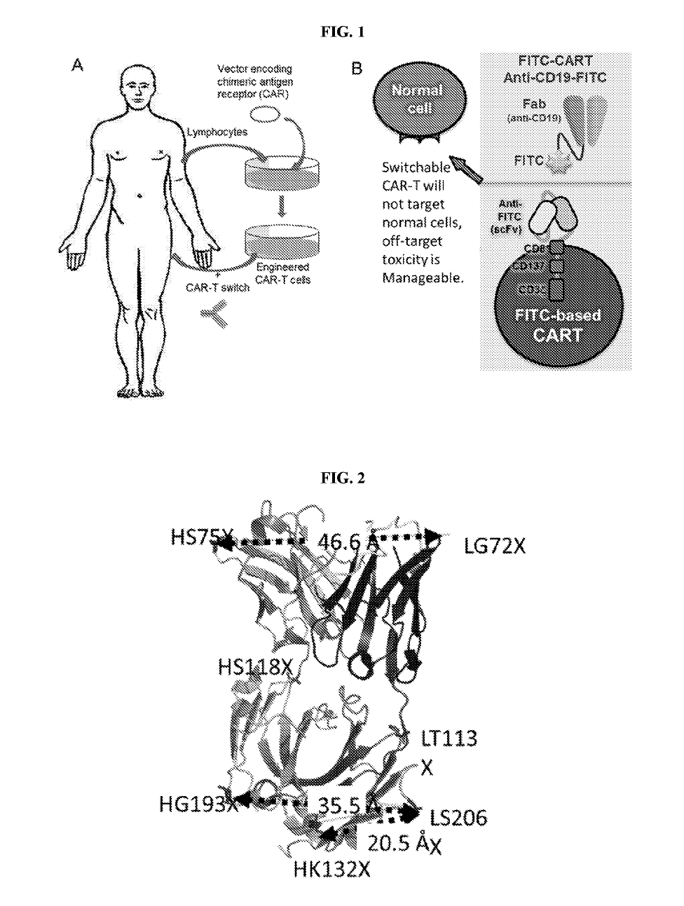

These CAR-EC switches may be used with sCAR-ECs disclosed herein, as well as existing CAR T-cells for the treatment of a disease or condition, such as cancer, wherein the target cell is a malignant cell. Such treatment may be referred to herein as switchable immunotherapy, for which an exemplary schematic overview is depicted in FIG. 1. CAR-ECs and respective switches disclosed herein may be particularly useful for the treatment of acute myeloid leukemia (AML). Despite decades of clinical efforts and research, AML is still considered incurable and the five-year survival rate is less than 30% for adult patients. Induction chemotherapy alone inevitably results in relapse within a median time frame of 4 to 8 months, which necessitates prolonged post-remission treatment with either consolidation chemotherapy or allogeneic hematopoietic cell transplantation (HCT). Relapse of AML is believed to be related to the small population of so-called leukemic stem cells (LSC), which are relatively quiescent and resistant to conventional chemotherapy, and are capable of self-renewal and regeneration into rapidly proliferating blasts. Therefore, therapeutic approaches that are effective against both blasts and LSCs are highly desirable to advance the overall survival rate for AML. Endogenous T cells in AML patients are often functionally suppressed, which may limit the use of other cancer immunotherapeutics, such as bispecific T cells engagers (e.g. BiTE), in AML patients. Conventional CD19-targeting CAR-T cells show long-term persistence in targeting normal B cells after tumor clearance, causing irreversible B-cell aplasia in recent clinical trials, making use of either of these T cell therapies sub-optimal. Targeting other known AML-associated myeloid antigens, such as C-Type Lectin-like Molecule-1 (CLL1), CD123, and CD33, poses a greater risk of adverse side effects due to chronic myelosuppression because the expression of these antigens are also ubiquitous on most normal myeloid cells and other hematopoietic compartments. Furthermore, it is likely that conventional CAR-T therapy targeting a single AML antigen will also be highly susceptible to the relapse of escape variants as the immunophenotypic heterogeneity of AML is well known. Therefore, it is of great importance to develop a general method to regulate the activity and specificity of CAR-T cells in order to safely apply the CAR-T therapy for prolonged remission and survival of AML patients.

Disclosed herein are switches for use in regulating the activity of a CAR-EC. Generally, the switch comprises (a) a chimeric antigen receptor-interacting domain (CAR-ID); and (b) a target interacting domain (TID). The switch may further comprise one or more linkers. The TID may be based on or derived from a polypeptide. The TID may comprise an antibody or antibody fragment. The TID may be modified to comprise one or more unnatural amino acids. Alternatively, or additionally, the TID may comprise a small molecule. The CAR-ID may comprise a small molecule. The CAR-ID may comprise a hapten.

Disclosed herein are switches for regulating the activity of a CAR-EC, the switch comprising (a) a CAR-ID that interacts with a CAR on the sCAR-EC; and (b) a TID comprising an unnatural amino acid, wherein the TID interacts with a surface molecule on a target cell. The CAR-ID and TID may be attached/connected through the unnatural amino acid.

Disclosed herein are compositions comprising a plurality of switches for regulating the activity of a CAR-EC, wherein a switch of the plurality of switches comprises (a) a CAR-ID that interacts with a CAR on the sCAR-EC; and (b) a TID comprising a polypeptide, wherein the CAR-ID is attached to the same amino acid residue of the TID in at least 60% of the switches.

Methods of producing the switches and switch intermediates disclosed herein may advantageously provide for control of sCAR-EC cell activity, titration of off-target reactivity, abrogation of TLS, attenuation of CRS, and/or optimization of CAR-EC switch binding by affinity, valency, geometry, linker length and/or linker chemistry through site-specific conjugation of CAR-EC switch components/regions.

Disclosed herein are methods of producing a switch for regulating the activity of a CAR-EC. The method may comprise (a) obtaining a TID comprising an unnatural amino acid; and (b) attaching a CAR-ID to the TID, thereby producing the switch.

Further disclosed herein are methods of producing a switch for regulating the activity of a CAR-EC comprising (a) contacting a CAR-ID with a TID; and (b) producing the switch by attaching the CAR-ID to a predetermined site on the TID.

Further disclosed herein are methods of producing a switch for regulating the activity of a CAR-EC comprising (a) contacting a plurality of CAR-IDs with a plurality of TIDs; and (b) attaching one or more CAR-IDs of the plurality of CAR-IDs to one or more TIDs of the plurality of TIDs, thereby producing a plurality of switches, wherein at least about 60% of the switches are structurally homologous.

Further disclosed herein are methods of producing a switch for regulating the activity of a CAR-EC comprising (a) contacting a plurality of CAR-IDs with a plurality of TIDs; and (b) attaching a CAR-ID of the plurality of CAR-IDs to a TID of the plurality of TIDs, thereby producing a plurality of switches, wherein the CAR-ID is attached to the same amino acid residue of the TID in at least 60% of the switches.

Further disclosed herein are methods of producing a switch of Formula IV: X-L1-L2-Y or Formula IVA: Y-L2-L1-X comprising (a) coupling L1 to X to produce a first intermediate of Formula IIA: L1-X, wherein: i. X comprises a chimeric antigen receptor-interacting domain (CAR-ID) that interacts with a CAR on an effector cell; and ii. L1 comprises a first linker before being coupled to X; (b) coupling L2 to Y to produce a second intermediate of Formula VA: Y-L2, wherein: i. Y comprises a TID that interacts with a surface molecule on a target cell; and ii. L2 comprises a second linker before being coupled to X; and (c) linking the first intermediate to the second intermediate, thereby producing the switch of Formula IV (X-L1-L2-Y) or Formula IVA (Y-L2-L1-X).

Disclosed herein are switch intermediates. The switch intermediate may comprise (a) a CAR-ID comprising a small molecule, wherein the CAR-ID interacts with a CAR on the CAR-EC; and (b) a linker connected to the CAR-ID, wherein the linker does not comprise a region that interacts with the CAR-EC and the linker does not comprise a region that interacts with a surface molecule on a target cell.

Further disclosed herein is a switch intermediate comprising (a) a CAR-ID comprising a small molecule, wherein the CAR-ID interacts with a CAR on the CAR-EC; and (b) a linker connected to the CAR-ID, wherein the linker comprises an aminooxy group, azide group and/or cyclooctyne group at one or more termini.

Further disclosed herein is a switch intermediate comprising (a) a TID comprising an unnatural amino acid, wherein the TID interacts with a surface molecule on a target cell; and (b) a linker connected to the TID, wherein the linker does not comprise a region that directly interacts with the CAR-EC and the linker does not comprise a region that directly interacts with the target cell.

Further disclosed herein is a switch intermediate comprising (a) a TID comprising a polypeptide or a small molecule, wherein the TID interacts with a surface molecule on a target cell; and (b) a linker connected to the TID, wherein the linker comprises an aminooxy group, azide group and/or cyclooctyne group at one or more termini.

Further disclosed herein are methods of producing a switch intermediate for regulating the activity of a CAR-EC comprising (a) contacting a TID with a linker, the linker comprising an aminooxy group, azide group and/or cyclooctyne group at one or more termini; and (b) attaching the linker to the TID, thereby producing the switch intermediate.

Further disclosed herein are methods of producing a switch intermediate for regulating the activity of a CAR-EC comprising (a) contacting a CAR-ID with a linker, the linker comprising an aminooxy group, azide group and/or cyclooctyne group at one or more termini; and (b) attaching the linker to the CAR-ID, thereby producing the switch intermediate.

Further disclosed herein are universal CAR-EC platforms. The CAR-EC platforms may comprise one or more CAR-EC switches, CAR-ECs, CAR-EC intermediates, and linkers. The CAR-EC may comprise a CAR comprising an ultra-high affinity antibody or antibody fragment (e.g. scFv) to the switch.

Methods of treating a disease or condition comprising administering the CAR-EC switches, disclosed herein, may provide for a titratable response, improved safety and/or cessation of CAR-EC activity by reducing or ceasing administration of the CAR-EC switch. In contrast to other approaches of controlling CAR-EC activity, which “turn off” CAR-EC activity by competing with the target cell surface molecule for binding the CAR, the CAR-EC switches disclosed herein, generally function as CAR-EC activators or “on” switches.

Further disclosed herein are switchable CAR-EC (sCAR-EC) platforms including CAR-EC switches and effector cells comprising universal CAR that can bind multiple CAR-EC switches, providing for sequential targeting of one or more types of target cells (e.g. treatment of heterogeneous tumors). Unless otherwise note, “sCAR-EC” and “CAR-EC” are used interchangeably and may refer to a sCAR-EC. The CAR may comprise an ultra-high affinity antibody or antibody fragment (e.g. scFv) to the switch. Methods of producing the CAR-EC switches disclosed herein may advantageously provide for control of CAR-EC cell activity, titration of off-target reactivity, abrogation of TLS, attenuation of CRS, and/or optimization of CAR-EC switch binding by affinity, valency, geometry, length and/or chemistry through site-specific attachment of the TID and CAR-ID.

Disclosed herein are methods of selecting an optimal sCAR platform, comprising: attaching a first CAR-ID to a first site of a TID that binds a first cell surface molecule on a first target cell to produce a first switch; attaching a second CAR-ID to a second site of a second TID that binds a second cell surface molecule on a second target cell to produce a second switch; contacting the first target cell with the first switch and a first CAR-EC expressing a first CAR; contacting the second target cell with the second switch and a second CAR-EC expressing a second CAR; and comparing a first cytotoxic effect of the first switch and the first CAR-EC on the first target cell to a second cytotoxic effect of the second switch and the second CAR-EC on the second target cell; and selecting the first switch and first CAR-EC or the second switch and the second CAR-EC as the optimal sCAR platform based on comparing the first cytotoxic effect to the second cytotoxic effect. The first CAR-ID and the second CAR-ID may be the same. The first TID and the second TID may be the same. The first site and the second site may be different. The first site and the second site may be the same. Unless otherwise noted, “sCAR platform”, “sCAR-EC platform”, and “CAR-EC” platform are used interchangeably and may refer to an optimal switchable CAR platform.

Further disclosed herein are CARs comprising an extracellular domain, a transmembrane domain, and an intracellular signaling domain, wherein the extracellular domain comprises: a region that interacts with a CAR switch; and a hinge domain. The CAR switch may comprise a hapten. The hapten may be FITC or a derivative thereof. The region that interacts with a CAR switch may be an anti-FITC antibody or antibody fragment. Unless otherwise noted, CAR and switchable CAR (sCAR) are used interchangeably and may refer to a sCAR.

Disclosed herein are sTCR switches comprising: a CAR-ID; and a sTCR or portion thereof. The CAR-ID may be a small molecule. The CAR-ID may be a non-peptidic molecule. The CAR-ID may be a hapten. The hapten may be FITC or a derivative thereof.

Unless otherwise specified, the terms “switch” and “CAR-EC switch”, as used herein, are used interchangeably and may refer to a FITC switch. The TID of the switch may comprise an antibody portion. The antibody portion of the switch may comprise at least a portion of an antibody or an entire antibody. For example, the antibody portion of the switch may comprise at least a portion of a heavy chain, a portion of a light chain, a portion of a variable region, a portion of a constant region, a portion of a complementarity determining region (CDR), or a combination thereof. The antibody portion of the switch may comprise at least a portion of the Fc (fragment, crystallizable) region. The antibody portion of the switch may comprise at least a portion of the complementarity determining region (e.g., CDR1, CDR2, CDR3). The antibody portion of the switch may comprise at least a portion of the Fab (fragment, antigen-binding) region.

Before the present methods, kits and compositions are described in greater detail, it is to be understood that this invention is not limited to particular method, kit or composition described, as such may, of course, vary. It is also to be understood that the terminology used herein is for the purpose of describing particular embodiments only, and is not intended to be limiting, since the scope of the present invention will be limited only by the appended claims. Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods, kits and compositions are provided for producing sCAR-EC platforms and CAR-EC switches used to bring an effector cell together with a target in a subject. These methods, kits and compositions find therapeutic use in a number of diseases. For example, heterogeneous tumors and blood cell malignancies (e.g. AML and CLL) may be more effectively treated with a CAR-EC platform when the length, valency and orientation of the CAR-EC switch linkage as well as the CAR-EC switch cell targeting moiety is optimized. Heterogeneous tumors may be more effectively treated with multiple CAR-EC switches that target more than one tumor antigens. Advantages and features of the invention will become apparent to those persons skilled in the art upon reading the details of the compositions and methods as more fully described below.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### CAR-EC Switch

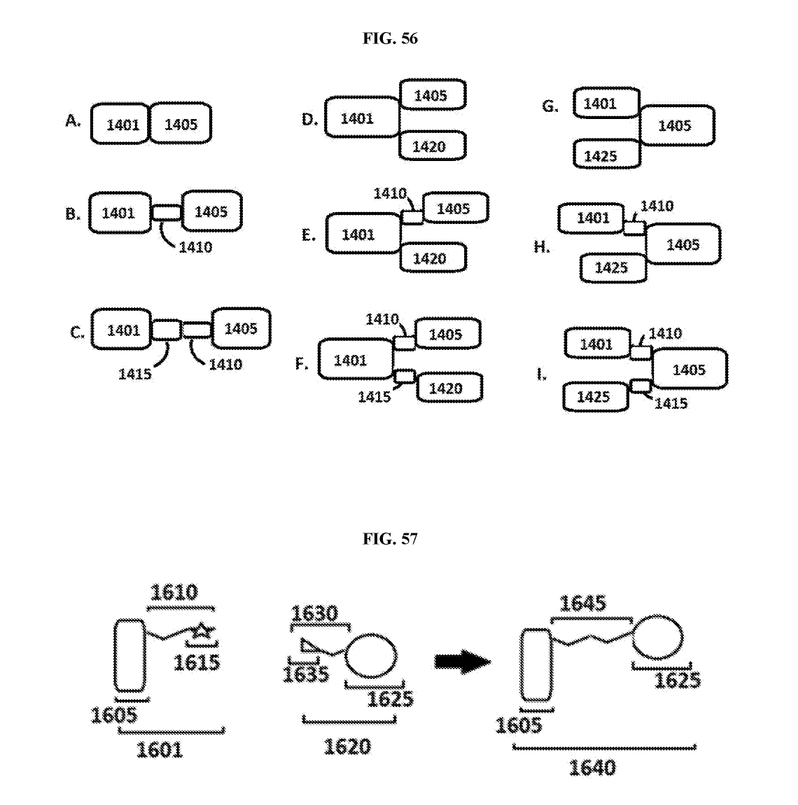

Disclosed herein are switches (e.g., chimeric antigen receptor-effector cell switches, CAR-EC switches), methods of producing such switches, and uses thereof. Generally, a switch may comprise (a) a chimeric antigen receptor-interacting domain (CAR-ID); and (b) a target interacting domain (TID). The switch may further comprise one or more additional CAR-IDs. The switch may further comprise one or more additional TIDs. The switch may further comprise one or more linkers. FIG. 56A-I depict exemplary switches. As shown in FIG. 56A, a switch may comprise a CAR-ID (1401) attached to a TID (1405). As shown in FIG. 56B, a switch may comprise a CAR-ID (1401), a linker (1410) and a TID (1405). The linker (1410) may attach the CAR-ID (1401) to the TID (1405). As shown in FIG. 56C, a switch may comprise a CAR-ID (1401), a first linker (1415), a second linker (1410) and a TID (1405). The first linker (1415) and second linker (1410) may be connected to each other. In addition, the first linker (1415) may be attached to the CAR-ID (1401) and the second linker (1410) may be attached to the TID (1405), thereby resulting in attachment of the CAR-ID (1401) to the TID (1405). The first linker (1410) and the second linker (1415) may be the same. The first linker (1410) and the second linker (1415) may be different.

The switch may comprise a CAR-ID and two or more TIDs. As shown in FIG. 56D, a switch may comprise a CAR-ID (1401), a first TID (1405), and a second TID (1420). The first TID (1405) and the second TID (1420) may be attached to the CAR-ID (1401). As shown in FIG. 56E, a switch may comprise a CAR-ID (1401), a linker (1410), a first TID (1405), and a second TID (1420). The linker (1410) may attach the first TID (1405) to the CAR-ID (1401). The second TID (1420) may be attached to the CAR-ID (1401). As shown in FIG. 56F, a switch may comprise a CAR-ID (1401), a first linker (1410), a second linker (1415), a first TID (1405), and a second TID (1420). The first linker (1410) may attach the first TID (1405) to the CAR-ID (1401). The second linker (1415) may attach the second TID (1420) to the CAR-ID (1401). The first TID (1405) and a second TID (1420) may be the same. The first TID (1405) and a second TID (1420) may be different. The first linker (1410) and the second linker (1415) may be the same. The first linker (1410) and the second linker (1415) may be different. The switch may further comprise one or more additional CAR-IDs. The switch may further comprise one or more additional TIDs. The switch may further comprise one or more linkers.

The switch may comprise a TID and two or more CAR-IDs. As shown in FIG. 56G, a switch may comprise a TID (1405), a first CAR-ID (1401), and a second CAR-ID (1425). The first CAR-ID (1401) and the second CAR-ID (1425) may be attached to the TID (1405). As shown in FIG. 56H, a switch may comprise a TID (1405), a linker (1410), a first CAR-ID (1401), and a second CAR-ID (1425). The linker (1410) may attach the first CAR-ID (1401) to the TID (1405). The second CAR-ID (1425) may be attached to the TID (1405). As shown in FIG. 561, a switch may comprise a TID (1405), a first linker (1410), a second linker (1415), a first CAR-ID (1401), and a second CAR-ID (1425). The first linker (1410) may attach the first CAR-ID (1401) to the TID (1405). The second linker (1415) may attach the second CAR-ID (1425) to the TID (1405). The first CAR-ID (1401) and the second CAR-ID (1425) may be the same. The first CAR-ID (1401) and the second CAR-ID (1425) may be different. The first linker (1410) and the second linker (1415) may be the same. The first linker (1410) and the second linker (1415) may be different. The switch may further comprise one or more additional CAR-IDs. The switch may further comprise one or more additional TIDs. The switch may further comprise one or more linkers.

The CAR-ID may be attached to the TID. Attachment of the CAR-ID to the TID may occur by any method known in the art. For example, the CAR-ID may be attached to the TID by fusion, insertion, grafting, or conjugation. The CAR-ID may be fused to the TID. The CAR-ID may be inserted into the TID. The CAR-ID may be conjugated to the TID. The CAR-ID may be linked to the TID.

A switch may comprise (a) a chimeric antigen receptor-interacting domain (CAR-ID); and (b) a target interacting domain (TID). The CAR-ID may comprise FITC. Switches that comprise a CAR-ID comprising a hapten and a TID comprising a small molecule may be referred to as hapten-small molecule switches. Switches that comprise a CAR-ID comprising a hapten and a TID comprising an antibody or antibody fragment may be referred to as hapten-antibody switches.

A switch may comprise a chimeric antigen receptor-interacting domain (CAR-ID); and (b) a target interacting domain (TID). The CAR-ID may interact with a chimeric antigen receptor (CAR) on an effector cell. The TID may interact with a surface molecule on a target. The TID may comprise an unnatural amino acid. A TID may comprise a polypeptide that is based on or derived from an antibody or antibody fragment. The antibody or antibody fragment may be modified to contain one or more unnatural amino acids. The CAR-ID may be attached to the TID. The CAR-ID may be site-specifically attached to the TID. The CAR-ID may be site-specifically attached to the unnatural amino acid in the TID. Switches that comprise a CAR-ID comprising a small molecule and a TID comprising an antibody or antibody fragment may be referred to as small molecule-antibody switches. The CAR-ID may be fused to the TID. The CAR-ID may be inserted into the TID. The TID may be inserted into the CAR-ID. The CAR-EC switch may further comprise one or more linkers. The one or more linkers may attach the CAR-ID to the TID. The CAR-EC switch may further comprise one or more unnatural amino acids. The CAR-ID may comprise one or more unnatural amino acids. The TID may comprise one or more unnatural amino acids. The CAR-ID and the TID may comprise one or more unnatural amino acids. The CAR-ID may be attached to the TID via the one or more unnatural amino acids in the CAR-ID. The CAR-ID may be attached to the TID via the one or more unnatural amino acids in the TID. The CAR-ID may be attached to the TID via the one or more unnatural amino acids in the CAR-ID and one or more unnatural amino acids in the TID.

### Chimeric Antigen Receptor Interacting Domain (CAR-ID)

The switches disclosed herein may comprise one or more chimeric antigen receptor-interacting domains (CAR-IDs). The switches disclosed herein may comprise two or more CAR-IDs. The switches disclosed herein may comprise three or more CAR-IDs. The switches disclosed herein may comprise four or more CAR-IDs. The switches disclosed herein may comprise 5, 6, 7, 8, 9, 10 or more CAR-IDs. The two or more CAR-IDs may be the same. At least two of the three or more CAR-IDs may be the same. The two or more CAR-IDs may be different. At least two of the three or more CAR-IDs may be different. The number of CAR-IDs may be optimized for safety and efficacy. For example, one or two CAR-IDs per TID may yield efficient CAR-EC activation while three or four CAR-IDs per TID may result in nonspecific activation of the CAR-EC may result in nonspecific activation of the CAR-EC.

The CAR-ID may be a naturally-occurring molecule. The CAR-ID may be an artificial or synthetic molecule. At least a portion of a CAR-ID may be synthetic. The CAR-ID may comprise a polypeptide that is not naturally occurring. The CAR-ID may be an organic molecule. The CAR-ID may be inorganic molecule.

The CAR-ID may be a small molecule. The small molecule may be an organic compound. The small molecule may have a size on the order of about 10⁻⁸ m, about 10⁻⁹ m, about 10⁻¹⁰ m. The small molecule may have a size of less than about 10⁻⁷ m. The small molecule may have a size of less than about 10⁻⁸ m. The small molecule may have a size of less than about 10⁻⁹ m. The small molecule may have a size of less than about 10⁻¹⁰ m. The small molecule may have a size of less than about 10⁻¹¹ m. The small molecule may have a mass of less than about 5000 Da, less than about 4500 Da, less than about 4000 Da, less than about 3500 Da, less than about 3000 Da, less than about 2500 Da, less than about 2000 Da, less than about 1500 Da, less than about 1000 Da, less than about 900 D, less than about 500 Da or less than about 100 Da. In some instances, the small molecule does not comprise a polypeptide. In some instances, the small molecule does comprise two or more amino acids that are linked by an amide bond. The small molecule may be a chemical compound.

The CAR-ID may be selected from DOTA, dinitrophenol, quinone, biotin, aniline, atrazine, an aniline-derivative, o-aminobenzoic acid, p-aminobenzoic acid, m-aminobenzoic acid, hydralazine, halothane, digoxigenin, benzene arsonate, lactose, trinitrophenol, biotin, FITC, or a derivative thereof. The CAR-ID may be a quinone or a derivative thereof. The CAR-ID may be DOTA or a derivative thereof. The CAR-ID may be dinitrophenol or a derivative thereof. The CAR-ID may be biotin or a derivative thereof. The CAR-ID may comprise a hapten. The CAR-ID may induce an immune response when attached to a larger carrier molecule, such as a protein, antibody or antibody fragment. The CAR-ID may be FITC or a derivative thereof. The CAR-ID may comprise biotin. The CAR-ID may comprise dinitrophenol.

Alternatively, the CAR-ID does not comprise a hapten. The CAR-ID may be selected from a steroid, a vitamin, a vitamer, a metabolite, an antibiotic, a monosaccharide, a disaccharide, a lipid, a fatty acid, a nucleic acid, an alkaloid, a glycoside, a phenzine, a polyketide, a terpene, and a tetrapyrrole, and portions thereof, and combinations thereof. The CAR-ID may be a penicillin drug or a derivative thereof.

The CAR-ID may be linked and/or conjugated to the target interacting domain. The target interacting domain may be a targeting antibody or antibody fragment and the CAR-ID may be linked and/or conjugated to an amino acid of the targeting antibody or antibody fragment. The amino acid of the targeting antibody or antibody fragment may be an unnatural amino acid. The targeting antibody or antibody fragment may comprise a light chain and/or heavy chain selected from SEQ ID NOS: 10-31 and the unnatural amino acids may be located at respective sites shown in Table 1. Unless otherwise noted, amino acids are counted from the amino acid of the N-terminus of each variable region to the C-terminus of the constant region.

TABLE 1

| Antigen | clone | A | B | C | D | E | F |
|---|---|---|---|---|---|---|---|
| CD19 | FMC63 | LG68 | HS74 | LT109 | HA121 | LS202 | HK136 |
| CD22 | hLL2 | LG74 | HS75 | LT114 | HA117 | LS207 | HK132 |
| | M971 | LG68 | HS78 | LT109 | HA125 | LS202 | HK140 |
| Her2 | Herceptin | LG68 | HS75 | LT109 | HA121 | LS202 | HK136 |
| CLL1 | 1075.7 | LG69 | HS75 | LA110 | HA124 | LS203 | HK139 |
| CD33 | hM195 | LG72 | HS75 | LT113 | HA117 | LS206 | HK132 |
| | Hp67.6 | LG72 | HP75 | LT113 | HA117 | LS206 | HK132 |
| CD123 | 26292 | LG68 | HS75 | LT109 | HA116 | LS202 | HK131 |
| | 32716 | LR72 | HS75 | LT113 | HA119 | LS206 | HK134 |

L = light chain, H = heavy chain, S = serine, G = glycine, R = arginine, T = threonine, A = alanine and K = lysine

### Target Interacting Domain (TID)

The switches disclosed herein may comprise one or more TIDs. The switches disclosed herein may comprise two or more TIDs. The switches disclosed herein may comprise three or more TIDs. The switches disclosed herein may comprise four or more TIDs. The switches disclosed herein may comprise 5, 6, 7, 8, 9, 10 or more TIDs. The two or more TIDs may be the same. At least two of the three or more TIDs may be the same. The two or more TIDs may be different. At least two of the three or more TIDs may be different.

The switch intermediates disclosed herein may comprise one or more TIDs. The switch intermediates disclosed herein may comprise two or more TIDs. The switch intermediates disclosed herein may comprise three or more TIDs. The switch intermediates disclosed herein may comprise four or more TIDs. The switch intermediates disclosed herein may comprise 5, 6, 7, 8, 9, 10 or more TIDs. The two or more TIDs may be the same. At least two of the three or more TIDs may be the same. The two or more TIDs may be different. At least two of the three or more TIDs may be different.

The TID may bind to a cell surface molecule on a target. The cell surface molecule may comprise an antigen. The cell surface molecule may be selected from a protein, a lipid moiety, a glycoprotein, a glycolipid, a carbohydrate, a polysaccharide, a nucleic acid, an MHC-bound peptide, or a combination thereof. The cell surface molecule may comprise parts (e.g., coats, capsules, cell walls, flagella, fimbrae, and toxins) of bacteria, viruses, and other microorganisms. The cell surface molecule may be expressed by the target cell. The cell surface molecule may not be expressed by the target cell. By way of non-limiting example, the cell surface molecule may be a ligand expressed by a cell that is not the target cell and that is bound to the target cell or a cell surface molecule of the target cell. Also, by non-limiting example, the cell surface molecule may be a toxin, exogenous molecule or viral protein that is bound to a cell surface or cell surface receptor of the target cell. The cell surface molecule may be a tumor associated antigen (TAA). The cell surface molecule may be a cancer cell associated antigen. The cancer may be AML. The cancer cell associated antigen may be selected from CD19, CD22, Her2, CLL1, CD33, CD123, BCMA, CS1, EGFR, EGFRVIII, CD20, and CEA.

The TID may be a targeting antibody or antibody fragment. The targeting antibody or antibody fragment may be an immunoglobulin (Ig). The Ig may selected from an IgG, an IgA, an IgD, an IgE, an IgM, a fragment thereof or a modification thereof. The Ig may be IgG. The IgG may be IgG1. The IgG may be IgG2. The IgG may have one or more Fc mutations for modulating endogenous T cell FcR binding to the CAR-EC switch. The IgG may have one or more Fc mutations for removing the Fc binding capacity to the FcR of FcR-positive cells. Removal of the Fc binding capacity may reduce the opportunity for crosslinking of the CAR-EC to FcR positive cells, wherein crosslinking of the CAR-EC to FcR positive cells would activate the CAR-EC in the absence of the target cell. As such, modulating the endogenous T cell FcR binding to the CAR-EC switch may reduce an ineffective or undesirable immune response. The one or more Fc mutations may remove a glycosylation site. The one or more Fc mutations may be selected from E233P, L234V, L235A, delG236, A327G, A330S, P331S, N297Q and any combination thereof. The one or more Fc mutations may be in IgG1. The one or more Fc mutations in the IgG1 may be L234A, L235A, or both. Alternatively, or additionally, the one or more Fc mutations in the IgG1 may be L234A, L235E, or both. Alternatively, or additionally, the one or more Fc mutations in the IgG1 may be N297A. Alternatively, or additionally, the one or more mutations may be in IgG2. The one or more Fc mutations in the IgG2 may be V234A, V237A, or both.

The targeting antibody or antibody fragment may be an Fc null Ig or a fragment thereof.

As used herein, the term “antibody fragment” refers to any form of an antibody other than the full-length form. Antibody fragments herein include antibodies that are smaller components that exist within full-length antibodies, and antibodies that have been engineered. Antibody fragments include, but are not limited to, Fv, Fc, Fab, and (Fab′)2, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, and bispecific antibodies. Unless specifically noted otherwise, statements and claims that use the term “antibody” or “antibodies” may specifically include “antibody fragment” and “antibody fragments.”

The targeting antibody fragment may be human, fully human, humanized, human engineered, non-human, and/or chimeric antibody. The non-human antibody may be humanized to reduce immunogenicity to humans, while retaining the specificity and affinity of the parental non-human antibody. Chimeric antibodies may refer to antibodies created through the joining of two or more antibody genes which originally encoded for separate antibodies. A chimeric antibody may comprise at least one amino acid from a first antibody and at least one amino acid from a second antibody, wherein the first and second antibodies are different. At least a portion of the antibody or antibody fragment may be from a bovine species, a human species, or a murine species. At least a portion of the antibody or antibody fragment may be from a rat, a goat, a guinea pig or a rabbit. At least a portion of the antibody or antibody fragment may be from a human. At least a portion of the antibody or antibody fragment antibody may be from cynomolgus monkey.

The targeting antibody or antibody fragment may be based on or derived from an antibody or antibody fragment from a mammal, bird, fish, amphibian, or reptile. Mammals include, but are not limited to, carnivores, rodents, elephants, marsupials, rabbits, bats, primates, seals, anteaters, cetaceans, odd-toed ungulates and even-toed ungulates. The mammal may be a human, non-human primate, mouse, sheep, cat, dog, cow, horse, goat, or pig.

The targeting antibody or an antibody fragment may target an antigen selected from, by non-limiting example, CD19, CD22, Her2, CLL1, CD33, CD123, BCMA, CS1, EGFR, EGFRVIII, CD20, and CEA or a fragment thereof.

The TID may comprise an anti-CS1 antibody or fragment thereof. The light chain of the anti-CS1 antibody or fragment thereof may comprise SEQ ID NO: 10 and optionally SEQ ID NO: 53 or a homologous amino acid sequence. The homologous amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 10 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 53 . The heavy chain of the anti-CS1 antibody or fragment thereof may comprise SEQ ID NO: 11 and optionally SEQ ID NO: 52 or a homologous amino acid sequence. The amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 11 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 53.

The TID may comprise an anti-Her2 antibody or fragment thereof. The light chain of the anti-Her2 antibody or fragment thereof may comprise SEQ ID NO: 12 and optionally SEQ ID NO: 53 or a homologous amino acid sequence. The homologous amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 12 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% to SEQ ID NO: 53 . The heavy chain of the anti-Her2 antibody or fragment thereof may comprise SEQ ID NO: 13 and optionally SEQ ID NO: 52 or a homologous amino acid sequence. The amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 13 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 52.

The TID may comprise an anti-BCMA antibody or fragment thereof. The light chain of the anti-BCMA antibody or fragment thereof may comprise SEQ ID NO: 14 and optionally SEQ ID NO: 53 or a homologous amino acid sequence. The homologous amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 14 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 53. The heavy chain of the anti-BCMA antibody or fragment thereof may comprise SEQ ID NO: 15 and optionally SEQ ID NO: 52 or a homologous amino acid sequence. The amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 15 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 52.

The TID may comprise an anti-CD19 antibody or fragment thereof. The light chain of the anti-CD19 antibody or fragment thereof may comprise SEQ ID NO: 16 and optionally SEQ ID NO: 53 or a homologous amino acid sequence. The homologous amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 16 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 53. The heavy chain of the anti-CD19 antibody or fragment thereof may comprise SEQ ID NO: 17 and optionally SEQ ID NO: 52 or a homologous amino acid sequence. The amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 17 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 52.

The TID may comprise an anti-CLL1 antibody or fragment thereof. The light chain of the anti-CLL1 antibody or fragment thereof may comprise SEQ ID NO: 18 and optionally SEQ ID NO: 53 or a homologous amino acid sequence. The homologous amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 18 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 53. The heavy chain of the anti-CLL1 antibody or fragment thereof may comprise SEQ ID NO: 19 and optionally SEQ ID NO: 52 or a homologous amino acid sequence. The amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 19 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 52.

The TID may comprise an anti-CD33 antibody or fragment thereof. The light chain of the anti-CD33 antibody or fragment thereof may be selected from SEQ ID NOS: 20 and 22, and optionally SEQ ID NO: 53 and a homologous amino acid sequence. The homologous amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NOS: 20 or 22 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 53. The heavy chain of the anti-CD33 antibody or fragment thereof may be selected from SEQ ID NO: 21 and 23, and optionally SEQ ID NO: 52 and a homologous amino acid sequence. The amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NOS: 21 and 23 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 52.

The TID may comprise an anti-CD123 antibody or fragment thereof. The light chain of the anti-CD123 antibody or fragment thereof may be selected from SEQ ID NOS: 24 and 26, and optionally SEQ ID NO: 53 and a homologous amino acid sequence. The homologous amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NOS: 24 or 26 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 53. The heavy chain of the anti-CD123 antibody or fragment thereof may be selected from SEQ ID NO: 25 and 27, and optionally SEQ ID NO: 52 and a homologous amino acid sequence. The amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NOS: 25 and 27 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 52.

The TID may comprise an anti-CD22 antibody or fragment thereof. The light chain of the anti-CD22 antibody or fragment thereof may be selected from SEQ ID NOS: 28 and 30, and optionally SEQ ID NO: 53 and a homologous amino acid sequence. The homologous amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NOS: 28 and 30 and optionally about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 53. The heavy chain of the anti-CD22 antibody or fragment thereof may be selected from SEQ ID NO: 29 and 31 and optionally SEQ ID NO: 52and a homologous amino acid sequence. The amino acid sequence may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NOS: 29 and 31 about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous to SEQ ID NO: 52.

The CAR-EC switches disclosed herein may comprise one or more unnatural amino acids. The one or more CAR-IDs may comprise one or more unnatural amino acids. The one or more TIDs may comprise one or more unnatural amino acids. The one or more linkers may comprise one or more unnatural amino acids. Attachment of the CAR-ID to the TID may occur via the one or more unnatural amino acids. The one or more linkers may link the one or more CAR-IDs to the one or more TIDs site-specifically through the one or more unnatural amino acids. Alternatively, or additionally, the one or more linkers may link the one or more TIDs to the one or more TIDs site-specifically, wherein an unnatural amino acid is not required to link the one or more TIDs to the one or more TIDs. The TID may be linked to 1, 2, 3, 4, 5 or more unnatural amino acids on the TID. The TID may be linked to 1, 2, 3, 4, 5 or more unnatural amino acids on the TID site-specifically. Alternatively, the TID may be linked to 1, 2, 3, 4, 5 or more unnatural amino acids on the TID. The TID may be linked to 1, 2, 3, 4, 5 or more unnatural amino acids on the TID site-specifically.

The CAR-ID may comprise one or more unnatural amino acids. The CAR-IDs disclosed herein may comprise 1, 2, 3, 4, 5, 6, 7, 8, 9, 10 or more unnatural amino acids. The TID may comprise one or more unnatural amino acids. The targeting antibodies or antibody fragments disclosed herein may comprise 2, 3, 4, 5, 6, 7, 8, 9, 10 or more unnatural amino acids. The unnatural amino acid may react with the linker to create a chemical bond.

The one or more unnatural amino acids may be inserted between two naturally occurring amino acids in the TID. The one or more unnatural amino acids may replace one or more naturally occurring amino acids in the TID. The one or more unnatural amino acids may be incorporated at the N terminus of the TID. The one or more unnatural amino acids may be incorporated at the C terminus of the TID. The one or more unnatural amino acids maybe incorporated at an internal site of the TID. The unnatural amino acid may be incorporated distal to the region of the TID that interacts with a molecule on or from a target. The unnatural amino acid may be incorporated proximal to the region of the TID that interacts with a molecule on or from a target. The unnatural amino acid may be incorporated at a site intermediate to the region of the TID that interacts with a molecule on or from a target. The unnatural amino acid may be incorporated in the region of the TID that interacts with a molecule on or from a target.

The one or more unnatural amino acids may replace one or more amino acids in the TID. The one or more unnatural amino acids may replace any natural amino acid in the TID.

The one or more unnatural amino acids may be incorporated in a light chain of the immunoglobulin from which the TID is based or derived. The one or more unnatural amino acids may be incorporated in a heavy chain of the immunoglobulin from which the TID is based or derived. The one or more unnatural amino acids may be incorporated in a heavy chain and a light chain of the immunoglobulin from which the TID is based or derived. The one or more unnatural amino acids may replace an amino acid in the light chain of the immunoglobulin from which the TID is based or derived. The one or more unnatural amino acids may replace an amino acid in a heavy chain of the immunoglobulin from which the TID is based or derived. The one or more unnatural amino acids may replace an amino acid in a heavy chain and a light chain of the immunoglobulin from which the TID is based or derived.

The one or more unnatural amino acids may replace a glycine of a light chain of the immunoglobulin from which the TID is based or derived. The one or more unnatural amino acids may replace an arginine of a light chain of the immunoglobulin from which the TID is based or derived. The one or more unnatural amino acids may replace a serine of a light chain of the immunoglobulin from which the TID is based or derived. The one or more unnatural amino acids may replace a threonine of a light chain of the immunoglobulin from which the TID is based or derived. The one or more unnatural amino acids may replace an alanine of a light chain of the immunoglobulin from which the TID is based or derived. The one or more unnatural amino acids may replace an alanine of a heavy chain of the immunoglobulin from which the TID is based or derived. The one or more unnatural amino acids may replace a serine of a heavy chain of the immunoglobulin from which the TID is based or derived. The one or more unnatural amino acids may replace a lysine of a heavy chain of the immunoglobulin from which the TID is based or derived. The one or more unnatural amino acids may replace a proline of a heavy chain of the immunoglobulin from which the TID is based or derived.

The one or more unnatural amino acids may replace an amino acid of the TID, wherein the TID is an anti-CD19 antibody or fragment thereof. The one or more unnatural amino acids may replace a glycine of a light chain of the anti-CD19 antibody or fragment thereof. The one or more unnatural amino acids may replace a threonine of a light chain of the anti-CD19 antibody or fragment thereof. The one or more unnatural amino acids may replace a serine of a light chain of the anti-CD19 antibody or fragment thereof. The one or more unnatural amino acids may replace a serine of a heavy chain of the anti-CD19 antibody or fragment thereof. The one or more unnatural amino acids may replace an alanine of a heavy chain of the anti-CD19 antibody or fragment thereof. The one or more unnatural amino acids may replace a lysine of a heavy chain of the anti-CD19 antibody or fragment thereof. The antibody or antibody fragment may be an anti-CD19 antibody or fragment thereof, wherein the one or more unnatural amino acids may replace one or more amino acids of a light chain of the anti-CD19 antibody or fragment thereof. The light chain of the anti-CD19 antibody or fragment thereof may comprise SEQ ID NO: 16 and optionally SEQ ID NO: 53. The one or more unnatural amino acids may replace one or more amino acids of SEQ ID NO: 16 and optionally SEQ ID NO: 53. The one or more amino acids of SEQ ID NO: 16 and optionally SEQ ID NO: 53 may be selected from the group consisting of: G68, K107, T109, E152, S156, K169 and S202. The one or more unnatural amino acids may replace one or more amino acids of a heavy chain of the anti-CD19 antibody or fragment thereof. The heavy chain of the anti-CD19 antibody or fragment thereof may comprise SEQ ID NO: 17 and optionally SEQ ID NO: 52. The one or more unnatural amino acids may replace one or more amino acids of SEQ ID NO: 17 and optionally SEQ ID NO: 52. The one or more amino acids of SEQ ID NO: 17 and optionally SEQ ID NO: 52 may be selected from the group consisting of S74, A121, and K136.

The one or more unnatural amino acids may replace an amino acid of the TID, wherein the TID is an anti-CD22 antibody or fragment thereof. The one or more unnatural amino acids may replace a glycine of a light chain of the anti-CD22 antibody or fragment thereof. The one or more unnatural amino acids may replace a threonine of a light chain of the anti-CD22 antibody or fragment thereof. The one or more unnatural amino acids may replace a serine of a light chain of the anti-CD22 antibody or fragment thereof. The one or more unnatural amino acids may replace a serine of a heavy chain of the anti-CD22 antibody or fragment thereof. The one or more unnatural amino acids may replace an alanine of a heavy chain of the anti-CD22 antibody or fragment thereof. The one or more unnatural amino acids may replace a lysine of a heavy chain of the anti-CD22 antibody or fragment thereof. The antibody or antibody fragment may be an anti-CD22 antibody or fragment thereof, wherein the one or more unnatural amino acids may replace one or more amino acids of a light chain of the anti-CD22 antibody or fragment thereof. The light chain of the anti-CD22 antibody or fragment thereof may comprise SEQ ID NO: 30 and optionally SEQ ID NO: 53. The one or more unnatural amino acids may replace one or more amino acids of SEQ ID NO: 30 and optionally SEQ ID NO: 53. The one or more amino acids of SEQ ID NO: 30 and optionally SEQ ID NO: 53 may be selected from the group consisting of: G74, T114, and S207. The one or more unnatural amino acids may replace one or more amino acids of a heavy chain of the anti-CD22 antibody or fragment thereof. The heavy chain of the anti-CD22 antibody or fragment thereof may comprise SEQ ID NO: 31 and optionally SEQ ID NO: 52. The one or more unnatural amino acids may replace one or more amino acids of SEQ ID NO: 31 and optionally SEQ ID NO: 52. The one or more amino acids of SEQ ID NO: 31 and optionally SEQ ID NO: 52 may be selected from the group consisting of S75, A117, and K132. The light chain of the anti-CD22 antibody or fragment thereof may comprise SEQ ID NO: 28 and optionally SEQ ID NO: 53. The one or more unnatural amino acids may replace one or more amino acids of SEQ ID NO: 28 and optionally SEQ ID NO: 53. The one or more amino acids of SEQ ID NO: 28 and optionally SEQ ID NO: 53 may be selected from the group consisting of: G68, T109, and S202. The one or more unnatural amino acids may replace one or more amino acids of a heavy chain of the anti-CD22 antibody or fragment thereof. The heavy chain of the anti-CD22 antibody or fragment thereof may comprise SEQ ID NO: 29 and optionally SEQ ID NO: 52. The one or more unnatural amino acids may replace one or more amino acids of SEQ ID NO: 29 and optionally SEQ ID NO: 52. The one or more amino acids of SEQ ID NO: 29 and optionally SEQ ID NO: 53 may be selected from the group consisting of S78, A125, and K140.

The one or more unnatural amino acids may replace an amino acid of the TID, wherein the TID is an anti-Her2 antibody or fragment thereof. The one or more unnatural amino acids may replace a glycine of a light chain of the anti-Her2 antibody or fragment thereof. The one or more unnatural amino acids may replace a threonine of a light chain of the anti-Her2 antibody or fragment thereof. The one or more unnatural amino acids may replace a serine of a light chain of the anti-Her2 antibody or fragment thereof. The one or more unnatural amino acids may replace a serine of a heavy chain of the anti-Her2 antibody or fragment thereof. The one or more unnatural amino acids may replace an alanine of a heavy chain of the anti-Her2 antibody or fragment thereof. The one or more unnatural amino acids may replace a lysine of a heavy chain of the anti-Her2 antibody or fragment thereof. The antibody or antibody fragment may be an anti-Her2 antibody or fragment thereof, wherein the one or more unnatural amino acids may replace one or more amino acids of a light chain of the anti-Her2 antibody or fragment thereof. The light chain of the anti-Her2 antibody or fragment thereof may comprise SEQ ID NO: 12 and optionally SEQ ID NO: 53. The one or more unnatural amino acids may replace one or more amino acids of SEQ ID NO: 12 and optionally SEQ ID NO: 53. The one or more amino acids of SEQ ID NO: 12 and optionally SEQ ID NO: 53 may be selected from the group consisting of: G68, T109, S202. The one or more unnatural amino acids may replace one or more amino acids of a heavy chain of the anti-Her2 antibody or fragment thereof. The heavy chain of the anti-Her2 antibody or fragment thereof may comprise SEQ ID NO: 13 and optionally SEQ ID NO: 52. The one or more unnatural amino acids may replace one or more amino acids of SEQ ID NO: 13 and optionally SEQ ID NO: 52. The one or more amino acids of SEQ ID NO: 13 and optionally SEQ ID NO: 53 may be selected from the group consisting of S75, A121, and K136.

The one or more unnatural amino acids may replace an amino acid of the TID, wherein the TID is an anti-CLL1 antibody or fragment thereof. The one or more unnatural amino acids may replace a glycine of a light chain of the anti-CLL1 antibody or fragment thereof. The one or more unnatural amino acids may replace a threonine of a light chain of the anti-CLL1 antibody or fragment thereof. The one or more unnatural amino acids may replace a serine of a light chain of the anti-CLL1 antibody or fragment thereof. The one or more unnatural amino acids may replace a serine of a heavy chain of the anti-CLL1 antibody or fragment thereof. The one or more unnatural amino acids may replace an alanine of a heavy chain of the anti-CLL1 antibody or fragment thereof. The one or more unnatural amino acids may replace a lysine of a heavy chain of the anti-CLL1 antibody or fragment thereof. The antibody or antibody fragment may be an anti-CLL1 antibody or fragment thereof, wherein the one or more unnatural amino acids may replace one or more amino acids of a light chain of the anti-CLL1 antibody or fragment thereof. The light chain of the anti-CLL1 antibody or fragment thereof may comprise SEQ ID NO: 18 and optionally SEQ ID NO: 53. The one or more unnatural amino acids may replace one or more amino acids of SEQ ID NO: 18 and optionally SEQ ID NO: 53. The one or more amino acids of SEQ ID NO: 18 and optionally SEQ ID NO: 53 may be selected from the group consisting of: G69, A110, and S203. The one or more unnatural amino acids may replace one or more amino acids of a heavy chain of the anti-CLL1 antibody or fragment thereof. The heavy chain of the anti-CLL1 antibody or fragment thereof may comprise SEQ ID NO: 19 and optionally SEQ ID NO: 52. The one or more unnatural amino acids may replace one or more amino acids of SEQ ID NO: 19 and optionally SEQ ID NO: 52. The one or more amino acids of SEQ ID NO: 19 and optionally SEQ ID NO: 52 may be selected from the group consisting of S75, A124, and K139.

The one or more unnatural amino acids may replace an amino acid of the TID, wherein the TID is an anti-CD33 antibody or fragment thereof. The one or more unnatural amino acids may replace a glycine of a light chain of the anti-CD33 antibody or fragment thereof. The one or more unnatural amino acids may replace a threonine of a light chain of the anti-CD33 antibody or fragment thereof. The one or more unnatural amino acids may replace a serine of a light chain of the anti-CD33 antibody or fragment thereof. The one or more unnatural amino acids may replace a serine of a heavy chain of the anti-CD33 antibody or fragment thereof. The one or more unnatural amino acids may replace an alanine of a heavy chain of the anti-CD33 antibody or fragment thereof. The one or more unnatural amino acids may replace a lysine of a heavy chain of the anti-CD33 antibody or fragment thereof. The antibody or antibody fragment may be an anti-CD33 antibody or fragment thereof, wherein the one or more unnatural amino acids may replace one or more amino acids of a light chain of the anti-CD33 antibody or fragment thereof. The light chain of the anti-CD33 antibody or fragment thereof may comprise SEQ ID NO: 22 and optionally SEQ ID NO: 53. The one or more unnatural amino acids may replace one or more amino acids of SEQ ID NO: 22 and optionally SEQ ID NO: 53. The one or more amino acids of SEQ ID NO: 22 and optionally SEQ ID NO: 53 may be selected from the group consisting of: G72, T113, and S206. The one or more unnatural amino acids may replace one or more amino acids of a heavy chain of the anti-CD33 antibody or fragment thereof. The heavy chain of the anti-CD33 antibody or fragment thereof may comprise SEQ ID NO: 23 and optionally SEQ ID NO: 52. The one or more unnatural amino acids may replace one or more amino acids of SEQ ID NO: 23 and optionally SEQ ID NO: 52. The one or more amino acids of SEQ ID NO: 23 and optionally SEQ ID NO: 52 may be selected from the group consisting of S75, A117, and K132. The light chain of the anti-CD33 antibody or fragment thereof may comprise SEQ ID NO: 20 and optionally SEQ ID NO: 53. The one or more unnatural amino acids may replace one or more amino acids of SEQ ID NO: 20 and optionally SEQ ID NO: 53. The one or more amino acids of SEQ ID NO: 20 and optionally SEQ ID NO: 53 may be selected from the group consisting of: G72, T113, and S206. The one or more unnatural amino acids may replace one or more amino acids of a heavy chain of the anti-CD33 antibody or fragment thereof. The heavy chain of the anti-CD33 antibody or fragment thereof may comprise SEQ ID NO: 21 and optionally SEQ ID NO: 52. The one or more unnatural amino acids may replace one or more amino acids of SEQ ID NO: 21 and optionally SEQ ID NO: 52. The one or more amino acids of SEQ ID NO: 21 and optionally SEQ ID NO: 52 may be selected from the group consisting of P75, A117, and K132.

The one or more unnatural amino acids may replace an amino acid of the TID, wherein the TID is an anti-CD123 antibody or fragment thereof. The one or more unnatural amino acids may replace a glycine of a light chain of the anti-CD123 antibody or fragment thereof. The one or more unnatural amino acids may replace a threonine of a light chain of the anti-CD123 antibody or fragment thereof. The one or more unnatural amino acids may replace a serine of a light chain of the anti-CD123 antibody or fragment thereof. The one or more unnatural amino acids may replace a serine of a heavy chain of the anti-CD123 antibody or fragment thereof. The one or more unnatural amino acids may replace an alanine of a heavy chain of the anti-CD123 antibody or fragment thereof. The one or more unnatural amino acids may replace a lysine of a heavy chain of the anti-CD123 antibody or fragment thereof. The antibody or antibody fragment may be an anti-CD123 antibody or fragment thereof, wherein the one or more unnatural amino acids may replace one or more amino acids of a light chain of the anti-CD123 antibody or fragment thereof. The light chain of the anti-CD123 antibody or fragment thereof may comprise SEQ ID NO: 24 and optionally SEQ ID NO: 53. The one or more unnatural amino acids may replace one or more amino acids of SEQ ID NO: 24 and optionally SEQ ID NO: 53. The one or more amino acids of SEQ ID NO: 24 and optionally SEQ ID NO: 53 may be selected from the group consisting of: R72, T113, and S206. The one or more unnatural amino acids may replace one or more amino acids of a heavy chain of the anti-CD123 antibody or fragment thereof. The heavy chain of the anti-CD123 antibody or fragment thereof may comprise SEQ ID NO: 25 and optionally SEQ ID NO: 52. The one or more unnatural amino acids may replace one or more amino acids of SEQ ID NO: 25 and optionally SEQ ID NO: 52. The one or more amino acids of SEQ ID NO: 25 and optionally SEQ ID NO: 52 may be selected from the group consisting of S75, A119, and K134. The light chain of the anti-CD123 antibody or fragment thereof may comprise SEQ ID NO: 26 and optionally SEQ ID NO: 53. The one or more unnatural amino acids may replace one or more amino acids of SEQ ID NO: 26 and optionally SEQ ID NO: 53. The one or more amino acids of SEQ ID NO: 26 and optionally SEQ ID NO: 53 may be selected from the group consisting of: G68, T109, and S202. The one or more unnatural amino acids may replace one or more amino acids of a heavy chain of the anti-CD123 antibody or fragment thereof. The heavy chain of the anti-CD123 antibody or fragment thereof may comprise SEQ ID NO: 27 and optionally SEQ ID NO: 52. The one or more unnatural amino acids may replace one or more amino acids of SEQ ID NO: 27 and optionally SEQ ID NO: 52. The one or more amino acids of SEQ ID NO: 27 and optionally SEQ ID NO: 52 may be selected from the group consisting of S75, A116, and K131.

The one or more unnatural amino acids may be encoded by a codon that does not code for one of the twenty natural amino acids. The one or more unnatural amino acids may be encoded by a nonsense codon (stop codon). The stop codon may be an amber codon. The amber codon may comprise a UAG sequence. Herein, “UAG” and “TAG” may be used interchangeably in reference to amber codons. The stop codon may be an ochre codon. The ochre codon may comprise a UAA sequence. The stop codon may be an opal or umber codon. The opal or umber codon may comprise a UGA sequence. The one or more unnatural amino acids may be encoded by a four-base codon.

The one or more unnatural amino acids may be p-acetylphenylalanine (pAcF or pAcPhe). The one or more unnatural amino acids may be selenocysteine. The one or more unnatural amino acids may be p-fluorophenylalanine (pFPhe). The one or more unnatural amino acids may be selected from the group comprising p-azidophenylalanine (pAzF), p-azidomethylphenylalanine(pAzCH₂F), p-benzoylphenylalanine (pBpF), p-propargyloxyphenylalanine (pPrF), p-iodophenylalanine (pIF), p-cyanophenylalanine (pCNF), p-carboxylmethylphenylalanine (pCmF), 3-(2-naphthyl)alanine (NapA), p-boronophenylalanine (pBoF), o-nitrophenylalanine (oNiF), (8-hydroxyquinolin-3-yl)alanine (HQA), selenocysteine, and (2,2′-bipyridin-5-yl)alanine (BipyA). The one or more unnatural amino acids may be 4-(6-methyl-s-tetrazin-3-yl)aminopheynlalanine.

The one or more unnatural amino acids may be β-amino acids (β3 and β2), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, N-methyl amino acids, or a combination thereof.

Additional examples of unnatural amino acids include, but are not limited to, 1) various substituted tyrosine and phenylalanine analogues such as O-methyl-L-tyrosine, p-amino-L-phenylalanine, 3-nitro-L-tyrosine, p-nitro-L-phenylalanine, m-methoxy-L-phenylalanine and p-isopropyl-L-phenylalanine; 2) amino acids with aryl azide and benzophenone groups that may be photo-cross-linked; 3) amino acids that have unique chemical reactivity including acetyl-L-phenylalanine and m-acetyl-L-phenylalanine, O-allyl-L-tyrosine, O-(2-propynyl)-L-tyrosine, p-ethylthiocarbonyl-L-phenylalanine and p-(3-oxobutanoyl)-L-phenylalanine; 4) heavy-atom-containing amino acids for phasing in X-ray crystallography including p-iodo and p-bromo-L-phenylalanine; 5) the redox-active amino acid dihydroxy-L-phenylalanine; 6) glycosylated amino acids including b-N-acetylglucosamine-O-serine and a-N-acetylgalactosamine-O-threonine; 7) fluorescent amino acids with naphthyl, dansyl, and 7-aminocoumarin side chains; 8) photocleavable and photoisomerizable amino acids with azobenzene and nitrobenzyl Cys, Ser, and Tyr side chains; 9) the phosphotyrosine mimetic p-carboxymethyl-L-phenylalanine; 10) the glutamine homologue homoglutamine; and 11) 2-aminooctanoic acid. The unnatural amino acid may be modified to incorporate a chemical group. The unnatural amino acid may be modified to incorporate a ketone group.

The one or more unnatural amino acids may comprise at least one oxime, carbonyl, dicarbonyl, hydroxylamine group or a combination thereof. The one or more unnatural amino acids may comprise at least one carbonyl, dicarbonyl, alkoxy-amine, hydrazine, acyclic alkene, acyclic alkyne, cyclooctyne, aryl/alkyl azide, norbornene, cyclopropene, trans-cyclooctene, or tetrazine functional group or a combination thereof.

The one or more unnatural amino acids may be incorporated into the TID and/or the CAR-ID by methods known in the art. Cell-based or cell-free systems may be used to alter the genetic sequence of the TID and/or the CAR-ID, thereby producing the TID and/or the CAR-ID with one or more unnatural amino acids. Auxotrophic strains may be used in place of engineered tRNA and synthetase. The one or more unnatural amino acids may be produced through selective reaction of one or more natural amino acids. The selective reaction may be mediated by one or more enzymes. In one non-limiting example, the selective reaction of one or more cysteines with formylglycine generating enzyme (FGE) may produce one or more formylglycines (see Rabuka et al., Nature Protocols 7:1052-1067 (2012), which is incorporated by reference in its entirety).

The one or more unnatural amino acids may take part in a chemical reaction to form a linker. The chemical reaction to form the linker may be a bioorthogonal reaction. The chemical reaction to form the linker may be click chemistry.

Additional unnatural amino acids are disclosed in Liu et al. (Annu Rev Biochem, 79:413-44, 2010), Wang et al. (Angew Chem Int Ed, 44:34-66, 2005) and PCT application numbers PCT/US2012/039472, PCT/US2012/039468, PCT/US2007/088009, PCT/US2009/058668, PCT/US2007/089142, PCT/US2007/088011, PCT/US2007/001485, PCT/US2006/049397, PCT/US2006/047822 and PCT/US2006/044682, all of which are incorporated by reference in their entireties.

Alternatively or additionally, the TID may comprise a small molecule. The small molecule may be an organic compound. The small molecule may have a size on the order of about 10⁻⁸ m, about 10⁻⁹ m, about 10⁻¹⁰ m. The small molecule may have a size of less than about 10⁻⁷ m. The small molecule may have a size of less than about 10⁻⁸ m. The small molecule may have a size of less than about 10⁻⁹ m. The small molecule may have a size of less than about 10⁻¹⁰ m. The small molecule may have a size of less than about 10⁻¹¹ m. The small molecule may have a mass of less than about 5000 Da, less than about 4500 Da, less than about 4000 Da, less than about 3500 Da, less than about 3000 Da, less than about 2500 Da, less than about 2000 Da, less than about 1500 Da, less than about 1000 Da, less than about 900 D, less than about 500 Da or less than about 100 Da. In some instances, the small molecule does not comprise a polypeptide. In some instances, the small molecule does comprise two or more amino acids that are linked by an amide bond. The small molecule may be a chemical compound.

### Linkers

The switches disclosed herein may comprise one or more linkers. The switches disclosed herein may comprise two or more linkers. The switches disclosed herein may comprise three or more linkers. The switches disclosed herein may comprise four or more linkers. The switches disclosed herein may comprise 5, 6, 7, 8, 9, 10 or more linkers. The two or more linkers may be the same. At least two of the three or more linkers may be the same. The two or more linkers may be different. At least two of the three or more linkers may be different. The linker may be a bifunctional linker. The linker may be a heterobifunctional linker. The linker may be a homobifunctional linker. The linker may further comprise one or more polyethylene glycol (PEG) subunits. The linker may comprise at least four PEG subunits. The linker may comprise at least 10 PEG subunits. The linker may comprise at least 20 PEG subunits. The linker may comprise at least 30 PEG subunits. The linker may comprise an azide at one end. The linker may comprise an aminooxy at one end. The linker may be an azide-PEG-aminooxy linker. The linker may comprise cyclooctyne at one end. The linker may be a PEG-cyclooctyne linker. The linker may comprise triazole. The triazole may be a 1,2,3-triazole or a 1,2,4-triazole. The linker may be a NHS-ester linker. The linker may be a TriA linker. The linker may be attached to the CAR-ID by oxime ligation.

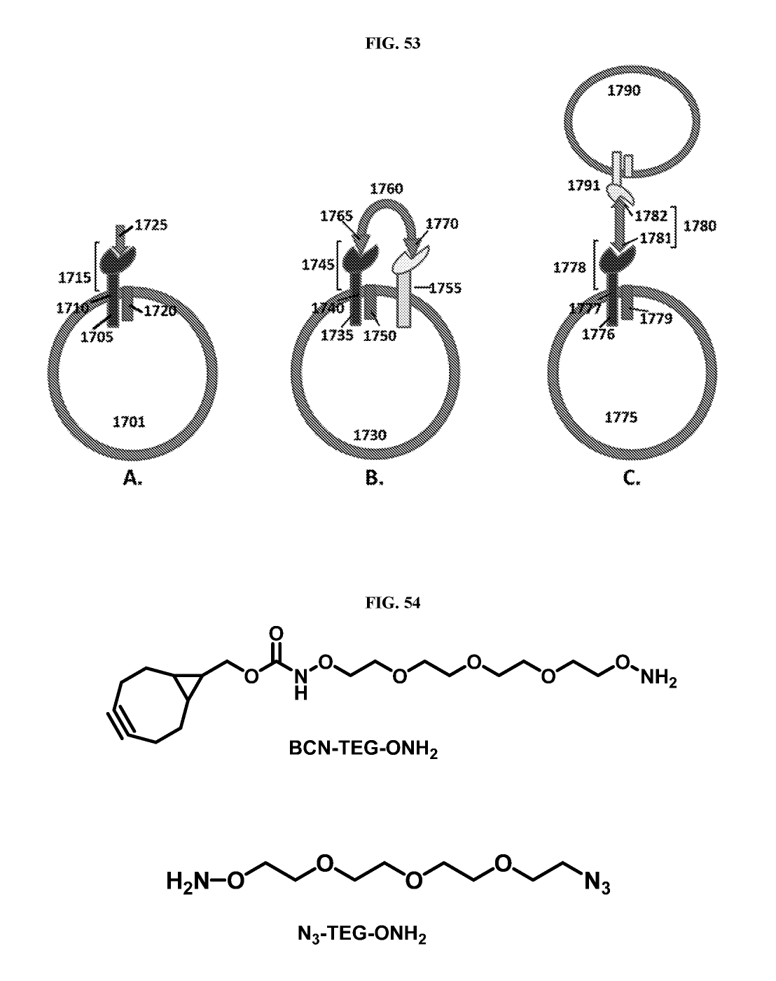

FIG. 53A-FIG. 53C shows schematics of exemplary CAR regulator-CAR-EC interactions. As shown in FIG. 53A, a chimeric antigen receptor effector cell (CAR-EC) (1701) may comprise a chimeric antigen receptor (1704) and a costimulatory molecule (1720). The CAR (1704) may comprise an external domain (1715), a transmembrane domain (1710) and an internal domain (1705). The CAR-EC regulator (1725) may interact with the external domain (1715) of the CAR (1704). The attachment of the CAR-EC regulator (1725) to the CAR (1704) may induce apoptosis of CAR-EC. The attachment of the CAR-EC regulator (1725) to the CAR (1704) may induce activation-induced cell death of CAR-EC. The attachment of the CAR-EC regulator (1725) to the CAR (1704) may induce autophagy of CAR-EC. The attachment of the CAR-EC regulator (1725) to the CAR (1704) may induce down regulation of the CAR. The attachment of the CAR-EC regulator (1725) to the CAR (1704) may prevent the CAR-EC switch from attaching to the CAR.

As shown in FIG. 53B, a CAR-EC (1730) may comprise a CAR (1731), a costimulatory molecule (1750) and a surface molecule (1755). The CAR (1731) may comprise an external domain (1745), a transmembrane domain (1740) and an internal domain (1735). The CAR-EC regulator (1760) may comprise a first end (1765) that interacts with the external domain (1715) of the CAR (1731) and a second end (1770) that interacts with the surface molecule (1755) on the CAR-EC. The attachment of the one end of the CAR-EC regulator (1760) to the CAR (1731) and the surface molecule (1755) of the CAR-EC (1730) may induce apoptosis of CAR-EC. The attachment of the one end of the CAR-EC regulator (1760) to the CAR (1731) and the surface molecule (1755) of the CAR-EC (1730) may induce activation-induced cell death of CAR-EC. The attachment of the one end of the CAR-EC regulator (1760) to the CAR (1731) and the surface molecule (1755) of the CAR-EC (1730) may induce autophagy of CAR-EC. The attachment of the one end of the CAR-EC regulator (1760) to the CAR (1731) and the surface molecule (1755) of the CAR-EC (1730) may induce down regulation of the CAR. The attachment of one end of the CAR-EC regulator (1765) to the CAR (1731) may prevent the CAR-EC switch from attaching to the CAR.

As shown in FIG. 53C, a CAR-EC (1775) may comprise a CAR (1774) and a costimulatory molecule (1779). The CAR (1774) may comprise an external domain (1778), a transmembrane domain (1777) and an internal domain (1776). The CAR-EC regulator (1780) may comprise a first region (1781) that interacts with the external domain (1778) of the CAR (1774) on the effector cell. The CAR-EC regulator (1780) may further comprise a second region (1782) that interacts with a surface molecule (1791) on another cell (1790). The cell (1790) may secrete cytokines or other molecules that can interact with the CAR-EC. The interaction of the cytokines or other molecules with the CAR-EC may induce apoptosis of CAR-EC. The interaction of the cytokines or other molecules with the CAR-EC may induce activation-induced cell death of CAR-EC. The interaction of the cytokines or other molecules with the CAR-EC may induce autophagy of CAR-EC. The interaction of the cytokines or other molecules with the CAR-EC may induce down regulation of the CAR. The attachment of one end of the CAR-EC regulator (1781) to the CAR (1774) may prevent the CAR-EC switch from attaching to the CAR.

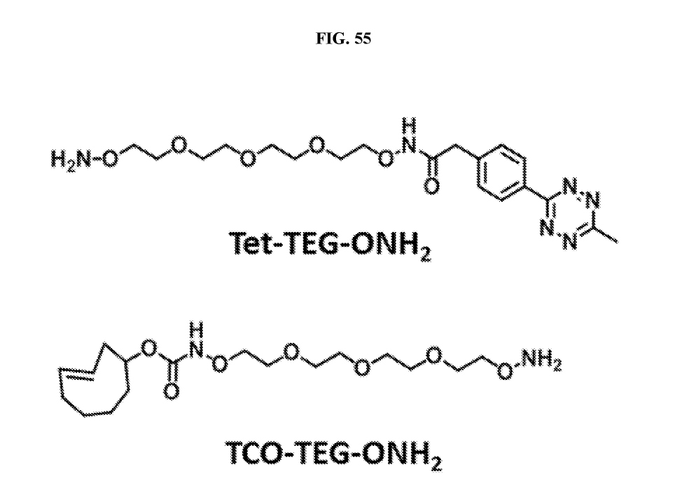

FIG. 54 depicts exemplary heterobifunctional linkers. FIG. 55 shows a general scheme for synthesizing bifunctional linkers. Additional exemplary linkers and methods of constructing linkers can be found in WO2014/153002, which is incorporated by reference in its entirety.

The linker may be attached to a CAR-ID. The linker may be attached to a TID. The linker may attach a CAR-ID to a TID. The one or more linkers may attach the one or more CAR-IDs to the one or more TIDs. The one or more linkers may attach the one or more CAR-IDs to the one or more TIDs in a site-specific manner. Attachment in a site-specific manner may comprise attaching the one or more CAR-IDs to a predetermined site on the one or more TIDs. Alternatively, or additionally, attachment in a site-specific manner may comprise attaching the one or more CAR-IDs to an unnatural amino acid in the one or more TIDs. The one or more linkers may attach the one or more CAR-IDs to the one or more TIDs in a site-independent manner. Attachment in a site-independent manner may comprise attaching the one or more CAR-IDs to a random site on the one or more TIDs. The CAR-ID may be attached to 1, 2, 3, 4, 5 or more TIDs in a site-specific manner. The CAR-ID may be attached to 1, 2, 3, 4, 5 or more TIDs in a site-independent manner. Alternatively, the TID may be attached to 1, 2, 3, 4, 5 or more CAR-IDs in a site-specific manner. Attachment in a site-specific manner may comprise attaching the one or more TIDs to a predetermined site on the one or more CAR-IDs. The TID may be attached to 1, 2, 3, 4, 5 or more CAR-IDs in a site-independent manner. Attachment in a site-independent manner may comprise attaching the one or more TIDs to a random site on the one or more CAR-IDs.

The one or more linkers may be coupled to the CAR-ID, the TID, or a combination thereof. The one or more linkers may be coupled to the CAR-ID to form one or more switch intermediates of the Formula IIA: L1-X or Formula II: X-L1, wherein X is the CAR-ID and L1 is the linker. The one or more linkers may be coupled to the CAR-ID by an oxime. The one or more linkers may be coupled to the CAR-ID by a cyclooctyne, cyclopropene, aryl/alkyl azides, trans-cyclooctene, norborene, tetrazine, or a combination thereof. The one or more linkers may be coupled to the CAR-ID by a covalent bond, non-covalent bond, ionic bond, or a combination thereof. The one or more linkers may be coupled to the TID to form one or more switch intermediates of the Formula IIIA: L1-Y or Formula III: Y-L1, wherein Y is the TID and L1 is the linker. The one or more linkers may be coupled to the TID by an oxime. The one or more linkers may be coupled to the TID by a cyclooctyne, cyclopropene, aryl/alkyl azides, trans-cyclooctene, norborene, tetrazine, or a combination thereof. The one or more linkers may be coupled to the TID by a covalent bond, non-covalent bond, ionic bond, or a combination thereof.

The TID may comprise one or more amino acids. The one or more amino acids may comprise a natural amino acid. The linker may couple with one or more natural amino acids on the TID. The one or more amino acids may comprise one or more unnatural amino acids. The linker may couple with one or more unnatural amino acids on the TID. The linker may couple with an amino acid which is the product of site-specific mutagenesis. The linker may couple with a cysteine which is the product of site-specific mutagenesis. The linker (e.g., substituted maleimide) may couple with a cysteine which is the product of site-specific mutagenesis, as well as a native cysteine residue. Two linkers, each with complementary reactive functional groups, may couple with one another.

The one or more linkers may be a cleavable linker. The one or more linkers may be a non-cleavable linker. The one or more linkers may be a flexible linker. The one or more linkers may be an inflexible linker. The linker may be a bifunctional linker. A bifunctional linker may comprise a first functional group on one end and a second functional group on the second end. The bifunctional linker may be heterobifunctional linker. A heterobifunctional linker may comprise a first functional group on one end and a second functional group on the second end, wherein the first functional group and the second functional group are different. The bifunctional linker may be a homobifunctional linker. A homobifunctional linker may comprise a first functional group on one end and a second functional group on the second end, wherein the first functional group and the second functional group are the same.

The linker may comprise a chemical bond. The linker may comprise a functional group. The linker may comprise a polymer. The polymer may be a polyethylene glycol. The linker may comprise an amino acid.

The linker may comprise one or more functional groups. The linker may comprise two or more functional groups. The linker may comprise three or more functional groups. The linker may comprise four or more functional groups. The linker may comprise 5, 6, 7, 8, 9, 10 or more functional groups. The linker may be a bifunctional ethylene glycol linker.

The linker may comprise ethylene glycol. The linker may comprise about 1, about 2, about 3, about 4, about 5, about 6, about 7, about 8, about 9, about 10, about 11, about 12, about 13, about 14, about 15, about 16, about 17, about 18, about 19 or about 20 or more ethylene glycol subunits. The linker may comprise 4 or more ethylene glycol subunits. The linker may comprise 8 or more ethylene glycol subunits. The linker may comprise 10 or more ethylene glycol subunits. The linker may comprise 12 or more ethylene glycol subunits. The linker may comprise 15 or more ethylene glycol subunits. The linker may comprise 20 or more ethylene glycol subunits. The linker may comprise 25 or more ethylene glycol subunits. The linker may comprise 30 or more ethylene glycol subunits. The linker may comprise 35 or more ethylene glycol subunits.

The linker may comprise PEG. The linker may comprise about 1, about 2, about 3, about 4, about 5, about 6, about 7, about 8, about 9, about 10, about 11, about 12, about 13, about 14, about 15, about 16, about 17, about 18, about 19 or about 20 or more polyethylene glycol (PEG) subunits. The linker may comprise 4 or more polyethylene glycol (PEG) subunits. The linker may comprise 8 or more PEG subunits. The linker may comprise 10 or more PEG subunits. The linker may comprise 12 or more PEG subunits. The linker may comprise 15 or more PEG subunits. The linker may comprise 20 or more PEG subunits. The linker may comprise 25 or more PEG subunits. The linker may comprise 30 or more PEG subunits. The linker may comprise 35 or more PEG subunits.

The linker may comprise a triazole. The triazole may be a 1,2,3-triazole. The triazole may be a 1,2,4-triazole.

The linker may comprise an aryl or a heteroaryl. The linker may comprise an aryl. The aryl may be phenyl. The phenyl may be disubstituted. The disubstituted phenyl may be 1,4-disubstituted phenyl. The disubstituted phenyl may be 1,3-disubstituted phenyl. The phenyl may be trisubstituted. The phenyl may be tetrasubstituted. Two of the substituents of the substituted phenyl may be NO₂. In some instances, the linker does not comprise a benzyl substituent.

The linker may comprise one or more PEG units. The linker may comprise multiple PEG units. The linker may comprise 2 or more PEG units. The linker may comprise 3 or more PEG units. The linker may comprise 4 or more PEG units. The linker may comprise 5 or more PEG units. The linker may comprise 6 or more PEG units. The linker may comprise 7 or more PEG units. The linker may comprise 8 or more PEG units. The linker may comprise 9 or more PEG units. The linker may comprise 10 or more PEG units. The linker may comprise 11 or more PEG units. The linker may comprise 12 or more PEG units. The linker may comprise 13 or more PEG units. The linker may comprise 14 or more PEG units.

The linker may comprise an amide on one end. The linker may comprise an amide on one end and an amine on the other end. The linker may comprise an amide on one end and a triazole on the other end.

The one or more linkers may comprise a 1,4-dicarboxylic moiety. The one or more linkers may comprise a 1,3-dinitro substituted phenyl moiety.

The one or more linkers may comprise one or more reactive functional groups. The reactive functional group may react with a complementary reactive functional group on a coupling partner. The reaction of the reactive functional group on the linker to a complementary reactive functional group on a coupling partner may occur prior to incorporation of the linker into the CAR-EC switch.

The linker may comprise at least one reactive functional group selected from alkoxy-amine, hydrazine, aryl/alkyl azide, alkyne, alkene, tetrazine, dichlorotriazine, tresylate, succinimidyl carbonate, benzotriazole carbonate, nitrophenyl carbonate, trichlorophenyl carbonate, carbonylimidazole, succinimidyl succinate, maleimide, vinylsulfone, haloacetamide, and disulfide. The alkene may be selected from norbornene, trans-cyclooctene, and cyclopropene. The linker may comprise at least one alkoxy amine. The linker may comprise at least one azide. The linker may comprise at least one cyclooctyne. The linker may comprise at least one tetrazine.

The one or more linkers may comprise an alkoxy-amine (or aminooxy) group, azide group and/or cyclooctyne group at one or more termini. The one or more linkers may comprise an alkoxy-amine at one terminus and an azide group at the other terminus. The one or more linkers may comprise an alkoxy-amine at one terminus and a cyclooctyne group at the other terminus. The alkoxy-amine may form a stable oxime with a ketone group on an amino acid. The alkoxy-amine may form a stable oxime with a ketone group on an unnatural amino acid. The ketone group may be on a p-acetyl phenylalanine (pAcF).

One or more linkers may be formed by reaction of reactive functional group on the CAR-ID with a complementary reactive functional group of a linker that is attached to the TID. One or more linkers may be formed by reaction of an amino acid or another reactive functional group on the TID with a complementary reactive functional group of a linker that is attached to the CAR-ID. One or more linkers may be formed by reaction of a linker that is attached to the CAR-ID with another linker that is attached to the TID. FIG. 57 shows a schematic of producing a linker by reaction of reactive functional groups on two switch intermediates. As shown in FIG. 57, a first switch intermediate (1601) comprising a CAR-ID (1605) and a first linker (1610) is contacted with a second switch intermediate (1620) comprising a TID (1625) and a second linker (1630). The reactive functional group (1615) of the first linker (1610) reacts with the second functional group (1635) of the second linker (1635) to produce a new linker (1645). The reaction of the two switch intermediates (1601, 1620) results in the formation of a switch (1640) comprising the CAR-ID (1605) connected to the TID (1625) via the new linker (1645).

The linker may be the product of a bioorthogonal reaction. For example, amino acids that contain ketone, azide, alkyne, alkene, and tetrazine side chains can be genetically encoded in response to nonsense and frameshift codons. These side chains can act as chemical handles for bioorthogonal conjugation reactions (Kim et al., Curr Opin Chem Bio 17:412-419 (2013), which is incorporated by reference in its entirety). The linker may comprise an oxime, a tetrazole, a Diels Alder adduct, a hetero Diels Alder adduct, an aromatic substitution reaction product, a nucleophilic substitution reaction product, an ester, an amide, a carbamate, an ether, a thioether, or a Michael reaction product. The linker may be a cycloaddition product, a metathesis reaction product, a metal-mediated cross-coupling reaction product, a radical polymerization product, an oxidative coupling product, an acyl-transfer reaction product, or a photo click reaction product. The cycloaddition may be a Huisgen-cycloaddition. The cycloaddition may be a copper-free [3+2] Huisgen-cycloaddition. The cycloaddition may be a Diels-Alder reaction. The cycloaddition may be a hetero Diels-Alder reaction. The linker may be the product of an enzyme-mediated reaction. The linker may be a product of a transglutaminase-mediated reaction, non-limiting examples of which are described in Lin et al., J. Am. Chem. Soc. 128:4542-4543 (2006) and WO 2013/093809. The linker may comprise a disulfide bridge that connects two cysteine residues, such as ThioBridge™ technology by PolyTherics. The linker may comprise a maleimide bridge that connects two amino acid residues. The linker may comprise a maleimide bridge that connects two cysteine residues.

Two or more linkers may be linked. The two or more linkers may be linked through one or more copper-free reactions. The two or more linkers may be linked through one or more cycloadditions. The two or more linkers may be linked through one or more Huisgen-cycloadditions. The two or more linkers may be linked through one or more copper-free [3+2] Huisgen-cycloadditions. The two or more linkers may be linked through one or more copper-containing reactions. The two or more linkers may be linked through one or more Diels Alder reactions. The two or more linkers may be linked through one or more hetero Diels Alder reactions.

CAR-EC switches may be optimized by adjusting linker length. CAR-EC switches may comprise linkers of different lengths. Linkers may be relatively short. Linkers may be relatively long. The one or more linkers may be between about 1 angstroms (Å) to about 120 Å in length. The one or more linkers may be between about 5 Å to about 105 Å in length. The one or more linkers may be between about 10 Å to about 100 Å in length. The one or more linkers may be between about 10 Å to about 90 Å in length. The one or more linkers may be between about 10 Å to about 80 Å in length. The one or more linkers may be between about 10 Å to about 70 Å in length. The one or more linkers may be between about 15 Å to about 45 Å in length. The one or more linkers may be equal to or greater than about 4, 5, 6, 7, 8, 9, 10, 11, 12, 13, 14, 15, 16, 17, 18, 19, 20, 25, 27, 30 or more angstroms in length. The one or more linkers may be equal to or greater than about 10 Å in length. The one or more linkers may be equal to or greater than about 15 angstroms in Å. The one or more linkers may be equal to or greater than about 20 Å in length. The one or more linkers may be equal to or less than about 110, 100, 90, 85, 80, 75, 70, 65, 60, 55, 50, 45, 43, 42, 41, 40, 39, 38, 37, 36, 35, 34, 33, 32, 31, 30 or fewer Å in length. The one or more linkers may be equal to or less than about 100 Å in length. The one or more linkers may be equal to or less than about 80 Å in length. The one or more linkers may be equal to or less than about 60 Å in length. The one or more linkers may be equal to or less than about 40 Å in length.

The total length of the linkers may be between about 1 Å to about 120 Å. The total length of the linkers may be between about 5 Å to about 105 Å. The total length of the linkers may be between about 10 Å to about 100 Å. The total length of the linkers may be between about 10 Å to about 90 Å. The total length of the linkers may be between about 10 Å to about 80 Å. The total length of the linkers may be between about 10 Å to about 70 Å. The total length of the linkers may be between about 15 Å to about 45 Å. The total length of the linkers may be equal to or greater than about 4, 5, 6, 7, 8, 9, 10, 11, 12, 13, 14, 15, 16, 17, 18, 19, 20, 25, 27, 30 or more Å. The total length of the linkers may be equal to or greater than about 10 Å. The total length of the linkers may be equal to or greater than about 15 Å. The total length of the linkers may be equal to or greater than about 20 Å. The total length of the linkers may be equal to or less than about 110, 100, 90, 85, 80, 75, 70, 65, 60, 55, 50, 45, 43, 42, 41, 40, 39, 38, 37, 36, 35, 34, 33, 32, 31, 30 or fewer Å. The total length of the linkers may be equal to or less than about 100 Å. The total length of the linkers may be equal to or less than about 80 Å. The total length of the linkers may be equal to or less than about 60 Å. The total length of the linkers may be equal to or less than about 40 Å. The total length of the linkers may be equal to or less than about 25 Å.The distance between the CAR-ID and the TID may be about 30 Å.

Disclosed herein are compositions comprising a plurality of switches, wherein a switch of the plurality of switches comprises (a) a CAR-ID; (b) a TID; and (c) a linker, wherein at least about 60% of the switches of the plurality of switches are structurally homogeneous. At least about 60%, 61%, 62%, 63%, 64%, 65%, 66%, 67%, 68% or 69% of the switches of the plurality of switches may be structurally homogeneous. At least about 70%, 71%, 72%, 73%, 74%, 75%, 76%, 77%, 78% or 79% of the switches of the plurality of switches may be structurally homogeneous. At least about 80%, 81%, 82%, 83%, 84%, 85%, 86%, 87%, 88% or 89% of the switches of the plurality of switches may be structurally homogeneous. At least about 90%, 91%, 92%, 93%, 94%, 95%, 96%, 97%, 98% or 99% of the switches of the plurality of switches may be structurally homogeneous. Structurally homogeneous CAR-EC switches may be provided for by site-specifically linking the CAR-ID and the TID. The linker may be linked to a CAR-ID site-specifically. The linker may be linked to a TID site-specifically. A first site of the linker may be linked to a CAR-ID site-specifically and a second site of the linker may be linked to a TID site-specifically.

### Targets

Disclosed herein are CAR-EC switches comprising a CAR-ID and a TID that binds a cell surface molecule on a target cell. Generally, binding of the effector cell and the target cell to the CAR-EC switch construct brings the target cell into proximity with the effector cell sufficiently close for an activity of the effector cell to have an effect on the target cell. For example, when the T cell and the target cell are bound to the CAR-EC switch, the T cell may produce an immune response that has a cytotoxic effect on the target cell.

The CAR-EC switches may interact with a plurality of target cells. The target cell may be an infected cell. The target cell may be a pathogenically infected cell. The target cell may be a diseased cell. The target cell may be a genetically-modified cell. The target cell may not be a host cell. The target cell may come from an invading organism (e.g. yeast, worm, bacteria, fungi). Further disclosed herein are CAR-EC switches that interact with a molecule on a non-cell target. The non-cell target may be a virus or a portion thereof. The non-cell target may be a fragment of a cell. The non-cell target may be an extracellular matrix component or protein.

The target cell may be derived from a tissue. The tissue may be selected from brain, esophagus, breast, colon, lung, glia, ovary, uterus, testes, prostate, gastrointestinal tract, bladder, liver, thymus, bone and skin. The target cell may be derived from one or more endocrine glands. The endocrine gland may be a lymph gland, pituitary gland, thyroid gland, parathyroid gland, pancreas, gonad or pineal gland.

The target cell may be selected from a stem cell, a pluripotent cell, a hematopoietic stem cell or a progenitor cell. The target cell may a circulating cell. The target cell may be an immune cell.

The target cell may be a cancer stem cell. The target cell may be a cancer cell. The cancer cell may be derived from a tissue. The tissue may be selected from, by way of non-limiting example, a brain, an esophagus, a breast, a colon, a lung, a glia, an ovary, a uterus, a testicle, a prostate, a gastrointestinal tract, a bladder, a liver, a thyroid and skin. The cancer cell may be derived from bone. The cancer cell may be derived from blood. The cancer cell may be derived from a B cell, a T cell, a monocyte, a thrombocyte, a leukocyte, a neutrophil, an eosinophil, a basophil, a lymphocyte, a hematopoietic stem cell or an endothelial cell progenitor. The cancer cell may be derived from a CD19⁺ B lymphocyte. The cancer cell may be derived from a stem cell. The cancer cell may be derived from a pluripotent cell. The cancer cell may be derived from one or more endocrine glands. The endocrine gland may be a lymph gland, pituitary gland, thyroid gland, parathyroid gland, pancreas, gonad or pineal gland.

The cancer cell may be a CD19⁺ cell. The cancer cell may be a CD19⁺ B lymphocyte. The cancer cell may be a Her2⁺ cell. The Her2⁻ cell may be a Her2⁻ breast cancer cell. The target cell may be a BCMA⁺ cell. The cancer cell may be a BCMA⁺ multiple myeloma cell. The cancer cell may be a CS1⁺ cell. The CS1⁺ cell may be a multiple myeloma cell. The cancer cell may be an EGFRvIII-positive cell. The cancer cell may be an EGFRvIII-positive glioblastoma cell. The cancer cell may be a CD20⁺ cell. The cancer cell may be a CD22⁺ cell. The cancer cell may be a CD123⁺ cell. The cancer cell may be a CD33⁺ cell. The cancer cell may be a CEA-positive cell. The cancer cell may be a CLL1⁺ cell.

The cell surface molecule may be an antigen. The antigen may be at least a portion of a surface antigen or a cell surface marker on a cell. The antigen may be a receptor or a co-receptor on a cell. The antigen may refer to a molecule or molecular fragment that may be bound by a major histocompatibility complex (MHC) and presented to a TCR. The term “antigen” may also refer to an immunogen. The immunogen may provoke an adaptive immune response if injected on its own into a subject. The immunogen may induce an immune response by itself. The antigen may be a superantigen, T-dependent antigen or a T-independent antigen. The antigen may be an exogenous antigen. Exogenous antigens are typically antigens that have entered the body from the outside, for example by inhalation, ingestion, or injection. Some antigens may start out as exogenous antigens, and later become endogenous (for example, intracellular viruses). The antigen may be an endogenous antigen. The endogenous antigen may be an antigen that has been generated within cells as a result of normal cell metabolism, or because of pathogenic infections (e.g., viral, bacterial, fungal, parasitic). The antigen may be an autoantigen. The autoantigen may be a normal protein or complex of proteins (and sometimes DNA or RNA) that is recognized by the immune system of patients suffering from a specific autoimmune disease. These antigens should, under normal conditions, not be the target of the immune system, but, due to genetic and/or environmental factors, the normal immunological tolerance for such an antigen is not present in these patients. The antigen may be present or over-expressed due to a condition or disease. The condition or disease may be a cancer or a leukemia. The condition may be an inflammatory disease or condition. The condition or disease may be a metabolic disease. The condition may be a genetic disorder.

The cell surface molecule may be an antigen that has been designated as a tumor antigen. Tumor antigens or neoantigens may be antigens that are presented by MHC I or MHC II molecules on the surface of tumor cells. These antigens may sometimes be presented by tumor cells and never by the normal cells. In this case, they are called tumor-specific antigens (TSAs) and, in general, result from a tumor-specific mutation. More common are antigens that are presented by tumor cells and normal cells, and they are called tumor-associated antigens (TAAs). Cytotoxic T lymphocytes that recognize these antigens may be able to destroy the tumor cells before they proliferate or metastasize. Tumor antigens may also be on the surface of the tumor in the form of, for example, a mutated receptor, in which case they may be recognized by B cells. Unless otherwise specified, the terms “tumor antigen,” “tumor specific antigen” and “tumor associated antigen,” are used interchangeably herein.

The cell surface molecule may be a receptor. The receptor may be an extracellular receptor. The receptor may be a cell surface receptor. By way of non-limiting example, the receptor may bind a hormone, a neurotransmitter, a cytokine, a growth factor or a cell recognition molecule. The receptor may be a transmembrane receptor. The receptor may be an enzyme-linked receptor. The receptor may be a G-protein couple receptor (GPCR). The receptor may be a growth factor receptor. By way of non-limiting example, the growth factor receptor may be selected from an epidermal growth factor receptor, a fibroblast growth factor receptor, a platelet derived growth factor receptor, a nerve growth factor receptor, a transforming growth factor receptor, a bone morphogenic protein growth factor receptor, a hepatocyte growth factor receptor, a vascular endothelial growth factor receptor, a stem cell factor receptor, an insulin growth factor receptor, a somatomedin receptor, an erythropoietin receptor and homologs and fragments thereof. The receptor may be a hormone receptor. The receptor may be an insulin receptor. By way of non-limiting example, the receptor may selected from an eicosanoid receptor, a prostaglandin receptor, an estrogen receptor, a follicle stimulating hormone receptor, a progesterone receptor, a growth hormone receptor, a gonadotropin-releasing hormone receptor, homologs thereof and fragments thereof. The receptor may be an adrenergic receptor. The receptor may be an integrin. The receptor may be an ephrin (Eph) receptor. The receptor may be a luteinizing hormone receptor. The cell surface molecule may be at least about 50% homologous to a luteinizing hormone receptor. The receptor may be an immune receptor. By way of non-limiting example, the immune receptor may be selected from a pattern recognition receptor, a toll-like receptor, a nucleotide oligomerization domain (NOD)-like receptor, a killer activation receptor, a killer inhibitory receptor, an Fc receptor, a B cell receptor, a complement receptor, a chemokine receptor and a cytokine receptor. By way of non-limiting example, the cytokine receptor may be selected from an interleukin receptor, an interferon receptor, a transforming growth factor receptor, a tumor necrosis factor receptor, a colony stimulating factor receptor, homologs thereof and fragments thereof. The receptor may be a receptor kinase. The receptor kinase may be a tyrosine kinase receptor. The receptor kinase may be a serine kinase receptor. The receptor kinase may be a threonine kinase receptor. By way of non-limiting example, the receptor kinase may activate a signaling protein selected from a Ras, a Raf, a PI3K, a protein kinase A, a protein kinase B, a protein kinase C, an AKT, an AMPK, a phospholipase, homologs thereof and fragments thereof. The receptor kinase may activate a MAPK/ERK signaling pathway. The receptor kinase may activate Jak, Stat or Smad.

The cell surface molecule may be a non-receptor cell surface protein. The cell surface molecule may be a cluster of differentiation proteins. By way of non-limiting example, the cell surface molecule may be selected from CD34, CD31, CD117, CD45, CD11b, CD15, CD24, CD114, CD182, CD14, CD11a, CD91, CD16, CD3, CD4, CD25, CD8, CD38, CD22, CD61, CD56, CD30, CD13, CD33, CD123, CD19, CD20 fragments thereof, and homologs thereof.

The cell surface molecule may be a molecule that does not comprise a peptide. The cell surface molecule may comprise a lipid. The cell surface molecule may comprise a lipid moiety or a lipid group. The lipid moiety may comprise a sterol. The lipid moiety may comprise a fatty acid. The antigen may comprise a glycolipid. The cell surface molecule may comprise a carbohydrate.

### Multivalent CAR-EC Switches

Exemplified herein are CAR-EC switches comprising a CAR-ID and a TID. However, one skilled in the art would understand that these switches could further comprise additional target interacting domains and/or additional CAR-IDs. One or more CAR-IDs may be linked/conjugated into one or more internal sites of the TID. One or more CAR-IDs may be linked/conjugated to one or more termini of the TID. Such switches are referred to herein as a “multivalent switch.”

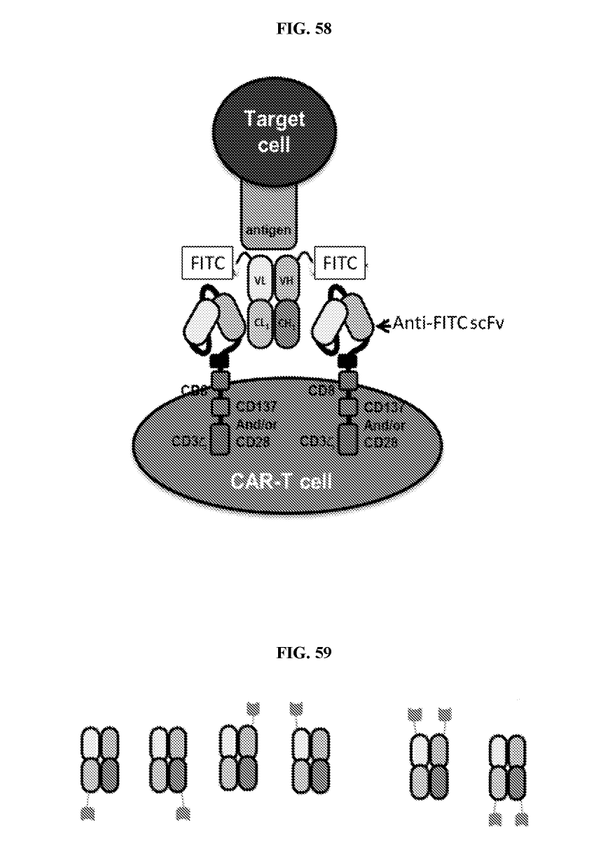

Multivalent switches are advantageous in CAR-T cell activation for at least the reason that multiple CARs are recruited for every one switch (and correspondingly one antigen) (FIG. 58). This is expected to increase the signal transduction, CAR-T cell activation, and target cell lysis. The multivalent switch may bind to a CAR with a longer hinge region than that of canonical CARs in order for the CAR to access multiple peptides of the switch. However, the multivalent switch may, alternatively or additionally, have an optimal geometry and/or length for efficient activity with the CAR, including a canonical CARs or CARs with short hinges.

A first CAR-ID may be linked or conjugated to a first domain of the TID and a second CAR-ID may be linked or conjugated to a second domain of the TID. The first domain and the second domain may be the same. The first domain and the second domain may be different. By way of non-limiting example, the first CAR-ID may be linked to a light chain of a targeting antibody or antibody fragment and a second CAR-ID may be linked to heavy chain of the targeting antibody or antibody fragment. The first CAR-ID may be conjugated to a first terminus of the targeting polypeptide and a second CAR-ID may be conjugated to a second terminus of the targeting polypeptide. By way of non-limiting example, the first CAR-ID may be conjugated to a C terminus of a light chain of a targeting antibody or antibody fragment and a second CAR-ID may be conjugated to an N terminus of a heavy chain of the targeting antibody or antibody fragment. The first CAR-ID may be fused to a terminus of the targeting polypeptide and a second CAR-ID may be linked/conjugated within a domain of the targeting polypeptide. The first CAR-ID and the second CAR-ID may be the same or similar, such that the CAR-EC switch may be used with a CAR-EC cell that expresses one CAR. The first CAR-ID and the second CAR-ID may be different, such that the CAR-EC switch may be used with a CAR-EC cell that expresses one or more CARs or multiple CAR-EC cells that express different CARs.

The switches disclosed herein may comprise one or more CAR-IDs. The switches disclosed herein may comprise two or more CAR-IDs. The switches disclosed herein may comprise three or more CAR-IDs. The switches disclosed herein may comprise 1, 2, 3, 4, 5, 6, 7 or more CAR-IDs. The switches disclosed herein may comprise one or more TIDs. The switches disclosed herein may comprise two or more TIDs. The switches disclosed herein may comprise three or more TIDs. The switches disclosed herein may comprise 1, 2, 3, 4, 5, 6, 7 or more TIDs. The one or more CAR-IDs may be linked and/or conjugated to the one or more TIDs via one or more linkers. Thus, the switches disclosed herein may comprise one or more linkers (e.g., L1, L2). The switches disclosed herein may comprise two or more linkers. The switches disclosed herein may comprise three or more linkers. The switches disclosed herein may comprise 1, 2, 3, 4, 5, 6, 7 or more linkers.

### Soluble T Cell Receptor Switches

TCR and biologics derived from TCRs, such as affinity matured soluble TCR (sTCR) and single chain TCR (scTCR) can also be used as a moiety for switch targeting. These reagents allow detection of intracellular proteins displayed on the cell surface in the context of MHC. An example of this targeting strategy is the affinity-matured sTCR, 1G4c113, which recognizes a peptide derived from the cancer/testes antigen NYESO-1 in the context of HLA-A201. A major limitation of antibody-based targeting is the restriction to cell surface targets; however many tumor associated antigens are intracellular, non-membrane bound proteins. One way to target intracellular proteins is by immunologic targeting using T cell receptors (TCRs). This exploits the mechanism in which a T cell naturally surveys the intracellular proteome, represented as peptides displayed by human leukocyte antigen (HLA, MHCI). However, affinity matured, tumor-specific TCRs transduced into adoptively transferred T cells have the potential to lose specificity and have caused serious adverse effects in the clinic due to off-target reactivity. For example, adoptive transfer of engineered T cells harboring a high avidity TCR against MAGE-A3 for melanoma and myeloma resulted in two deaths in the clinic due to off-target reactivity with the protein Titin in cardiac tissue. On-target, off-tumor toxicity has also been reported. An affinity matured TCR against CEA caused severe inflammatory colitis even at doses of T cells 100 fold lower than those typically given with autologous tumor infiltrating lymphocyte (TIL) therapy. Surprisingly, these serious safety concerns have not stopped researchers from initiating clinical trials for more than a dozen targets. Therefore, using an sTCR as a CAR-EC switch may be highly advantageous.

Off-target reactivity is potentiated in the TCR complex by cooperative binding between CD8 and MHCI. Monoclonal TCRs (mTCR) that are expressed in soluble form do not result in cooperative target binding from CD8 in the same way as those expressed in a T cell and therefore may not have the same level of off-target binding. The use of soluble mTCRs as switches for sCAR-T will allow dose titratable sCAR-T cell targeting of intracellular tumor associated antigens that may be turned off in the case of an adverse event.

The heterologous expression of mTCRs and their use as soluble therapeutics is well established. Natural TCRs have weak affinity (1-100 μM) for their targets as they are naturally tuned for cooperative binding of CD8. As such, directed evolution strategies have used phage display to produce mTCRs with affinities strong as 1 pM. Soluble TCRs have additional advantages over mAbs including the ability to target intracellular proteins, a small size allowing for improved tumor penetration, the ability to detect very low cell surface antigen densities, a fully human structure, and an inexpensive E. coli expression system.

Grafting molecules like FITC onto TCR-derived products like 1G4c113 enables the expansion of CAR-EC targeting to intracellular antigens not normally accessible by conventional antibody-derived switches.

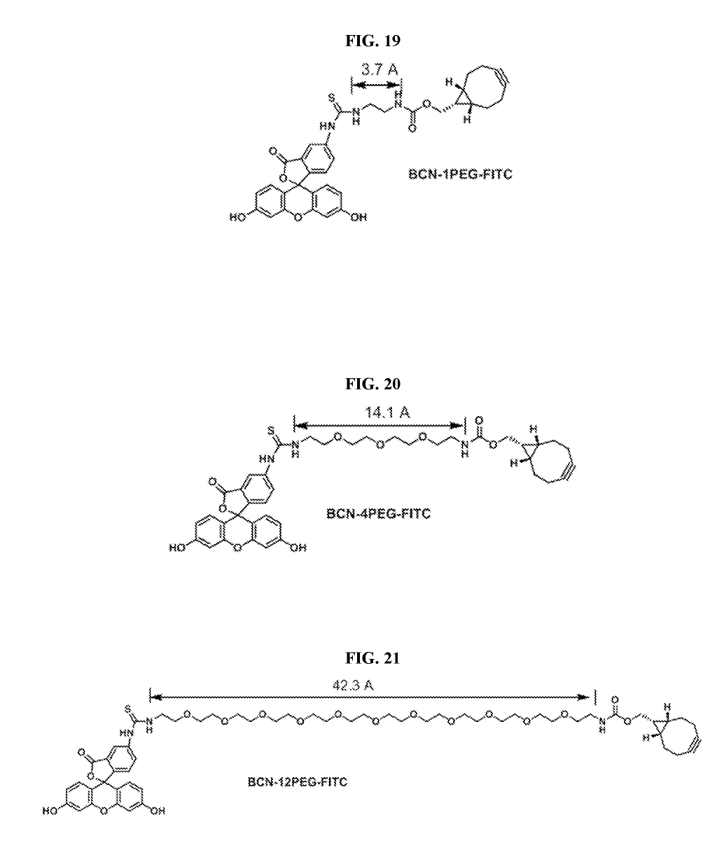

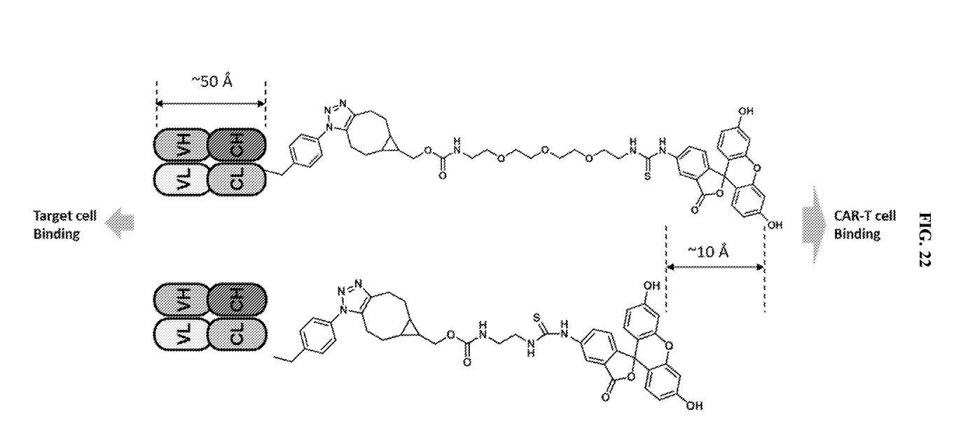

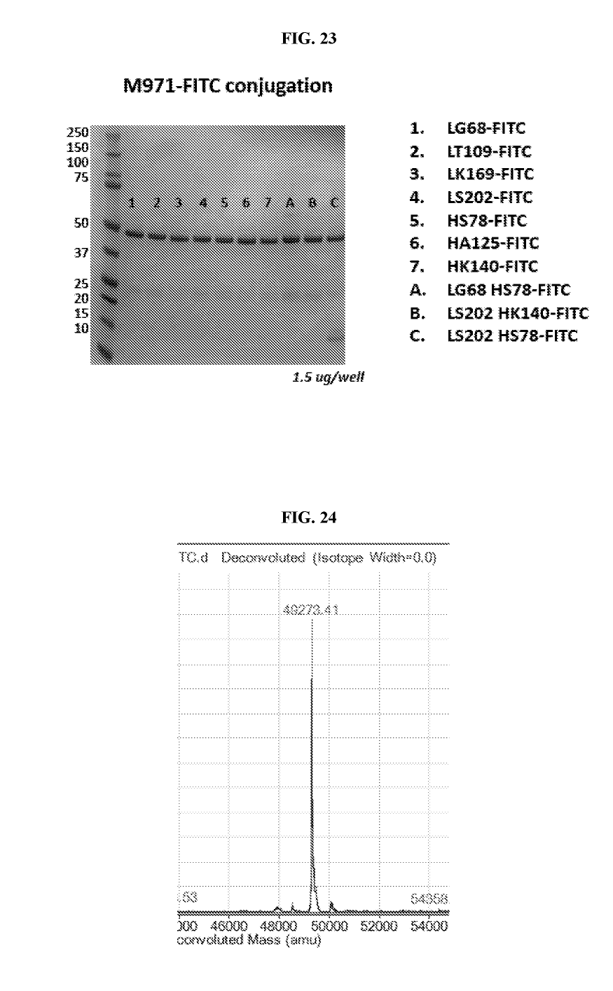

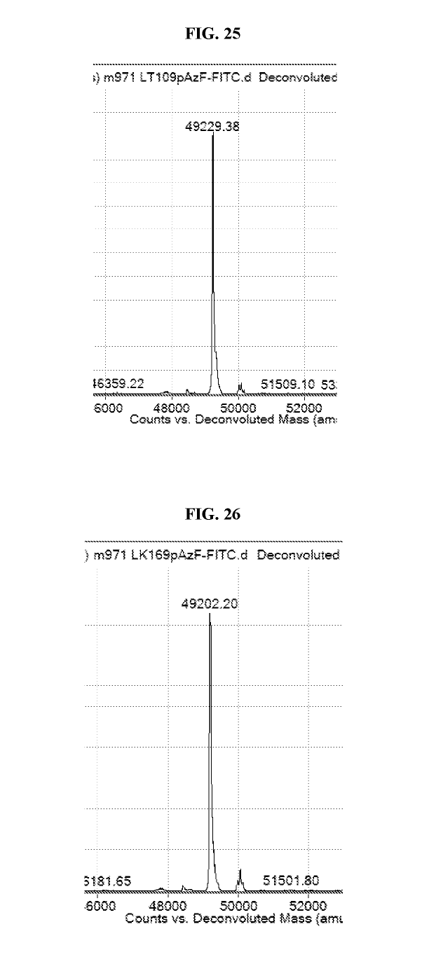

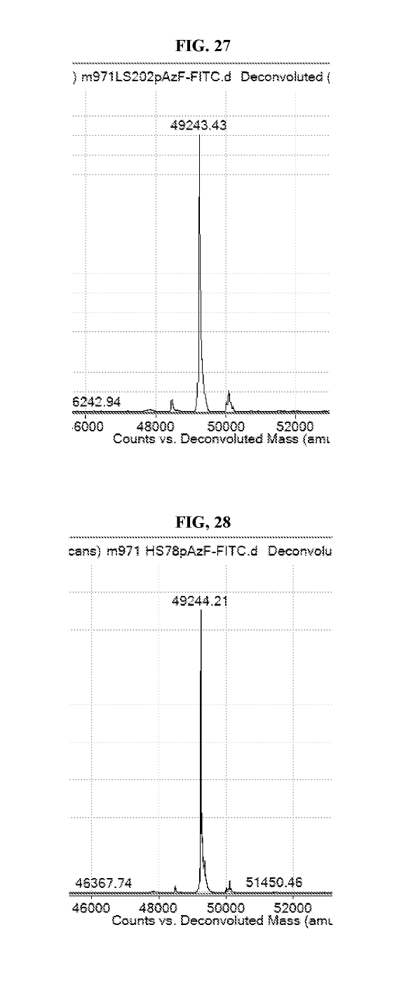

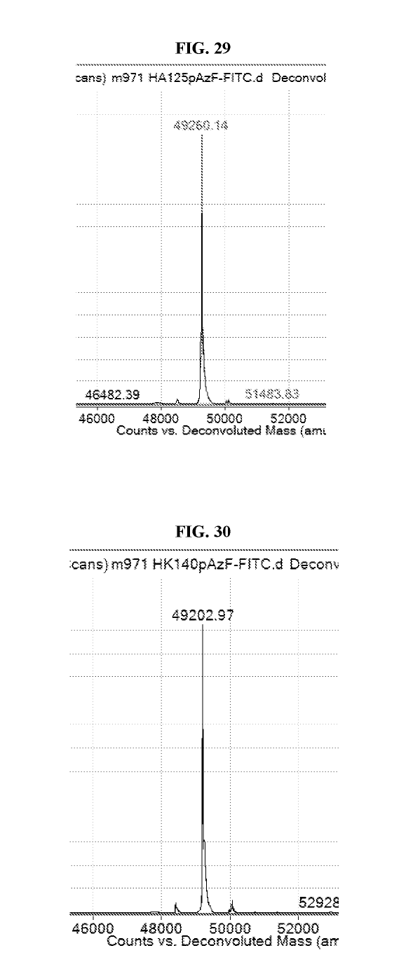

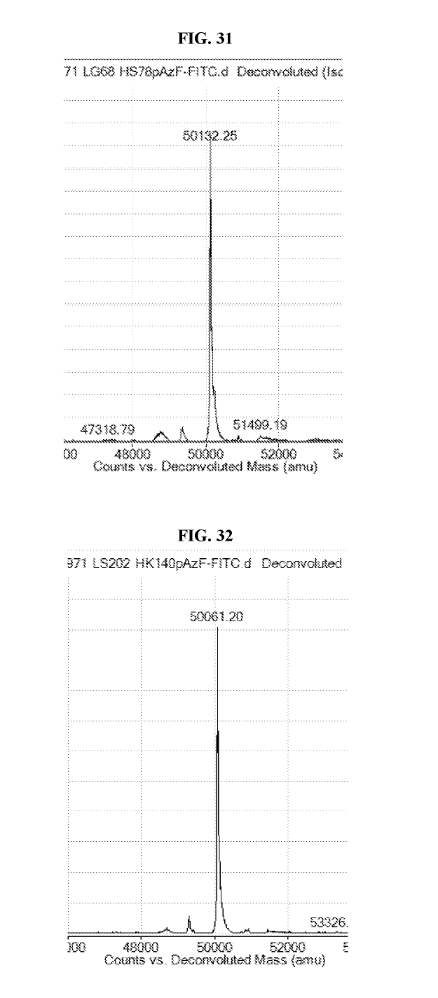

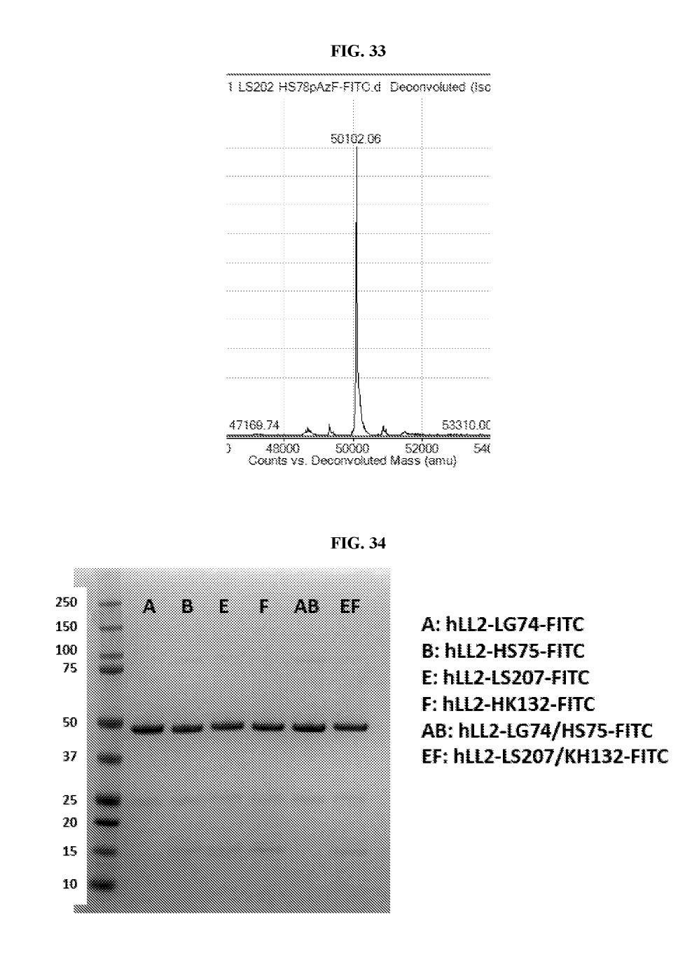

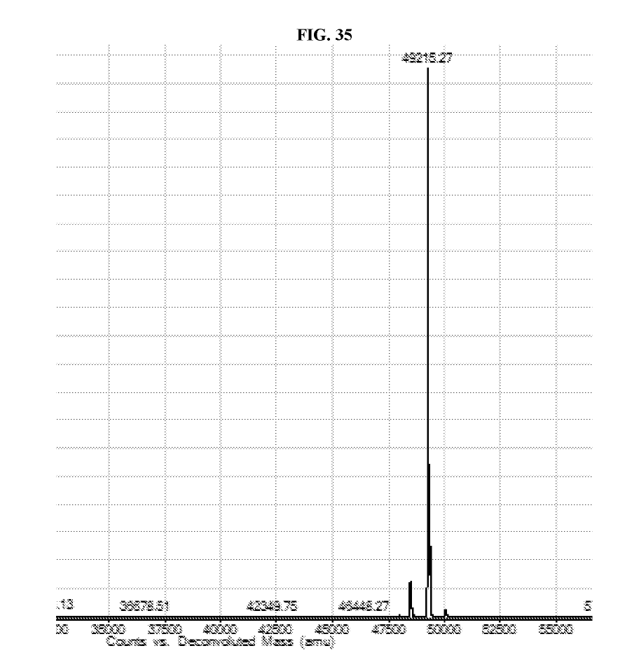

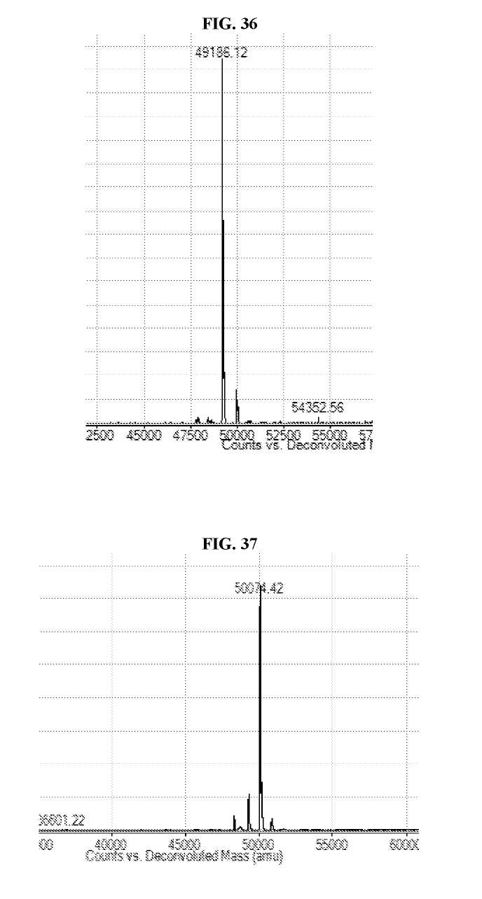

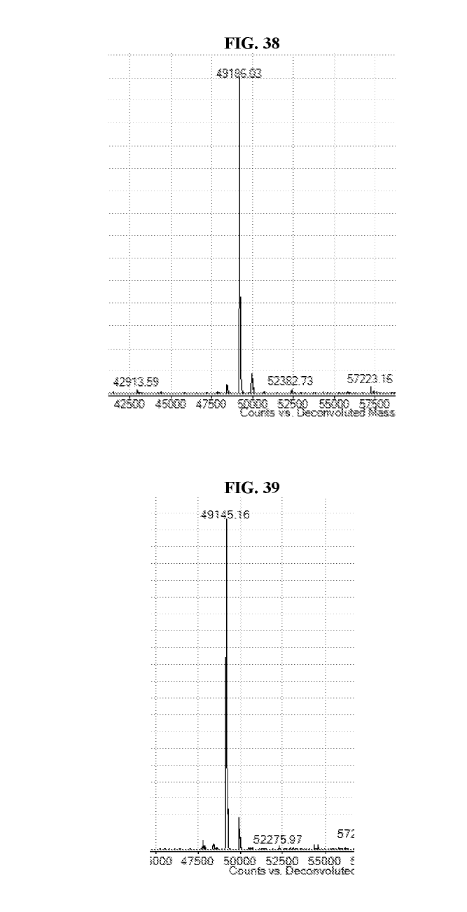

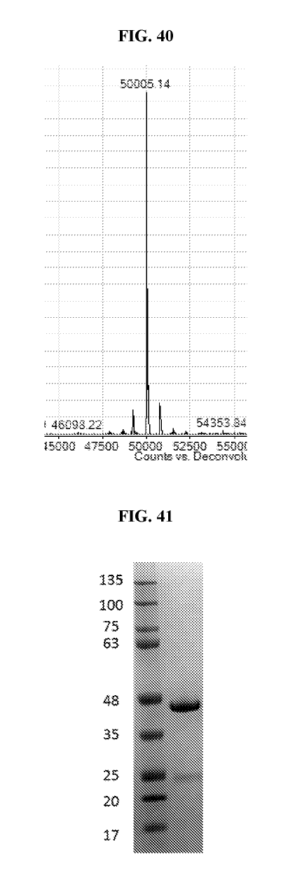

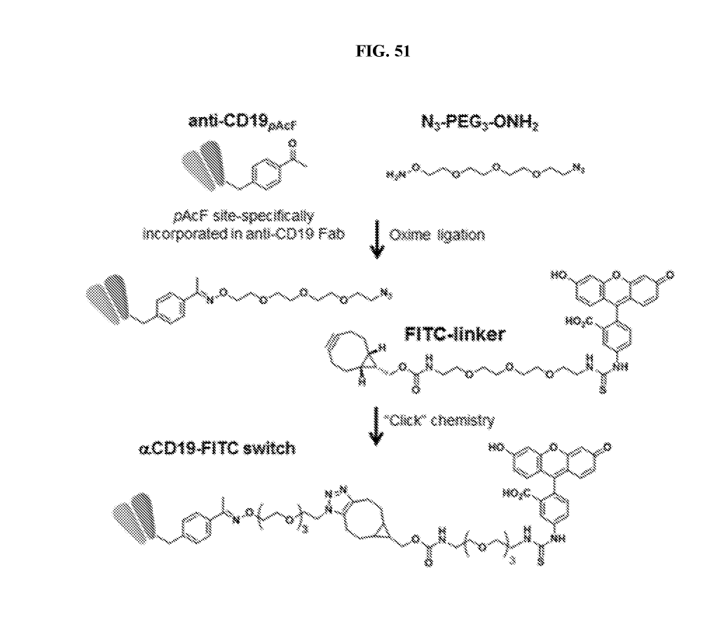

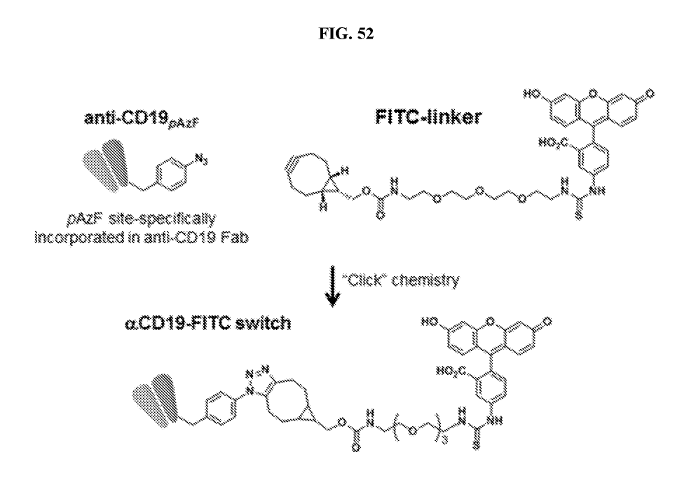

Disclosed herein are soluble T cell receptor (sTCR) switches comprising: a CAR-ID; and a sTCR or portion thereof. The CAR-ID may be linked or conjugated to a terminus of a domain of the sTCR. The CAR-ID may be linked or conjugated into an internal site of a domain of the sTCR. The domain of the sTCR may be selected from an α chain, a β chain, a γ chain, a δ chain, an ϵ chain and a ζ chain. The sTCR switch may further comprise a linker, wherein the linker links the CAR-ID to the sTCR or portion thereof. The linker may be selected from a linker depicted in FIG. 19-FIG. 22, FIG. 51, FIG. 52, FIG. 54, or FIG. 55. The CAR-ID may comprise a hapten. The hapten may be FITC or a derivative thereof. The CAR-ID may not comprise a peptide. The sTCR may comprise an unnatural amino acid. The CAR-ID may be linked or conjugated to the unnatural amino acid.

The sTCR switch may comprise a fusion of the CAR-ID (e.g. FITC) to the sTCR. The sTCR switch may comprise the CAR-ID, wherein the CAR-ID is linked or conjugated to the sTCR. sTCR switches may comprise the CAR-ID at an N-terminus of a TCRα chain, an N-terminus of a TCRβ chain, a C-terminus of the TCRα chain or a C-terminus of the TCRβ chain. The TCRα chain may be encoded by SEQ ID NO. 33. The TCRα chain may be greater than about 50%, about 60%, about 70%, about 80% or about 90% homologous to SEQ ID NO. 33. The TCR beta chain may be encoded by SEQ ID NO. 32. The TCR beta chain may be greater than about 50%, about 60%, about 70%, about 80% or about 90% homologous to SEQ ID NO. 32. Alternatively or additionally, the CAR-ID may be linked or conjugated within a chain/region of the soluble TCR. Additional structure based design may be employed to link/conjugate additional CAR-IDs to additional chains/regions of the TCR that are permissive to mutation.

The potential to target intracellular antigens opens the door to sCAR-T cells that can remodel the tumor microenvironment. For example, naturally occurring CD8 T cells have been recently identified that recognize FoxP3 or indoleamine-pyrrole 2,3-dioxygenase (IDO) expressing regulatory T cells (Treg) and myeloid-derived suppressor cells (MDSC), respectively. The cloning and heterologous expression of mTCRs from these cells as switches may enable a sCAR-T cell to deplete immunosuppressive cells within the tumor microenvironment. This may be a novel route to overcoming disease-mediated immunosuppression.

### Chimeric Antigen Receptor (CAR)

The switches disclosed herein may interact with a CAR on a CAR-EC, thereby regulating the activities of the CAR-EC. Generally, the interaction of the CAR-ID with the CAR may result in the activation of an immune response by the cell. The CAR may comprise an extracellular domain, a transmembrane domain and an intracellular domain. The extracellular domain may interact with the CAR-ID of the CAR-EC switch. The extracellular domain may comprise at least a portion of an antibody. In some instances, the antibody is not a full-length antibody. The extracellular domain may comprise at least a portion of an immunoglobulin or fragment thereof. The immunoglobulin or fragment thereof may be selected from a group comprising IgA1, IgA2, IgD, IgM, IgE, IgG1, IgG2, IgG3, IgG4, scFv, di-scFv, bi-scFv and Fab, Fc, F(ab′)₂, pFc′, a nanobody, an affibody, a DARPin, a diabody, a camelid, an engineered T cell receptor, or a monobody. The immunoglobulin may comprise IgG4.

The antibody may have a binding affinity of about 0.01 pM, about 0.02 pM, about 0.03 pM, about 0.04 pM, 0.05 pM, about 0.06 pM, about 0.07 pM, about 0.08 pM, about 0.09 pM, about 0.1 pM, about 0.2 pM, 0.3 pM, about 0.4 pM, about 0.5 pM, about 0.6 pM, about 0.7 pM, about 0.8 pM, about 0.9 pM or about 1 pM, about 2 pM, about 3 pM, about 4 pM, about 5 pM, about 6 pM, about 7 pM, about 8 pM, about 9 pM, about 10 pM, about 0.01 nM, about 0.02 nM, about 0.03 nM, about 0.04 nM, about 0.05 nM, about 0.06 nM, about 0.07 nM, about 0.08 nM, about 0.09 nM, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 2 nM, about 3 nM, about 4 nM, about 5 nM, about 6 nM, about 7 nM, about 8 nM, about 9 nM, about 10 nM, about 12nM, about 14 nM, about 16 nM, about 18 nM, about 20 nM, about 22 nM, about 24 nM, about 26 nM, about 28 nM or about 30 nM. The extracellular domain may comprise at least a portion of a single chain variable fragment (scFv). The extracellular domain may comprise avidin or a fragment thereof. The extracellular domain may not comprise avidin or fragment thereof. The antibody may comprise an anti-FITC antibody or fragment thereof. The anti-FITC antibody may be an anti-FITC scFv. The anti-FITC scFv may be selected from 4-4-20, 4D5Flu, 4M5.3 and FITC-E2. The anti-FITC scFv may be encoded by a sequence selected from SEQ ID NOs: 1-4.

The antibody to FITC or fragment thereof may have a binding affinity for FITC less than 0.1 pM. The antibody to FITC or fragment thereof may have a binding affinity for FITC between about 0.1 pM and about 1 pM. The antibody to FITC or fragment thereof may have a binding affinity for FITC between about 1 pM and about 10 pM. The antibody to FITC or fragment thereof may have a binding affinity for FITC of about 10 pM, about 20 pM, about 30 pM, about 40 pM, about 50 pM, about 60 pM, about 70 pM, about 80 pM, about 90 pM or about 100 pM. The antibody to FITC or fragment thereof may have a binding affinity for FITC of about 100 pM, about 200 pM, about 300 pM, about 400 pM, about 500 pM, about 600 pM, about 700 pM, about 800 pM, about 900 pM or about 1 nM. The antibody to FITC or fragment thereof may have a binding affinity for FITC of about 1 nM, about 2 nM, about 3 nM, about 4 nM, about 5 nM, about 6 nM, about 7 nM, about 8 nM, about 9 nM or about 10 nM. The antibody to FITC or fragment thereof may have a binding affinity for FITC of about 10 nM, about 15 nM, about 20 nM, about 25 nM, about 30 nM, about 35 nM, about 40 nM, about 45 nM or about 50 nM. The antibody to FITC or fragment thereof may have a binding affinity for FITC greater than 50 nM. The antibody to FITC may comprise an anti-FITC scFv or fragment thereof. The anti-FITC scFv may be selected from a group comprising 4-4-20, 4D5Flu, 4M5.3 and FITC-E2. The binding affinity of 4-4-20 may be about 0.2 nM. The binding affinity of 4D5Flu may be about 20 nM. The binding affinity of 4M5.3 may be about 0.3 pM. The binding affinity of FITC-E2 may be about 0.3 nM.

The transmembrane domain and/or the intracellular domain may comprise at least a portion of a cytoplasmic signaling domain. The intracellular domain may comprise at least a portion of a signaling molecule selected from the group comprising CD3ξ, CD28, and 4-1BB. The intracellular domain may comprise an Fc receptor or a portion thereof. The Fc receptor or portion thereof may be CD16 or a portion thereof. The signaling molecule may comprise CD3ξ. The signaling molecule may comprise CD28. The signaling molecule may comprise 4-1BB. The intracellular domain may comprise at least a portion of CD3ξ. The intracellular domain may comprise at least a portion of CD28, The intracellular domain may comprise at least a portion of 4-1BB, The intracellular domain may comprise at least a portion of OX-40, The intracellular domain may comprise at least a portion of CD30, The intracellular domain may comprise at least a portion of CD40, The intracellular domain may comprise at least a portion of CD2. The intracellular domain may comprise at least a portion of CD27. The intracellular domain may comprise at least a portion of PD-1. The intracellular domain may comprise at least a portion of ICOS. The intracellular domain may comprise at least a portion of lymphocyte function-associated antigen-1 (LFA-1). The intracellular domain may comprise at least a portion of CD7. The intracellular domain may comprise at least a portion of homologous to lymphotoxins, inducible expression, competes with herpesvirus glycoprotein D for herpes virus entry mediator, a receptor expressed on T lymphocytes (LIGHT). The intracellular domain may comprise at least a portion of NKG2C. The intracellular domain may comprise at least a portion of B7-H3. The intracellular domain may comprise at least a portion of a cytoplasmic signaling domain from one or more signaling molecules. The intracellular domain may comprise at least a portion of two or more cytoplasmic signaling domains. The two or more cytoplasmic signaling domains may be from two or more different signaling molecules. The intracellular domain may comprise at least a portion of three or more cytoplasmic signaling domains. The intracellular domain may comprise at least a portion of four or more cytoplasmic signaling domains. The intracellular domain may comprise at least a portion of a ligand that binds to one or more signaling molecules. The intracellular domain may comprise at least a portion of a ligand that binds to CD83.

The CAR may comprise a hinge domain. The hinge domain may be located in the extracellular domain of the CAR. The hinge domain may be located between the transmembrane domain and a region that interacts with a chimeric antigen receptor switch. The hinge may comprise a portion of the extracellular domain. The hinge may comprise a portion of the transmembrane domain. The hinge may be flexible (e.g. the hinge may be a linear sequence of amino acids with no known secondary structure in which the torsion angles or rotation around the bonds of the polypeptide backbone have the freedom to occupy many different orientations). The hinge may be rigid (e.g. the hinge comprises a beta sheet, coiled coil structure, or otherwise rigid structure in which the torsion angles or rotation around the bonds of the polypeptide backbone have defined preference to occupy a limited number of orientations). The hinge may provide a length, orientation, geometry or flexibility to the CAR that is necessary for an optimal immunological synapse. The optimal immunological synapse may provide for an optimal distance and/or orientation between the CAR-EC and the target cell. The optimal immunological synapse may provide for optimal and/or maximal cytotoxicity against the target cell. The hinge may comprise about 1, about 2, about 3, about 4, about 5, about 6, about 7, about 8, about 9, about 10, about 12, about 14, about 16, about 18, about 20, about 25, about 30, about 35, about 40, about 45, about 50, about 55, about 60, about 65, about 70, about 75, about 80, about 90 or about 100 amino acids. The hinge may comprise a sequence selected from SEQ ID NOS: 34-37. The hinge may comprise a sequence that is at least about 50% homologous to a sequence selected from 34-37.

The CAR may be expressed at relatively low levels (˜10,000 to ˜100,000 copies per cell) on the CAR-EC. The CAR may be expressed at less than about 10,000 copies per cell. The CAR may be expressed at relatively high levels on the CAR-EC (more than ˜500,000 copies per cell). The CAR may be expressed at moderate levels (˜100,000 to ˜500,000 copies per cell). The CAR may be expressed under the control of a promoter selected from EF1a, IL-2, CMV, and synthetic promoters designed to increase or decrease CAR expression. The promoter may be constitutive. The promoter may be inducible.

### Multivalent CARS

Valency can also be engineered into a CAR hinge. Disclosed herein are CARs and systems thereof wherein a first cysteine of a first chimeric antigen receptor and a second cysteine of a second chimeric antigen receptor form a disulfide bond, resulting in multimerization of the first chimeric antigen receptor and the second chimeric antigen receptor. By way of non-limiting example, a monovalent switches may recruit two CARs through a disulfide that forms in the hinge region of the CAR. The hinge domain may have a sequence selected from SEQ ID NOS: 34-37. The hinge domain may have a sequence that is at least about 50%, about 60%, about 70%, about 80% or about 90% homologous to a sequence selected from SEQ ID NOS: 34-37. The CAR may comprise an extracellular domain having a region that binds a CAR switch. The CAR switch may comprise a hapten, wherein the hapten interacts with the chimeric antigen receptor. The hapten may be selected from FITC, dinitrophenol and biotin. The hapten may be FITC or a derivative thereof.

The hinge may be a CD8-derived hinge (SEQ ID NO. 34) which is expected to be monovalent. The hinge may be derived from the hinge region of an IgG molecule. The IgG molecule may be selected from IgG1, IgG4 or a mutated IgG4 (IgG4m). The IgG4 hinge (SEQ ID NO: 31) may not participate in interchain disulfides but instead has intrachain disulfide bonds which do not dimerize the CAR. The hinge may be considered functionally monovalent. The IgG1 and IgG4m hinge (SEQ ID NO:32) may contain a serine to proline mutation which enables it to participate in interchain disulfide bonds which covalently dimerizes the hinge region (FIG. 41). These hinges may be used to study both the distance constraints of an immunological synapse (by testing the long CD8-derived hinge vs the short IgG4 derived hinge) and the valency effect (by testing the short IgG4 derived hinge vs an IgG4m derived CAR) of the CAR and/or switch. Other hinges may comprise CH2 and/or CH3 of IgG1, IgG2, IgG3, or IgG4 molecules, or portions thereof, or combinations thereof. The hinge may be derived from CD28. The hinge may dimerize.

coCARS/iCARs

The switchable CARs and switches disclosed herein may encompass inhibitory chimeric antigen receptor (iCAR)-T cell switches and switchable iCAR-T cells for targeting an immune response to specific cells (e.g. diseased cells) and minimizing an immune attack on healthy cells. The sCARs and switches disclosed herein may also encompass co-stimulatory chimeric antigen receptor (coCAR)-T cell switches for use with switchable coCAR-T cells for targeting an immune response to target cells (e.g. diseased cells) and maximizing an immune attack on these cells. iCAR-T cell switches and coCAR-T cell switches comprise a CAR-ID and a TID. Compositions disclosed herein may comprise a plurality of switches for modulating a CAR-EC, wherein a first switch that interacts with a first antigen on a first target cell and a first CAR on the CAR-EC; and a second switch that interacts with a second antigen on a second target cell and a second CAR on the CAR-EC. The plurality of switches may be used with existing CAR-T cells and with CAR-ECs that express a canonical CAR and/or an iCAR. The plurality of switches may be used with existing CAR-T cells and with CAR-ECs that express a canonical CAR and/or a coCAR.

The sCAR-EC cells disclosed herein may comprise a first sCAR and a second sCAR. The first sCAR may be a canonical CAR and the second sCAR may be an iCAR. The first sCAR may be a canonical CAR and the second sCAR may be a coCAR.

The iCAR may comprise a chimeric receptor which provides an inhibitory signal to CAR-T cells. The iCAR may comprise a cytoplasmic domain selected from PD-1, NAG-3, TIM-3, and CTLA-4. The iCAR may be expressed by the same cell as a canonical (activating) CAR. Activation of the iCAR may tune down a canonical CAR signal and/or activity. The specificity of the iCAR can be used to protect tissues in which CAR-T cell activity is not desirable. iCAR activity may be controlled by a switch, referred to as an “iCAR switch” herein. Similarly, canonical (activating) CAR activity may be controlled by the first and/or second switch, referred to as an “aCAR switch” herein. A switchable iCAR-T cell enables targeting of antigens that may be unsafe to target with a canonical or CAR-T cell.

To mount an immune response, the aCAR switch binds a positive, or “A” antigen on a target cell that is to be attacked (e.g. cancer cell) and the canonical CAR, stimulating cytotoxic activity towards the target cell through activation of the canonical CAR. To protect normal tissue, the iCAR switch binds a negative, or “B”, antigen on a cell that is to be avoided by T cells (e.g. a healthy cell) and the iCAR, inhibiting immune activity through signaling of the iCAR. The “B” antigen may be ubiquitously expressed on normal tissue but down-regulated in most malignant cells. The “A” antigen may be over-expressed in malignant cells relative to normal tissue. The B antigen may be opioid binding protein/cell adhesion molecule-like gene (OPCML). The B antigen may be selected from hyaluronidase 2 (HYAL2), deleted in colorectal cancer (DCC), and scaffold/matrix attachment region binding protein 1 (SMAR1).

The coCAR may comprise a chimeric receptor which provides a co-stimulatory signal to CAR-T cells. The coCAR may comprise a cytoplasmic domain selected from CD137 and/or CD28. The coCAR may be expressed by the same cell as a canonical (activating) CAR. Activation of the coCAR may enhance and/or synergize a canonical CAR signal and/or activity. The coCAR may increase cytotoxicity towards a target cell relative to the cytotoxicity towards a target cell generated by a CAR-T cell that only expresses a canonical CAR-T cell. coCAR activity may be controlled by a switch, referred to as an “coCAR switch” herein. Similarly, canonical CAR activity may be controlled by the first and/or second switch, referred to as an “aCAR switch” herein.

### Non-Antibody CARS

The chimeric receptors disclosed herein may comprise a non-antibody extracellular domain that interacts with the CAR-ID. The extracellular domain may be a non-antibody protein or a non-antibody peptide. Unlike canonical CARs, the extracellular domain may not comprise an antibody or antibody fragment. The chimeric receptor binding partner may be non-antibody protein or peptide.

### Epsilon CAR

Further disclosed herein are CARs comprising: an extracellular domain that interacts with an anti-CD3 antibody or fragment thereof on the switch; a transmembrane domain; and an intracellular domain, wherein at least a portion of the transmembrane domain or at least a portion of the intracellular domain is not based on or derived from a CD3 protein. The extracellular domain may comprise a CD3 extracellular domain or portion thereof. The extracellular domain may comprise a CD3ϵ extracellular domain or portion thereof. The extracellular domain may comprise a CD3δ extracellular domain or portion thereof. The extracellular domain may comprise a CD3γ extracellular domain or portion thereof. The extracellular domain may comprise a CD3ζ extracellular domain or portion thereof. The extracellular domain may comprise an a chain of TCR extracellular domain or portion thereof. The extracellular domain may comprise a pre-α chain of TCR extracellular domain or portion thereof. The extracellular domain may comprise a β chain of TCR extracellular domain or portion thereof.

### Chimeric Antigen Receptor Effector Cells (CAR-EC)

The methods, platforms and kits disclosed herein may comprise one or more CAR-EC or uses thereof. The CAR-ECs disclosed herein express a CAR. The CAR may be any CAR disclosed herein. Wherein the methods, platforms or kits comprise two or more effector cells, the two or more effector cells may be of the same cell type. The two or more effector cells may be of a different cell type. The two or more effector cells may be of the same cell lineage. The two or more effector cells may be of different cell lineages. The two or more effector cells may comprise two or more identical CARs. The two or more effector cells may comprise two or more different CARs. The two or more effector cells may comprise two or more similar CARs.

The effector cell may be a T cell. The effector cell may be a cell of a T cell lineage. The effector cell may be a mature T cell. The effector cell may be a precursor T cell. The effector cell may be a cytotoxic T cell. The effector cell may be a naive T cell. The effector cell may be a memory stem cell T cell (T_{MSC}). The effector cell may be a central memory T cell (T_{CM}). The effector cell may be an effector T cell (TE). The effector cell may be a CD4+ T cell. The T cell may be a CD8+ T cell. The effector cell may be a CD4+ and CD8+ cell. The effector cell may be an alpha-beta T cell. The effector cell may be a gamma-delta T cell. The effector cell may be a natural killer T cell. The effector cell may be a helper T cell.

While preferred embodiments of the present disclosure describe methods, kits and platforms comprising T cells, one skilled in the art may also understand that other cell types may be used in place of a T cell. The effector cell may be an effector cell that has an effect on a target or target cell when brought into proximity of the target or target cell. The effector cell may be a cell that has a cytotoxic effect on a target or target cell when brought into proximity of the target or target cell. The effector cell may be an immune cell. The effector cell may be selected from a B cell, a monocyte, a thrombocyte, a leukocyte, a neutrophil, an eosinophil, a basophil, or a lymphocyte. The effector cell may be a lymphocyte. The effector cell may be a macrophage. The effector cell may be a phagocytic cell. The effector cell may be an effector B cell. The effector cell may be a natural killer cell. The effector cell may isolated or derived from a subject suffering from a disease or condition. The effector cell may be a cell derived from a subject to be treated with a CAR-EC switch or CAR-EC platform disclosed herein.

The T cell may express a chimeric antigen receptor encoded by one or more polynucleotides based on or derived from SEQ ID NOS: 1-4. The polynucleotide may be at least about 5%, 10%, 15%, 20%, 25%, 30%, 35%, 40%, 45%, 50%, 55%, 60%, 65%, 70%, 75%, 80%, 85%, 90% or 95% identical to one or more polynucleotides based on or derived from SEQ ID NOS: 1-4. The polynucleotide may be at least about 70% identical to one or more polynucleotides based on or derived from SEQ ID NOS: 1-4. The polypeptide encoded by one or more polynucleotides may be based on or derived from SEQ ID NOS: 1-4. The polypeptide may be encoded by a polynucleotide that is at least about 5%, 10%, 15%, 20%, 25%, 30%, 35%, 40%, 45%, 50%, 55%, 60%, 65%, 70%, 75%, 80%, 85%, 90% or 95% identical to one or more polynucleotides based on or derived from SEQ ID NOS: 1-4. The polynucleotide may be constitutively expressed. The polynucleotide may be conditionally expressed.

Disclosed herein are methods of producing a chimeric antigen receptor effector cell (CAR-EC), the methods comprising introducing one or more polynucleotides encoding a CAR or a CAR-complex into an effector cell. The effector cell may be a T cell. Introducing one or more polynucleotides encoding a CAR or a CAR-complex into an effector cell may comprise transfecting the effector cell with the one or more polynucleotides. Introducing one or more polynucleotides encoding a CAR or a CAR-complex into an effector cell may comprise virally infecting the effector cell with one or more viruses comprising the one or more polynucleotides encoding a CAR disclosed herein. The virus may be a lentivirus. The virus may be an adenovirus. The virus may be a retrovirus. The virus may be an adeno-associated virus. The virus may be a self-complementary adeno-associated virus (scAAV). The virus may be a modified human immunodeficiency (HIV) virus. The virus may be a modified herpes simplex virus (HSV) virus. Other methods of producing the CAR-EC may comprise a method of transferring one or more polynucleotides encoding a CAR into a cell, wherein the methods comprise adding a transposon, a zinc finger nuclease, a transcription activator-like effector nuclease (TALEN), or a clustered regularly-interspaced short palindromic repeat (CRISPR) to the cell. The transposon may be a sleeping beauty transposon.

### Tumor Infiltrating Lymphocytes (TILs)

The effector cell may be a tumor-infiltrating lymphocyte (TIL). TILs are a type of white blood cell found in tumors. TILs are implicated in killing tumor cells, and the presence of lymphocytes in tumors is often associated with better clinical outcomes. To obtain TILs, autologous lymphocytes may be isolated from patients' tumors and grown to very large numbers of cells in vitro. Prior to TIL treatment, the subject may be given nonmyeloablative chemotherapy to deplete native lymphocytes (“lymphodepletion”) that can suppress tumor killing. Once lymphodepletion is complete, the subject may be infused with the TILs. TILs may be administered in combination with interleukin 2 (IL-2).

The present application provides for TILs that are modified to express a CAR and applications thereof (e.g. CAR-TIL therapy). Disclosed herein are T cells (e.g. TILs) modified to express a CAR. Further disclosed herein are methods for treating a condition in a subject in need thereof, comprising administering a TIL, wherein the TIL expresses a CAR. The CAR may be a co-receptor of a T cell receptor (TCR) expressed by the TIL. The CAR may associate with a TCR of the TIL. The CAR may enhance TCR activation. The CAR may have intracellular signaling domains that are activated upon association and/or interaction with a TCR, wherein the TCR is bound to an antigen on a target cell. These methods may be referred to as CAR-TIL therapy. An advantage of this application is to utilize the specificity of endogenous TCRs of the engineered T cells (e.g. antigen specific MHC), circumventing the need to introduce artificial tumor targeting moieties (e.g. antibody-based switches) used in conventional CAR-T approaches, for the recognition of the target tumor cells. The endogenous TCRs expressed on tumor-specific T cells are heterogeneous, but may be pre-selected for specifically targeting tumor-associated peptide antigens bound to MHCs on tumor cells. Moreover, the diverse repertoire of the endogenous, tumor specific TCRs are suitable to target heterogeneous tumors.

### CAR-EC Platform

Disclosed herein are CAR-EC platforms comprising a an effector cell, wherein the effector cell comprises a polynucleotide encoding a CAR and a CAR-EC switch, wherein the CAR-EC switch comprises a CAR-ID and a TID and wherein the CAR-EC switch binds a cell surface molecule on a target cell. The CAR-EC switch may be selected from any CAR-EC switches disclosed herein.

The CAR-EC platforms may comprise two or more CAR-EC switches. The CAR-EC platforms may comprise 3, 4, 5, 6, 7, 8, 9, 10, 11, 12, 13, 14, 15, 16, 17, 18, 19, 20 or more CAR-EC switches. The CAR-EC platforms may comprise may comprise more than 20, more than 25, more than 30, more than 35, more than 40, more than 45 or more than 50 CAR-EC switches. The two or more switches may be selected from one or more CAR-EC switches disclosed herein or a combination thereof.

The CAR-EC platforms disclosed herein may further comprise a first CAR-EC switch and a second CAR-EC switch, wherein the first CAR-EC switch comprises a first CAR-ID and a first TID and the second CAR-EC switch comprises a second CAR-ID and a second TID. The first CAR-ID and the second CAR-ID may be the same. The first CAR-ID and the second CAR-ID may be different. The first CAR-ID and the second CAR-ID may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous. The first TID and the second TID may be the same. The first TID and the second TID may be different. The first TID and the second TID may be about 99%, about 98%, about 97%, about 96%, about 95%, about 92%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% or about 2% homologous.

### Kits, Vectors and Polynucleotides

Disclosed herein are kits comprising one or more CAR-EC switches disclosed herein. The kit may further comprise two or more CAR-EC switches. The kit may comprise three CAR-EC switches. The kit may comprise about 3, 4, 5, 6, 7, 8, 9, 10, 12, 15, 20, 24, 30, 35, 48, 50, 55, 60, 65, 70, 75, 80, 85, 90, 96, 100, 120, 150, 200, 300, 384, 400, 500, 600, 700, 800, 900 or 1000 CAR-EC switches. The kit may be employed for biological research. The kit may be used for diagnosing a disease or a condition. The kit may be used for treating a disease or condition. The CAR-EC switches of the kit may be used with CAR-EC cells disclosed herein or existing CAR T-cells clinically used or tested. The kit may further comprise one or more effector cells. The kit may further comprise one or more CAR-EC cells. The CAR-EC cell may be a T cell. The T cell may express one or more CARs. The kit may further comprise a polynucleotide encoding one or more CARs. The kit may further comprise a vector comprising a polynucleotide encoding one or more CARs. The CAR may be selected from any of the CARs disclosed herein. The kit may comprise one or more polynucleotides encoding a CAR-EC switch disclosed herein or a portion thereof (e.g. antibody, antibody fragment, peptide).

Further disclosed herein are vectors and polynucleotides encoding the target interacting domain (TID) of the CAR-EC switch. The polynucleotides may be DNA. The polynucleotides may be RNA. Unless otherwise specified, the terms “polynucleotide” and “vector,” as used herein, are used interchangeably. The TID may be an antibody or antibody fragment. The vector may comprise a sequence encoding a heavy chain of the antibody or antibody fragment. The vector may comprise a sequence encoding a light chain of the antibody or antibody fragment. The vector may comprise the sequence encoding the light chain of the antibody or antibody fragment and the sequence encoding the heavy chain of the antibody or antibody fragment. The light chain and the heavy chain may be expressed from the same vector. The light chain and the heavy chain may be expressed from two separate vectors.

Disclosed herein are vectors and polynucleotides encoding CARS, wherein the CARs comprise an extracellular domain that binds to a peptide of a CAR-EC switch. The extracellular domain may comprise an antibody or antibody fragment. The antibody or antibody fragment may bind a CAR-ID of a CAR-EC. The CAR-ID may be a small molecule. The CAR-ID may be a hapten. The CAR-ID may be FITC or a derivative thereof.

Vectors comprising sequences encoding CARS and/or CAR-EC switches and portions thereof, disclosed herein, may be selected from any commercially available expression vector. The expression vector may be a prokaryotic expression vector. The expression vector may be a eukaryotic expression vector. The expression vector may be a mammalian expression vector. The expression vector may be a viral expression vector. The expression vector may have a constitutive promoter for constitutive expression of the CAR and/or CAR-EC switch encoding sequences. The expression vector may have an inducible promoter for conditional expression of the CAR and/or CAR-EC switch encoding sequences.

Therapeutic Use

Disclosed herein are methods, platforms and kits for treating a disease or condition in a subject in need thereof, the method comprising administering a CAR-EC switch to the subject, wherein the CAR-EC switch comprises: a CAR-ID; and a TID. Disclosed herein are methods of treating a disease or condition in a subject in need thereof, the method comprising administering any one of the CAR-EC switches disclosed herein.

The methods may comprise administering a CAR-EC cell and one or more CAR-EC switches. The methods may comprise administering about 1, 2, 3, 4, 5, 6, 7, 8, 9, 10, 12, 15, 20, 24, 30, 35, 48, 50, 55, 60, 65, 70, 75, 80, 85, 90, 96, 100, 120, 150, 200, 300, 384, 400, 500, 600, 700, 800, 900, 1000 or more CAR-EC switches. The methods may comprise administering two or more CAR-EC switches. The two or more CAR-EC switches may comprise the same CAR-ID. The two more CAR-EC switches may comprise the same TID. The two or more CAR-EC switches may comprise one or more different CAR-IDs. The two more CAR-EC switches may comprise one or more different TIDs. The methods may comprise a plurality of CAR-EC cells and one or more CAR-EC switches. Administering the CAR-EC cell may comprise intravenous CAR-EC delivery. Administering the CAR-EC cell may comprise intraperitoneal CAR-EC delivery. Administering the CAR-EC cell may comprise intravenous CAR-EC delivery and intraperitoneal CAR-EC delivery. Administering the CAR-EC cell may occur once. Administering the CAR-EC cell may occur more than once (e.g. repeat injection). The CAR-ECs may be sorted to enrich a memory population of CAR-ECs before administering the CAR-ECs. The CAR-ECs may be subjected to iterative stimulation to enrich the memory population, as opposed to recursive stimulation which promotes exhaustion, provide for a long-lived, persistent phenotype. This rationale is based on natural acute infections with enrich long-lived memory cells through a 1-2 week long contraction phase that occurs after the challenge has been cleared. Similarly, the sCAR-T cell system in which adoptively transferred cells are rested following stimulation may more closely recapitulate a physiological duration of T cell activation.

The methods may comprise administering one or more CAR-ECs to a subject in need thereof and then administering one or CAR-EC switches to a subject in need thereof. The amount or dose of CAR-EC switch may affect the magnitude of the CAR-ECs response toward the target cells, therefore the amount or dose of the CAR-EC switch may be titrated for a desired effect. For example, tumors may be targeted by titration of CAR-EC switch to achieve suitable therapeutic index. The response may be titrated “on” to avoid CRS and TLS events, providing for personalized therapy. Furthermore, administration of a switch can be terminated in case of an adverse event, control of CAR-EC cell activity, titration of off-target reactivity, abrogation of TLS, or attenuation of CRS. The amount or dose may start at one level for a specified time period and then the amount or dose may be increased or decreased to a second level for a second specified time period. For example, the initial amount or dose of the CAR-EC switch may be the lowest dose necessary to eliminate the tumor. The amount or dose of the CAR-EC switch may then be increased to a larger dose in order to eliminate any remaining tumor cells. The methods may comprise terminating the administration of the CAR-EC switch once the tumor cells are eliminated. The methods may comprise re-administering the CAR-EC switch if the tumor cells re-occur in the patient or if the patient relapses.

The methods may comprise administering one or more CAR-ECs. The methods may comprise administering one or more T cells. The one or more effector cells may be selected from T cell is selected from a naive T cell, a memory stem cell T cell, a central memory T cell, an effector memory T cell, a helper T cell, a CD4⁺ T cell, a CD8⁺ T cell, a CD8⁻CD4⁺ T cell, an αβ T cell, a γδ T cell, a cytotoxic T cell, a natural killer T cell, a natural killer cell, and a macrophage.

The CAR-EC switch may have a therapeutic effect that is at least partially dependent on bringing an effector cell in proximity of a target cell. The therapeutic effect on the intended indication of the CAR-EC switch may be at least partially due to the CAR-EC switch recruiting an effector cell to the target cell. The therapeutic effect on the intended indication of the CAR-EC switch may be predominantly due to the CAR-EC switch recruiting an effector cell to the target cell. The therapeutic effect of the CAR-EC switch may be at least partially dependent on stimulating an immune response in the CAR-EC cell.

Administering the CAR-EC switch may not have any therapeutic effect without further administering an effector cell. The CAR-EC switch may not have a significant, desirable and/or intended therapeutic effect without further administering an effector cell. The CAR-EC switch may not have any therapeutic effect towards an intended indication of the CAR-EC platform without further administering an effector cell. A portion or component of the CAR-EC switch (e.g. CAR-ID or TID) may not have a therapeutic effect towards the intended indication of the CAR-EC switch without being conjugated to a second portion or component of the CAR-EC switch (e.g. CAR-ID or TID). The dose of a portion or component of the CAR-EC switch (e.g. CAR-ID or TID) when administered as part of the CAR-EC platform to provide a therapeutic effect may not have a therapeutic effect when the portion or component of the CAR-EC switch is administered alone at that dose. The portion or component of the CAR-EC switch may not be intended to have any therapeutic effect besides recruiting the T cell to the target cell. Administering the portion or component of the CAR-EC switch alone may have a therapeutic effect on the target cell, wherein the therapeutic effect is negligible relative to the therapeutic effect of administering the CAR-EC switch and the CAR-EC. Administering the portion or component of the CAR-EC switch may have a therapeutic effect on the target cell, wherein the therapeutic effect is less than the therapeutic effect of administering the CAR-EC switch and the CAR-EC cell.

Disclosed herein are uses of CAR-EC switches disclosed herein to treat a disease or condition in a subject in need thereof. Further disclosed herein are uses of CAR-EC switches disclosed herein in the manufacture of a medicament for the treatment of a disease.

Disclosed herein is use of a CAR-EC switch comprising a CAR-ID, wherein the CAR-ID comprises FITC or a derivative thereof and a TID, wherein the TID comprises an anti-CD19 antibody or fragment thereof; and an effector cell comprising a CAR, wherein the CAR comprises an anti-FITC antibody, wherein the anti-CD19 antibody or fragment thereof binds CD19 on a lymphoblast, lymphocyte or B cell, to treat an ALL, a CLL, or a B-cell lymphoma.

Disclosed herein is use of a CAR-EC switch comprising a CAR-ID, wherein the CAR-ID comprises FITC or a derivative thereof and a TID, wherein the TID comprises an antibody or antibody fragment selected from an anti-CLL1 antibody or fragment thereof, an anti-CD33 antibody or fragment thereof, and an anti-CD123 antibody or fragment thereof; and an effector cell comprising a CAR, wherein the CAR comprises an anti-FITC antibody, wherein the antibody or antibody fragment binds CLL1, CD33 or CD123 on a lymphoblast, lymphocyte or B cell, to treat an (AML.

The disease or condition may be a cell proliferative disorder. The cell proliferative disorder may be selected from a solid tumor, a lymphoma, a leukemia, and a liposarcoma. The cell proliferative disorder may be acute, chronic, recurrent, refractory, accelerated, in remission, stage I, stage II, stage III, stage IV, juvenile or adult. The cell proliferative disorder may be selected from myelogenous leukemia, lymphoblastic leukemia, myeloid leukemia, an acute myeloid leukemia, myelomonocytic leukemia, neutrophilic leukemia, myelodysplastic syndrome, B-cell lymphoma, burkitt lymphoma, large cell lymphoma, mixed cell lymphoma, follicular lymphoma, mantle cell lymphoma, hodgkin lymphoma, recurrent small lymphocytic lymphoma, hairy cell leukemia, multiple myeloma, basophilic leukemia, eosinophilic leukemia, megakaryoblastic leukemia, monoblastic leukemia, monocytic leukemia, erythroleukemia, erythroid leukemia and hepatocellular carcinoma. The cell proliferative disorder may comprise a hematological malignancy. The hematological malignancy may comprise a B cell malignancy. The cell proliferative disorder may comprise a chronic lymphocytic leukemia. The cell proliferative disorder may comprise an acute lymphoblastic leukemia. The cell proliferative disorder may comprise a CD19⁺ Burkitt's lymphoma.

The disease or condition may be a cancer, a pathogenic infection, autoimmune disease, inflammatory disease, or genetic disorder.

In some instances, the one or more diseases comprises a cancer. The cancer may comprise a recurrent and/or refractory cancer. Examples of cancers include, but are not limited to, sarcomas, carcinomas, lymphomas or leukemias.

The cancer may comprise a neuroendocrine cancer. The cancer may comprise a pancreatic cancer. The cancer may comprise an exocrine pancreatic cancer. The cancer may comprise a thyroid cancer. The thyroid cancer may comprise a medullary thyroid cancer. The cancer may comprise a prostate cancer.

The cancer may comprise an epithelial cancer. The cancer may comprise a breast cancer. The cancer may comprise an endometrial cancer. The cancer may comprise an ovarian cancer. The ovarian cancer may comprise a stromal ovarian cancer. The cancer may comprise a cervical cancer.

The cancer may comprise a skin cancer. The skin cancer may comprise a neo-angiogenic skin cancer. The skin cancer may comprise a melanoma.

The cancer may comprise a kidney cancer.

The cancer may comprise a lung cancer. The lung cancer may comprise a small cell lung cancer. The lung cancer may comprise a non-small cell lung cancer.

The cancer may comprise a colorectal cancer. The cancer may comprise a gastric cancer. The cancer may comprise a colon cancer.

The cancer may comprise a brain cancer. The brain cancer may comprise a brain tumor. The cancer may comprise a glioblastoma. The cancer may comprise an astrocytoma.

The cancer may comprise a blood cancer. The blood cancer may comprise a leukemia. The leukemia may comprise a myeloid leukemia. The cancer may comprise a lymphoma. The lymphoma may comprise a non-Hodgkin's lymphoma.

The cancer may comprise a sarcoma. The sarcoma may comprise an Ewing's sarcoma.

Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis.

In some instances, the cancer is a lung cancer. Lung cancer may start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic leukemia.

Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

The cancer may comprise a solid tumor. The cancer may comprise a sarcoma. The cancer may be selected from a group consisting of a bladder cancer, a breast cancer, a colon cancer, a rectal cancer, an endometrial cancer, a kidney cancer, a lung cancer, melanoma, a myeloma, a thyroid cancer, a pancreatic cancer, a glioma, a malignant glioma of the brain, a glioblastoma, an ovarian cancer, and a prostate cancer. The cancer may have non-uniform antigen expression. The cancer may have modulated antigen expression. The antigen may be a surface antigen. The cancer may not comprise a myeloma. The cancer may not comprise a melanoma. The cancer may not comprise a colon cancer. The cancer may be acute lymphoblastic leukemia (ALL). The cancer may be relapsed ALL. The cancer may be refractory ALL. The cancer may be relapsed, refractory ALL. The cancer may be chronic lymphocytic leukemia (CLL). The cancer may be relapsed CLL. The cancer may be refractory CLL. The cancer may be relapsed, refractory CLL.

The cancer may comprise a breast cancer. The breast cancer may be triple positive breast cancer (estrogen receptor-, progesterone receptor-, and Her2-positive). The breast cancer may be triple negative breast cancer (estrogen receptor-, progesterone receptor-, and Her2-negative). The breast cancer may be estrogen receptor positive. The breast cancer may be estrogen receptor negative. The breast cancer may be progesterone receptor positive. The breast cancer may be progesterone receptor negative. The breast cancer may comprise a Her2 negative breast cancer. The breast cancer may comprise a low-expressing Her2 breast cancer. The breast cancer may comprise a Her2 positive breast cancer. Cell lines expressing Her2 have been well-characterized for antigen density, reflecting clinical immunohistochemistry characterization which classifies malignancies as 0 (<20,000 Her2 antigens per cell), 1+ (100,000 Her2 antigens per cell), 2+ (500,000 Her2 antigens per cell), and 3+ (>2,000,000 Her2 antigens per cell). The present invention provides for methods of treating breast cancers of these classifications. The breast cancer may comprise a breast cancer classified as Her2 0. The breast cancer may comprise a breast cancer classified as Her2 1+. The breast cancer may comprise a breast cancer classified as Her2 2+. The breast cancer may comprise a breast cancer classified as a Her2 3+.

The disease or condition may be a pathogenic infection. Pathogenic infections may be caused by one or more pathogens. In some instances, the pathogen is a bacterium, fungi, virus, or protozoan.

Exemplary pathogens include but are not limited to: Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, or Yersinia. In some cases, the disease or condition caused by the pathogen is tuberculosis and the heterogeneous sample comprises foreign molecules derived from the bacterium Mycobacterium tuberculosis and molecules derived from the subject. In some instances, the disease or condition is caused by a bacterium is tuberculosis, pneumonia, which may be caused by bacteria such as Streptococcus and Pseudomonas, a foodborne illness, which may be caused by bacteria such as Shigella, Campylobacter and Salmonella, and an infection such as tetanus, typhoid fever, diphtheria, syphilis and leprosy. The disease or condition may be bacterial vaginosis, a disease of the vagina caused by an imbalance of naturally occurring bacterial flora. Alternatively, the disease or condition is a bacterial meningitis, a bacterial inflammation of the meninges (e.g., the protective membranes covering the brain and spinal cord). Other diseases or conditions caused by bacteria include, but are not limited to, bacterial pneumonia, a urinary tract infection, bacterial gastroenteritis, and bacterial skin infection. Examples of bacterial skin infections include, but are not limited to, impetigo which may be caused by Staphylococcus aureus or Streptococcus pyogenes; erysipelas which may be caused by a streptococcus bacterial infection of the deep epidermis with lymphatic spread; and cellulitis which may be caused by normal skin flora or by exogenous bacteria.

The pathogen may be a fungus, such as, Candida, Aspergillus, Cryptococcus, Histoplasma, Pneumocystis, and Stachybotrys. Examples of diseases or conditions caused by a fungus include, but are not limited to, jock itch, yeast infection, ringworm, and athlete's foot.

The pathogen may be a virus. Examples of viruses include, but are not limited to, adenovirus, coxsackievirus, Epstein-Barr virus, Hepatitis virus (e.g., Hepatitis A, B, and C), herpes simplex virus (type 1 and 2), cytomegalovirus, herpes virus, HIV, influenza virus, measles virus, mumps virus, papillomavirus, parainfluenza virus, poliovirus, respiratory syncytial virus, rubella virus, and varicella-zoster virus. Examples of diseases or conditions caused by viruses include, but are not limited to, cold, flu, hepatitis, AIDS, chicken pox, rubella, mumps, measles, warts, and poliomyelitis.

The pathogen may be a protozoan, such as Acanthamoeba (e.g., A. astronyxis, A. castellanii, A. culbertsoni, A. hatchetti, A. polyphaga, A. rhysodes, A. healyi, A. divionensis), Brachiola (e.g., B connori, B. vesicularum), Cryptosporidium (e.g., C. parvum), Cyclospora (e.g., C. cayetanensis), Encephalitozoon (e.g., E. cuniculi, E. hellem, E. intestinalis), Entamoeba (e.g., E. histolytica), Enterocytozoon (e.g., E. bieneusi), Giardia (e.g., G. lamblia), Isospora (e.g., I. belli), Microsporidium (e.g., M. africanum, M. ceylonensis), Naegleria (e.g., N. fowleri), Nosema (e.g., N. algerae, N. ocularum), Pleistophora, Trachipleistophora (e.g., T. anthropophthera, T. hominis), and Vittaforma (e.g., V. corneae).

The disease or condition may be an autoimmune disease or autoimmune related disease. An autoimmune disorder may be a malfunction of the body's immune system that causes the body to attack its own tissues. Examples of autoimmune diseases and autoimmune related diseases include, but are not limited to, Addison's disease, alopecia areata, ankylosing spondylitis, antiphospholipid syndrome (APS), autoimmune aplastic anemia, autoimmune hemolytic anemia, autoimmune hepatitis, autoimmune myocarditis, Behcet's disease, celiac sprue, Crohn's disease, dermatomyositis, eosinophilic fasciitis, erythema nodosum, giant cell arteritis (temporal arteritis), Goodpasture's syndrome, Graves' disease, Hashimoto's disease, idiopathic thrombocytopenic purpura (ITP), IgA nephropathy, juvenile arthritis, diabetes, juvenile diabetes, Kawasaki syndrome, Lambert-Eaton syndrome, systemic lupus erythematosus (SLE), mixed connective tissue disease (MCTD), multiple sclerosis, myasthenia gravis, pemphigus, polyarteritis nodosa, type I, II, & III autoimmune polyglandular syndromes, polymyalgia rheumatica, polymyositis, psoriasis, psoriatic arthritis, Reiter's syndrome, relapsing polychondritis, rheumatoid arthritis, sarcoidosis, scleroderma, Sjogren's syndrome, sperm & testicular autoimmunity, stiff person syndrome, Takayasu's arteritis, temporal arteritis/giant cell arteritis, ulcerative colitis, uveitis, vasculitis, vitiligo, and Wegener's granulomatosis.

The disease or condition may be an inflammatory disease. Examples of inflammatory diseases include, but are not limited to, alveolitis, amyloidosis, angiitis, ankylosing spondylitis, avascular necrosis, Basedow's disease, Bell's palsy, bursitis, carpal tunnel syndrome, celiac disease, cholangitis, chondromalacia patella, chronic active hepatitis, chronic fatigue syndrome, Cogan's syndrome, congenital hip dysplasia, costochondritis, Crohn's Disease, cystic fibrosis, De Quervain's tendinitis, diabetes associated arthritis, diffuse idiopathic skeletal hyperostosis, discoid lupus, Ehlers-Danlos syndrome, familial mediterranean fever, fascitis, fibrositis/fibromyalgia, frozen shoulder, ganglion cysts, giant cell arteritis, gout, Graves' Disease, HIV-associated rheumatic disease syndromes, hyperparathyroid associated arthritis, infectious arthritis, inflammatory bowel syndrome/irritable bowel syndrome, juvenile rheumatoid arthritis, lyme disease, Marfan's Syndrome, Mikulicz's Disease, mixed connective tissue disease, multiple sclerosis, myofascial pain syndrome, osteoarthritis, osteomalacia, osteoporosis and corticosteroid-induced osteoporosis, Paget's Disease, palindromic rheumatism, Parkinson's Disease, Plummer's Disease, polymyalgia rheumatica, polymyositis, pseudogout, psoriatic arthritis, Raynaud's Phenomenon/Syndrome, Reiter's Syndrome, rheumatic fever, rheumatoid arthritis, sarcoidosis, sciatica (lumbar radiculopathy), scleroderma, scurvy, sickle cell arthritis, Sjogren's Syndrome, spinal stenosis, spondyloisthesis, Still's Disease, systemic lupus erythematosis, Takayasu's (Pulseless) Disease, Tendinitis, tennis elbow/golf elbow, thyroid associated arthritis, trigger finger, ulcerative colitis, Wegener's Granulomatosis, and Whipple's Disease.

Methods of treatment disclosed herein may comprise off-target activity as measured by cytokine levels. The method may reduce the off-target activity, as measured by cytokine levels, when compared to other CAR-EC therapies. The method may reduce the off-target activity as measured by IFNγ levels. Other off-target activities that may be reduced include toxic lymphophenia, fatal cytolysis of solid tumor targets and chronic hypogammaglobulinemia for hematological targets. Methods of treatment and compositions disclosed herein may be used to treat a cancer comprising CD19-mediated B cell aplasia. The methods and compositions may minimize the CD19-mediated B cell aplasia. The method may avoid long-term B-cell aplasia.

The CAR-EC platforms, methods and compositions disclosed herein may be used to treat a heterogeneous tumor or a heterogeneous blood cell malignancy in a subject in need thereof. The “pan-B cell” marker CD20 is the most prevalently targeted antigen for B cell neoplasms and the FDA-approved antibody, rituximab, is a vital component in the treatment of many leukemias and lymphomas. However, resistance mechanisms related to modulation of CD20 antigen expression occurs in a significant number of patients. It is clear that targeting with either CD19 or CD20 antigen alone is insufficient for a curative therapy. The methods disclosed herein provide for construction and administration of two or more switches with different specificities (e.g. an anti-CD19 antibody CAR-EC switch and an anti-CD20 antibody CAR-EC switch). The methods disclosed herein provide for construction and administration of two or more switches with different specificities (e.g. an anti-CD19 antibody CAR-EC switch and an anti-CD22 antibody CAR-EC switch). This methodology may offer a significant advantage against the propensity for relapse in the clinic while avoiding persistent loss of B cells. A heterogeneous tumor or heterogeneous blood cell malignancy may also be treated with an anti-CD19 antibody CAR-EC switch and an anti-CD22 antibody CAR-EC switch. One or more CAR-EC switches may be administered sequentially or simultaneously. A second switch targeting a second cell surface molecule on the target cell may be administered after down regulation of a first cell surface molecule on the target cell that is targeted by a first switch.

The CAR-EC switch may be administered with one or more additional therapeutic agents. The one or more additional therapeutic agents may be selected from a group consisting of an immunotherapy, a chemotherapy and a steroid. The one or more additional therapeutic agents may be a chemotherapy drug. The chemotherapy drug may be an alkylating agent, an antimetabolite, an anthracycline, a topoisomerase inhibitor, a mitotic inhibitor, a corticosteroid or a differentiating agent. The chemotherapy drug may be selected from actinomycin-D, bleomycin, altretamine, bortezomib, busulfan, carboplatin, capecitabine, carmustine, chlorambucil, cisplatin, cladribine, clofarabine, cyclophosphamide, cytarabine, dacarbazine, daunorubicin, docetaxel, doxorubicin, epirubicin, etoposide, estramustine, floxuridine, fludarabine, fluorouracil, gemcitbine (Gemzar), hydroxyurea, idarubicin, ifosfamide, irinotecan (Camptosar), ixabepilone, L-asparaginase, lomustine, mechlorethamine, melphalan, 6-mercaptopurine, methotrexate, mitomycin-C, paclitaxel (Taxol), pemetrexed, pentostatin, streptozocin, temozolomide, teniposide, thioguanine, thiotepa, topotecan (Hycamtin), vincristine, vinblastine, vinorelbine, retinoids, tretinoin (ATRA or Atralin®), bexarotene (Targretin®) and arsenic trioxide (Arsenox®). The chemotherapy may be administered as a pill to swallow, as an injection into the muscle or fat tissue, intravenously, topically or directly into a body cavity.

The one or more additional therapeutic agents may comprise an angiogenesis inhibitor. The angiogenesis inhibitor may be selected from bevacizumab, itraconazole, carboxyamidotriazole, TNP-470, CM101, IFN alpha, IL-12, platelet factor 4, suramin, SU5416, thrombospondin, a VEGFR antagonist, an angiostatic steroid with heparin, CAR-ECilage-derived angiogenesis inhibitory factor, matrix metalloprotease inhibitors, angiostatin, endostatin, sorafenib, sunitinib, pazopanib, everolimus, 2-methoxyestradiol, tecogalan, tetrathiomolybdate, thalidomide, prolactin, αvβ₃ inhibitor, linomide, tasquinimod, soluble VEGFR-1, soluble NRP-1, angiopoietin 2, vasostatin, calreticulin, TIMP, CDAI, Meth-1, Meth-2, interferon-alpha, interferon-beta, interferon-gamma, CXCL10, IL-4, IL-12, IL-18, prothrombin, antithrombin III fragment, prolactin, VEGI, SPARC, osteopontin, maspin, canstatin, proliferin-related protein and restin.

The one or more additional therapeutic agents may comprise a hormone therapy. The hormone therapy may be selected from an anti-estrogen (e.g. fulvestrant (Faslodex®), tamoxifen, toremifene (Fareston®)); an aromatase inhibitor (e.g. anastrozole (Arimidex®), exemestane (Aromasin®), letrozole (Femara®)); a progestin (e.g. megestrol acetate (Megace®)); an estrogen; an anti-androgen (e.g. bicalutamide (Casodex®), flutamide (Eulexin®), nilutamide (Nilandron®)); a gonadotropin-releasing hormone (GnRH) or luteinizing hormone-releasing hormone (LHRH) agonist or analog (e.g. leuprolide (Lupron®), goserelin (Zoladex®)).

The one or more additional therapeutic agents may comprise a steroid. The steroid may be a corticosteroid. The steroid may be cortisol or a derivative thereof. The steroid may be selected from prednisone, methylprednisolone (Solumedrol) or dexamethasone.

The CAR-EC switch may be administered with one or more additional therapies. The one or more additional therapies may comprise laser therapy. The one or more additional therapies may comprise radiation therapy. The one or more additional therapies may comprise surgery.

Disclosed herein are platforms, kits and methods for treating a disease or condition in a subject. The subject may be a healthy subject. The subject may be suffering from a disease or condition. The subject may be suffering from more than one disease or condition. The subject may be suffering from chronic lymphocytic leukemia. The subject may be suffering from acute lymphoblastic leukemia. The subject may be an animal. The subject may be a mammal. The mammal may be a human, a chimpanzee, a gorilla, a monkey, a bovine, a horse, a donkey, a mule, a dog, a cat, a pig, a rabbit, a goat, a sheep, a rat, a hamster, a guinea pig or a mouse. The subject may be a bird or a chicken. The subject may be a human. The subject may be a child. The child may be suffering from acute lymphoblastic leukemia. The subject may be less than 6 months old. The subject may be about 1 year old, about 2 years old, about 3 years old, about 4 years old, about 5 years old, about 6 years old, about 7 years old, about 8 years old, about 9 years old, about 10 years old, about 11 years old, about 12 years old, about 13 years old, about 14 years old, about 15 years old, about 18 years old, about 20 years old, about 25 years old, about 30 years old, about 35 years old, about 40 years old, about 45 years old, about 50 years old, about 55 years old, about 60 years old, about 65 years old, about 70 years old, about 75 years old, about 80 years old, about 85 years old, about 90 years old, about 95 years old, about 100 years old or about 105 years old.

### Method of Clearing CAR-Effector Cells

Further disclosed herein are methods of clearing CAR-EC cells in a subject, comprising administering a CAR-EC off-switch. The CAR-EC off switch may comprise an antibody or antibody fragment that targets a cell surface marker on the effector cell. The CAR-EC off-switch may comprise a small molecule that is bound by the CAR of the CAR-EC. The CAR-EC off-switch may comprise a hapten (e.g. FITC) that is bound by the CAR of the CAR-EC. The CAR-EC off switch may comprise a CAR-ID that is bound by the CAR of the CAR-EC.

The CAR-EC off switch may be conjugated to a drug or a toxin. The drug or toxin may be selected from maytasine (e.g. DM1, DM4), monomethylauristatin E, monomethylauristatin F, Ki-4.dgA, dolastatin 10, calicheamicin, SN-38, duocarmycin, irinotecan, ricin, saporin, gelonin, poke weed antiviral protein, pseudomonas aeruginosa exotoxin A or diphtheria toxin. The toxin may comprise a poison, a bacterial toxin (e.g. bacterial toxins causing tetanus, diphtheria), a plant toxin or animal toxin. The toxin may be a snake venom. The toxin may comprise vinblastine. The toxin may comprise auristatin. The toxin may be contained in a liposome membrane-coated vesicle. Wherein the toxin is contained in a liposome membrane-coated vesicle, the antibody is attached to the vesicle.

Alternatively or additionally, the effector cell expresses a viral protein or fragment thereof that is not a cell surface marker. The effector cell expressing a viral protein or fragment thereof may be targeted with a drug. Wherein the effector cell comprises a viral protein or fragment thereof, the drug may be selected from a group comprising abacavir, acyclovir, acyclovir, adefovir, amantadine, amprenavir, ampligen, arbidol, atazanavir, atripla, balavir, boceprevirertet, cidofovir, combivir, darunavir, delavirdine, didanosine, docosanol, edoxudine, efavirenz, emtricitabine, enfuvirtide, entecavir, an entry inhibitor, famciclovir, a fixed dose combination antiretroviral drug, fomivirsen, fosamprenavir, foscarnet, fosfonet, a fusion inhibitor, ganciclovir, ibacitabine, imunovir, idoxuridine, imiquimod, indinavir, inosine, integrase inhibitor, interferon type III, interferon type II, interferon type I, interferon, lamivudine, lopinavir, loviride, maraviroc, moroxydine, methisazone, nelfinavir, nevirapine, nexavir, nucleoside analogue, oseltamivir, peginterferon alfa-2a, penciclovir, peramivir, pleconaril, podophyllotoxin, protease inhibiro, raltegravir, a reverse transcriptase inhibitor, ribavirin, rimantadine, ritonavir, pyramidine, saquinavir, sofosbuvir, stavudine, a synergistic enhancer retroviral durg, tea tree oil, telaprevir, tenofovir, tenofovir disoproxil, tipranavir, trifluridine, trizivir, tromantadine, truvada, valaciclovir, vicriviroc, vidarabine, viramidine, zacitabine, zanamivir or zidovudine. The drug may be ganciclovir. The drug may be acyclovir.

### Pharmaceutical Compositions

Disclosed herein is a pharmaceutical composition comprising one or more of the CAR-EC switches disclosed herein. The compositions may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl-or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™. series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Pharmaceutical compositions described herein may be formulated for controlled or sustained delivery in a manner that provides local concentration of the product (e.g., bolus, depot effect) and/or increased stability or half-life in a particular local environment. The compositions may comprise the formulation of CAR-EC switches, polypeptides, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained-or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies comprising an ultralong CDR3). See, for example, the description of controlled release porous polymeric microparticles for the delivery of pharmaceutical compositions in WO 93/15722. Suitable materials for this purpose include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al, Biopolymers, 22: 547-556 (1983)), poly(2-hydroxyethyl-methacrylate) (Langer et al, J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which may be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This may be determined by routine screening in animal models of the target disorder or, if such models are unavailable, in normal animals. Microencapsulation of recombinant proteins for sustained release has been performed successfully with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010. The sustained-release formulations of these proteins were developed using poly-lactic-coglycolic acid (PLGA) polymer due to its biocompatibility and wide range of biodegradable properties. The degradation products of PLGA, lactic and glycolic acids may be cleared quickly within the human body. Moreover, the degradability of this polymer may be depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41. Additional examples of sustained release compositions include, for example, EP 58,481A, U.S. Pat. No. 3,887,699, EP 158,277A, Canadian Patent No. 1176565, U. Sidman et al., Biopolymers 22, 547 [1983], R. Langer et al., Chem. Tech. 12, 98 [1982], Sinha at al., J. Control. Release 90, 261 [2003], Zhu at al., Nat. Biotechnol. 18, 24 [2000], and Dai at al., Colloids Surf B Biointerfaces 41, 117 [2005].

Bioadhesive polymers are also contemplated for use in or with compositions of the present disclosure. Bioadhesives are synthetic and naturally occurring materials able to adhere to biological substrates for extended time periods. For example, Carbopol and polycarbophil are both synthetic cross-linked derivatives of poly(acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine. Hyaluronic acid is found in the extracellular tissue matrix of vertebrates, including in connective tissues, as well as in synovial fluid and in the vitreous and aqueous humor of the eye. Esterified derivatives of hyaluronic acid have been used to produce microspheres for use in delivery that are biocompatible and biodegradable (see, for example, Cortivo et al., Biomaterials (1991) 12:727-730; EP 517,565; WO 96/29998; Illum et al., J. Controlled Rel. (1994) 29:133-141).

Both biodegradable and non-biodegradable polymeric matrices may be used to deliver compositions of the present disclosure, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which may be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see, for example, WO 04/009664, WO 05/087201, Sawhney, et al., Macromolecules, 1993, 26, 581-587) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono-di-and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775, 4,675,189 and 5,736,152 and (b) diffusional systems in which a product permeates at a controlled rate from a polymer such as described in U.S. Pat. Nos. 3,854,480, 5,133,974 and 5,407,686. Liposomes containing the product may be prepared by methods known methods, such as for example (DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; JP 83-118008; U.S. Pat. Nos. 4,485,045 and 4,544,545; and EP 102,324).

Alternatively or additionally, the compositions may be administered locally via implantation into the affected area of a membrane, sponge, or other appropriate material on to which a CAR-EC switch disclosed herein has been absorbed or encapsulated. Where an implantation device is used, the device may be implanted into any suitable tissue or organ, and delivery of a CAR-EC switch, nucleic acid, or vector disclosed herein may be directly through the device via bolus, or via continuous administration, or via catheter using continuous infusion.

A pharmaceutical composition comprising a CAR-EC switch disclosed herein may be formulated for inhalation, such as for example, as a dry powder. Inhalation solutions also may be formulated in a liquefied propellant for aerosol delivery. In yet another formulation, solutions may be nebulized. Additional pharmaceutical composition for pulmonary administration include, those described, for example, in WO 94/20069, which discloses pulmonary delivery of chemically modified proteins. For pulmonary delivery, the particle size should be suitable for delivery to the distal lung. For example, the particle size may be from 1 μm to 5 μm; however, larger particles may be used, for example, if each particle is fairly porous.

Certain formulations containing CAR-EC switches disclosed herein may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also may be employed.

Another preparation may involve an effective quantity of a CAR-EC switch disclosed herein in a mixture with non-toxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

### CAR-EC Switch Production Methods

### Methods of Producing Switches and Switch Intermediates

Disclosed herein are methods of producing CAR-EC switches. Generally, the method comprises attaching a CAR-ID to a TID. Alternatively, the method may comprise attaching a switch intermediate comprising a CAR-ID and a linker to a TID. The method may comprise attaching a switch intermediate comprising a TID and a linker to a CAR-ID. The method may comprise attaching a first switch intermediate comprising a CAR-ID and a first linker to a second switch comprising a TID and a second linker. Attachment of the CAR-ID to the TID may occur in a site-specific manner. Attachment in a site-specific manner may comprise attaching the CAR-ID to a predetermined site on the TID. Attachment in a site-specific manner may comprise attaching the TID to a predetermined site on the CAR-ID. Attachment of the CAR-ID to the TID may occur in a site-independent manner. Attachment in a site-independent manner may comprise attaching the CAR-ID to a random site on the TID. Attachment in a site-independent manner may comprise attaching the TID to a random site on the CAR-ID. The method may further comprise attaching one or more additional CAR-IDs to the TID. The method may further comprise attaching or more additional TIDs to the CAR-ID. The method may further comprise using one or more additional linkers to connect the TID to the CAR-ID. Attaching the CAR-ID to the TID may comprise conducting one or more chemical reactions.

The method of producing a switch may comprise linking a TID based on or derived from an antibody or antibody fragment to a CAR-ID or a switch intermediate comprising a CAR-ID to produce a CAR-EC switch comprising (a) the TID; (b) one or more linkers; and (c) the CAR-ID, The one or more linkers may link the TID to the CAR-ID. Linking the TID to the CAR-ID may occur in a site-specific manner. The CAR-ID may be attached to a predetermined site on the TID via the one or more linkers. The TID may be attached to a predetermined site on the CAR-ID via the one or more linkers.

Disclosed herein are methods of producing a switch of Formula I: X-L1-Y or Formula IA: Y-L1-X, wherein X is a CAR-ID, Y is a TID and L1 is a linker. X may be a CAR-binding small molecule and Y may be an antibody or antibody fragment. X may be a CAR-binding small molecule that does not comprise a peptide and Y may be a peptide that does not comprise an antibody or antibody fragment. X may be a CAR-binding small molecule that does not comprise a peptide and Y may be a targeting small molecule that does not comprise a peptide. The method may comprise conducting one or more reactions to attach the CAR-ID to a predetermined site in the TID. Conducting the one or more reactions to attach the CAR-ID to the TID may comprise mixing a plurality of CAR-IDs with a plurality of TIDs. The method may comprise attaching one end of the linker to the TID, followed by attachment of the other end of the linker to the CAR-ID. The method may comprise attaching one end of the linker to the CAR-ID, followed by attachment of the other end of the linker to the TID. Attachment of the linker to the TID may occur in a site-specific manner. The linker may be attached to a predetermined amino acid of the TID. The amino acid may be an unnatural amino acid. The linker may comprise a functional group that interacts with the amino acid. Attachment of the linker to the TID may occur in a site-independent manner. The linker may be randomly attached to the TID. The linker may comprise a functional group that reacts with a functional group in the TID. Attachment of the linker to the CAR-ID may occur in a site-specific manner. Attachment of the linker to the CAR-ID may occur in a site-independent manner. The linker may comprise a functional group that reacts with a functional group in the CAR-ID. Conducting the one or more reactions to attach the CAR-ID to the TID may comprise conducting an oxime ligation.

Alternatively, or additionally, the method may comprise conducting a reaction to attach the linker or a precursor of the linker to the CAR-ID to produce a switch intermediate comprising the linker conjugated to the CAR-ID. The switch intermediate may have the Formula II: X-L1 or Formula IIA: L1-X, wherein X is the CAR-ID and L1 is the linker or precursor of the linker. The linker may be conjugated to the CAR-ID in a site-specific manner. The linker may be conjugated to the CAR-ID in a site-independent manner. Conducting the one or more reactions to attach the CAR-ID to the TID may comprise attaching the linker portion of the switch intermediate to the TID. Conducting the one or more reactions to attach the CAR-ID to the TID may comprise contacting a plurality of switch intermediates comprising the linker or linker precursor conjugated to the CAR-ID with a plurality of TIDs. Attachment of the linker portion of the switch intermediate to the TID may occur in a site-specific manner. The TID may comprise one or more unnatural amino acids. The linker portion of the switch may be attached to the TID via the one or more unnatural amino acids. Attachment of the linker portion of the switch intermediate may occur in a site-independent manner.

Alternatively, or additionally, the method may comprise conducting a reaction to attach the linker or a precursor of the linker to the TID to produce a switch intermediate comprising the linker or precursor of the linker conjugated to the TID. The switch intermediate may be of Formula III: Y-L1 or Formula IIIA: L1-Y, wherein Y is the TID and L1 is the linker or linker precursor. The linker may be conjugated to the TID in a site-specific manner. The linker may be conjugated to the TID in a site-independent manner. Conducting the one or more reactions to attach the CAR-ID to the TID may comprise attaching the linker portion of the switch intermediate to the CAR-ID. Conducting the one or more reactions to attach the CAR-ID to the TID may comprise contacting a plurality of switch intermediates comprising the linker or linker precursor conjugated to the TID with a plurality of CAR-IDs. Attachment of the linker portion of the switch intermediate to the CAR-ID may occur in a site-specific manner. Attachment of the linker portion of the switch intermediate may occur in a site-independent manner.

The method may comprise coupling one or more linkers to the TID to produce a switch intermediate of Formula III: Y-L1 or Formula IIIA: L1-Y, wherein Y is the TID and L1 is the linker; and conjugating the switch intermediate to the CAR-ID, thereby producing the CAR-EC switch. The switch intermediate may be conjugated to the CAR-ID in a site-specific manner. The switch intermediate may be conjugated to the CAR-ID in a site-independent manner. The method may further comprise incorporating one or more unnatural amino acids into the CAR-ID and/or TID. The switch intermediate may be conjugated to the CAR-ID in a site-specific manner through the use of the unnatural amino acid.

The method may comprise coupling one or more linkers to the CAR-ID to produce a switch intermediate of Formula II: X-L1 or Formula IIA: L1-X, wherein X is the CAR-ID and L1 is the linker; and conjugating the switch intermediate to the TID, thereby producing the CAR-EC switch. The switch intermediate may be conjugated to the TID in a site-specific manner. The switch intermediate may be conjugated to the TID in a site-independent manner. The method may further comprise incorporating one or more unnatural amino acids into the CAR-ID and/or TID. The switch intermediate may be conjugated to the TID in a site-specific manner through the use of the unnatural amino acid.

Conjugating the switch intermediate of Formula II: X-L1 or Formula IIA: L1-X, wherein X is the CAR-ID and L1, to the TID may comprise forming an oxime. Conjugating the switch intermediate of Formula III: Y-L1 or Formula IIIA: L1-Y, wherein Y is the TID and L1, to the CAR-ID may comprise forming an oxime. Forming an oxime may comprise conducting one or more reactions under acidic conditions. Forming an oxime may comprise conducting one or more reactions under slightly acidic conditions. Forming an oxime may comprise conducting one or more reactions under slightly neutral conditions.

A method of producing a switch may comprise (a) producing a TID comprising an unnatural amino acid; (b) attaching a first linker to the TID to produce a first switch intermediate comprising the TID and the first linker; (c) attaching a second switch intermediate comprising a CAR-ID and a second linker to the first switch intermediate, thereby producing the switch. The unnatural amino acid may be p-acetylphenalanine (pAcF). The unnatural amino acid may be p-azidophenylalanine (pAzF) The TID may comprise a polypeptide based on or derived from an antibody or antibody fragment. The antibody may be selected from the group consisting of an anti-CD19 antibody, an anti-CD22 antibody, an anti-CD20 antibody, an anti-EGFR antibody, an anti-EGFRvIII antibody, an anti-Her2 antibody, an anti-CS1 antibody, an anti-BCMA antibody, an anti-CEA antibody, an anti-CLL-1 antibody and an anti-CD33 antibody. The antibody may be an anti-CD19 antibody. The antibody may be an anti-EGFR antibody. The antibody may be an anti-CD20 antibody. The antibody may be an anti-HER2 antibody. The antibody may be an anti-CS1 antibody. The antibody may be an anti-CD123 antibody. The TID may comprise an antibody fragment. The antibody may comprise an amino acid sequence of any one of SEQ ID NOs: 10-31 and optionally SEQ ID NOs: 52 and 53. The antibody may be encoded by a nucleotide sequence of any one of SEQ ID NOs: 5-9. The first linker may be a bifunctional linker. The linker may be a heterobifunctional linker. The linker may comprise one or more polyethylene glycol (PEG) subunits. The first linker may comprise cyclooctyne. The first linker may be a PEG-cyclooctyne linker. The linker may comprise an azide. The first linker may comprise triazole. The triazole may be 1,2,3-triazole. The triazole may be 1,2,4-triazole. The first linker may comprise an azide-PEG-aminoxy linker. The first linker may be attached to a ketone of the unnatural amino acid. The first linker may be attached to the TID via oxime ligation. The CAR-ID may comprise a small molecule. The CAR-ID may comprise FITC. The second linker may be a bifunctional linker. The linker may be a heterobifunctional linker. The linker may comprise one or more polyethylene glycol (PEG) subunits. The second linker may comprise cyclooctyne. The second linker may be a PEG-cyclooctyne linker. The linker may comprise an azide. The second linker may comprise triazole. The triazole may be 1,2,3-triazole. The triazole may be 1,2,4-triazole. The second linker may be a PEG-cyclooctyne linker. The second switch intermediate may be attached to the first switch intermediate via a click chemistry reaction. The second switch intermediate may be attached to the first switch intermediate through a cycloaddition reaction. The cycloaddition reaction may be a [3+2] cycloaddition reaction.

Conjugating the linker to the CAR-ID to produce the switch may comprise forming one or more bonds between the linker and the CAR-ID. Conjugating the linker to the TID to produce the switch may comprise forming one or more bonds between the linker and the TID. The one or more bonds may comprise an ionic bond, a covalent bond, a non-covalent bond or a combination thereof. Additional methods of conjugating the linker the CAR-ID and the TID may be performed as described in Roberts et al., Advanced Drug Delivery Reviews 54:459-476 (2002), which is included by reference in its entirety.

The CAR-ID may comprise any of the CAR-IDs disclosed herein. For example, the CAR-ID may comprise a small molecule. The CAR-ID may comprise FITC. The CAR-ID may be selected from the group consisting of DOTA, dinitrophenol, quinone, biotin, aniline, atrazine, an aniline-derivative, o-aminobenzoic acid, p-aminobenzoic acid, m-aminobenzoic acid, hydralazine, halothane, digoxigenin, benzene arsonate, lactose, trinitrophenol, biotin and derivatives thereof. The TID may comprise any of the TIDs disclosed herein. For example, the TID may comprise a small molecule. The TID may comprise 2-[3-(1,3-dicarboxypropyl)ureido] pentanedioic acid or a derivative thereof. The TID may comprise folate. The TID may be based on or derived from an antibody or antibody fragment. The antibody or antibody fragment may comprise anti-CD19. The antibody or antibody fragment may be selected from the group comprising anti-CD20, anti-CD22, anti-CD33, anti-BMCA, anti-CEA, anti-CLL1, anti-CS1, anti-EGFR, anti-Her2, anti-CD33, and anti-EGFRvIII. The linker may comprise any of the linkers disclosed herein. For example, the linker may comprise an aminooxy group, azide group cyclooctyne group, or a combination thereof at one or more termini. The linker may be a bifunctional linker. The linker may be a heterobifunctional linker. The linker may comprise one or more PEG subunits.

Disclosed herein are methods of producing a switch of Formula IV: X-L1-L2-Y, wherein in X is a CAR-ID, L1 is a first linker, L2 is a second linker and Y is a TID. The method may comprise (a) coupling L1to X to produce a first switch intermediate of Formula II: X-L1; (b) coupling L2 to Y to produce a second switch intermediate of Formula V: L2-Y; and (c) linking the first switch intermediate of Formula II to the second switch intermediate of Formula: V, thereby producing the switch of Formula IV.

Disclosed herein are methods of producing a switch of Formula IVA: Y-L2-L1-X, wherein Y is a TID, L1 is a first linker, L2 is a second linker and X is a CAR-ID. The method may comprise (a) coupling L1 to X to produce a first switch intermediate of Formula IIA: L1-X; (b) coupling L2 to Y to produce a second switch intermediate of Formula VA: Y-L2; and (c) linking the first intermediate of Formula IIA to the second intermediate of Formula VA, thereby producing the CAR-EC switch of Formula IVA.

The methods may further comprise incorporating one or more unnatural amino acids into X and/or Y. The L1 may be coupled to X in a site-specific manner. The L1 may be coupled to X in a site-specific manner through the one or more unnatural amino acids. L2 may be coupled to Y in a site-specific manner. The L2 may be coupled to Y in a site-specific manner through the one or more unnatural amino acids. The method may further comprise modifying a nucleic acid encoding X to produce one or more amber codons in X. The method may further comprise modifying a nucleic acid encoding Y to produce one or more amber codons in Y.

Conjugating the linker to the CAR-ID to produce the first switch intermediate may comprise forming one or more bonds between the linker and the CAR-ID. Conjugating the linker to the TID to produce the second switch intermediate may comprise forming one or more bonds between the linker and the TID. The one or more bonds may comprise an ionic bond, a covalent bond, a non-covalent bond or a combination thereof. Additional methods of conjugating the linker the CAR-ID and the TID may be performed as described in Roberts et al., Advanced Drug Delivery Reviews 54:459-476 (2002), which is included by reference in its entirety.

Linking the first switch intermediate to the second switch intermediate may comprise a Huisgen-cycloaddition, a Diels-Halder reaction, a hetero Diels-Alder reaction or an enzyme-mediated reaction. Linking the first switch intermediate to the second switch intermediate may produce an oxime, a tetrazole, a Diels Alder adduct, a hetero Diels Alder adduct, an aromatic substitution reaction product, a nucleophilic substitution reaction product, an ester, an amide, a carbamate, an ether, a thioether, a Michael reaction product, cycloaddition product, a metathesis reaction product, a metal-mediated cross-coupling reaction product, a radical polymerization product, an oxidative coupling product, an acyl-transfer reaction product, or a photo click reaction product. Linking the first switch intermediate to the second switch intermediate may produce a disulfide bridge or a maleimide bridge.

L1 and/or L2 may comprise a linker selected from a bifunctional linker, a cleavable linker, a non-cleavable linker, an ethylene glycol linker, a bifunctional ethylene glycol linker, a flexible linker, or an inflexible linker. L1 and/or L2 may comprise a linker selected from the group comprising cyclooctyne, cyclopropene, aryl/alkyl azides, trans-cyclooctene, norborene, and tetrazines. A terminus of L1 and/or a terminus of L2 may comprise an alkoxy-amine. A terminus of L1 and/or a terminus of L2 may comprise an azide or cyclooctyne group. X may be coupled to L1 by a chemical group selected from a cyclooctyne, cyclopropene, aryl/alkyl azide, trans-cyclooctene, norborene, and tetrazine. Linking the first switch intermediate (X-L1 or L1-X) and second switch intermediate (Y-L2 or L2-Y) may comprise conducting one or more copper-free reactions. Linking the first switch intermediate (X-L1 or L1-X) and second switch intermediate (Y-L2 or L2-Y) may comprise conducting one or more copper-containing reactions. Linking the first switch intermediate (X-L1 or L1-X) and second switch intermediate (Y-L2 or L2-Y) may comprise one or more cycloadditions. Linking the first switch intermediate (X-L1 or L1-X) and second switch intermediate (Y-L2 or L2-Y) may comprise one or more Huisgen-cycloadditions. Linking the first switch intermediate (X-L1 or L1-X) and second switch intermediate (Y-L2 or L2-Y) may comprise one or more Diels Alder reactions. Linking the first switch intermediate (X-L1 or L1-X) and second switch intermediate (Y-L2 or L2-Y) may comprise one or more Hetero Diels Alder reaction.

The methods disclosed herein may comprise coupling one or more linkers to one or more TIDs, CAR-IDs or combinations thereof to produce one or more switch intermediates. The switch intermediate may comprise a TID attached to a linker (e.g., TID switch intermediate). The switch intermediate may comprise a CAR-ID attached to a linker (e.g., CAR-ID switch intermediates). The methods may comprise coupling a first linker to TID to produce a TID switch intermediate. The methods may comprise coupling a linker to a CAR-ID to produce a CAR-ID switch intermediate.

Coupling of the one or more linkers to the TID and the CAR-ID may occur simultaneously. Coupling of the one or more linkers to the TID and the CAR-ID may occur sequentially. Coupling of the one or more linkers to the TID and the CAR-ID may occur in a single reaction volume. Coupling of the one or more linkers to the TID and the CAR-ID may occur in two or more reaction volumes.

Coupling one or more linkers to the TID and/or the CAR-ID may comprise forming one or more oximes between the linker and the TID and/or the CAR-ID. Coupling one or more linkers to the TID and/or the CAR-ID may comprise forming one or more stable bonds between the linker and the TID and/or the CAR-ID. Coupling one or more linkers to the TID and/or the CAR-ID may comprise forming one or more covalent bonds between the linker and the TID and/or the CAR-ID. Coupling one or more linkers to the TID and/or the CAR-ID may comprise forming one or more non-covalent bonds between the linker and TID and/or the CAR-ID. Coupling one or more linkers to the TID and/or the CAR-ID may comprise forming one or more ionic bonds between the linker and the TID and/or the CAR-ID.

Coupling one or more linkers to the TID and/or the CAR-ID may comprise site specifically coupling one or more linkers to the TID and/or the CAR-ID. Site-specific coupling may comprise linking the one or more linkers to the unnatural amino acid of the TID and/or the CAR-ID. Linking the one or more linkers to the unnatural amino acid of the TID and/or the CAR-ID may comprise formation of an oxime. Linking the one or more linkers to the unnatural amino acid of the TID and/or the CAR-ID may comprise, by way of non-limiting example, reacting a hydroxylamine of the one or more linkers with an aldehyde or ketone of an amino acid. The amino acid may be an unnatural amino acid.

Conducting the one or more reactions to site-specifically link the CAR-ID to the TID, to site-specifically attach the linker or a precursor of the linker to the CAR-ID, to site-specifically attach the linker or a precursor of the linker to the TID, to site-specifically attach the CAR-ID switch intermediate to the TID, to site-specifically attach the TID switch intermediate to the CAR-ID or to site-specifically attach the TID switch intermediate to the CAR-ID switch intermediate may comprise conducting one or more reactions selected from a copper-free reaction, a cycloadditions, a Huisgen-cycloaddition, a copper-free [3+2] Huisgen-cycloaddition, a copper-containing reaction, a Diels Alder reactions, a hetero Diels Alder reaction, metathesis reaction, a metal-mediated cross-coupling reaction, a radical polymerization, an oxidative coupling, an acyl-transfer reaction, a photo click reaction, an enzyme-mediated reaction, a transglutaminase-mediated reaction.

The switches disclosed herein may comprise a CAR-ID comprising FITC or a derivative thereof. The method of producing such switches may comprise coupling a linker or precursor thereof, a switch intermediate comprising a TID (e.g., TID switch intermediate), or a TID to the CAR-ID. Coupling the linker or precursor thereof, the TID switch intermediate to the CAR-ID may comprise conjugation of an isothiocyanate of FITC to the linker or precursor thereof, TID switch intermediate or TID. The TID may be based on or derived from a polypeptide. The polypeptide may be an antibody or antibody fragment. Coupling a TID to the CAR-ID may comprise conjugating the isothiocyanate of FITC to an amino acid of the TID. The amino acid may be a lysine. The method may comprise coupling or more CAR-IDs to the TID. The method may comprise conjugating FITC from two or more CAR-IDs to two or more amino acids of the TID. The two or more amino acids may be lysine.

Producing a switch disclosed herein may comprise ester coupling. Ester coupling may comprise forming an amide bond between the CAR-ID and the TID. Ester coupling may comprise forming an amide bond between a switch intermediate and the TID. The switch intermediate may comprise a CAR-ID attached to a linker. The amide bond may be formed between the linker of the switch intermediate and the TID. The linker may be a NHS-ester linker. The amide bond may be formed between the linker of the switch intermediate and an amino acid of the TID. The CAR-ID may comprise a small molecule. The small molecule may be FITC. The TID may be based on or derived from a polypeptide. The polypeptide may be an antibody or antibody fragment. The TID may comprise a small molecule.

The method of producing a switch disclosed herein may comprise: (a) obtaining a switch intermediate comprising (i) a CAR-ID; and (ii) a linker; and (b) contacting the switch intermediate with a TID, thereby producing the switch. Contacting the switch intermediate with the TID may comprise performing an ester coupling reaction. The linker may comprise a NHS-ester linker. The TID may comprise one or more amino acids. Performing the ester coupling reaction may comprise forming an amide bond between the NHS-ester linker of the switch intermediate and the one or more amino acids of the TID. The method may further comprise producing a plurality of switches. Two or more switches of the plurality of switches may comprise two or more switch intermediates attached to two or more different amino acids of the TID. For example, a first switch intermediate may be attached to a lysine residue of a first TID and a second switch intermediate may be attached to a glycine residue of a second TID. Two or more switches of the plurality of switches may comprise two or more switch intermediates attached to the same amino acid of the TID. For example, the two or more switch intermediates may be attached to a lysine residue of a first and second TID. Two or more switches of the plurality of switches may comprise two or more switch intermediates attached to the same amino acid located at two or more different positions in the TID. For example, a first switch intermediate may be attached to lysine 10 of a first TID and the second switch intermediate may be attached to lysine 45 of a second TID. Two or more switches of the plurality of switches may comprise two or more switch intermediates attached to the same amino acid located at the same position in the TID. For example, a first switch intermediate may be attached to lysine 10 of a first TID and the second switch intermediate may be attached to lysine 10 of a second TID.

Methods of producing a switch disclosed herein may comprise using one or more unnatural amino acids. The method may comprise incorporating one or more unnatural amino acids into the CAR-ID. The CAR-ID may be based on or derived from a polypeptide that can interact with a CAR on an effector cell. The polypeptide may be a non-antibody based polypeptide. Generally, a non-antibody based polypeptide is a polypeptide that does not comprise an antibody or antibody fragment. The unnatural amino acid may be incorporated into the non-antibody based polypeptide. The unnatural amino acid may replace an amino acid of the non-antibody based polypeptide. Alternatively, or additionally, the method may comprise incorporating one or more unnatural amino acids into the TID. The TID may be based on or derived from a polypeptide. The polypeptide may be an antibody. The polypeptide may be a non-antibody based polypeptide. The unnatural amino acid may be incorporated into the polypeptide. The unnatural amino acid may replace an amino acid of the polypeptide.

The method of producing the switch may further comprise modifying one or more amino acid residues in polypeptide from which the CAR-ID is based or derived. The method of producing the switch may comprise modifying one or more amino acid residues in polypeptide from which the TID is based or derived. Modifying the one or more amino acid residues may comprise mutating one or more nucleotides in the nucleotide sequence encoding the polypeptide. Mutating the one or more nucleotides in the nucleotide sequence encoding may comprise altering a codon encoding an amino acid to a nonsense codon.

Incorporating one or more unnatural amino acids into the polypeptide from which the CAR-ID is based or derived may comprise modifying one or more amino acid residues in the polypeptide to produce one or more amber codons in the antibody or antibody fragment. Incorporating one or more unnatural amino acids into the polypeptide from which the TID is based or derived may comprise modifying one or more amino acid residues in the polypeptide to produce one or more amber codons in the antibody or antibody fragment.

The one or more unnatural amino acids may be incorporated into the polypeptide in response to an amber codon. The one or more unnatural amino acids may be site-specifically incorporated into the polypeptide.

Incorporating one or more unnatural amino acids into the polypeptide from which the CAR-ID and the TID are based or derived may comprise use of one or more genetically encoded unnatural amino acids with orthogonal chemical reactivity relative to the canonical twenty amino acids to site-specifically modify the antibody, antibody fragment, or targeting peptide. Incorporating one or more unnatural amino acids may comprise the use of one or more tRNA synthetases. The tRNA synthetase may be an aminoacyl tRNA synthetase. The tRNA synthetase may be a mutant tRNA synthesis. Incorporating one or more unnatural amino acids may comprise a tRNA/tRNA synthetase pair. The tRNA/tRNA synthetase pair may comprise a tRNA/aminoacyl-tRNA synthetase pair. The tRNA/tRNA synthetase pair may comprise a tRNATyr/tyrosyl-tRNA synthetase pair. Incorporating the one or more unnatural amino acids may comprise use of an evolved tRNA/aminoacyl-tRNA synthetase pair to site-specifically incorporate one or more unnatural amino acids at defined sites in the polypeptide in response to one or more amber nonsense codon.

Additional methods for incorporating unnatural amino acids include, but are not limited to, methods disclosed in Chatterjee et al. (A Versatile Platform for Single- and Multiple-Unnatural Amino Acid Mutagenesis in Escherichia coli, Biochemistry, 2013), Kazane et al. (J Am Chem Soc, 135(1):340-6, 2013), Kim et al. (J Am Chem Soc, 134(24):9918-21, 2012), Johnson et al. (Nat Chem Biol, 7(11):779-86, 2011) and Hutchins et al. (J Mol Biol, 406(4):595-603, 2011).

A method of producing a switch for activating a chimeric antigen receptor-effector cell (CAR-EC) may comprise (a) obtaining a target interacting domain (TID) comprising an unnatural amino acid; and (b) attaching a chimeric antigen receptor-interacting domain (CAR-ID) to the TID, thereby producing the switch.

Attaching the CAR-ID to the TID may comprise one or cycloadditions. The one or more cycloadditions may comprise a Huisgen cycloaddition. The one or more cycloadditions may comprise a [3+2] cycloaddition. The one or more cycloadditions may comprise a [3+2] Huisgen cycloaddition. The one or more cycloadditions may comprise a copper-free cycloaddition. Attaching the CAR-ID to the TID may comprise a copper free reaction. Attaching the CAR-ID to the TID may comprise one or more copper-containing reactions. Attaching the CAR-ID to the TID may comprise one or more Diels Alder reactions. Attaching the CAR-ID to the TID may comprise one or more hetero Diels Alder reactions. Attaching the CAR-ID to the TID may comprise one or more ester couplings. Attaching the CAR-ID to the TID may comprise one or more isothiocyanate couplings. Attaching the CAR-ID to the TID may comprise attaching the CAR-ID to an amino acid of TID. The amino acid may be an unnatural amino acid. Attaching the CAR-ID to the TID may comprise one or more bioorthogonal reactions. The CAR-ID may be attached to the TID in a site-specific manner. The CAR-ID may be attached to a predetermined site in the TID. The CAR-ID may be attached to the TID in a site-independent manner.

The method may further comprise attaching a first linker to the TID to produce first switch intermediate. Attaching the first linker to the TID may comprise one or cycloadditions. Attaching the first linker to the TID may comprise a copper free reaction. Attaching the first linker to the TID may comprise one or more copper-containing reactions. Attaching the first linker to the TID may comprise one or more Diels Alder reactions. Attaching the first linker to the TID may comprise one or more hetero Diels Alder reactions. Attaching the first linker to the TID may comprise one or more ester couplings. Attaching the first linker to the TID may comprise oxime ligation. Attaching the first linker to the TID may comprise forming one or more oximes between the first linker and the TID. Attaching the first linker to the TID may comprise forming one or more stable bonds between the first linker and the TID. Attaching the first linker to the TID may comprise forming one or more covalent bonds between the first linker and the TID. Attaching the first linker to the TID may comprise forming one or more non-covalent bonds between the first linker and the TID. Attaching the first linker to the TID may comprise forming one or more ionic bonds between the first linker and the TID. Attaching the first linker to the TID may comprise attaching the linker to an amino acid of TID. The amino acid may be an unnatural amino acid. Attaching the first linker to the TID may comprise one or more bioorthogonal reactions.

Attaching the CAR-ID to the TID may comprise attaching the first switch intermediate to the CAR-ID. Attaching the first switch intermediate to the CAR-ID may comprise one or cycloadditions. The one or more cycloadditions may comprise a Huisgen cycloaddition. The one or more cycloadditions may comprise a [3+2] cycloaddition. The one or more cycloadditions may comprise a [3+2] Huisgen cycloaddition. The one or more cycloadditions may comprise a copper-free cycloaddition. Attaching the first switch intermediate to the CAR-ID may comprise a copper free reaction. Attaching the first switch intermediate to the CAR-ID may comprise one or more copper-containing reactions. Attaching the first switch intermediate to the CAR-ID may comprise one or more Diels Alder reactions. Attaching the first switch intermediate to the CAR-ID may comprise one or more hetero Diels Alder reactions. Attaching the first switch intermediate to the CAR-ID may comprise one or more ester couplings. Attaching the first switch intermediate to the CAR-ID may comprise one or more isothiocyanate couplings.

The method may further comprise attaching a second linker to the CAR-ID to produce a second switch intermediate. Attaching the second linker to the CAR-ID may comprise one or cycloadditions. Attaching the second linker to the CAR-ID may comprise a copper free reaction. Attaching the second linker to the CAR-ID may comprise one or more copper-containing reactions. Attaching the second linker to the CAR-ID may comprise one or more Diels Alder reactions. Attaching the second linker to the CAR-ID may comprise one or more hetero Diels Alder reactions. Attaching the second linker to the CAR-ID may comprise one or more ester couplings. Attaching the second linker to the CAR-ID may comprise oxime ligation. Attaching the second linker to the CAR-ID may comprise forming one or more oximes between the second linker and the CAR-ID. Attaching the second linker to the CAR-ID may comprise forming one or more stable bonds between the second linker and the CAR-ID. Attaching the second linker to the CAR-ID may comprise forming one or more covalent bonds between the second linker and the CAR-ID. Attaching the second linker to the CAR-ID may comprise forming one or more non-covalent bonds between the second linker and the CAR-ID. Attaching the second linker to the CAR-ID may comprise forming one or more ionic bonds between the second linker and the CAR-ID.

Attaching the CAR-ID to the TID may comprise attaching the second switch intermediate to the TID. Attaching the second switch intermediate to the TID may comprise one or cycloadditions. The one or more cycloadditions may comprise a Huisgen cycloaddition. The one or more cycloadditions may comprise a [3+2] cycloaddition. The one or more cycloadditions may comprise a [3+2] Huisgen cycloaddition. The one or more cycloadditions may comprise a copper-free cycloaddition. Attaching the second switch intermediate to the TID may comprise a copper free reaction. Attaching the second switch intermediate to the TID may comprise one or more copper-containing reactions. Attaching the second switch intermediate to the TID may comprise one or more Diels Alder reactions. Attaching the second switch intermediate to the TID may comprise one or more hetero Diels Alder reactions. Attaching the second switch intermediate to the TID may comprise one or more ester couplings. Attaching the second switch intermediate to the TID may comprise one or more isothiocyanate couplings. Attaching the second switch intermediate to the TID may comprise attaching the linker to an amino acid of CAR-ID. The amino acid may be an unnatural amino acid. Attaching the second switch intermediate to the TID may comprise one or more bioorthogonal reactions.

Attaching the CAR-ID to the TID may comprise attaching the first switch intermediate to the second switch intermediate. Attaching the first switch intermediate to the second switch intermediate may comprise one or cycloadditions. The one or more cycloadditions may comprise a Huisgen cycloaddition. The one or more cycloadditions may comprise a [3+2] cycloaddition. The one or more cycloadditions may comprise a [3+2] Huisgen cycloaddition. The one or more cycloadditions may comprise a copper-free cycloaddition. Attaching the first switch intermediate to the second switch intermediate may comprise a copper free reaction. Attaching the first switch intermediate to the second switch intermediate may comprise one or more copper-containing reactions. Attaching the first switch intermediate to the second switch intermediate may comprise one or more Diels Alder reactions. Attaching the first switch intermediate to the second switch intermediate may comprise one or more hetero Diels Alder reactions. Attaching the first switch intermediate to the second switch intermediate may comprise one or more ester couplings. Attaching the first switch intermediate to the second switch intermediate may comprise one or more isothiocyanate couplings.

### Purification of CAR-EC Switches and Portions Thereof

Disclosed herein are methods of purifying CAR-EC switches disclosed herein, comprising separating the CAR-EC switches disclosed herein from components of a CAR-EC switch production system (e.g. cellular debris, free amino acids). Purifying the CAR-EC switch may comprise use of one or more concentrator columns, electrophoresis, filtration, centrifugation, chromatography or a combination thereof. Chromatography may comprise size-exclusion chromatography. Additional chromatography methods include, but are not limited to, hydrophobic interaction chromatography, ion exchange chromatography, affinity chromatography, metal binding, immunoaffinity chromatography, and high performance liquid chromatography or high pressure liquid chromatography. Electrophoresis may comprise denaturing electrophoresis or non-denaturing electrophoresis.

The CAR-EC switches may comprise one or more peptide tags. The methods of purifying CAR-EC switches may comprise binding one or more peptide tags of the CAR-EC switches to a capturing agent. The capturing agent may be selected from an antibody, a column, a bead and a combination thereof. The one or more tags may be cleaved by one or more proteases. Examples of tags include, but are not limited to, polyhistidine, FLAG® tag, HA, c-myc, V5, chitin binding protein (CBP), maltose binding protein (MBP), and glutathione-S-transferase (GST).

The methods may further comprise lyophilization or ultracentrifugation of the CAR-IDs, TIDs and/or the CAR-EC switches.

The purity of the CAR-IDs, TIDs and/or the CAR-EC switches may be equal to or greater than 50%, 55%, 60%, 65%, 70%, 75%, 80%, 85%, 90%, 91%, 92%, 93%, 94%, 95%, 96%, 97%, 98%, 99% or more. The purity of CAR-IDs, TIDs and/or the CAR-EC switches may be equal to or greater than 85%. The purity of the CAR-IDs, targeting polypeptides and/or the CAR-EC switches may be equal to or greater than 90%. The purity of the CAR-IDs, TIDs and/or the CAR-EC switches may be equal to or greater than 95%. The purity of the CAR-IDs, TIDs and/or the CAR-EC switches may be equal to or greater than 97%.

The methods of producing CAR-EC switches disclosed herein may comprise producing CAR-EC switches that are structurally homogeneous. The method of producing the CAR-EC switch from a polynucleotide may result in one or more CAR-EC switches that have the same or similar form, features, binding affinities (e.g. for the CAR or the target), geometry and/or size. The homogeneity of the CAR-EC switches may be equal to or greater than 50%, 55%, 60%, 65%, 70%, 75%, 80%, 85%, 90%, 91%, 92%, 93%, 94%, 95%, 96%, 97%, 98%, 99% or more. The homogeneity of the CAR-EC switches may be equal to or greater than 85%. The homogeneity CAR-EC switches may be equal to or greater than 90%. The homogeneity of the CAR-EC switches may be equal to or greater than 95%. The homogeneity of the CAR-EC switches may be equal to or greater than 97%. The homogeneity may be a structural homogeneity. The homogeneity may be a structural homogeneity prior to administering the cell to a subject. The homogeneity may be a structural homogeneity prior to modifications to the CAR-EC switch by cellular activities (methylation, acetylation, glycosylation, etc.). These high percentages of homogeneity may provide a more predictable effect of the CAR-EC switch. These high percentages of homogeneity may provide for less off-target effects of the CAR-EC switch, when combined with a CAR-EC to treat a condition in a subject.

### Methods of Optimization

Disclosed herein are methods of optimizing a CAR switch for maximum safety and efficacy in a subject. Further disclosed herein are methods of optimizing treatment of a condition in a subject comprising optimizing a CAR switch. Also, disclosed herein are methods of optimizing a sCAR receptor platform for maximum safety and efficacy in a subject. Further disclosed herein are methods of optimizing treatment of a condition in a subject comprising optimizing a sCAR platform.

### Optimizing CAR-EC Activation

Disclosed herein are methods of producing an optimal sCAR platform, comprising: incorporating a first CAR-ID to a first site of a TID that binds a first cell surface molecule on a first target cell to produce a first switch; incorporating a second CAR-ID to a second site of a second TID that binds a second cell surface molecule on a second target cell to produce a second switch; contacting the first target cell with the first switch and a first CAR-EC expressing a first CAR; contacting the second target cell with the second switch and a second CAR-EC expressing a second CAR; and comparing a first cytotoxic effect of the first switch and the first CAR-EC on the first target cell to a second cytotoxic effect of the second switch and the second CAR-EC on the second target cell; and selecting the first switch and first CAR-EC or the second switch and the second CAR-EC as the optimal sCAR platform based on comparing the first cytotoxic effect to the second cytotoxic effect.

Further disclosed herein are methods of producing an optimal sCAR, comprising: incorporating a first CAR-ID to a first site of a TID that binds a first cell surface molecule on a first target cell to produce a first switch; incorporating a second CAR-ID to a second site of a second TID that binds a second cell surface molecule on a second target cell to produce a second switch; contacting the first target cell with the first switch and a first CAR-EC expressing a first CAR; contacting the second target cell with the second switch and a second CAR-EC expressing a second CAR; and comparing a first effect of the first switch and the first CAR-EC on the first chimeric antigen receptor effector cell to a second effect of the second switch and the second CAR-EC on the second CAR-EC; and selecting the first switch and first CAR-EC or the second switch and the second CAR-EC as the optimal switchable chimeric antigen receptor platform based on comparing the first effect to the second effect.

The first CAR-ID and the second CAR-ID may be the same. The first TID and the second TID may be the same. The first site and the second site may be the same. The first site and the second site may be different. The methods may further comprise incorporating one or more additional CAR-IDs to the first and/or second TID to produce a first multivalent switch and/or a second multivalent switch.

The first CAR and the second CAR may be the same. The first CAR and the second CAR may be different. The first CAR and the second CAR may differ by a domain selected from an extracellular domain, a transmembrane domain, an intracellular domain and a hinge domain. A first hinge domain of the first chimeric antigen receptor and a second hinge domain of the second chimeric antigen receptor may differ by a feature selected from flexibility, length, amino acid sequence and combinations thereof.

Contacting the first target cell and/or contacting the second target cell may occur in vitro. Contacting the first target cell and/or contacting the second target cell may occur in vivo. By way of non-limiting example, contacting the first target cell and/or contacting the second target cell may occur in an in vivo model, such as a mouse. The in vivo model may have a condition or disease. The condition or disease may be a tumor or a cancer. Comparing the first cytotoxic effect to the second cytotoxic effect may comprise comparing a feature selected from viability of target cells, expression/production of activation markers (e.g., production of cytokines) by the first CAR-EC and/or second CAR-EC, viability of off-target cells, tumor burden, and health of a subject an in vivo model. The method may further comprise comparing the first cytotoxic effect and or the second cytotoxic effect to that of a canonical CAR cytotoxic effect, wherein the canonical CAR is a non-switchable CAR (e.g., not controlled by a CAR switch).

The first target cell and the second target cell may express the same cell surface molecule. The first target cell and the second target cell may express different levels of the same cell surface molecule, resulting in different cell surface molecule (e.g., antigen) densities. The method may comprise comparing the first cytotoxic effect on the first target cell to the first cytotoxic effect on the second target cell. The method may comprise comparing the first cytotoxic effect on the first target cell to the second cytotoxic effect on the second target cell. The method may comprise comparing the first cytotoxic effect on the first target cell to the second cytotoxic effect on the first target cell.

The methods of optimizing may comprise modulating the distance and geometry of the immunological synapse, chimeric receptor binding affinity for the switch, valency and location of the CAR-IDs on the switch, and the density of chimeric receptors on the CAR-EC surface. The optimizing may result in activating the CAR-EC to an activation level that results in a desired CAR-EC fate or phenotype. Demonstrated herein are methods of FITC-grafting to create switches with a range of geometries, lengths, and valences which can be used to systematically optimize the sCAR immunological synapse (see, e.g., Example 1).

### Optimizing the Immunological Synapse—Length and Geometry

The methods disclosed herein comprise developing sCAR-T cell systems with switch-mediated control over the immunological synapse formed by the switch between the sCAR and target cell, wherein the immunological synapse may be defined as the junction between the CAR-EC and the target cell.

The methods may comprise modulating the length of the immunological synapse or the distance between the CAR-EC and the target cell. The methods may comprise modulating the length or size of the switch. The methods may comprise modulating the length or size of the CAR extracellular domain. Modulating the length or size of the CAR extracellular domain may comprise modulating the length of the CAR hinge.

The methods may comprise modulating the length of the immunological synapse by modulating the location of the CAR-IDs on the switch. In some cases, the methods comprise designing switches with the CAR-IDs placed distal to an antigen binding domain of the TID. In some cases, the methods comprise designing switches with the CAR-IDs placed distal to an antigen binding domain of the targeting antibody or antibody fragment. Distal may be the C terminus of the targeting antibody or antibody fragment. Distal may be the constant region of the targeting antibody or antibody fragment. In some cases, the methods comprise designing switches with the CAR-IDs placed proximal to the TID. In some cases, the methods comprise designing switches with the CAR-IDs placed proximal to the antigen binding domain of the targeting moiety. Proximal may be the N terminus of the targeting antibody or antibody fragment. Proximal may be the variable region of the targeting antibody or antibody fragment. In some cases, the methods comprise designing switches with the CAR-IDs placed intermediate to an antigen binding domain of the TID. In some cases, the methods comprise designing switches with the CAR-IDs placed intermediate to the antigen binding domain of the targeting antibody or antibody fragment. In some cases, the methods comprise designing a switch such that the CAR-ID is located at the C-terminus of the TID (e.g Fab) to enable sufficient length to display the CAR-ID to the sCAR-T while avoiding steric hindrance from a relatively large cell surface molecule. Thus, modulating the length of the switch and/or CAR extracellular domain may afford varying levels of sCAR-T cell activation. In some cases, switches with the CAR-ID proximal to the antigen binding domain may stimulate less CAR-EC activation than switches with the CAR-ID distal to the antigen binding domain. In other cases, switches with the CAR-ID distal to the antigen binding domain may stimulate less CAR-EC activation than switches with the CAR-ID proximal to the antigen binding domain. This underscores the necessity to empirically design switches with defined structures and valencies enabled by site specific fusing/grafting methodologies described herein. This is a significant advantage compared with previous reports that use non-specific conjugation to create switches which produce heterogeneous mixtures of conjugates. These reports do not provide methods of optimizing the sCAR immunological synapse for any antigen.

As shown in Example 15, CD19 targeting switches with FITC grafted proximal to the antigen binding interface of the FMC63 Fab, may be superior to switches with FITC grafted at the C-terminus. Although the epitope of anti-CD19 antibody FMC63 and corresponding structure of the CD19 antigen are not known, this may be due to a decreased distance between target cell and sCAR-T cell. In the physiological immunological synapse formed by the native T cell receptor (TCR), the distance between the T cell and antigen presenting cell is approximately 150 Å. This distance is critical to sterically exclude inhibitory phosphatases such as CD45 and CD148 from the synapse which act to dephosphorylate signaling molecules and down regulate T cell activation. It is likely that the longer synapse contributed by the C-terminal switches (65 Å longer than the N-terminal switches by length of Fab) is unable to sterically exclude these inhibitory molecules, resulting in less productive sCAR signaling. Thus, the methods disclosed herein may comprise modulating the distance of the immunological synapse by modulating the length of the switch and/or CAR extracellular domain such that the distance of the immunological synapse is not greater than about 150 Å, about 175 Å, or about 200 Å.

In some embodiments, the methods comprise modulating the length of the CAR hinge. The methods may comprise activating a first CAR-EC comprising a first CAR with a first hinge and activating a second CAR-EC comprising a second CAR with a second hinge and comparing an activity of the first CAR-EC to that of the second CAR-EC. The activity, by way of non-limiting example, may be selected from cytokine release, expression of a phenotypic marker, proliferation, senescence, and migration/trafficking. The first CAR hinge may be a long hinge and the second CAR hinge may be a short hinge. In some cases, the switch may provide greater sCAR-T activity when paired with a CAR that has a long hinge versus a short hinge. In some cases, the switch may provide lesser sCAR-T activity when paired with a CAR that has a long hinge versus a short hinge. The hinge may be a flexible hinge. A flexible hinge may be a linear sequence of amino acids with no known secondary structure in which the torsion angles or rotation around the bonds of the polypeptide backbone have the freedom to occupy many different orientations. The hinge may be a rigid or structured hinge. A rigid or structured hinge may be a linear sequence of amino acids that form a defined secondary structure in which the torsion angles or rotation around the bonds of the polypeptide backbone have defined preferences to occupy a limited number of orientations. The long hinge may have a length of about 20 to about 200 amino acids, about 20 to about 100 amino acids, about 30 to about 100 amino acids, about 40 to about 100 amino acids, or about 45 to about 100 amino acids. The long hinge may comprise a portion of a CD8 protein. The portion of the CD8 protein may be between about 4 amino acids and about 100 amino acids. The portion of the CD8 protein may be about 45 amino acids. The short hinge may be a flexible hinge. The short hinge may be a rigid or structured hinge. The short hinge may have a length of about 1 to about 20 amino acids, about 5 to about 20 amino acids, or about 10 to about 20 amino acids. The short hinge may comprise a portion of an immunoglobulin. The immunoglobulin may be an IgG. The immunoglobulin may be an IgG4. The IgG4 may be mutated (IgG4m). The portion of the immunoglobulin may be between about 1 amino acid and about 20 amino acids. The portion of the immunoglobulin may be about 12 amino acids.

The methods of optimizing may further comprise accounting for the size and structure of the cell surface molecule (e.g., antigen) on the target cell, demonstrated by site-specific FITC conjugation on the anti-CD22 antibody, m971. In this case, the m971 antibody has a membrane proximal epitope on CD22 and several large modular domains which may sterically preclude binding. Correspondingly, the m971 switches were optimal with FITC placed at sites distal from the antigen binding interface. Thus, the distance of the immunological synapse is a key parameter to consider when designing switches and highlights the requirement for empirical design as described herein.

The methods disclosed herein may comprise varying the geometry of the immunological synapse. The geometry may be defined or referred to herein as the orientation of the bio-orthogonal immunological synapse. The methods disclosed herein comprise optimizing geometry of the switch. The methods disclosed herein comprise optimizing geometry of the switch to be compatible with a CAR. The CAR may be a universal CAR. Optimizing the geometry of the switch may comprise selecting one or more sites on the TID for CAR-ID conjugation. The first site and/or second site may be selected from an N terminus of the TID, a C terminus of the TID, and an internal site of the TID. The first site and/or second site may be selected from an N terminus of the antibody or antibody fragment, a C terminus of the antibody or antibody fragment, and an internal site of the antibody or antibody fragment. The first site and/or second site may be selected from a light chain of the antibody or antibody fragment and a heavy chain of the antibody or antibody fragment. The first site and/or second site may be selected from a variable region of the antibody or antibody fragment and a constant region of the antibody or antibody fragment. The first site and/or second site may be selected from a VL domain, a CL domain, a VH domain, a CH1 domain, a CH2 domain, a CH3 domain, and a hinge domain of the antibody or antibody fragment. Incorporating the first/second chimeric antigen receptor binding peptide may comprise a method selected from fusing, grafting, conjugating, linking, and combinations thereof.

Optimizing the geometry of the switch may further comprise incorporating one or more linkers in the switch. Optimizing the geometry of the switch may further comprise comparing two or more linkers. The two or more linkers may differ by a feature selected from flexibility, length, amino acid sequence, and combinations thereof. The method may comprise incorporating a first linker to the first site, wherein the first linker links the first chimeric antigen receptor binding peptide to the first targeting moiety. The method may further comprise incorporating a second linker to the second site wherein the second linker links the second chimeric antigen receptor binding peptide to the second targeting moiety. The first linker and the second linker may be the same. The first linker and the second linker may be different. The first linker and the second linker may differ by a feature selected from flexibility, length, and combinations thereof. The first and second linker may be selected from those depicted in FIG. 19 FIG. 20, FIG. 51, FIG. 52, FIG. 54, and FIG. 55.

### Optimizing CAR-EC Phenotype, Activation, Fate and Progeny

CAR-T cell expansion and trafficking in humans has been shown to be predictive of clinical responses in clinical trials. The methods disclosed herein may comprise optimizing CAR-EC phenotype, activation, fate and progeny. Optimizing CAR-EC activation may comprise optimizing the switch dose. The methods may comprise administering a first dose of a switch to a subject and a second dose of the switch to the subject and comparing CAR-EC cytokine release, CAR-EC expansion/fate, CAR-EC trafficking to disease sites, CAR-EC proliferation, and any combination thereof. The method may further comprise continuing administering the first dose or the second dose in the subject or administering a third dose to the subject, after the comparing. For example, as presented herein, it was shown that switch dose may be used to control CAR-EC cytokine release in the Nalm-6^{Luc/GFP} xenograft model. Because the Nalm-6 tumor lacks CD80 and CD86 co-receptors, it is difficult to treat and has become a standard for CAR-T therapy adjudication. In vivo expansion and trafficking of sCAR-T cells to sites of disease was demonstrated to be reliant on switch dosing. Importantly, serum levels of human cytokines IL-2, TNFα, IFNγ, and MCP1 were controlled in a dose-dependent manner by anti-CD19 AB-FITC dose. A key finding of these studies was that subjects treated with lower doses of switch (0.05 mg/kg) could provide complete clearance of Nalm-6^{Luc/GFP} with a dose escalation protocol and with lower levels of cytokine release. This was likely due to a decreased tumor burden at the time of dose-escalation. This indicates that low dose switch treatment combined with dose escalations may be an effective method of mitigating CRS and TLS in the treatment of patients with high tumor burdens. Thus, the methods may comprise administering a first dose of switch before a relapse in the subject and a second dose after the relapse. The second dose may be higher than the first dose.

The methods may comprise administering a first dose of a switch to a subject and a second dose of the switch to the subject and comparing CAR-EC phenotype after the first dose to CAR-EC phenotype after the second dose. In the sCAR-T cell system persistence is critical to enable re-dosing strategies in the case of relapse. Persistence of sCAR-T cells may also be promoted through rest phases in which switch dosing is withheld to prevent exhaustion related to persistent T cell signaling. Thus, the methods may further comprise comparing effector memory T (TEMRA) cell quantity in the subject before administering the first/second switch to the subject to after administering the first/second switch to the subject.

The methods may comprise optimizing the switch or sCAR-T platform to control CAR-EC fate. The methods may comprise optimizing the switch or sCAR-T platform to optimally activate the CAR-EC, thereby optimizing CAR-EC fate. For example, optimally activating the CAR-EC may cause it to become an effector memory T cell, as opposed to partially activating or over-activating, which can lead to death, senescence, or anergy of the CAR-EC. The methods may comprise activating a CAR with a first switch and activating the CAR with a second switch and comparing CAR-EC fate after the first switch to CAR-EC fate after the second switch. The methods may comprise administering a first switch to a subject and a second switch to the subject and comparing CAR-EC fate after the first switch to CAR-EC fate after the second switch. The methods may comprise administering a first dose of a switch to a subject and a second dose of the switch to the subject and comparing CAR-EC fate after the first dose to CAR-EC fate after the second dose.

### Optimizing Switch Valency

Using methods of optimizing sCAR-T configurations, sCAR-T systems disclosed herein show similar in vitro T cell activation, functional cytokine release, and cell-killing sensitivity and specificity, and in vivo tumor elimination comparable to the efficacy of conventional CARS. The examples disclosed herein demonstrate that the methods of optimization may be highly dependent on switch and CAR hinge design, and less dependent on the targeting modality. Disclosed herein is the development of binary sCAR-T cells which function on multiple inputs (e.g., multiple orthogonal pairs) that enable precise control over sCAR-T cell function.

Methods of optimizing the CAR-EC platform or CAR-EC switch may comprise incorporating more than one CAR-ID in to the switch to produce a multivalent switch. In some cases, a bivalent switch may be preferable to a monovalent switch. In some cases, the monovalent switch may be preferable to the bivalent switch. The methods of optimizing may comprise comparing a first effect of a CAR-EC on a target cell wherein the first CAR-ID is a first distance from the second CAR-ID to a second effect of the CAR-EC on the target cell wherein the first CAR-ID is a second distance from the second CAR-ID. The first or second distance may be between about 5 Å and about 100 Å. The first or second distance may be between about 8 Å and about 80 Å. The first or second distance may be between about 10 Å and about 50 Å. The first or second distance may be between about 10 Å and about 40 Å. The first or second distance may be between about 10 Å and about 30 Å. The first or second distance may be about 12 Å. The first or second distance may be about 24 Å. The ranges disclosed herein encompass all intervening integers and fractions thereof (e.g. 5, 5.1, 5.2, 5.3, 5.4, 5.5, 5.6, 5.7, 5.8, 5.9, 6, 7, 8, 9, 10,).

### Optimizing the CAR and CAR-EC

The methods of optimizing may comprise optimizing CAR density on the membranes of the CAR-ECs. Optimizing CAR density on the membranes of the CAR-ECs may comprise modulating the expression of the CAR. This can be done, for example, by engineering cells that express a CAR under a high or low expression promoter. Alternatively, or additionally, CARs may be designed to multimerize on the CAR-EC membrane, creating “rafts” of CARs that stimulate a greater effect (e.g., cytotoxic effect) in the CAR-EC than a single CAR, or even two CARs, alone. This may be achieved, by way of non-limiting example, by incorporating a cysteine residue into the first chimeric antigen receptor and/or the second chimeric antigen receptor in order to multimerize the first chimeric antigen receptor and/or the second chimeric antigen receptor through a disulfide bond.

The first/second CAR-ECs may be derived from a T cell (e.g., genetically modified T cell or differentiated from a T cell). The CAR-EC may be a T cell. The CAR-EC may be a cell of a T cell lineage. The CAR-EC may be a mature T cell. The CAR-EC may be a precursor T cell. The CAR-EC may be a cytotoxic T cell. The CAR-EC may be a naive T cell. The CAR-EC may be a memory stem cell T cell (T_{MSC}). The CAR-EC may be a central memory T cell (T_{CM}). The CAR-EC may be an effector T cell (TE). The CAR-EC may be a CD4+ T cell. The CAR-EC may be a CD8+ T cell. The CAR-EC may be a CD4+ and CD8+ cell. The CAR-EC may be an alpha-beta T cell. The CAR-EC may be a gamma-beta T cell. The CAR-EC may be a natural killer T cell. The CAR-EC may be a helper T cell. The CAR-EC may be a neutrophil. The neutrophil may be a CD34⁺ neutrophil. The neutrophil may be engineered or genetically modified to have greater cytotoxic capacity than a naturally-occurring neutrophil.

The methods may further comprise consideration for switch compatibility with the cell surface molecule and the CAR. The methods may comprise testing compensatory mutations in switches and CARs in the development of a single, universal sCAR.

Further disclosed herein are optimized sCAR platforms, comprising: a CAR-EC switch comprising a CAR-ID and a TID; and a CAR-EC that expresses a CAR, wherein the sCAR-EC platform is produced/derived by methods of producing an optimal sCAR platform disclosed herein.

### EXAMPLES

The following illustrative examples are representative of embodiments of the software applications, systems, and methods described herein and are not meant to be limiting in any way.

### Example 1

### Design Optimization of Multiple sCAR T Cells Targeting Breast Cancers

The standard of care for patients with Her2⁺ cancer, trastuzumab, is not approved in patients with low levels of Her2 expression (Her2 1+), which occurs in ˜35% of breast cancer patients and represents a major unmet medical need. This example demonstrates that optimal sCAR-T cell-switch combinations potently lysed Her2 positive tumors, including Her2 1+ tumors, both in vitro and in vivo with efficacy that is comparable to the conventional anti-Her2 CAR-T cells. Activity of these switches depended strongly on the orientation of the bio-orthogonal immunological synapse, which was determined by location of the tag incorporation in the switch.

To develop switches that redirect the activity of sCAR-T cells to Her2-expressing cancer cells, anti-Her2 (4D5 Fab) switches were conjugated to FITC at defined sites in the variable or constant regions of antibody antigen binding fragment (Fab). These tag positions were chosen to provide switches that formed the ternary complex between the sCAR-T cell, switch, and the target cell with a diverse range of distance and orientation, thus allowing the empirical optimization of immunological synapse.

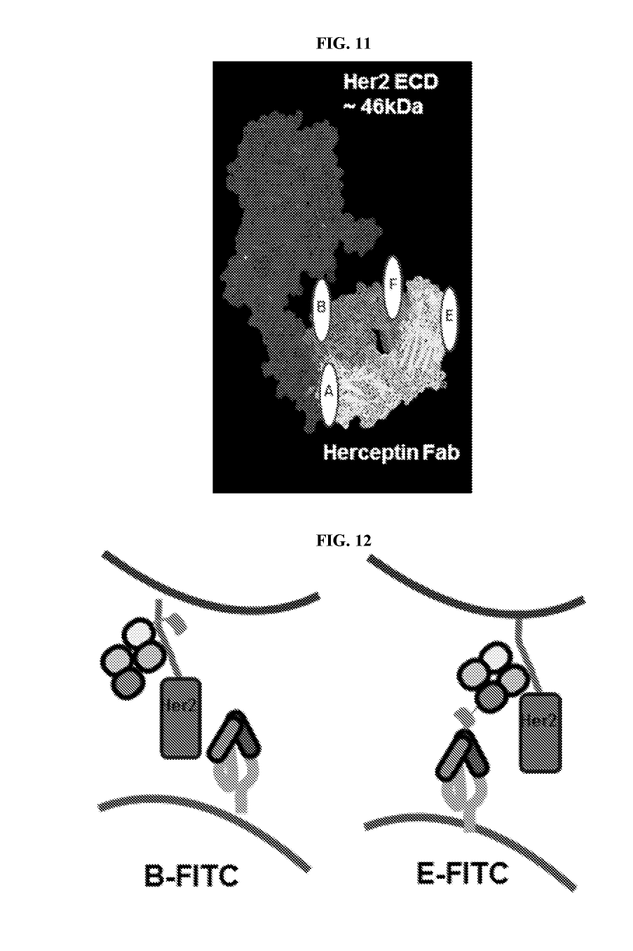

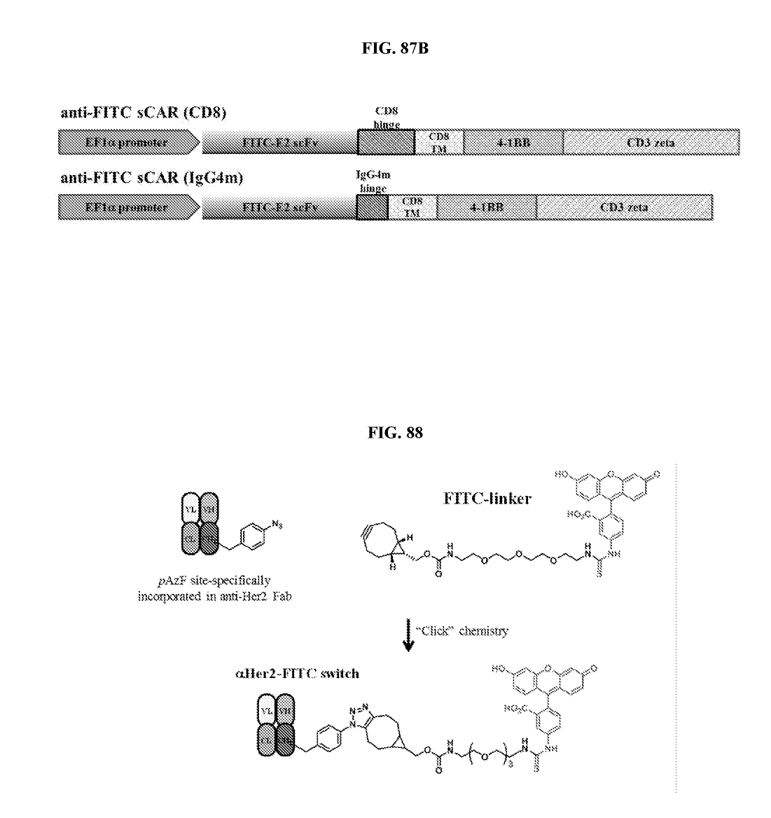

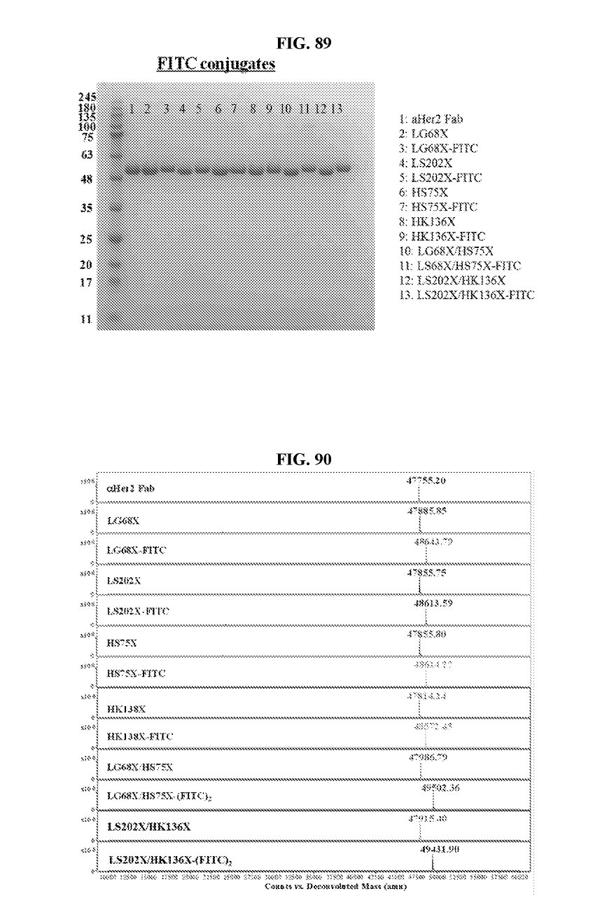

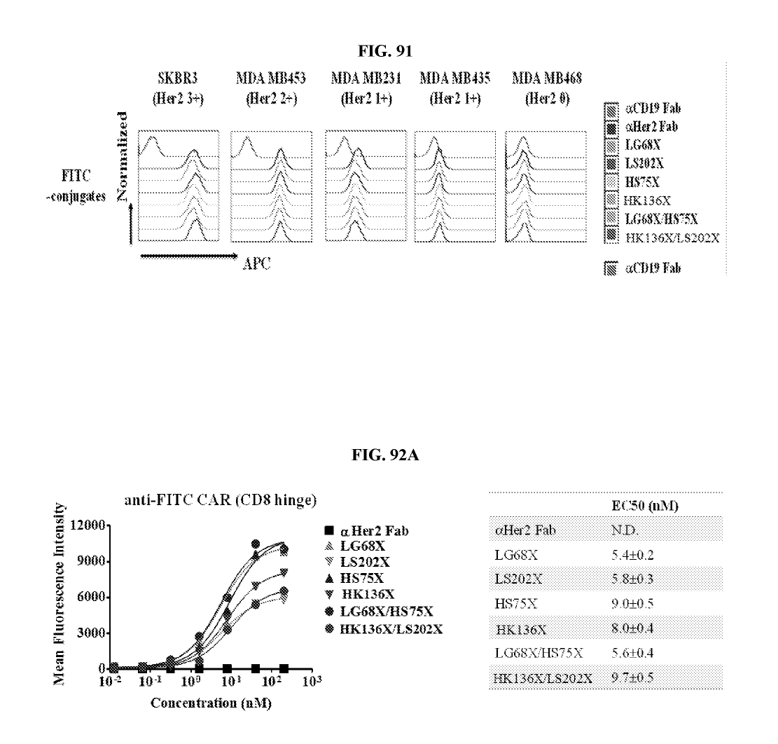

For FITC-based switches, unnatural amino acid (UAA) methodology was deployed to site-specifically conjugate FITC to the 4D5 Fab. Briefly, a mutant 4D5 Fab with the TAG nonsense codon at select residues was co-expressed in Escherichia coli (E. coli) with an orthogonal Methanococcus jannaschii-derived tRNA/aminoacyl-tRNA synthetase (tRNA_{CUA}/pAzFRS) pair that selectively incorporates p-azidophenylalanine (pAzF) into proteins in response to the TAG codon. The pAzF residue was individually incorporated at light chain residues G68 or S202 (LG68X or LS202X), or a heavy chain residues S75 or K136 (HS75X or HK136X) to create four monovalent switches (FIG. 87 and FIG. 11). In addition, to create two bivalent switches, pAzF residues were incorporated at both LG68X and HS75X, or LS202X and HK136X simultaneously. The LG68X and HS75X sites are located in the framework region III of the variable domain proximal to the antigen binding interface of 4D5 Fab, outside of the complementary determine regions. The LS202X and HK136X sites are in the constant region of the 4D5 Fab distal from the antigen binding interface. To chemically conjugate the Fab, a linker-modified FITC harboring a cyclooctyne group (BCN-PEG4-FITC) was attached via a “Click” reaction (FIG. 88). Conjugations proceeded to >95% as determined by SDS-PAGE gel (FIG. 89) and high resolution MS (FIG. 90 and Table 2).

TABLE 2
QTOF-MS characterization of different anti-Her2
Fab and the FITC conjugates

| | Non-reducing condition | |
|---|---|---|
| | (−DTT) | Reducing condition (+DTT) |
| Constructs | Expected/Observed Mass | Expected/Observed Mass |
| Wild type | 47762/47755 | 23457/23454 (light chain) |
| | | 24305/24303 (heavy chain) |
| LG68X | 47893/47886 | 23588 (23562^{a})/23559^{a} (LG68X light |
| | | chain) |
| | | 24305/24303 (heavy chain) |
| LG68X-FITC | 48651/48644 | 24345/24343 (LG68X-FITC light chain) |
| | | 24305/24303 (heavy chain) |
| LS202X | 47863/47856 | 23558 (23532^{a})/23529^{a} (LS202X light |
| | | chain) |
| | | 24305/24303 (heavy chain) |
| LS202X-FITC | 48621/48614 | 24315/24313 (LS202X-FITC light chain) |
| | | 24305/24303 (heavy chain) |
| HS75X | 47863/47856 | 23457/23454 (light chain) |
| | | 24406 (24380^{a})/24378^{a} (HS75X heavy |
| | | chain) |
| HS75X-FITC | 48621/48614 | 23457/23454 (light chain) |
| | | 25163/25161 (HS75X-FITC heavy |
| | | chain) |
| HK136X | 47822/47815 | 23457/23454 (light chain) |
| | | 24365 (24339^{a})/24336^{a} (HK136X heavy |
| | | chain) |
| HK136X-FITC | 48580/48572 | 23457/23454 (light chain) |
| | | 25122/25120 (HK136X-FITC heavy |
| | | chain) |
| LG68X/HS75X | 47884/47987 | 23588 (23562^{a})/23559^{a} (LG68X light |
| | | chain) |
| | | 24406 (24380^{a})/24378^{a} (HS75X heavy |
| | | chain) |
| LG68X/HS75X- | 49510/49503 | 24345/24343 (LG68X-FITC light chain) |
| (FITC)₂ | | 25163/25161 (HS75X-FITC heavy |
| | | chain) |
| LS202X/HK136X | 47923/47915 | 23558 (23532^{a})/23529^{a} (LS202X light |
| | | chain) |
| | | 24365 (24339^{a})/24336^{a} (HK136X heavy |
| | | chain) |
| LS202X/HK136X- | 49439/49432 | 24315/24313 (LS202X-FITC light chain) |
| (FITC)₂ | | 25122/25120 (HK136X-FITC heavy |
| | | chain) |

FITC-based switches bound Her2 expressing cancer cells to a similar extent as wild type 4D5 Fab (FIG. 91 and Table 3) and did not bind to MDA MB468 cancer cells lacking Her2 expression.

TABLE 3
Comparison of binding activity of different FITC labelled anti-Her2 conjugates on breast cancer cells

| | | MFI^{a}/Relative binding index^{b} | | | | | | | |
|---|---|---|---|---|---|---|---|---|---|
| | | | | | | | | LG68/ | L202/ |
| Cell line | Her2 Level | αCD19 Fab | αHer2 Fab | LG68 | LS202 | HS75 | HK136 | HS75 | HK136 |
| SKBR3 | Her2 3+ | 144 | 13606/96 | 14299/99 | 18414/128 | 15513/108 | 17204/119 | 13575/94 | 20323/141 |
| MDA | Her2 2+ | 151 | 8645/57 | 8493/56 | 8539/57 | 8195/54 | 7133/47 | 7306/48 | 8332/55 |
| MB453 | | | | | | | | | |
| MDA | Her2 1+ | 89 | 586/7 | 411/5 | 439/5 | 456/5 | 444/5 | 427/5 | 416/5 |
| MB231 | | | | | | | | | |
| MDA | Her2 1+ | 94 | 228/2 | 203/2 | 193/2 | 213/2 | 216/2 | 216/2 | 222/2 |
| MB435 | | | | | | | | | |
| MDA | Her2 0 | 86 | 82/1 | 85/1 | 85/1 | 90/1 | 83/1 | 84/1 | 85/1 |
| MB468 | | | | | | | | | |

^{a}MFI: mean fluorescence intensity calculated by software FlowJo X10.0.6
^{b}Relative Binding index represent MFI of indicated antibody/MFI of anti-CD19 Fab

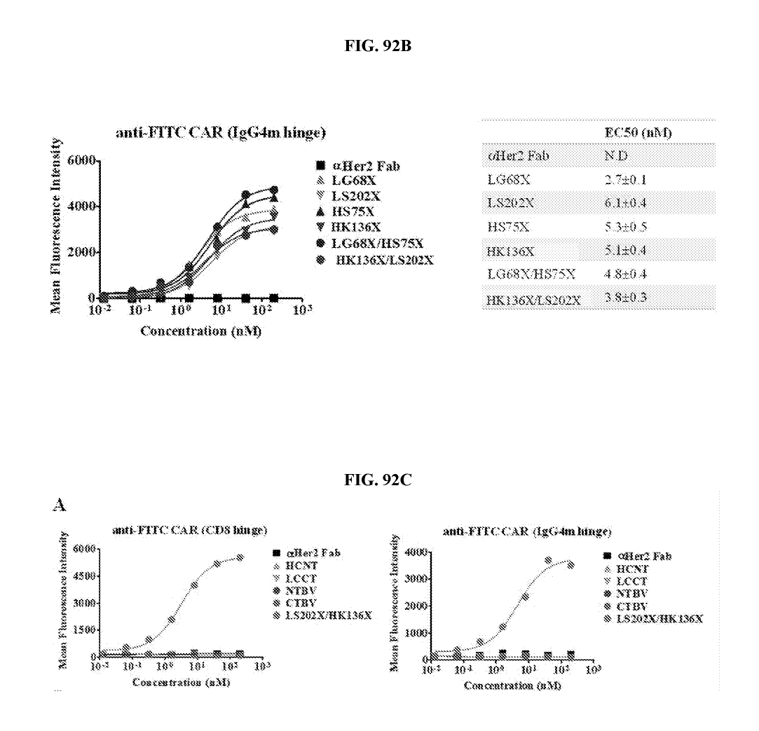

Next, the capacity of sCAR-T cells with CD8 or IgG4m hinges to bind their corresponding anti-Her2 switches was tested. FITC switches were used to stain anti-FITC sCAR-T cells, and detected with an APC-labeled anti-human κ-chain antibody specific for the constant region at the Fab. As shown in FIG. 92A-FIG. 92C, all the switches bound to anti-FITC sCAR-T cells with similar EC₅₀ (2.7 to 9.7 nM). The wild type 4D5 Fab (αHer2 Fab) or irrelevant antibody switches failed to bind sCAR-T cells, demonstrating the specificity of the sCAR to the specific FITC tag (FIG. 92C) Together with the target cell binding assay, we confirmed that site specific FITC conjugates preserved the binding specificity to the target cells as well as the tag recognition of 4D5-based switch by the sCAR-T cells.

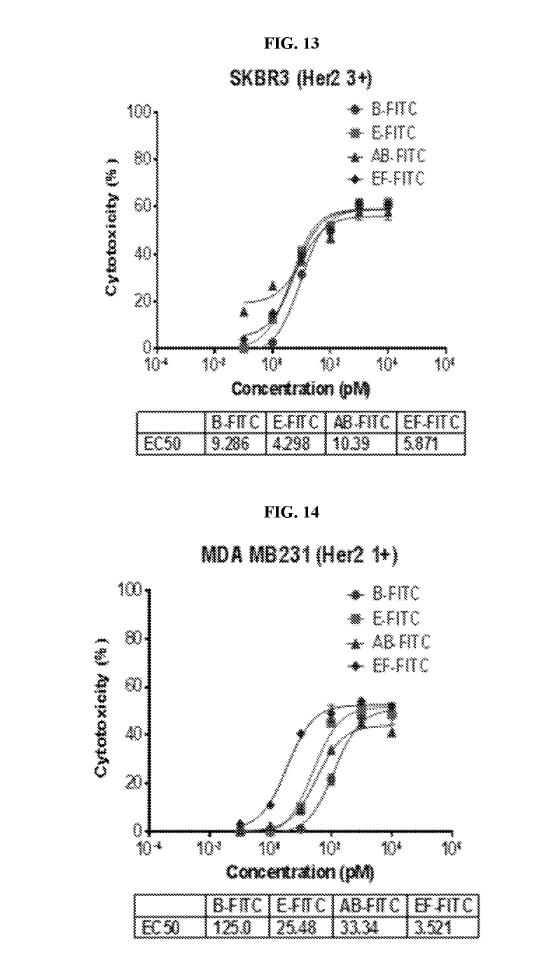

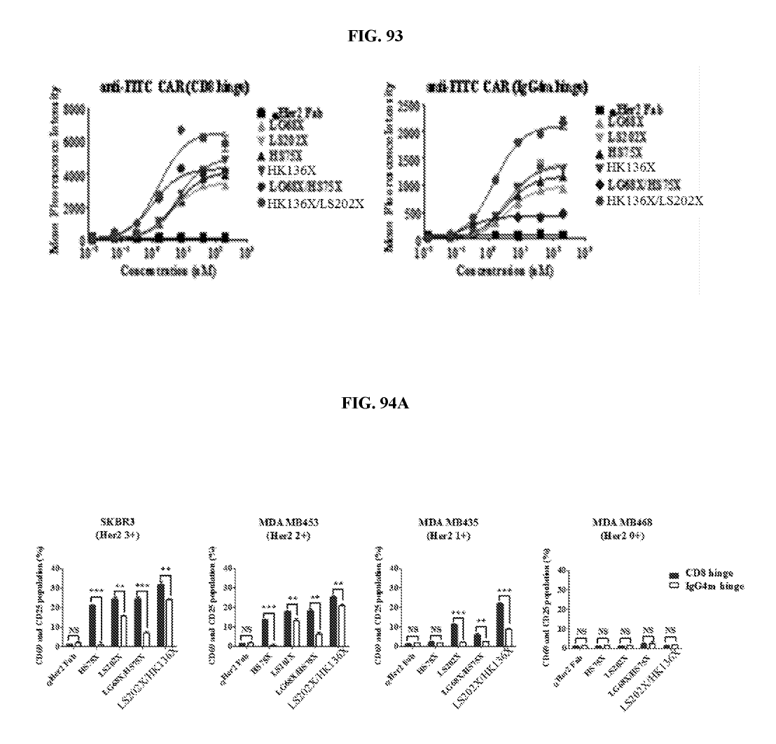

As shown in FIG. 93 and Table 4, switches with the FITC distal to the antigen binding interface formed ternary complex more effectively than switches where the tags were proximal to the antigen binding interface, demonstrated by increased MFI. Bivalent switches formed ternary complexes more efficiently compared with their monovalent counterparts, presumably due to increased avidity, with the greatest efficiency observed for the distal bivalent switches, LS202X/HK136X. While the ability to form ternary complexes did not correlate to increased cytotoxic effects in cells expressing high levels of Her2 (SKBR3, FIG. 13), it did correlate to increased cytotoxic effects in cells expressing low levels of Her2 (MDA MB231, FIG. 14).

TABLE 4
Abilities of different FITC labelled anti-Her2 conjugates to crosslink
anti-FITC CAR-T cells and Her2 extracellular domain (ECD)
EC₅₀ (nM)/Maximal binding (MFI × 100)

| | Anti FITC CAR-EC | Anti-FITC CAR-EC |
|---|---|---|
| | with CD8 hinge | with IgG4m hinge |
| Anti-Her2 Fab | N.D./1.3 ± 0.1 | N.D./0.8 ± 0.1 |
| LG68 | .2 ± 0.2/34.9 ± 2.3 | 3.6 ± 0.2/9.8 ± 1.2 |
| LS202 | 6.0 ± 0.3/41.9 ± 2.2 | 4.5 ± 0.4/14.3 ± 3.5 |
| HS75 | 5.3 ± 0.2/41.2 ± 3.2 | 3.1 ± 0.3/11.7 ± 0.1 |
| HK136 | 6.3 ± 0.2/49.6 ± 1.3 | 3.6 ± 0.2/13.7 ± 2.1 |
| LG68/HS75 | 1.0 ± 0.1/43.3 ± 1.1 | 0.2 ± 0.0/4.3 ± 0.3 |
| LS202/HK136 | 1.5 ± 0.1/65.4 ± 2.5 | 1.5 ± 0.1/21.1 ± 1.3 |

Abbreviations:
EC₅₀, half-maximal binding concentration;
N.D., not determined.
Data shown are a mean of duplicate samples ± SD.

Influence of Hinge Design on sCAR-T Cell Activity.

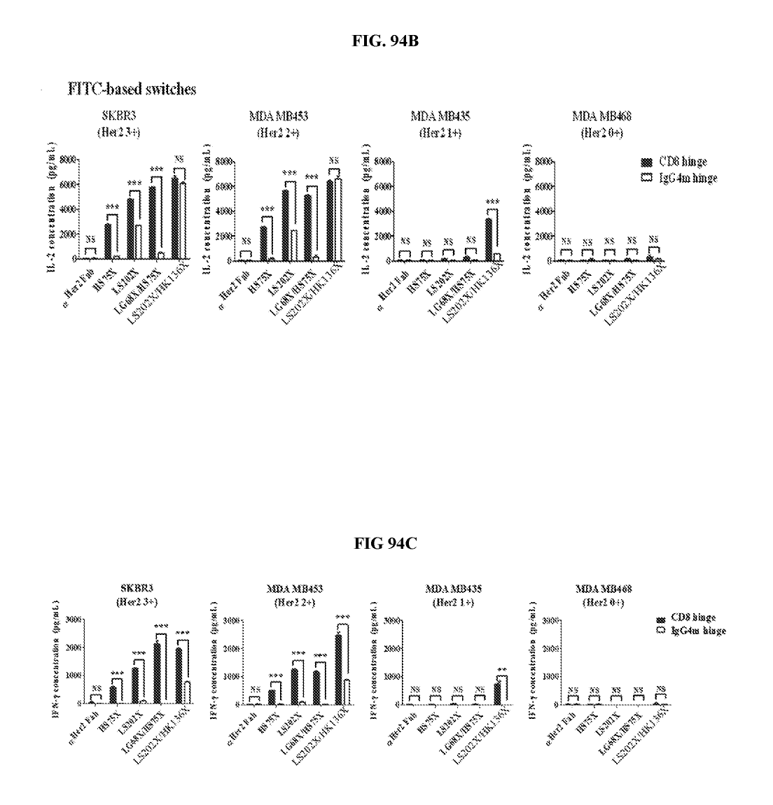

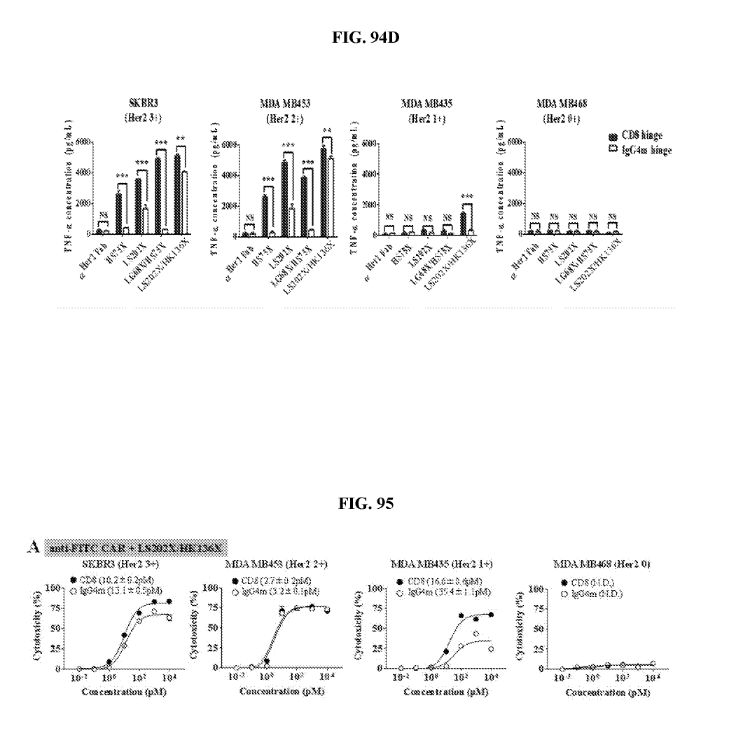

To determine if the results of the ternary complex assay correlated with sCAR-T activity, sCAR-T cell activation was tested with combinations of representative monovalent (HS75X and LS202X) and bivalent (LG68X/HS75X and LS202X/HK136X) switches at 100 pM against each Her2 expressing cancer cell. As shown in FIG. 94A-FIG. 94D, the CD8 hinge-based anti-FITC CAR-T cells afforded greater sCAR-T cell activation, indicated CD69/CD25 upregulation and increased levels of inflammatory cytokine release (IL-2, IFN-γ and TNF-α) compared to the IgG4m hinge-based sCAR-T cells for all the FITC switch designs. Among them, the LS202X/HK136X conjugates induced the most robust sCAR-T activation in agreement with the results from the ternary complex assay described above. This was most apparent on Her2 1+ cancer cells (MDA MB435, FIG. 94A-FIG. 94D). The increased level of activation seen with LS202X/HK136X compared to LG68X/HS75X could be the result of distal vs. proximal ligation relative to the antigen binding domain. Alternatively, the FITC-conjugates in the two representative bivalent switches are 20.5 Å (LS202X/HK136X) and 46.6 Å (LG68X/HS75X) apart. It is therefore also possible that distance between the FITC-conjugates may contribute to the ability of the switch to induce optimal sCAR-T cell activation, presumably also affecting the formation of an optimal immunological synapse. In this case, switch design may be optimized with compensatory designs that match the structural constraints of the sCAR.

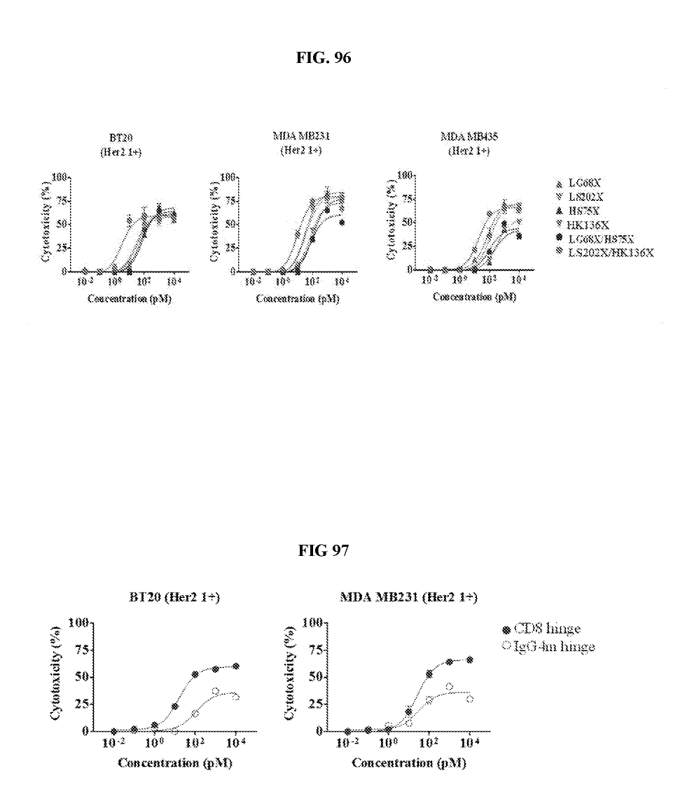

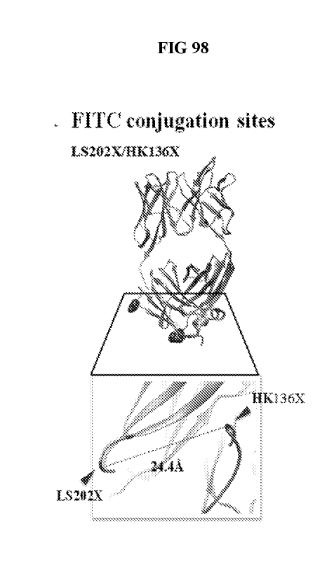

To further understand the differences observed for CD8 and IgG4m hinge designs for FITC sCARs, in vitro cytotoxicity was measured against a panel of breast cancer cells with different Her2 antigen expression levels. The LS202X/HK136X FITC switch was used to redirect anti-FITC sCAR-T cells, as this switch provided the greatest degree of ternary complex formation (FIG. 92-FIG. 93) and sCAR-T cell activation against Her2-positive cells (FIG. 94A-FIG. 94D). In agreement with previous studies, anti-FITC sCAR-T cells harboring the CD8 hinge induced greater levels of cytotoxicity compared with the IgG4m hinge (FIG. 95). Differences in the EC₅₀ of cytotoxicity for different hinges were nominal against cells with high levels of Her2 expression (SKBR3, Her2 3+; MDA MB453, Her2 2+). However, the effect of hinge design afforded a marked difference on cells at low levels of Her2 expression (MDA MB435, Her2 1+), corresponding with previous cellular activation studies (FIG. 94A-FIG. 94D). These findings were reproducible on Her2 1+ cancer cells BT20 and MDA MB231 (FIG. 97), providing further confirmation of the correlation with antigen density. The effectiveness of the CD8 hinge in the context of low level Her2 expression was confirmed with additional monovalent and bivalent switches (FIG. 96). The preference for one hinge or another may be the result of a limited quantity of immunological synapses that may be formed with cells expressing low levels of the target antigen, thereby increasing the relative contribution of each synapse to the response as a whole, thus placing a greater emphasis on optimal complex formation.

In vivo Anti-Tumor Efficacy of Conventional Anti-Her2 CAR-T and sCAR-T Cell Approaches

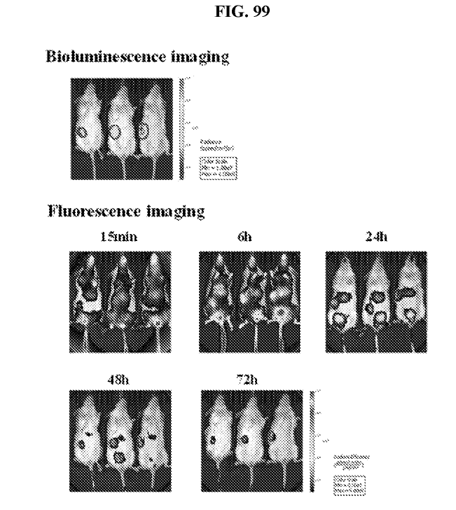

Murine xenograft models were used to test the in vivo anti-tumor efficacy of each optimized sCAR-T cell. First, a tumor distribution study was performed to assess the experimental half-life of Fabs in this xenograft system. Briefly, anti-Her2 Fab was labeled with IRDye800CW (LI-COR Biosciences) according to the manufacturer's protocol, and administered intravenously at 1 nmol per tumor-bearing mouse. Eight-week-old female NOD.Cg-Prkdc^{scid} Il2rg^{tm1Wjl}/SzJ (NSG) mice (Jackson Laboratory) were subcutaneously inoculated with 5×10⁶ MDA MB435/Her2 cells and tumors were allowed to reach 500 mm³ prior to Fab injections. Bioluminescent imaging of mice bearing tumors was achieved using the IVIS imaging by injecting mice intraperitoneally with 150 mg/kg of D-luciferin. The distribution of intravenous IRDye800 labeled anti-Her2 Fab as a correlate of tumor was assessed at 15 min, 6 h, 24 h, 48 h and 72 h post injection (FIG. 99). These data demonstrated that the anti-Her2 Fab was stable up to 24 hours, despite the average Fab half-life ranging from 1-2 hours.

The in vivo antitumor activity of each optimized sCAR-T cell was examined in mouse xenograft models using Her2 3+ (HCC1954), 2+ (MDA MB453) and 1+ (MDA MB231) cells. For Her2 3+ and 2+ xenografts, 5×10⁶ HCC1954 or MDA MB453 cells in 50% Matrigel were subcutaneously implanted into the right flank of the mice. For Her2 1+ models, 5×10⁶ MDA MB231 cells in 50% Matrigel were orthotopically injected into the abdominal mammary fat pad. 10 days later, the mice were infused IV with 30×10⁶ sCAR-T cells, followed by IV administration of corresponding switch antibodies or wild type antibodies at 0.5 mg/kg every other day for 7 times. In parallel, saline and conventional anti-Her2 CAR-T cells were injected as control groups. Mice were monitored and tumors were measured twice weekly by caliper. Tumor growth was monitored for 50 days. Both conventional and sCAR-T cells showed comparable tumor regression kinetics and completely eliminated both Her2 3+ and 2+ tumors by day 25; no relapse was observed during the course of the study (FIG. 50A and FIG. 50B). Treatment of sCAR-T cells with wild type 4D5 Fab had no effect on tumor growth. To test activity with lower antigen density Her2 1+ tumors, we used an orthotopic xenograft model with MDA MB231 cells. sCAR-T cells fully eradicated Her2 1+ tumors by day 30, and no tumor relapse occurred during the 50-days observation. The kinetics of tumor clearance were comparable to that of conventional anti-Her2 CAR-T cells (FIG. 50C). These results show that sCAR-T cells constructed with different designs can specifically target and eliminate Her2-expressing tumors with comparable efficacy to conventional CAR-T approach.

### Example 2

### UAA Incorporation Sites in Anti-Human CD33 Switch (Clone hM195)

Mutational positions in anti-human CD33 were chosen based on the crystal structure of the extracellular domain of human herceptin complexed with herceptin Fab (human IgG1 kappa, Protein Data Bank (PDB) ID 1N8Z) (see FIG. 2 and FIG. 87). HS75X and LG72 sites are proximal to the CDR loop of the antibody, and thus allow for a minimum distance between target cells and CAR-T cells upon the formation of pseudo-immunological synapse via switch molecules. HG193X, HK132, and LS206 sites are distal from antigen binding, and have been shown to be particularly beneficial when the switch is binding to membrane-proximal epitope of target antigen. Depending on the positions of epitope and binding orientation of antibody toward target antigens, the intermediate HS118X and LT113X sites may induce optimal crosslinking between target antigens and anti-FITC CAR. All sites described are located in highly conserved sequences in antibodies, are exposed on the surface of the Fab, and are conjugated with FITC-linkers that do not affect antibody binding.

### Example 3

### Expression and Characterization of Anti-CD33 hM195-LG72HS75-pAzF

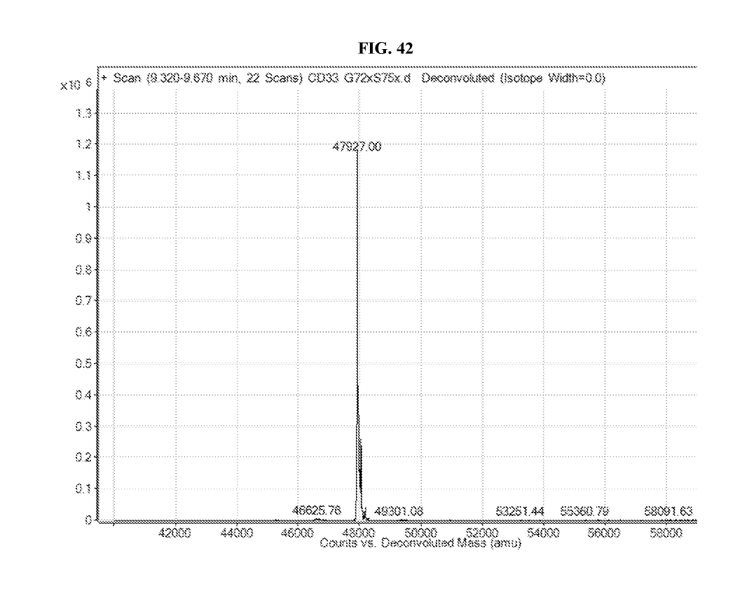

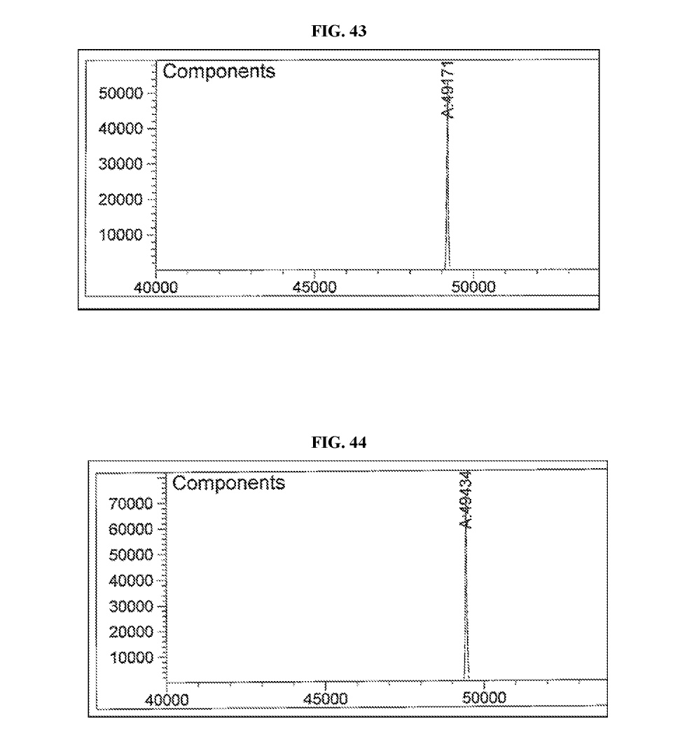

An expression plasmid containing the hM195-LG72HS75-TAG gene under an arabinose-inducible PBAD promoter was co-transformed with pUltra-pAzF/tRNATyrCUA into E. coli TOP10 cells. Cells were cultured in terrific broth (TB) media, supplemented with 100 μg/mL ampicillin, 50 μg/mL spectinomycin, and 2 mM of p-azidophenylalanine. Protein expression was induced at an OD600˜1.0 by addition of 0.2% arabinose and 1 mM isopropyl β-D-1-thiogalactopyranoside (IPTG), and cells were cultured at 37° C. for 16-24 h. Protein was released from pelleted cells by periplasmic lysis by shaking at 37° C. for 30 min in 20 mL lysis buffer (30 mM Tris-HCl, pH 7.5, 1 mM EDTA, 20% sucrose, 0.2 mg mL-1 lysozyme) per gram of wet cell pellet. Clarified lysate was applied to a 1 mL Protein G Sepharose 4 Fast Flow (GE Healthcare) column equilibrated with Buffer A (50 mM NaOAc, pH 5.0), and Fab was eluted with Buffer B (100 mM glycine, pH 2.8). Proteins were exchanged into Dulbecco's phosphate-buffered saline (DPBS) using centrifugal filtration with 30 kDa molecular weight cutoff (MWCO) filters, followed by characterization with SDS-PAGE and ESI-MS (see FIGS. 41 and 42).

hM195-LG72HS75-pAzF (1 mg/mL) was conjugated with either BCN-1PEG-FITC or BCN-4PEG-FITC linkers (50-fold molar excess) via 1,3-dipolar cycloaddition in phosphate-buffered saline (pH 7.4). The reaction was completed within 16 hours, as determined by ESI-MS. The excess linkers were removed by centrifugal filtration with 10 kDa MWCO filters (Amicon Ultra), (see FIGS. 43 and 44).

### Example 4

### Evaluation of FITC-Conjugated Anti-CD33 Switches

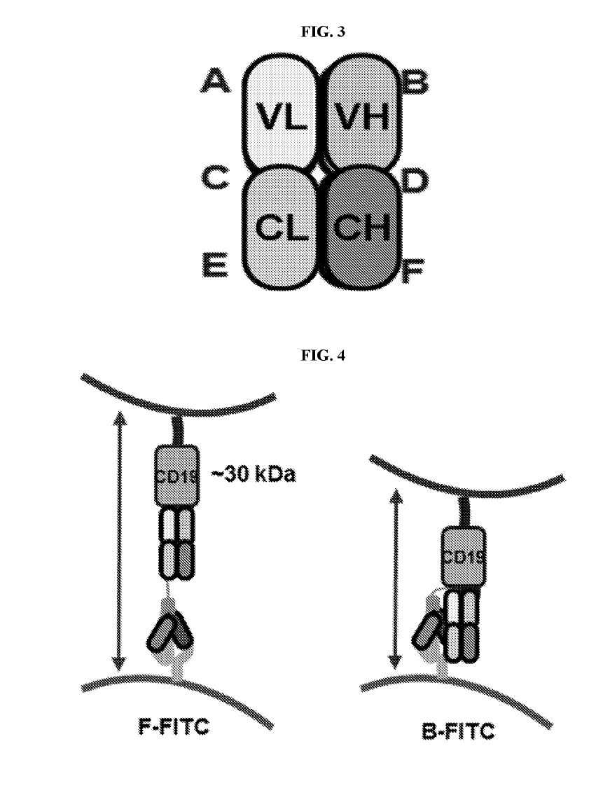

Two anti-CD33 switches were generated based on antibody clones, hM195 and hP67.6. Similar to previously described switches, six conjugation sites located in highly conserved sequences and exposed on the surface of the Fab proximal, medial, or distal to the antigen binding site were selected (FIG. 3 and Table 7).

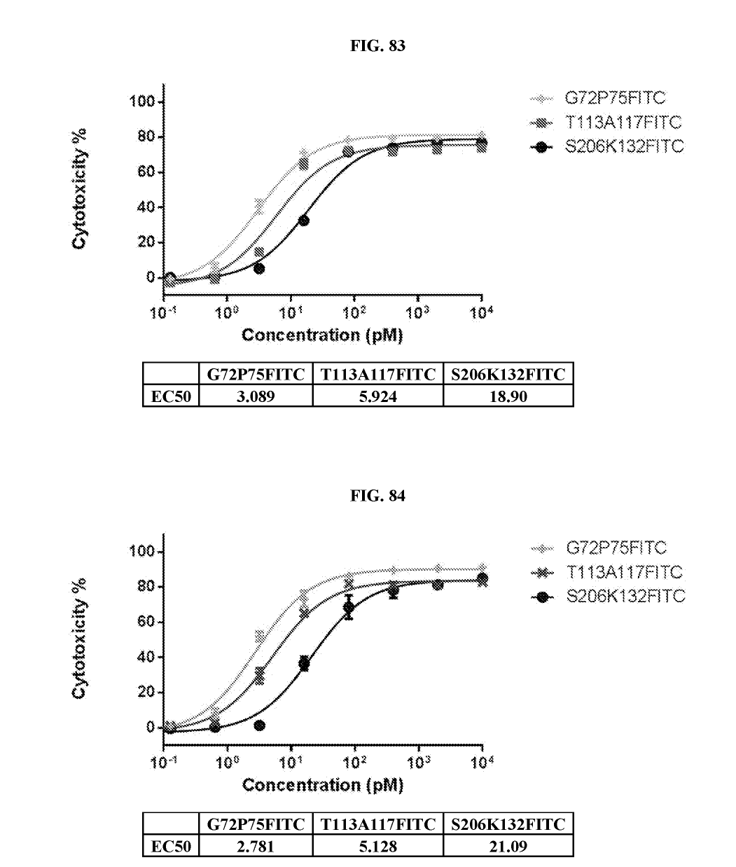

Cytotoxicity of anti-FITC CAR-T cells and switches with varying conjugation sites (Table 7) of FITC to hM195 and hP67.6 Fab were measured against CD33⁺ U937 and THP-1 cell lines, at an E:T=5:1 for 24 hrs. Results are shown in FIG. 82, FIG. 83, FIG. 84 and Tables 5, 6 and 7.

TABLE 5
% Cytotoxicity in Target cells (Data from FIG. 82)

| pM | G72FITC | | S75FITC | | A117FITC | | T113FITC | | S206FITC | | K132FITC | |
|---|---|---|---|---|---|---|---|---|---|---|---|---|
| 10000 | 55.7 | 62.1 | 57.6 | 60.3 | 58.8 | 58.4 | 54.8 | 57.9 | 50.5 | 56.7 | 57.5 | 56.2 |
| 2000 | 55.1 | 57.3 | 60.9 | 62.3 | 61.1 | 56.8 | 50.5 | 49.3 | 48.9 | 48.8 | 52.3 | 51.9 |
| 400 | 54.3 | 57 | 57 | 57.3 | 60.2 | 60.8 | 46.6 | 50.9 | 49 | 47.9 | 50.2 | 50.1 |
| 80 | 29.9 | 37.6 | 15.1 | 11.5 | 20.1 | 22.7 | 24.8 | 27.6 | 12.9 | 13.4 | 20.5 | 14.7 |
| 16 | 0.5 | 9.7 | 3.5 | 0.7 | −1 | −1.5 | 2.1 | 3.3 | 2.9 | 1.3 | 1.2 | −0.5 |
| 3.2 | 1.4 | 3.3 | 1.3 | 2.3 | 1.2 | 1.2 | −0.7 | −0.5 | 0.8 | 1.3 | −0.4 | −3.1 |
| 0.64 | −0.6 | 0.4 | 2 | 0.6 | 0.7 | −1.4 | −0.1 | −0.7 | −0.5 | −0.5 | −1.3 | −4.3 |
| 0.128 | 3.5 | 5.8 | 1.7 | 1.8 | 0 | 0.4 | −1.3 | 0.7 | 0.8 | 2.9 | −1.3 | −0.2 |
| 0.01 | 0 | 0 | 0 | 0 | 0 | 0 | 0 | 0 | 0 | 0 | 0 | 0 |

| pM | G72S75FITC | | T113A117FITC | | S206K132FITC | |
|---|---|---|---|---|---|---|
| 10000 | 63.7 | 63.8 | 54.9 | 55 | 55.4 | 53.4 |
| 2000 | 66.4 | 65.1 | 61.5 | 62.1 | 52.1 | 55.1 |
| 400 | 63.3 | 60.4 | 60.5 | 62.3 | 48.8 | 49.9 |
| 80 | 44.6 | 37.4 | 61.9 | 62.8 | 46.6 | 47.4 |
| 16 | 5.2 | 2.2 | 26.8 | 30.4 | 17.4 | 16.8 |
| 3.2 | 3.2 | −0.7 | 6.6 | 9.5 | 4.2 | 3.6 |
| 0.64 | 3.8 | 0.5 | 1.7 | −1 | 3 | 2.1 |
| 0.128 | 3.3 | 0.4 | 1.3 | 1.9 | 2.1 | 0.5 |
| 0.01 | 0 | 0 | 0 | 0 | 0 | 0 |

TABLE 6
% cytotoxicity in target cells (Raw data from FIG. 83)

| pM | G72P75FITC | | T113A117FITC | | S206K132FITC | |
|---|---|---|---|---|---|---|
| 10000 | 81.9 | 81 | 73.4 | 75 | 76.2 | 77.7 |
| 2000 | 80.8 | 78.5 | 73.6 | 72.3 | 76.1 | 78.1 |
| 400 | 80.1 | 78.5 | 71.7 | 71.9 | 72.5 | 75.3 |
| 80 | 79 | 78.1 | 73 | 71.9 | 70.9 | 72.6 |
| 16 | 70.6 | 72.2 | 66.4 | 62.9 | 32.7 | 32.6 |
| 3.2 | 37.9 | 43.1 | 15.5 | 13.9 | 7.1 | 3.7 |
| 0.64 | 3.8 | 7.6 | −1.5 | 0.1 | 0.5 | 1.6 |
| 0.128 | −0.2 | −0.9 | −2.7 | −2.8 | 0.1 | 0.4 |
| 0.01 | 0 | 0 | 0 | 0 | 0 | 0 |

TABLE 7
% Cytotoxicity in Target cells (Raw Data from FIG. 84)

| pM | G72P75FITC | | T113A117FITC | | S206K132FITC | |
|---|---|---|---|---|---|---|
| 10000 | 91.3 | 91.2 | 82.6 | 83 | 85 | 85.3 |
| 2000 | 91.5 | 90 | 80.7 | 81.7 | 80.8 | 82 |
| 400 | 90.4 | 89 | 80.1 | 82.4 | 75.3 | 81.4 |
| 80 | 85.7 | 87.1 | 81.3 | 82.6 | 64.1 | 73.4 |
| 16 | 70.2 | 76.6 | 64.4 | 66.1 | 34 | 39.3 |
| 3.2 | 50.3 | 53.9 | 26.5 | 32.7 | 2.1 | 0.6 |
| 0.64 | 6.2 | 10 | 2.2 | 3.7 | −0.1 | 0.8 |
| 0.128 | 1.3 | 0.3 | 1.7 | 0.6 | 0 | −0.7 |
| 0.01 | 0 | 0 | 0 | 0 | 0 | 0 |

Responses to hM195, were distinct from other previously described switches, as switches with conjugation sites in a middle position from the antigen binding site (i.e., CD-FITC) had better efficacy than switches with conjugation sites proximal or distal to the antigen binding site (i.e. AB-, EF-FITC). Dual conjugation of T113 and A117 showed superior cytotoxic effects relative to other switches tested in U937 cells. Opposite to hM195, hP67.6 switches with conjugation sites located proximally to the antigen binding site (i.e., AB-FITC) had better efficacy than switches with conjugation sites located distally to the antigen binding site (i.e. CD-, EF-FITC), suggesting a different position for the recognized antigen epitope. This difference in CAR-T activity with different conjugation sites suggests that distinct geometries are required for each antigen-antibody interaction to exert optimal effector function as with CD19 and CD22.

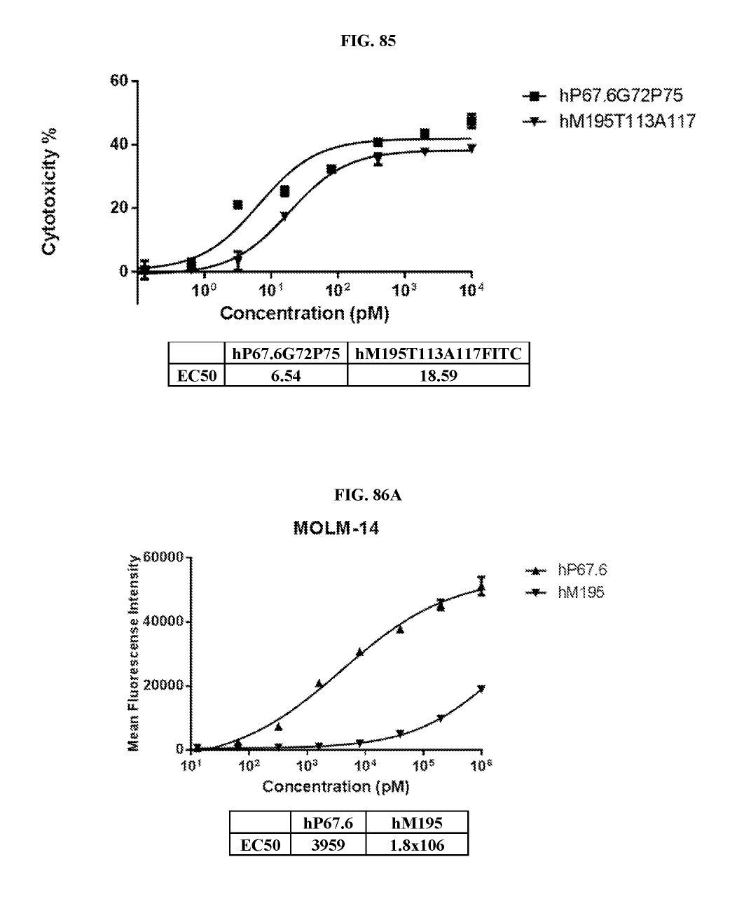

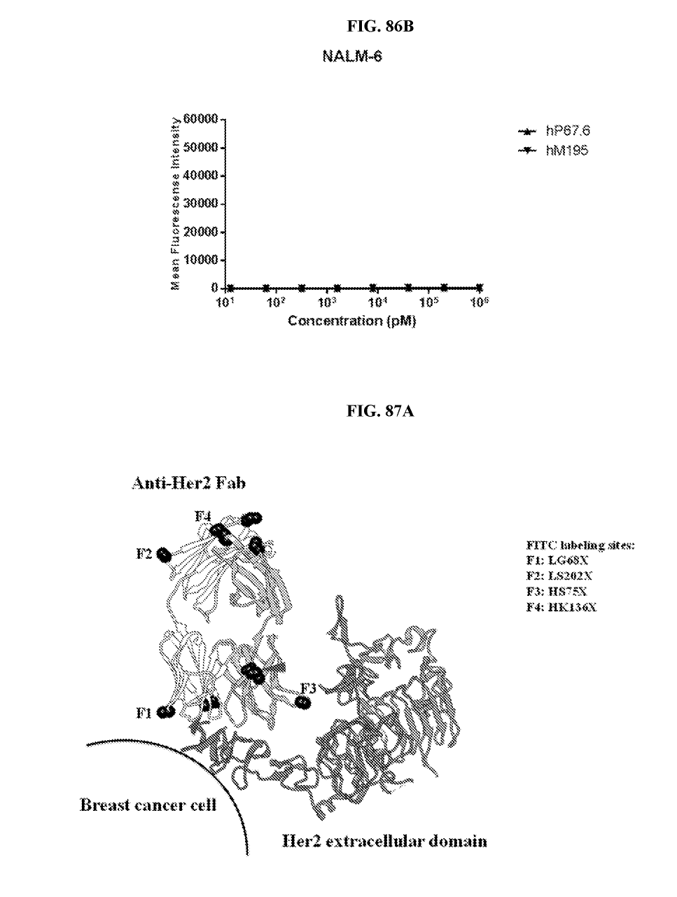

Comparisons of optimized hP67.6 and hM195 anti-CD33 switches demonstrated that the anti-CD33 hP67.6AB-FITC switch was approximately 3× more efficacious in MOLM14 (EC50=6.5 pM) than the optimized anti-CD33 hM195 CD-FITC switch (EC50=18.5 pM) (FIG. 85. raw data shown in Table 8). This is most likely due to lower hM195 affinity for CD33 as hP67.6 showed substantially increased recognition of CD33 compared to hM195, as measure by MFI (FIG. 86A and FIG. 86B, raw data shown in Table 9).

TABLE 8
% Cytotoxicity in target cells (Raw data from FIG. 85)

| | | |
|---|---|---|
| pM | hP67.6G72P75 | hM195T113A117 |

| | | | | |
|---|---|---|---|---|
| 10000 | 49 | 46 | 38.7 | 38.8 |
| 2000 | 44.6 | 42.8 | 36.7 | 38.6 |
| 400 | 40.9 | 40.8 | 34.2 | 36.7 |
| 80 | 33 | 31.8 | 32.9 | 31.7 |
| 16 | 26.4 | 24.4 | 18.2 | 16.6 |
| 3.2 | 21.6 | 20.6 | 1.4 | 5.5 |
| 0.64 | 3.5 | 0.8 | 0.3 | 1 |
| 0.128 | 2.7 | −1.4 | 0 | −0.9 |
| 0.01 | 0 | 0 | 0 | 0 |

TABLE 9
Mean fluorescence intensity (MFI) for anti-CD33
switches and MOLM-14 Cells
(Raw data from FIG. 86A)

| | | |
|---|---|---|
| pM | hP67.6 | hM195 |

| | | | | |
|---|---|---|---|---|
| 1000000 | 49100 | 53200 | 19000 | 18900 |
| 200000 | 46400 | 44100 | 9660 | 9974 |
| 40000 | 37900 | 37600 | 5386 | 4655 |
| 8000 | 31500 | 30000 | 2022 | 1891 |
| 1600 | 21000 | 20800 | 1072 | 969 |
| 320 | 7545 | 7230 | 795 | 721 |
| 64 | 2624 | 2511 | 713 | 692 |
| 12.8 | 689 | 657 | 709 | 647 |

### Example 5

### Evaluation of FITC-Conjugated Anti-CD19 Switches

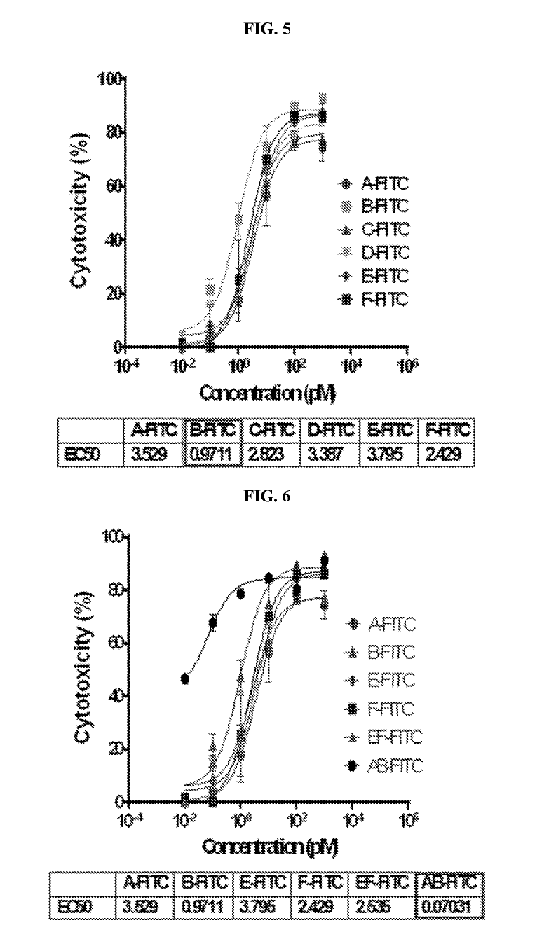

Cytotoxicity of anti-FITC CAR-T cells (41BB, 2^{nd} generation) and switches with varying conjugation sites of FITC to anti-CD19 Fab (see FIG. 3) were measured against CD19+ NALM6 at an effector cell (CAR-T) to target cell (NALM6) ratio of 5:1 (E:T=5:1) for 24 hrs a. Results are shown in FIG. 5-7.

As shown in FIG. 5, switches with FITC conjugated close to antigen binding region (e.g. B-FITC, FIG. 4) have better efficacy for targeting CD19 tumors than switches with FITC conjugated farther from the antigen binding region (e.g. F-FITC, FIG. 4).

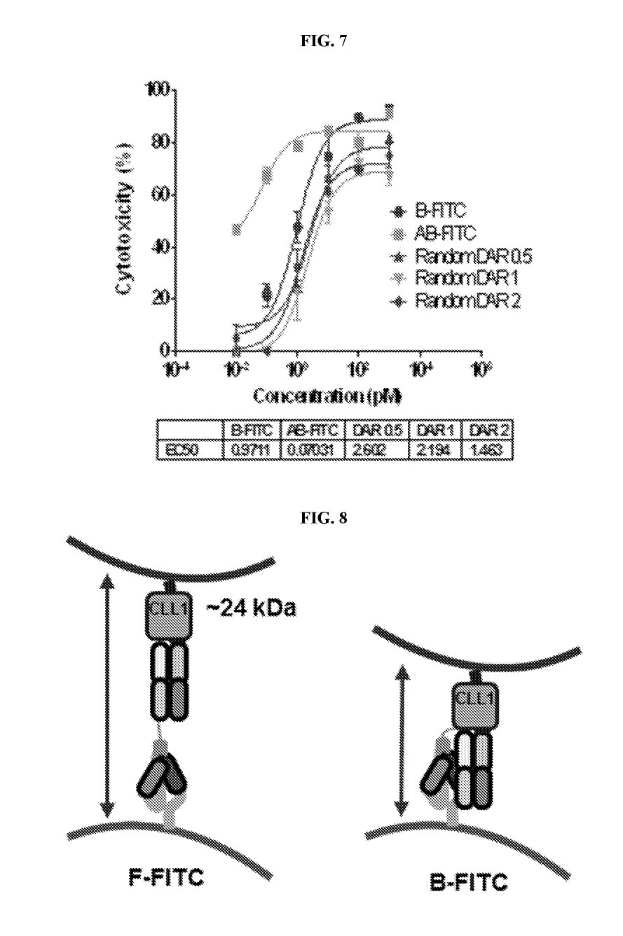

Dual FITC conjugation significantly improved the efficacy both in vitro, demonstrated by substantial decrease in the EC50 of AB-FITC compared to EF-FITC (FIG. 6) Optimized site-specific switch (AB-FITC) demonstrated superior activity over randomly conjugated switch regardless of drug to antibody ratio (DAR)-both in vitro (FIG. 7) and in vivo (data shown in future figures).

### Example 6

### Evaluation of FITC-Conjugated Anti-CLL1 Switches

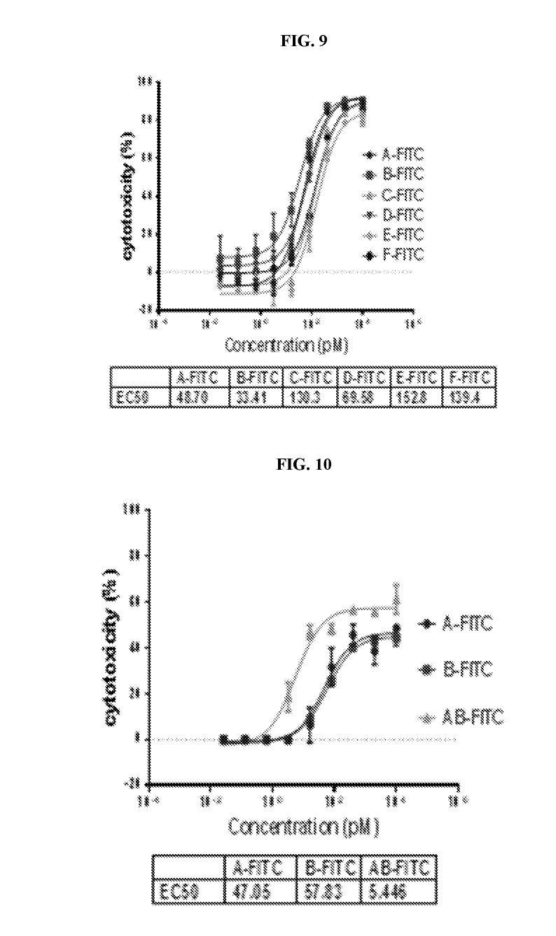

Cytotoxicity of anti-FITC CAR-T cells and switches with varying conjugation sites of FITC to anti-CLL1 Fab (see FIG. 8) were measured against the CLL1⁺ AML cell line, U937 at E:T=5:1 for 24 hrs. Results are shown in FIGS. 9-10.

Switches with FITC conjugated proximal to the antigen binding region (e.g. A and B from FIG. 8) have better efficacy for targeting AML cells than switches with FITC conjugated distal to antigen binding region (e.g. F from FIG. 8). Additional increases in activity were seen with bivalent, proximal FITC conjugations were seen (FIG. 10, AB-FITC).

This switchable approach may overcome the safety issues of AML-targeting conventional CAR-Ts (e.g. CD33 and CD123 CAR), which will likely cause severe chronic myelosuppression in patients.

### Example 7

### Comparison of FITC-Conjugated Anti-CLL1 Switches in Various CLL1 Positive Cell Lines

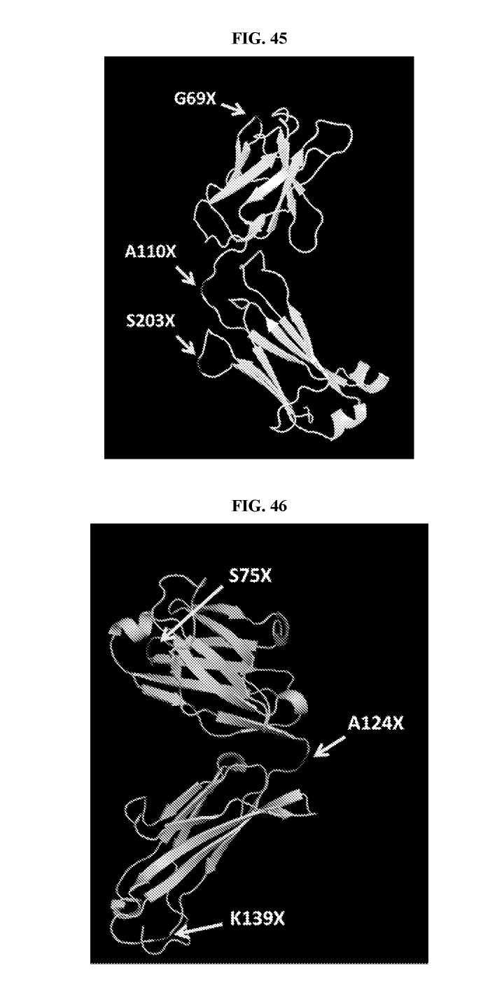

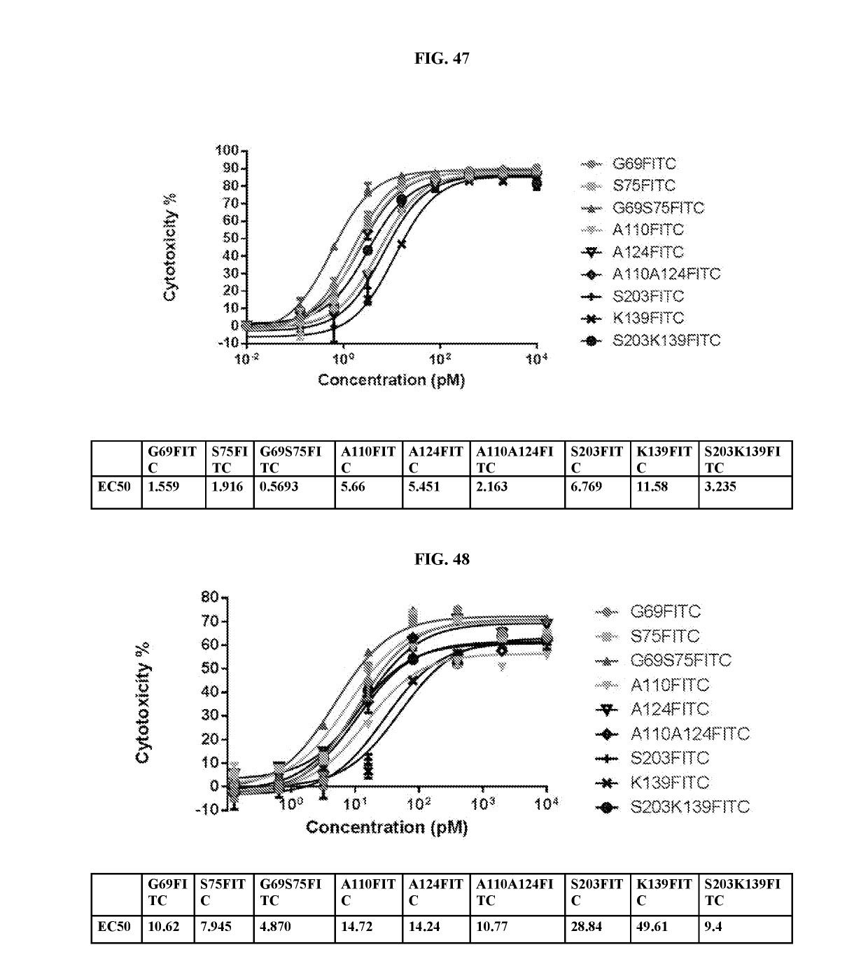

FITC-anti-CLL1 Fab switches were produced by conjugating FITC to one of the following sites depicted in FIG. 45-46 (LC SEQ ID NO. [18], LC S69 LC A110, LC S203, HC SEQ ID NO. [19] HC G75, HC A124, HC K139, and dual conjugation sites LC A110+HC A124 LCS203+HC K139 and G69+S75) in the anti-CLL1 1075.7 clone [Haematologica. 2010 January; 95(1): 71-78.]. These were tested with anti-FITC CAR-T cells in U937 (FIG. 47, raw data shown in Table 10) and HL60 cell lines (FIG. 48, raw data shown in Table 11). Viability with increasing switch dose was assessed by flow cytometry.

TABLE 10
Percent Toxicity in U937 Cells from FIG. 47

| pM of | | | | | | | | | | | | |
|---|---|---|---|---|---|---|---|---|---|---|---|---|
| Switch | G69FITC | | S75FITC | | G69S75FITC | | A110FITC | | A124FITC | | A110A124FITC | |
| 10000 | 90.1 | 91.1 | 89.4 | 88.6 | 87.5 | 89 | 89.5 | 87.4 | 85.3 | 86.5 | 87.4 | 86.6 |
| 2000 | 89.5 | 90.1 | 88.9 | 86.5 | 88.1 | 89 | 86.3 | 86.8 | 88.5 | 86.8 | 89.7 | 88.7 |
| 400 | 89 | 89 | 86 | 85.9 | 87.6 | 88.5 | 85.3 | 84.3 | 84.3 | 85 | 88.1 | 87.1 |
| 80 | 85.5 | 87.1 | 85.6 | 84.9 | 87.3 | 88.5 | 82 | 81.6 | 83.3 | 79.2 | 86.6 | 81.8 |
| 16 | 82.2 | 82.8 | 78.8 | 79.6 | 85.6 | 87 | 66.2 | 64.5 | 68.5 | 70.1 | 79.8 | 78 |
| 3.2 | 59.1 | 64.2 | 58.6 | 56.9 | 80.7 | 75.8 | 30.9 | 29.5 | 27.9 | 29.6 | 57.3 | 50.9 |
| 0.64 | 19.5 | 26 | 10.8 | 19 | 45.8 | 45.2 | 10.3 | 7.8 | 6.4 | 5.7 | 16.4 | 17.6 |
| 0.128 | 4.4 | 4.2 | −2.5 | 7.6 | 5.9 | 14.1 | −5.2 | 6.9 | 0.6 | 4.9 | 7.9 | 7.6 |
| 0.01 | 0 | 0 | 0 | 0 | 0 | 0 | 0 | 0 | 0 | 0 | 0 | 0 |

| pM of | | | | | | |
|---|---|---|---|---|---|---|
| Switch | S203FITC | | K139FITC | | S203K139FITC | |
| 10000 | 86 | 85.1 | 87.5 | 84.3 | 84 | 79 |
| 2000 | 86 | 82.5 | 82.8 | 82.4 | 87.7 | 84.9 |
| 400 | 85 | 84.5 | 81.9 | 83.6 | 86.9 | 83.4 |
| 80 | 81.1 | 77.5 | 79.1 | 78 | 84.3 | 82 |
| 16 | 64.5 | 67 | 45.4 | 48.3 | 72.9 | 72.3 |
| 3.2 | 15.1 | 28.3 | 15.1 | 15.6 | 43.2 | 43.5 |
| 0.64 | −6.6 | 5.9 | −29.1 | −1.6 | 11.7 | 11.7 |
| 0.128 | −1.8 | 5.6 | −0.3 | 0.9 | 13.4 | 3.7 |
| 0.01 | 0 | 0 | 0 | 0 | 0 | 0 |

TABLE 11
Percent Toxicity in HL60 cells from FIG. 48

| pM of | | | | | | | | | | | | |
|---|---|---|---|---|---|---|---|---|---|---|---|---|
| Switch | G69FITC | | S75FITC | | G69S75FITC | | A110FITC | | A124FITC | | A110A124FITC | |
| 10000 | 62.1 | 64.1 | 64.1 | 66 | 71.8 | 70.4 | 54.8 | 56.6 | 68.5 | 68.9 | 61.3 | 62.7 |
| 2000 | 64.1 | 66.5 | 63.2 | 62.9 | 65.6 | 67.2 | 50.4 | 51 | 63.2 | 66.4 | 56.6 | 58.6 |
| 400 | 74.9 | 75.2 | 71 | 70.6 | 71.9 | 69.8 | 51.4 | 54.1 | 70.1 | 71.8 | 56.5 | 55.5 |
| 80 | 69 | 71.6 | 73.7 | 73 | 76.2 | 73.7 | 58.4 | 59.2 | 59.6 | 63.3 | 61.9 | 64.2 |
| 16 | 50.1 | 39.3 | 51.4 | 49.3 | 56.2 | 58.2 | 26.3 | 26.4 | 35.6 | 37.5 | 32.7 | 39.4 |
| 3.2 | 0.2 | 4.8 | 13.5 | 10.5 | 26.4 | 25.6 | 2 | 2.2 | 14.8 | 14.8 | 4.6 | 8.2 |
| 0.64 | −1.5 | 1.4 | 7.3 | 8 | 2.2 | 6.2 | −0.8 | 1.3 | 9.5 | 7.1 | 1.2 | 2 |
| 0.128 | −4.8 | 0.7 | 8.5 | 1.8 | 0.9 | 5 | −3.4 | 5.1 | 5 | 5.7 | −3.3 | −2.2 |
| 0.01 | 0 | 0 | 0 | 0 | 0 | 0 | 0 | 0 | 0 | 0 | 0 | 0 |

| pM of | | | | | | |
|---|---|---|---|---|---|---|
| Switch | S203FITC | | K139FITC | | S203K139FITC | |
| 10000 | 57.2 | 59.6 | 60.6 | 62.8 | 63.1 | 62.7 |
| 2000 | 60 | 61.5 | 61.4 | 59 | 63 | 65.1 |
| 400 | 52.7 | 54.6 | 57.8 | 55.4 | 52.3 | 52.2 |
| 80 | 57.2 | 53.8 | 43.9 | 46.3 | 54.1 | 53.9 |
| 16 | 10 | 13.1 | 8.7 | 4.5 | 39.2 | 42.5 |
| 3.2 | −3.2 | 5.2 | 3.3 | 4.8 | 14.2 | 9.7 |
| 0.64 | −3.4 | 2.5 | 4 | 3.6 | 0.1 | −1.4 |
| 0.128 | −8 | 0.3 | −1.2 | 0.8 | −0.2 | 2.7 |
| 0.01 | 0 | 0 | 0 | 0 | 0 | 0 |

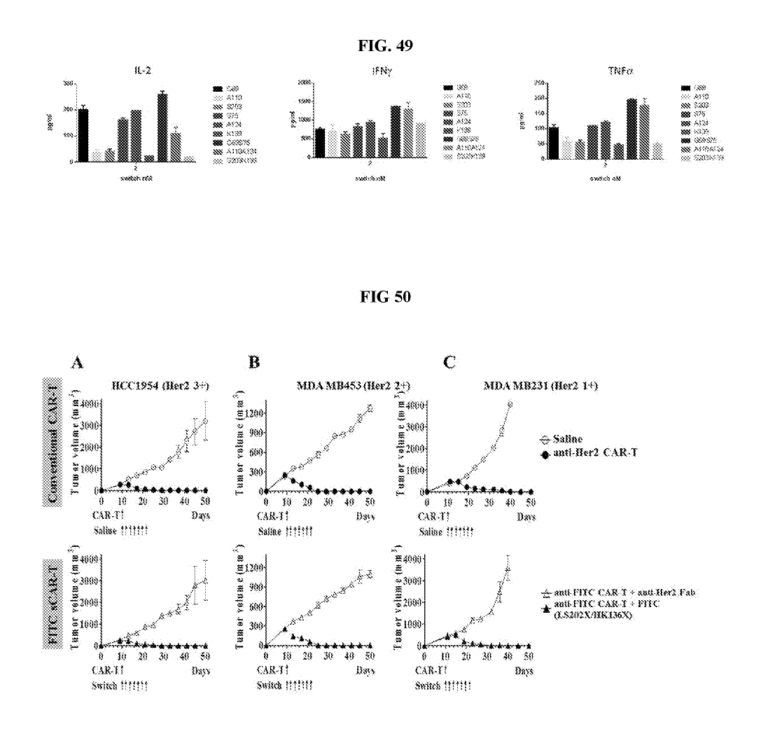

Dual conjugation at G69 and S75 showed superior cytotoxic effects relative to other switches tested in U937 and HL60 cells. In addition to lytic function, all FITC conjugates induced IFNγ, TNFα, and IL-2 release that correlated with the degree of cytotoxicity, with the AB-FITC switch yielding the highest cytokine induction (FIG. 49, Raw data shown in Table 12) These data resemble the results obtained for CD19-targeting switches.

TABLE 12
Cytokine concentrations (pg/mL) at 2 nM switch concentration (Raw data from FIG. 49)

| | G69 | | A110 | | S203 | | S75 | | A124 | | K139 | |
|---|---|---|---|---|---|---|---|---|---|---|---|---|
| IL2 | 212.88 | 189.41 | 42.87 | 30.99 | 47.2 | 33.59 | 155.74 | 167.07 | 196.32 | 198.02 | 21.94 | 21.04 |
| TNFa | 110.99 | 96.74 | 67.92 | 49.69 | 61.09 | 50.2 | 107.82 | 110.31 | 124.06 | 118.52 | 49.71 | 41.79 |
| IFNg | 791.11 | 724.88 | 838.34 | 575.63 | 594.73 | 674.43 | 770.7 | 885.45 | 973.79 | 886.5 | 603.97 | 417.51 |

| | G69S75 | | A110A124 | | S203K139 | |
|---|---|---|---|---|---|---|
| IL2 | 247.92 | 268.18 | 127.23 | 91.01 | 19.35 | 18.43 |
| TNFa | 193.34 | 197.03 | 193.15 | 159.39 | 45.79 | 52.24 |
| IFNg | 1372.9 | 1355.4 | 1419.1 | 1171.1 | 913.19 | 913.37 |

### Example 8

### In Vivo Efficacy Study of FITC Switch Targeting CLL-1 in a Subcutaneous AML Model

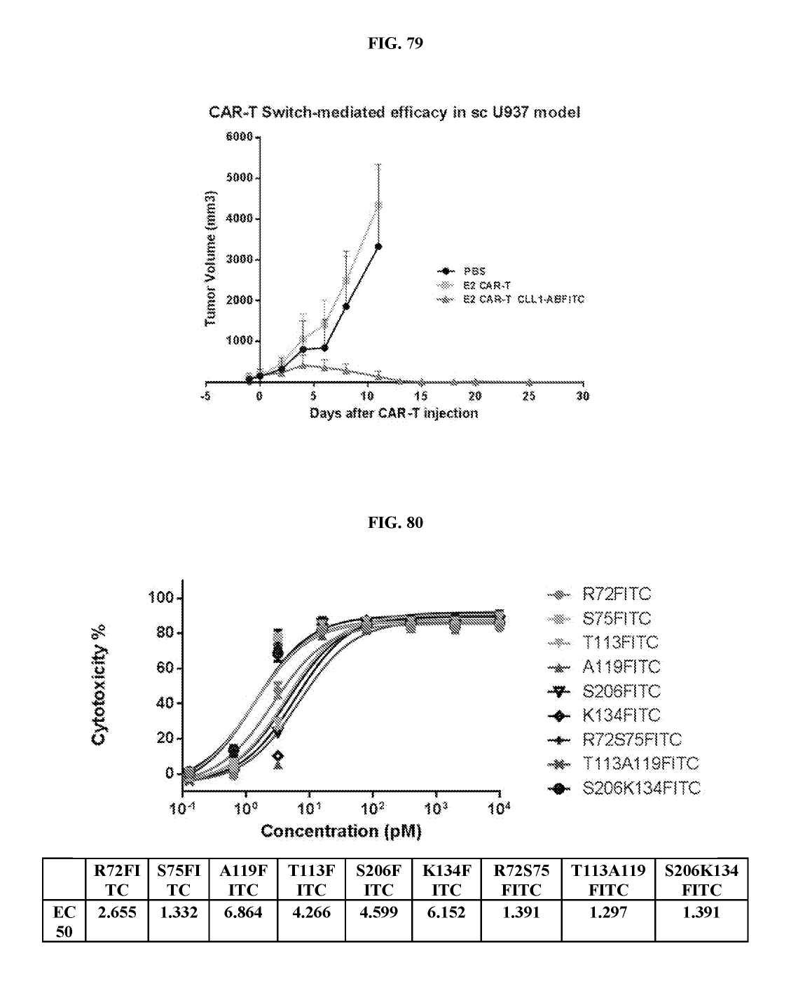

2×10⁶ U937 cells were injected subcutaneously (SC) into female NSG mice. Seven days later, mice were infused IV with 30×10⁶ anti-FITC CAR-T cells and switch treatment was initiated with anti-CLL1 AB-FITC conjugate at 1 mg/kg (red), anti-FITC CAR-T (yellow) or PBS (black) IV every day for a total of ten doses (FIG. 79).

Tumor growth was monitored twice a week by caliper measurements. One mouse from anti-CLL1 AB-FITC switch died soon after CAR-T cell injection, most likely due to syringe air bubbles. Three out of four mice infused with anti-FITC CAR-T cells and AB-FITC switch cleared the tumor by day 13 and by day 25 all mice showed no measurable tumor until the end of the experiment (Table 13). On the other hand, rapid tumor growth was observed in mice treated with PBS and were sacrificed by day 11 according to ethics criteria. Switch-mediated targeting of CLL-1⁺ AML, cell line resulted in complete regression of all tumors validating this antigen as a good candidate for AML sCAR-T therapy.

TABLE 13
U937-induced Tumor size (mm³) (Raw Data from FIG. 79)

| | PBS | | | E2 CAR-T | | | E2 CAR-T CLL1-FITC | | |
|---|---|---|---|---|---|---|---|---|---|
| Day | Mean | SD | mice | Mean | SD | mice | Mean | SD | mice |
| −1 | 66 | 79.1 | 5 | 90.6 | 66.8 | 5 | 90.2 | 117.8 | 5 |
| 0 | 146 | 171.4 | 5 | 159.7 | 106.1 | 5 | 164.2 | 98.6 | 5 |
| 2 | 312.5 | 285.5 | 5 | 444.2 | 186.1 | 5 | 228.4 | 201.3 | 4 |
| 4 | 800 | 701.7 | 5 | 1055.6 | 621.7 | 5 | 414.4 | 247.2 | 4 |
| 6 | 836.5 | 697.1 | 5 | 1412.2 | 604.4 | 5 | 356.3 | 199.5 | 4 |
| 8 | 1856.8 | 1353.9 | 5 | 2483.2 | 598 | 5 | 280.5 | 166.6 | 4 |
| 11 | 3331.3 | 2013.7 | 5 | 4350.1 | 881.2 | 5 | 130.6 | 135.2 | 4 |
| 13 | | | | | | | 28 | 55.7 | 4 |
| 15 | | | | | | | 3.5 | 7.1 | 4 |
| 18 | | | | | | | 5.9 | 11.8 | 4 |
| 20 | | | | | | | 16.4 | 32.7 | 4 |
| 25 | | | | | | | 0 | 0 | 4 |
| 30 | | | | | | | 0 | 0 | 4 |

### Example 9

### Evaluation of FITC-Conjugated Anti-CdD123 Switches

Two anti-CD123 switches were generated based on antibody clones 32716 and 26292. Similarly to CD19 and CLL1 clone development, six conjugation sites located in highly conserved sequences and exposed on the surface of the Fab proximal, medial, or distal to the antigen binding site were selected (FIG. 3 and Table 12).

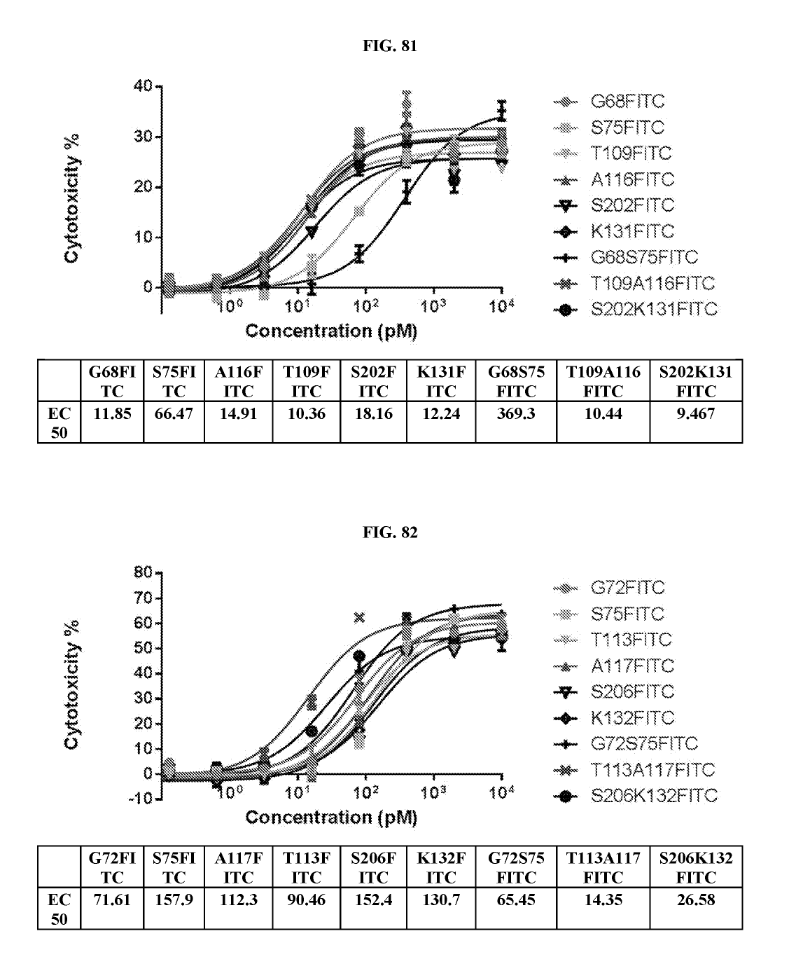

Cytotoxicity of anti-FITC CAR-T cells and switches with varying conjugation sites of FITC to 32716 and 26292 Fab were measured against CD123⁺ MOLM13, and KASUMI cell lines, at E:T=5:1 for 24 hrs. Results are shown in FIG. 80 (raw data shown in Table 14) and FIG. 81 (raw data shown in Table 15). In 32716, switches with conjugation sites in a close position from the antigen binding site (i.e., AB-FITC) had better efficacy than switches with conjugation sites further to the antigen binding site (i.e. CD-, EF-FITC), however no differences were seen in EC50 between monovalent and bivalent switches. Clone 26292 switches with conjugation sites further to the antigen binding site (i.e., EF-FITC) had better efficacy than switches with conjugation sites close to the antigen binding site and an enhancement in cytotoxicity was observed with bivalent switches (i.e. EF-FITC).

TABLE 14
% Toxicity in Target Cells (Raw Data from FIG. 80)

| pM | R72FITC | | S75FITC | | A119FITC | | T113FITC | | S206FITC | | K134FITC | |
|---|---|---|---|---|---|---|---|---|---|---|---|---|
| 10000 | 84.8 | 81.6 | 84.2 | 86.2 | 85.7 | 85.1 | 90.7 | 86.3 | 88.5 | 85.7 | 92.1 | 88.3 |
| 2000 | 83 | 82.9 | 83.5 | 85.8 | 81 | 83.5 | 88 | 86.1 | 85.2 | 85 | 89.4 | 84.5 |
| 400 | 83.2 | 82.8 | 83 | 85.3 | 82.9 | 82.7 | 87.1 | 86.8 | 85.3 | 85.2 | 88.2 | 84.8 |
| 80 | 83.9 | 81.9 | 82.7 | 84.9 | 81.1 | 82.5 | 86.7 | 87.3 | 86.3 | 85.7 | 88.4 | 86.4 |
| 16 | 81.3 | 81.6 | 81.5 | 82.3 | 78.8 | 78.7 | 84.9 | 85.5 | 86.1 | 85.2 | 85.7 | 84.5 |
| 3.2 | 44.4 | 50.6 | 79.4 | 76.4 | 4.1 | 6.6 | 30.7 | 27 | 25 | 22.6 | 11.3 | 9.5 |
| 0.64 | −0.3 | −1.6 | 7.3 | 2.9 | −0.3 | 1 | −0.3 | −1.2 | −0.4 | 0 | 0.6 | 3.1 |
| 0.128 | −0.9 | 1.6 | 1.7 | 0.7 | −2.2 | 1.6 | −2.9 | 0.6 | −0.6 | 0.9 | −0.1 | 0 |
| 0.01 | 0 | 0 | 0 | 0 | 0 | 0 | 0 | 0 | 0 | 0 | 0 | 0 |

| pM | R72S75FITC | | T113A119FITC | | S206K134FITC | |
|---|---|---|---|---|---|---|
| 10000 | 88 | 87.1 | 89.4 | 84.8 | 87.7 | 84.6 |
| 2000 | 85.1 | 88.1 | 86.5 | 84 | 84.4 | 83.3 |
| 400 | 85.8 | 89.2 | 84.8 | 83.4 | 85.3 | 85.6 |
| 80 | 85.5 | 88.3 | 85.7 | 81.3 | 84.6 | 84.3 |
| 16 | 84.8 | 88.3 | 83.3 | 81.3 | 82.5 | 81.7 |
| 3.2 | 73.1 | 80.4 | 77.5 | 73.7 | 71.9 | 65.6 |
| 0.64 | 2.2 | 8.8 | 8.2 | 9.6 | 15.3 | 11.8 |
| 0.128 | −3.1 | 0.8 | −2.9 | −5.3 | 2.7 | −0.1 |
| 0.01 | 0 | 0 | 0 | 0 | 0 | 0 |

TABLE 15
% Toxicity in Target Cells (Raw Data from FIG. 81)

| pM | G68FITC | | S75FITC | | A116FITC | | T109FITC | | S202FITC | | K131FITC | |
|---|---|---|---|---|---|---|---|---|---|---|---|---|
| 10000 | 30.3 | 27.4 | 26.4 | 28.4 | 27.8 | 29.8 | 24 | 23.1 | 24.1 | 24.4 | 26.6 | 27.2 |
| 2000 | 28 | 26 | 27.1 | 29.7 | 25.7 | 27.8 | 23.7 | 23 | 21.1 | 23.8 | 25.3 | 27.8 |
| 400 | 34.9 | 38.2 | 24.9 | 27 | 30.5 | 34 | 30.6 | 31.5 | 26 | 29.4 | 32.6 | 31.3 |
| 80 | 31.3 | 29.4 | 15.8 | 14.6 | 26.1 | 27.8 | 26.9 | 26.4 | 22.7 | 24.3 | 28.6 | 27.7 |
| 16 | 17.1 | 17.7 | 5.8 | 2.7 | 15.1 | 14.6 | 15.6 | 16.6 | 10.8 | 11.3 | 16.8 | 16.5 |
| 3.2 | 6.1 | 2.4 | −1.9 | −1 | 2.6 | 2.7 | 3.5 | 4.5 | −1.1 | −0.2 | 2.6 | 2.1 |
| 0.64 | 2 | −0.4 | −2.4 | −0.8 | −0.7 | −0.1 | 0.6 | 1 | −0.6 | −0.1 | 0.5 | 0.1 |
| 0.128 | 1.7 | −1.1 | −0.9 | 1.4 | −0.1 | 0 | 0.5 | 1.6 | 0.1 | 0.1 | 0.6 | 0 |
| 0.01 | 0 | 0 | 0 | 0 | 0 | 0 | 0 | 0 | 0 | 0 | 0 | 0 |

| pM | G68S75FITC | | T109A116FITC | | S202K131FITC | |
|---|---|---|---|---|---|---|
| 10000 | 33.9 | 36.5 | 27.1 | 30.8 | 28.5 | 26.1 |
| 2000 | 26.4 | 29.1 | 23.8 | 28.4 | 23.2 | 19.7 |
| 400 | 17.5 | 20.7 | 27.8 | 31.7 | 26.1 | 24.4 |
| 80 | 5.6 | 7.9 | 29.1 | 30.9 | 27.2 | 24.5 |
| 16 | −0.7 | 2.2 | 17.4 | 17.7 | 16.6 | 15.6 |
| 3.2 | 0.6 | 1.2 | 5.2 | 3.9 | 5.2 | 2.5 |
| 0.64 | 0 | −0.2 | 0.5 | 1.5 | 0.1 | 0.5 |
| 0.128 | 0.2 | 0.7 | −0.2 | 2.1 | −1.3 | −0.3 |
| 0.01 | 0 | 0 | 0 | 0 | 0 | 0 |

### Example 10

### Soluble TCR (sTCR) Switch Targeting

Expression of the cancer-testis antigen, NY-ESO-1 is restricted to the testes and no other normal tissue. However, NY-ESO-1 expression is found in a surprisingly large range of tumors and may be particularly useful in targeting melanoma and multiple myeloma.

A NY-ESO-1 switch is produced by linking FITC to an unnatural amino acid at to any predetermined sites on the TCRα and/or TCRβ chain. This construct is expressed and purified in high yields from E. coli. The switch is tested for its ability to recruit sCAR-T cells using in vitro cytotoxicity assays against the melanoma cell line, A375. Additional constructs are tested with grafting positions identified by structure-based design to assess the geometric constraints of TCR targeting in the context of sCAR-T cells. Notably, all sTCRs bind with the same relative orientation. Therefore, one optimal switch design works equally for any sTCR.

sTCR switches are expressed as follows:

TCR chains are expressed separately as inclusion bodies in the E. coli strain BL21-DE3(pLysS) by induction in mid-log phase with 0.5 mM isopropyl β-D-1-thioglactopyranoside (IPTG). Inclusion bodies are isolated by sonication, followed by successive wash and centrifugation steps using 0.5% Triton X-100. Finally, the inclusion bodies are dissolved in 6 M guanidine, 10 mM dithiothreitol (DTT), 10 mM ethylenediaminetetra-acetate (EDTA), buffered with 50 mM Tris, pH 8.1 and stored at −80° C. Soluble TCR is refolded by rapid dilution of a mixture of the dissolved α- and β-chain inclusion bodies into 5 M urea, 0.4 M L-arginine, 100 mM Tris pH 8.1, 3.7 mM cystamine, 6.6 mM β-mercapoethylamine (4° C.) to a final concentration of 60 mg/L.

sTCR switches are purified as follows:

The refold mixture is dialyzed for 24 h against 10 volumes of demineralized water, then against 10 volumes of 10 mM Tris pH 8.1 at 4° C. The refolded protein is then filtered and loaded onto a POROS 50HQ column (Applied Biosystems). The column is washed with 10 mM Tris, pH 8.1 prior to elution with a 0±500 mM NaCl gradient in the same buffer. Fractions are analysed by Coomassie-stained sodium dodecyl sulphate (SDS)±10% NuPAGE gels (Novagen, Wis.), and TCR-containing fractions are re-pooled and further purified by gel filtration on a Superdex 75PG 26/60 column (Amersham Biosciences, Uppsala, Sweden) pre-equilibrated in phosphate-buffered saline. Fractions comprising the main peak are re-pooled and further analyzed. The RNA-purified 1G4 dsTCR is analyzed by Coomassie-stained SDS±10% NuPAGE under reducing and non-reducing conditions, and an aliquot of protein was buffer exchanged into HEPES pH 7.4, 150 mM NaCl, 3 mM EDTA (HBSE) and concentrated prior to activity determination by surface plamon resonance (SPR, BIAcore).

Alternatively, TCR chains are overexpressed in E. coli and purified as follows. GFG020, GFG021, JMB002, GFG089, and GFG092, the pGMT7 expression plasmids encoding the JM22a-Jun, JM22b-Fos, JM22b-Fosbt, F5a-Jun, and F5b-Fos proteins, respectively, are transformed separately into E. coli, and single colonies are grown at 37° C. in TYP ˜ampicillin 100 μg/mL media to OD600 of 0.4 before inducing protein expression with 0.5 mM IPTG. Cells are harvested 3 h post-induction by centrifugation for 30 min at 4000 rpm in a Beckman J-6B. Cell pellets are resuspended in a buffer containing 50 mM Tris-HCl, 25% w/v sucrose, 1 mM EDTA, 0.1% w/v sodium azide, 10 mM DTT, pH 8.0. After an overnight freeze-thaw step, resuspended cells are sonicated in 1 minute bursts for a total of 10 min in a Milsonix XL2020 sonicator using a standard 12 mm diameter probe. Inclusion body pellets are recovered by centrifugation for 30 min at 13,000 rpm in a Beckman J2-21 centrifuge. Three detergent washes are then carried out to remove cell debris and membrane components. Each time the inclusion body pellet is homogenized in a Triton buffer ˜50 mM Tris-HCl, 0.5% Triton X-100, 200 mM NaCl, 10 mM EDTA, 0.1% w/v sodium azide, 2 mM DTT, pH 8.0 before being pelleted by centrifugation for 15 min at 13,000 rpm in a Beckman J2-21. Detergent and salt are then removed by a similar wash in the following buffer: 50 mM Tris-HCl, 1 mM EDTA, 0.1% w/v sodium azide, 2 mM DTT, pH 8.0. Finally, the inclusion bodies are solubilized in denaturant for 3-4 h at 48° C. JM22a-Jun, JM22b-Fos, JM22b-Fosbt, and F5a-Jun pellets are dissolved separately in a urea solution of 50 mM MES, 8 M urea, 10 mM EDTA, 2 mM DTT, pH 6.5, whereas F5b-Fos pellets are dissolved in a guanidine solution containing 50 mM MES, 6 M guanidine, 10 mM EDTA, 2 mM DTT, pH 6.5. Insoluble material is then pelleted by centrifugation for 30 min at 13,000 rpm in a Beckman J2-21, and the supernatant is divided into 1 mL aliquots and frozen. Solubilized, purified inclusion bodies are quantitated using a Bradford dye-binding assay (Biorad, Richmond, Calif.). For each chain, a yield of around 100 mg of purified inclusion body is obtained from 1 L of culture. From SDS-PAGE analysis, the purity of each inclusion body is estimated to be around 90%.

### Example 11

### Expression of TCR Chains

TCR chains are expressed separately according to the following protocol:

1. Pick colonies to set up a 50 ml starter culture in LB broth with ampicillin to run overnight at 37° C. in a shaker.

2. Seed large culture at 0.07 OD(600). Induce at 0.4 OD(600) with 0.5 mM IPTG (0.5-1.0 L 2xYT+Amp).

3. Pellet cultures by centrifuging at 6,000 rpm for 20 minutes at 4° C.

4. Freeze overnight at −80° C.

5. Resuspend with 25 ml to 50 ml of buffer (50 mM Tris-HCL [8.0], 25% w/v sucrose, 1 mM EDTA, 0.1% w/v NaN₃, 10 mM DTT)+0.5 mg/ml of lysozyme+1 pellet of protease inhibitors (crushed)/tube. Keep on ice.

6. Sonicate for 20 seconds on, 30 seconds off for a total of 15 minutes experimental time. Keep on ice.

7. Pellet by centrifuging at 13,000 rpm for 15 minutes and wash 3× times with detergent/salt buffer (50 mM Tris-HCL [8.0], 0.5% Triton X-100, 200 mM NaCl, 10 mM EDTA, 0.1% w/v NaN₃, 2 mM DTT).

8. Pellet by centrifuging at 13,000 rpm for 15 minutes and wash 2× twice with (50 mM Tris-HCL [8.0], 1 mM EDTA, 0.1% w/v NaN₃, 2 mM DTT).

9. Dissolve inclusion bodies in a buffer of 6 M guanidine, 10 mM DTT, 10 mM EDTA, 50 mM Tris [8.1] and store at −80° C.

To analyze protein in the lysed cells, collect 100 μl before spinning down the sonicated cells and 100 μl after spinning down the cells. Measure the absorbance at 280 nm (ABS280) cleared lysate after spinning down the cells and calculate the appropriate volume for 5 5 μg of protein (assume 1 mg/mL=1 ABS). Load this calculated amount of cleared lysate on the gel. Load ½ of this amount for each of the non-cleared samples.

For the remainder of the protocol:

1. Solubilize pellets in 5 mL 6 M guanidine, 10 mM dithiothreitol (DTT), 10 mM ethylenediaminetetra-acetate (EDTA), buffered with 50 mM Tris pH 8.1. (This is a 100× concentration from initial culture volume). Pellets are incubated at 48° C. degrees for 3-4 hours to solubilize. Alternatively, pellets are shaken at 37° C. for 4 hrs.

2. Spin to remove insoluble proteins.

3. Use supernatant to obtain protein concentration with BCA assay. Note the tolerance for guanidine in BCA (or Bradford) assay is 4M. As such, dilute the analyte to 4M. It is likely that an additional 3, 10× dilutions in 4M guanidine will be needed to in order for the protein concentration to fall in the measurable range.

4. Load 1-2 μg of all samples on gel for analysis.

5. To test refolding, combine 300 μg of each of the alpha α and β protein/guanidine solutions and dilute to 10 mL total volume with 5 M urea, 0.4 M L-arginine, 100 mM Tris pH 8.1, 3.7 mM cystamine (note not cysteamine), 6.6 mM β-mercapoethylamine to a final concentration of 60 mg/L. Keep at 4° C. If cystamine and mecaptoethylamine are not available, 2 mM DTT can be substituted. Aliquot and freeze remaining unused guanidine/protein solutions.

6. Add this solution to 10 KDa MWCO snakeskin tubing or a 12 mL pierce dialysis cassette.

7. Dialyze in 100 mL of water for 6-8 hours or overnight at 4° C.

8. Change buffer to 10 mM Tris pH 8.1 at 4° C., dialyze overnight.

9. Remove dialyzed solution, spin to clear.

10. To assess refolding, both a native and denaturing gel. When solubilized, refolded protein can proceed with His column or anion exchange to clean up and return to frozen aliquots to scale up refolding. Scale up will require Pierce dialysis snakeskin tubing 10 Kda MWCO.

1G4c113 TCR alpha and beta chains are expressed and refolded as described. Following the refolding samples are purified by loading onto MonoQ with 20 mM Tris (pH 8.1) and 50 mM NaCl. The refolded 1G4c113 TCR is eluted by a gradient to 20 mM Tris (pH 8.1) and 1M NaCl.

### Example 12

### In Vivo Efficacy of an AML-Targeting sCAR-T System

To synthesize AML-targeting switches, antibody Fab fragments of clone hM195 (anti-CD33)¹⁵, and 32716 (anti-CD123)¹⁶ are expressed in E. coli. Briefly, an orthogonal amber suppressor tRNA and aminoacyl-tRNA synthetase (aaRS) pair is co-expressed in E. coli with Fab genes containing a TAG codon at different positions, and cultures are allowed to grow and incorporate pAzF at introduced TAG codons. The mutant Fabs containing pAzF at different sites are then site-specifically modified with the cyclooctyne-FITC linker in a similar fashion described in the synthesis of anti-CD19 switch.

Next, the in vitro efficacy of newly conjugated switch molecules using various AML cell lines is compared. The dose-dependent cytotoxic activity of anti-FITC-CART cells is determined at different effector to target ratios. In addition, the dose-dependent activation of CAR-T cells is also confirmed by monitoring the secretion of inflammatory cytokines by ELISA. Since the target antigen as well as the corresponding epitope for each targeting antibody is different, the optimal FITC-conjugation site for each antibody switch is empirically determined—the optimized switches developed are used in future in vivo studies.

Next, the in vivo efficacy of the optimized switches using orthotopically engrafted HL60 cells in NSG mice is evaluated. In treatment groups, mice receive switch molecules intravenously and are dosed accordingly based on in vitro efficacy and in vivo PK data. In these studies, conventional anti-CD33-CART, and anti-CD123-CART are included for comparison of in vivo efficacy. Once comparable in vivo efficacies are confirmed in conventional and switchable CAR-T cells, a dose titration study is carried out with switches—to validate the dose-titratable therapeutic response of anti-FITC-sCAR-T cells. Furthermore, persistent CAR-T cells from each group (switchable and conventional CAR-T) undergo detailed immunophenotypic characterization. In addition to in vivo efficacy, the potential for on-target, off-tumor toxicity of therapy, focusing on the histopathology of infiltrating T cells in off-target organs is evaluated.

The in vivo efficacy of this system is further evaluated in a more clinically relevant disease model, the patient-derived xenograft (PDX) model. Using an approved IRB protocol, peripheral blood from AML patients is obtained from a Bone Marrow Transplant Unit at a local hospital. Mononuclear cells of the AML patients are separated by Ficoll gradient density centrifugation and characterized by flow cytometry. For the proposed PDX studies, NSG mice are used. In brief, NSG mice are infused with primary AML mononuclear cells (30˜50×10⁶ cells per mouse), and successful engraftment is determined by the presence of circulating blasts in peripheral blood (2 to 3 week post-injection). In parallel, a portion of the infused PBMCs is used to generate autologous engineered CAR-T cells. To evaluate the potential clinical advantage of a combined switch therapy, one group of mice receives a mixture of switches. The in vivo efficacy of switchable CAR-T therapy is determined by the elimination of AML blasts in peripheral blood and bone marrow, and prolonged survival compared to vehicle group. To test the myeloablative potential and hematopoietic toxicity of CAR-Ts, a mouse model with a humanized immune system is generated by injecting human fetal liver CD34⁺ cells into newborn NSG mice. Once engraftment is confirmed by flow cytometry, mice are infused with anti-FITC-CART cells and switches. In parallel, additional mice receive conventional CAR-T cells as controls. Persistent myeloablation is observed in conventional CAR-T groups, whereas the mice that received the switchable therapy recover their myeloid population within several weeks.

The efficacy of the switchable therapy is further evaluated in a heterogeneous AML patient-derived xenograft (PDX) model. In parallel, the safety profile of this system is evaluated in a humanized mice model in which human CD34⁺ hematopoietic stem cells are engrafted in immune-deficient mice, where a head-to-head comparison of the efficacy and safety profile of our AML-targeting switchable CAR-T platform is compared with a corresponding conventional CAR-T system.

### Example 13

### CD19 Surrogate B Cell Depletion Study

To determine the safety and efficacy of our platform in a more physiological relevant setting, the switchable CAR-T therapy was evaluated in a C57BL/6 surrogate model. Thus, a mouse surrogate switchable CAR-T cells using mouse T cells was established; an anti-mouse CD19 antibody (clone 1D3) (SEQ ID NOS. 16 and 17) was conjugated with FITC to target mouse B cells. The in vivo activity of the mouse surrogate anti-CD19 sCAR-T system was then tested for its ability to deplete normal B cells in wild type C57BL/6 mice. A conventional mCART19 was prepared and administered in parallel as a positive control (FIG. 60A). When CD3⁺ and CD19⁺ populations were monitored weekly in peripheral blood, mFITC-CART eliminated CD19-positive cells in a switch-dependent manner, such that terminating switch doses allowed for the repopulation of normal B cells, whereas the conventional mCART19 induced and maintained a persistent loss of CD19⁺ cells throughout the studies, recapitulating the persistent B-aplasia in patients treated with CART-19 therapy (FIG. 60B). These studies demonstrate that the switchable CAR-T platform can be customized to effectively target antigen specific cells, and unlike conventional CAR-T therapy, the engineered T cells can also be “switched-off” in the event of adverse effects (i.e. targeting of normal tissues or cells).

### Example 14

### Design and Synthesis of CAR T Cells and Switch Molecules

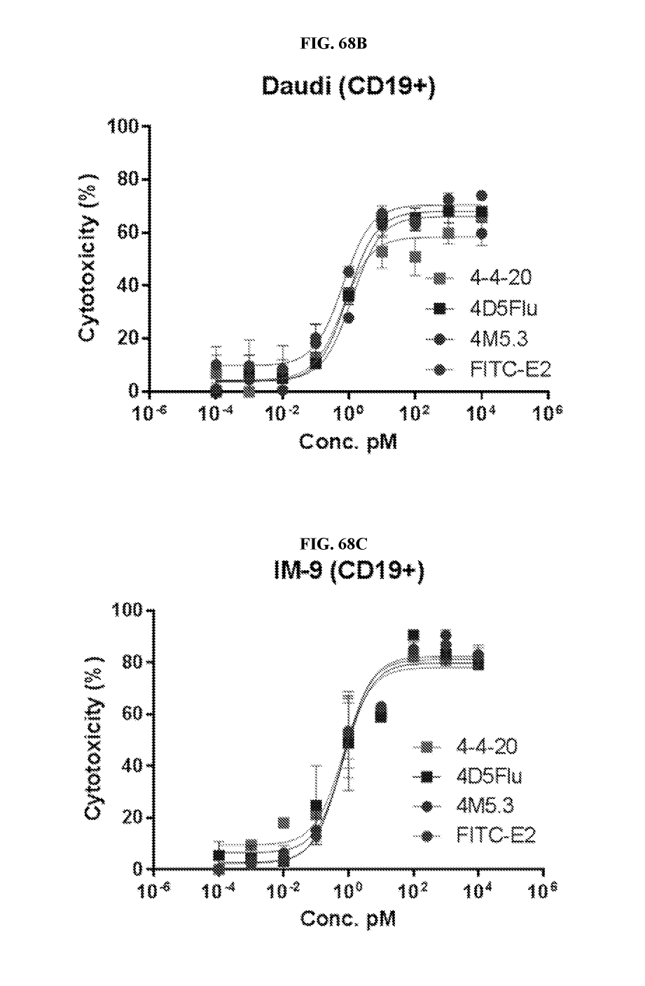

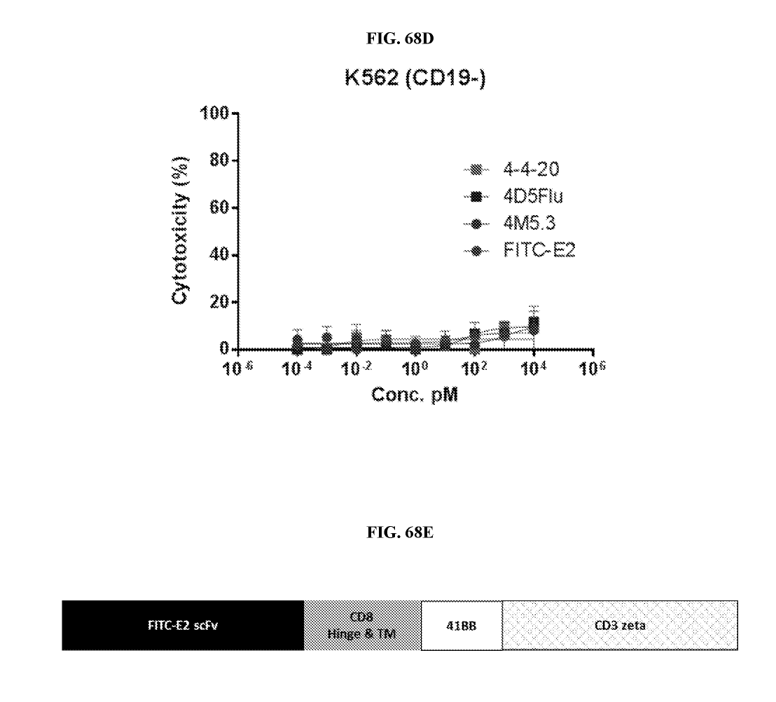

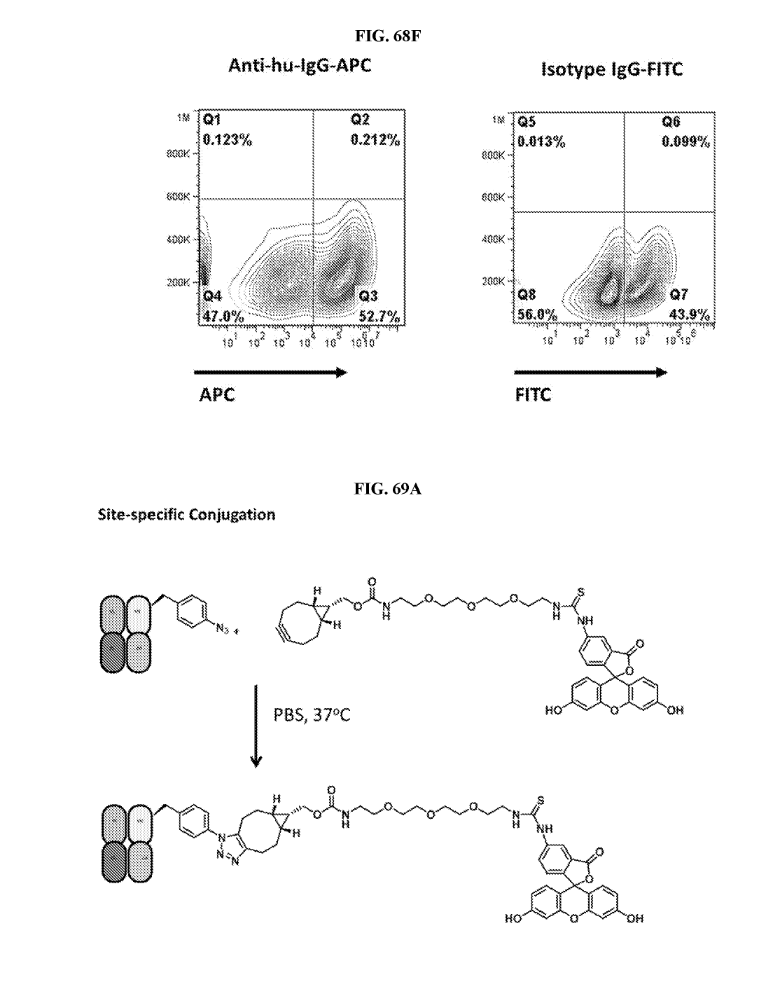

To redirect the specificity of CAR-T cells with a switch molecule, CAR-T cells were generated that bind the synthetic dye, fluorescein (FITC), which is physiologically absent and has demonstrated excellent selectivity in imaging agents and in antibody or small molecule-based detection. CAR-T were generated cells using a range of anti-FITC scFv sequences that differ in their affinities towards FITC, and found that all anti-FITC CAR-T cells elicit in vitro cytotoxicity with the same switch to a similar extent (FIG. 68A, raw data shown in table 29). Therefore, the fully human FITC-E2 scFv sequence was chosen for the anti-FITC CAR because it is expected to minimize the potential for immunogenicity. FITC-E2 scFv was inserted into a second-generation CAR expression cassette in a lentiviral vector that encodes the hinge and transmembrane region of the human CD8 followed by the cytoplasmic domains of human 41BB and CD3ζ (FIG. 68B, raw data shown in Table 30). Viral particles were produced and used to transduce activated human PBMCs. Seven days post-viral transduction, CAR expression varied from ˜40-60% as determined by flow cytometry using APC-labeled anti-human IgG and FITC-conjugated isotype antibody (FIG. 68C, raw data shown in Table 31).

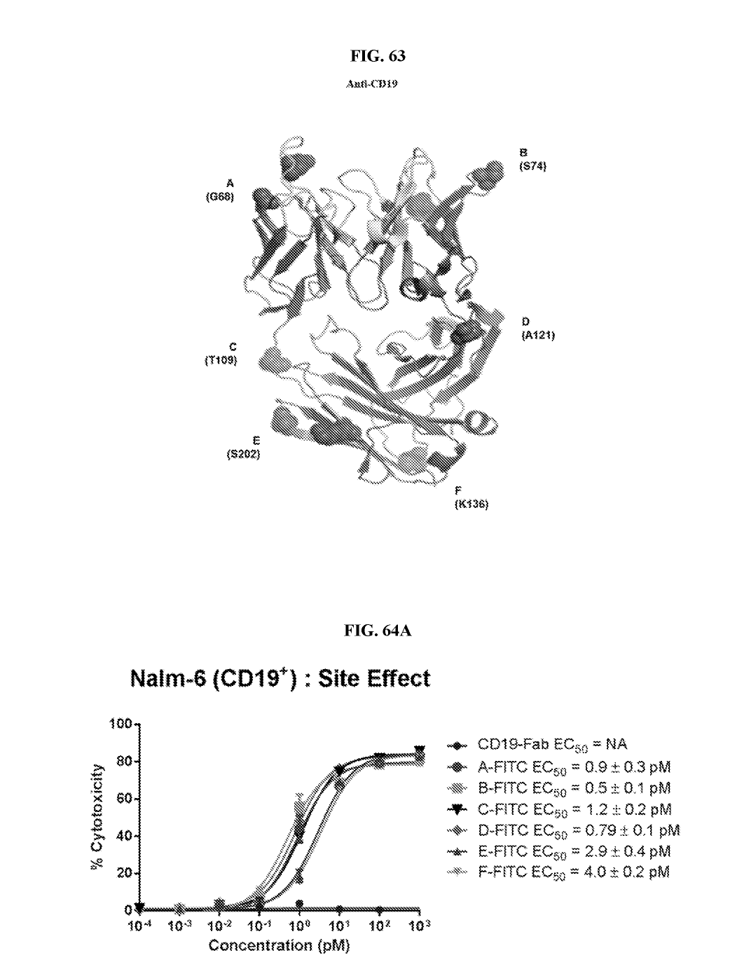

As an initial sCAR-T target, CD19 was chosen, an antigen that is highly expressed on B cell cancers. For the switch itself, the anti-CD19 specific monoclonal antibody, clone FMC63, was used which was previously used in a second generation CAR-T cells against B cell cancers. The Fab format was chosen over full length IgG due to its shorter half-life, which allows for better temporal control of CAR-T cell activity. To determine the effect of FITC conjugation site on the distance and geometry of the pseudo-immunological synapse formed between CAR, TAA and switch, a site-specific protein conjugation strategy was used. As with the generation of previously described switches, this method involves the genetic incorporation of noncanonical amino acids with bio-orthogonal chemical reactivity at defined positions in an antibody to generate chemically defined small molecule conjugates. Specifically, the noncanonical amino acid para-azidophenylalanine (pAzF) was incorporated individually at six surface exposed positions (A, G68; B, S74; C, T109; D, A121; E, S202; and F, K138) based on the crystal structure of a murine Fab 93f3 (PDB: 1T4K, FIG. 63A). The distinct location of each conjugation site relative to the antigen binding region (proximal A and B; medial C and D; distal E and F) in the anti-CD19 Fab is expected to afford geometrically distinct immunological synapses. In addition to monovalent conjugates, bivalent FITC conjugates, AB and EF, were also generated to determine the effect of valency on CAR-T cell activity.

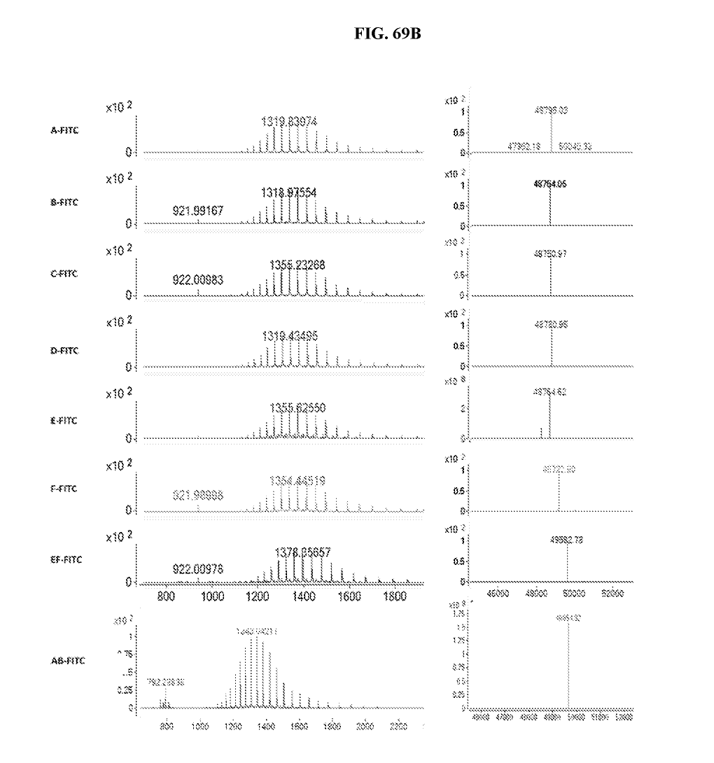

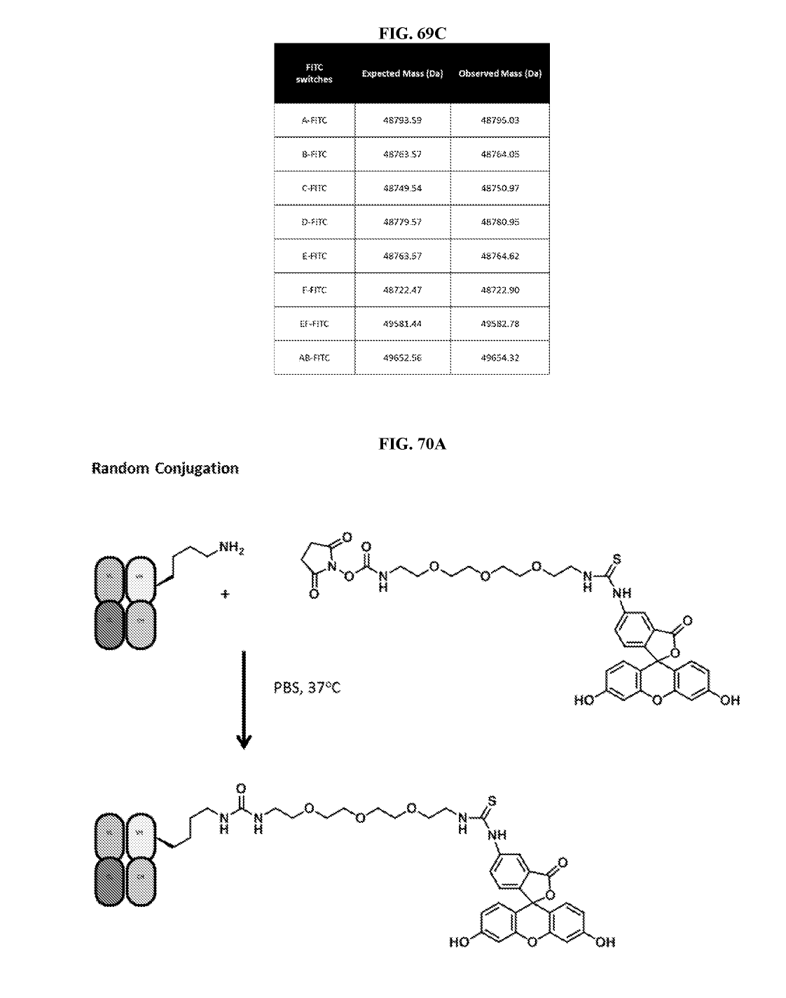

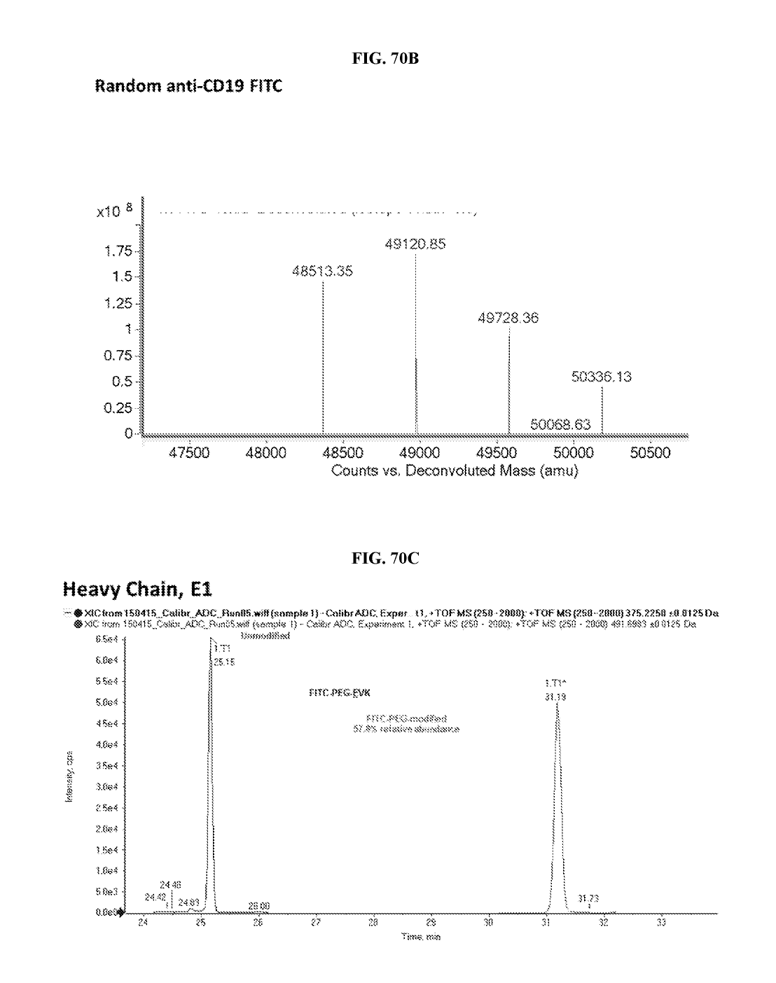

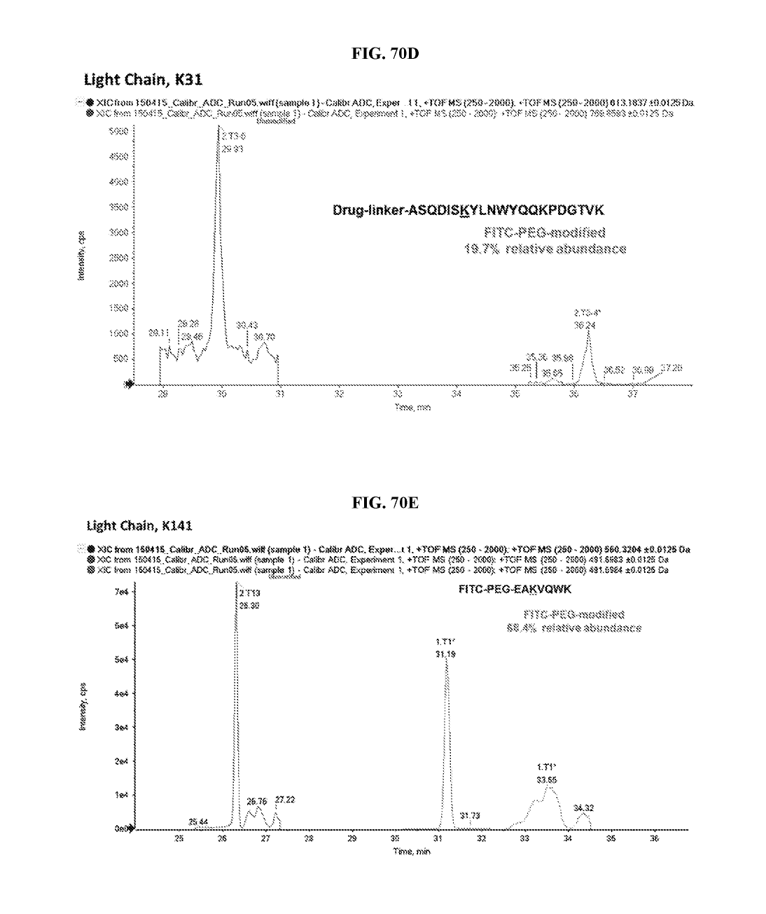

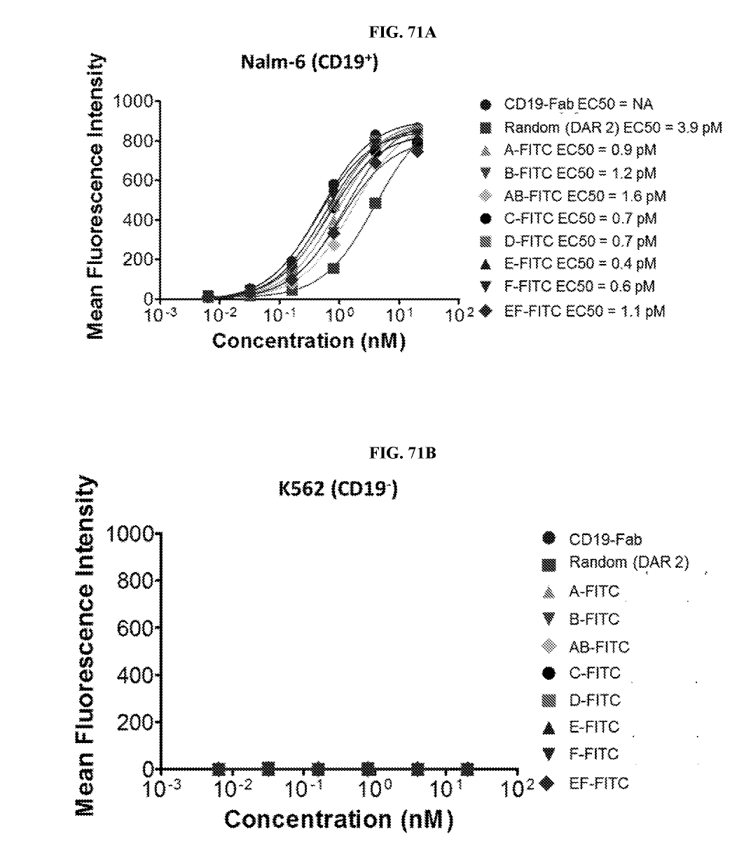

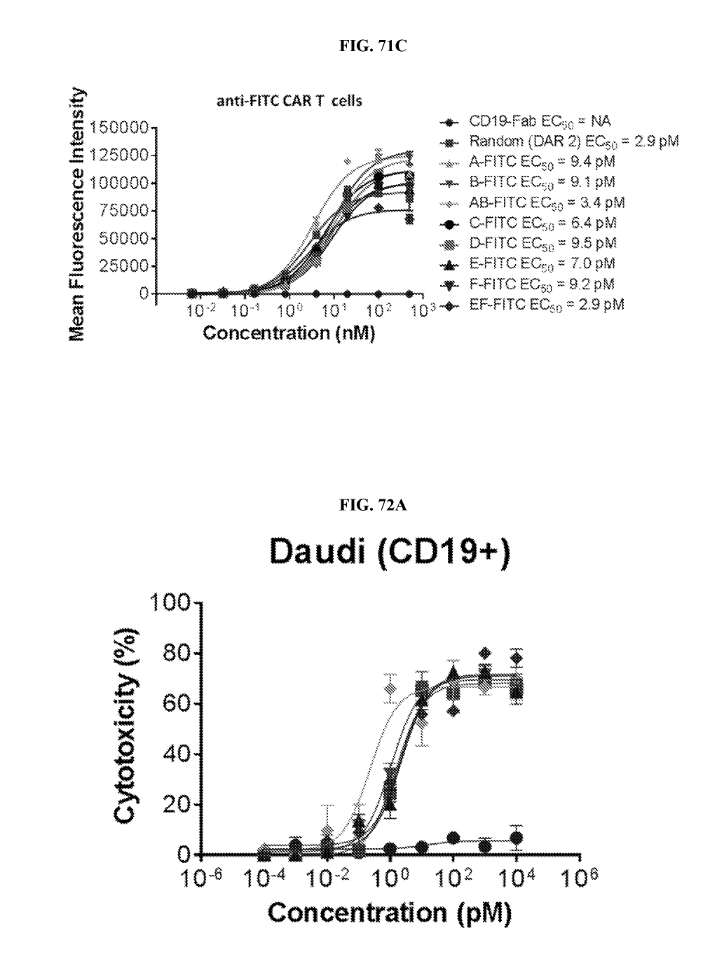

To express mutant Fabs, a plasmid encoding the FMC63 gene with an amber (TAG) codon at the desired position was co-transformed into E. coli with a plasmid harboring an orthogonal amber suppressor tRNA/aminoacyl-tRNA synthetase pair that was evolved to incorporate pAzF in response to the TAG codon. The purified Fabs were subsequently conjugated with a FITC linker with a terminal cyclooctyne group to allow for selective coupling to pAzF via a “click” reaction under neutral pH (PBS, pH 7.4) (FIG. 69A). The molecular weight and purity of each construct was analyzed by SDS-PAGE and mass spectrometry (MS), which confirmed that homogenous reaction products with a conjugation efficiency of >95% were generated (FIG. 69B). For comparison, a random FITC conjugate was generated using N-hydroxysuccinimide (NHS) chemistry, which yielded an average FITC to antibody ratio of 2 (FIG. 70A-B). Mass spectrometry analysis after tryptic digestion revealed ˜58% of the conjugates were modified at the heavy chain N-terminal amino group and the remaining conjugation occurred at lysine residues within the light chain, largely at K31 (˜20%) and K141 (˜68.4%, FIG. 70C-E). Next, the binding of FITC-labeled anti-CD19 switches was assessed by flow cytometry. As shown in FIG. 71A, the binding affinities of all site-specific FITC switches to CD19-positive target cells (Nalm-6) were comparable to the parental anti-CD19 Fab (0.4-1.6 nM), whereas slightly decreased binding was observed with the random conjugate (EC₅₀=4.0 nM)—a likely consequence of modification within the CDR1 of light chain at position K31 (FIG. 63). No binding was observed with CD19-negative cells (K562) (FIG. 71B). Bivalent FITC conjugates (EC₅₀=˜3 nM) exhibited 2 to 3-fold higher affinity for the anti-FITC CAR-T cells than monovalent FITC switches (EC₅₀=6-9 nM; FIG. 71C).

### Example 15

### Effect of FITC Conjugation Site and Valency on Anti-FITC CAR-T Cells In Vitro Activity

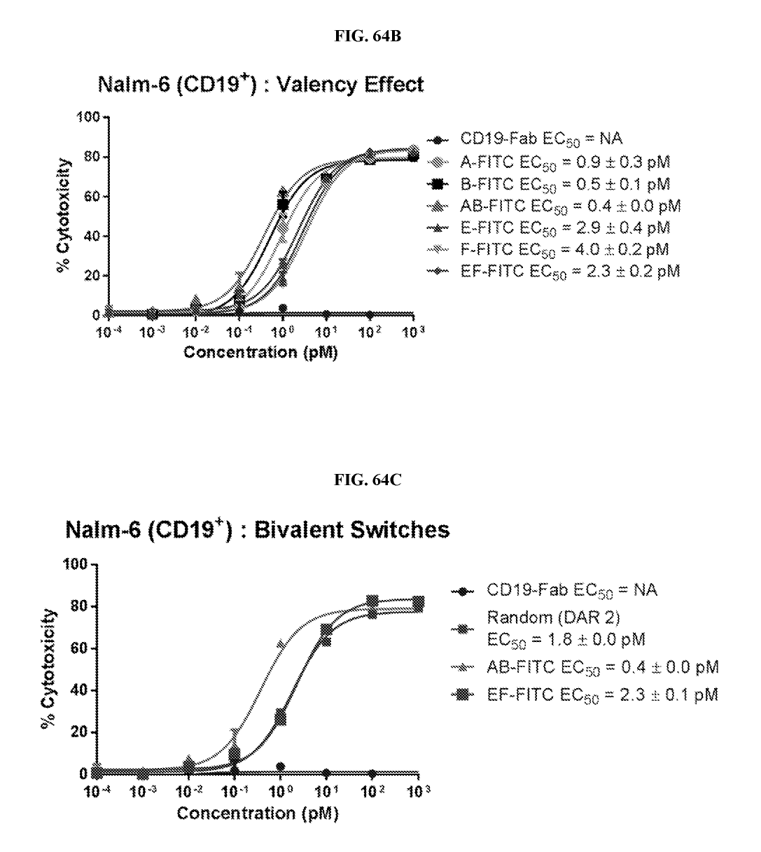

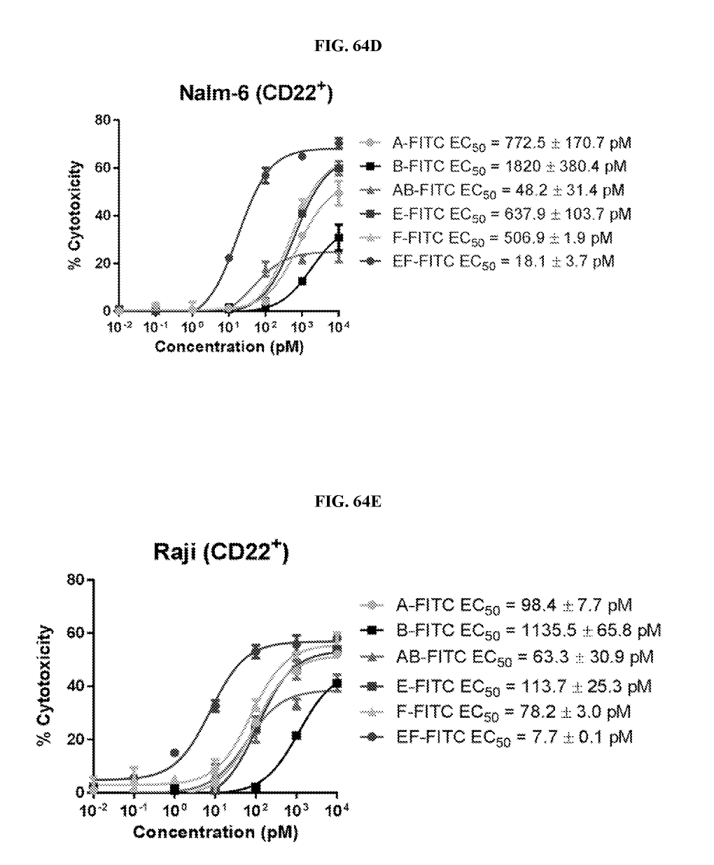

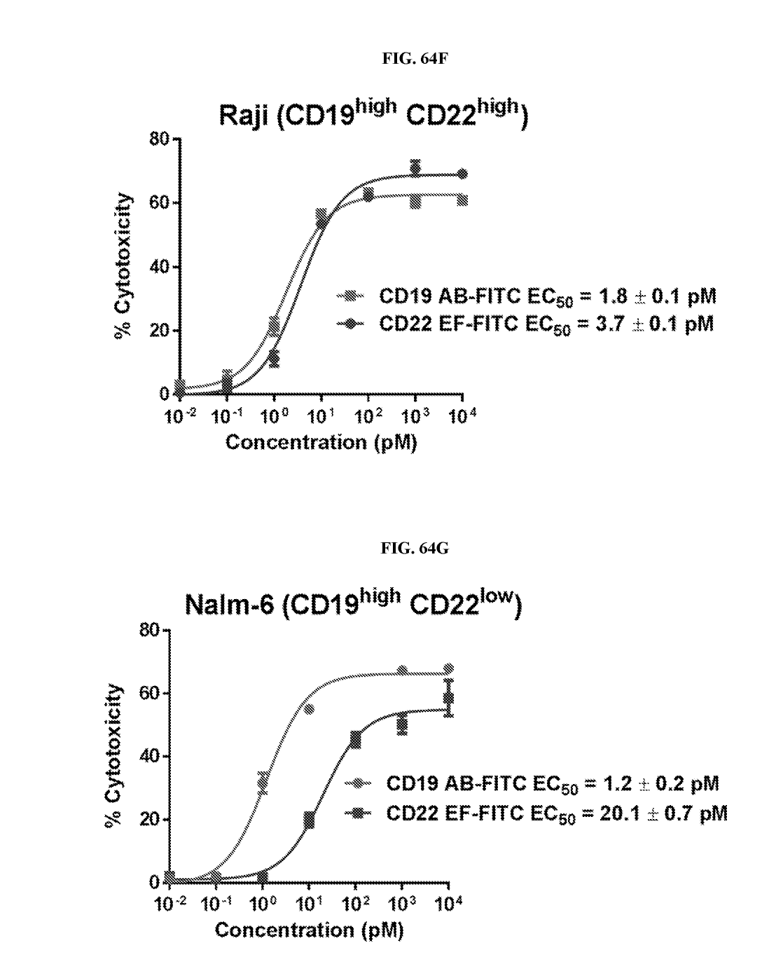

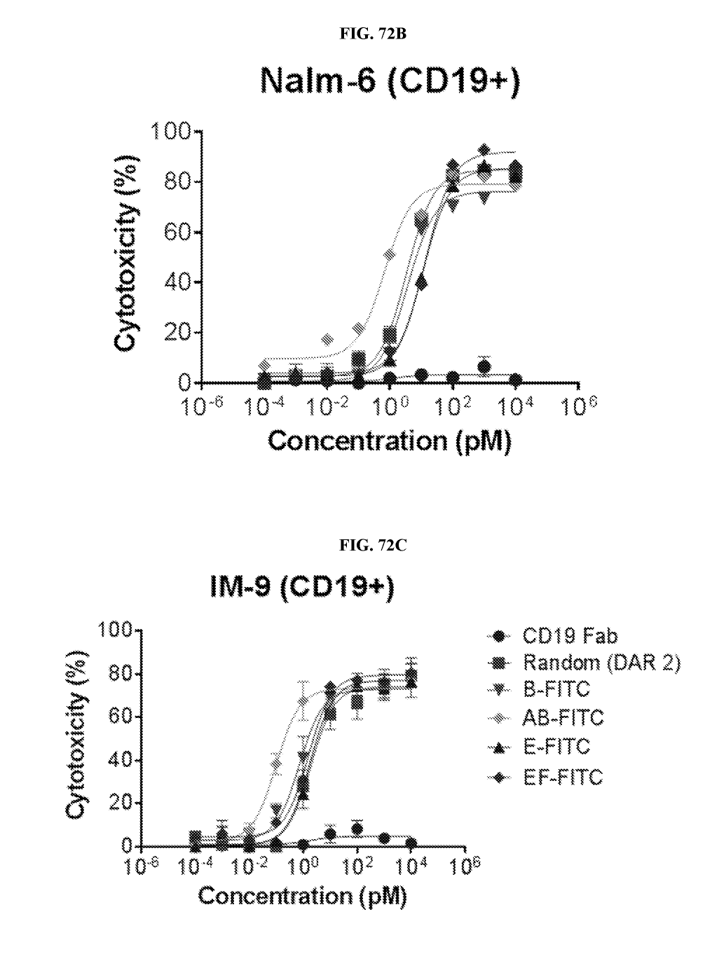

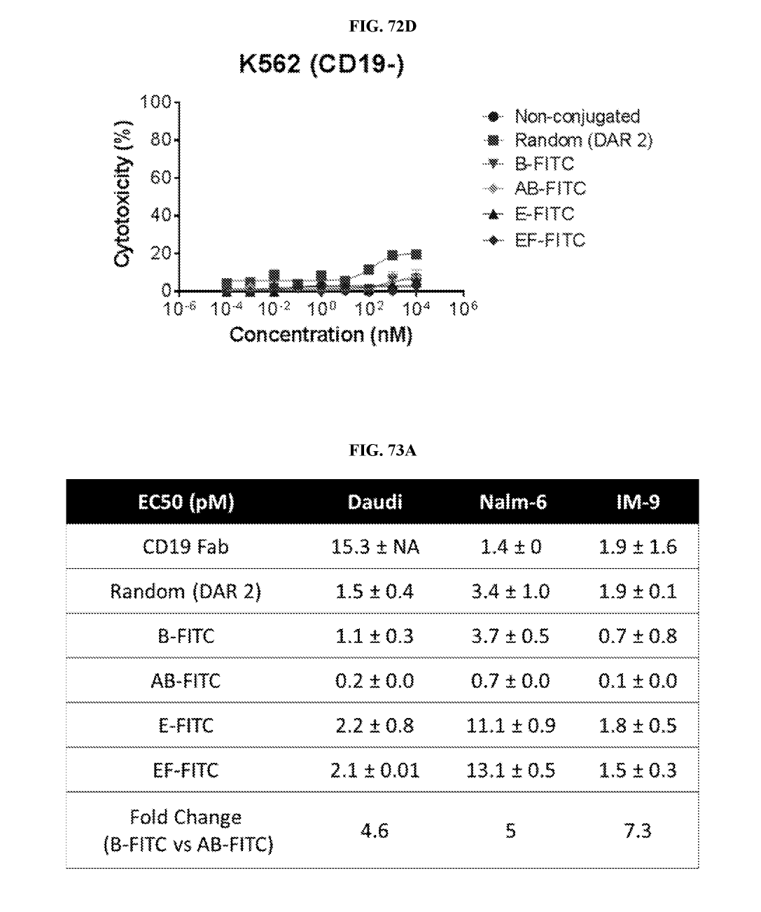

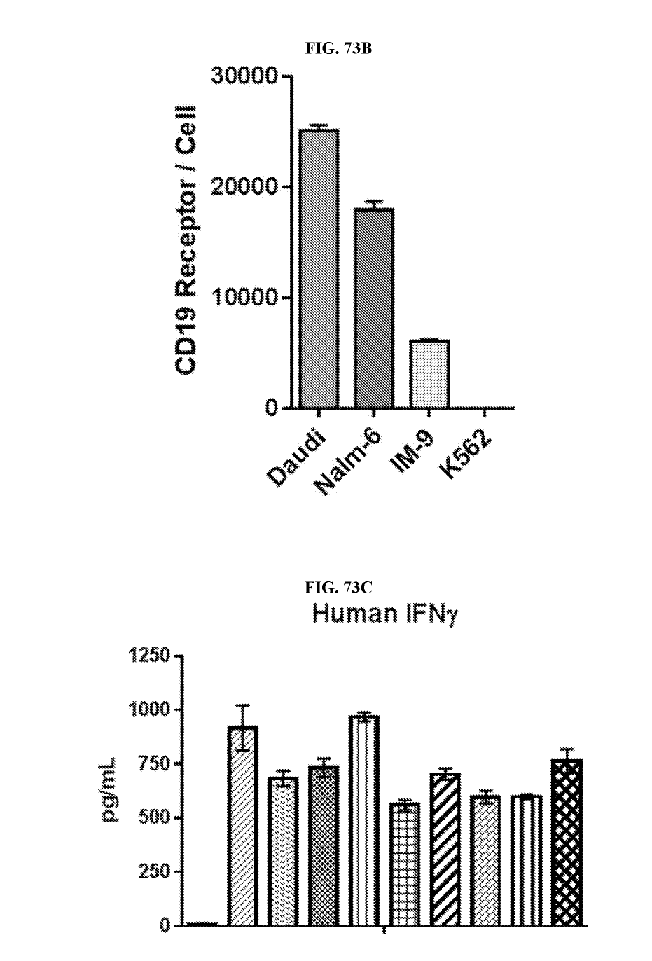

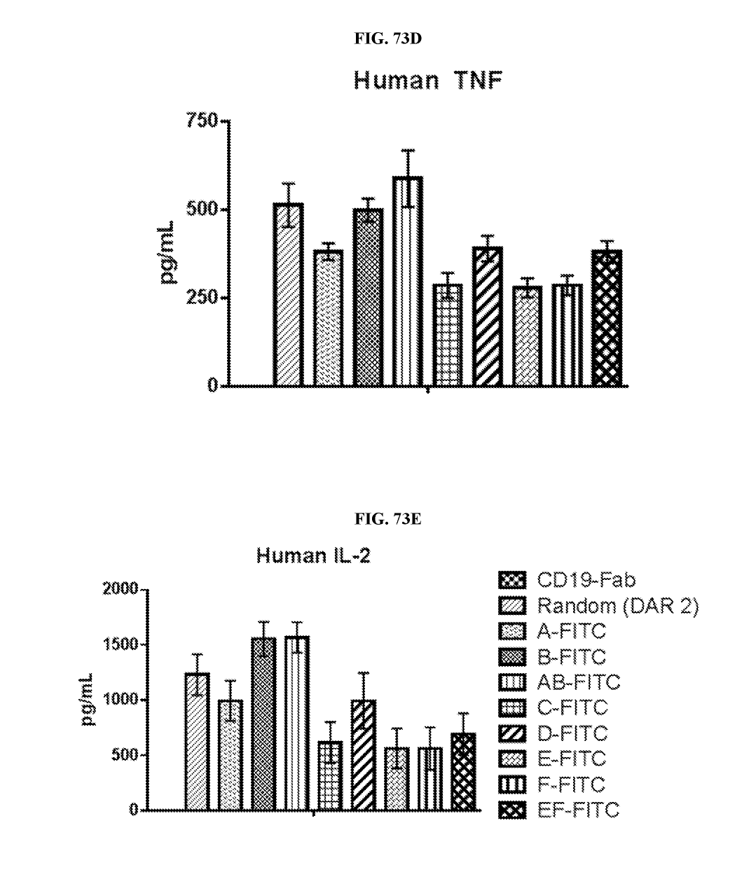

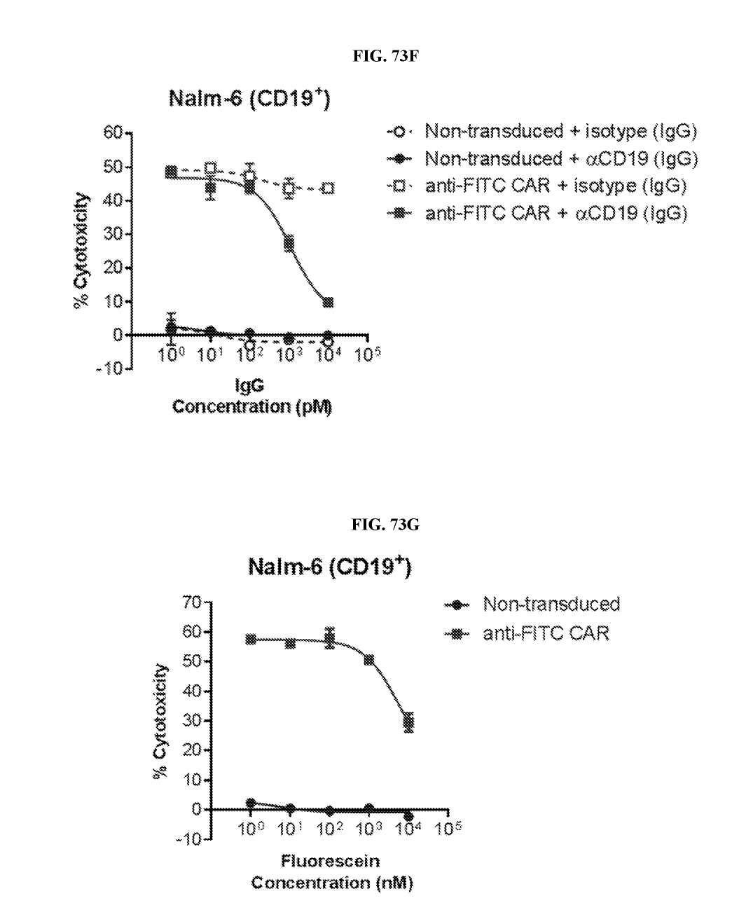

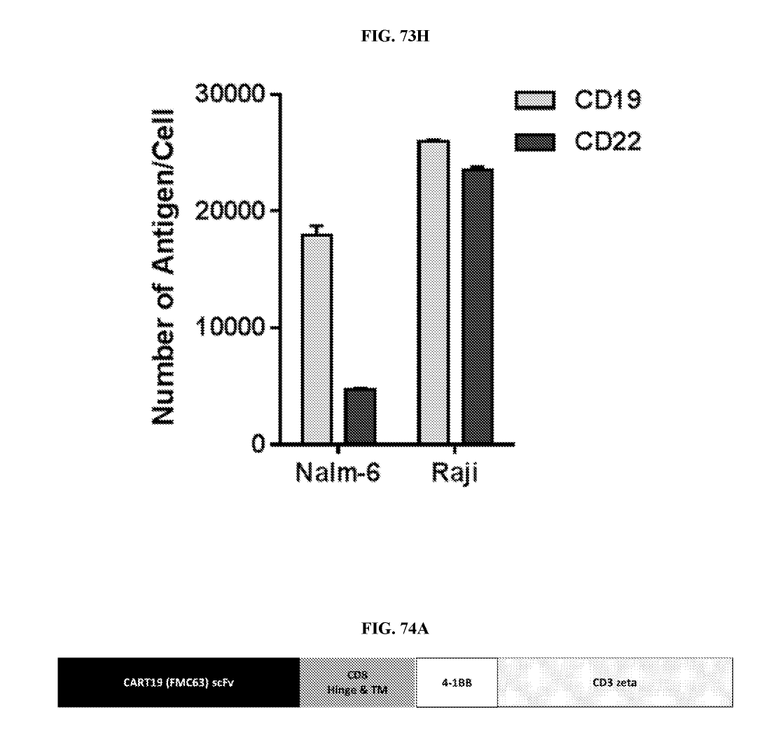

The ability of the anti-CD19 Fab switches to induce anti-FITC CAR-T cell effector functions was evaluated. Highly potent lytic activity was induced by all FITC conjugates against Nalm-6 cells (human CD19⁺ B-ALL line) in a dose dependent manner. However, differences in cytotoxicity were observed depending on conjugation sites—FITC conjugates proximal to the antigen binding region (EC₅₀; A=0.9±0.3 pM and B=0.5±0.1 pM) were more potent than switches conjugated at distal sites (EC₅₀; E=2.9±0.4 pM and F=4.0±0.2 pM) (FIG. 64A). This trend was consistently observed in cytotoxicity assays using different cancer cell lines (Daudi and IM-9) with varied CD19 expression levels and with different CAR-T cells generated from three healthy donors (FIG. 72A-D, raw data shown in Tables 33, 34, 35, and 36 and FIG. 73A-B). The bivalent switches (EC₅₀; AB=0.4±0.0 pM and EF=2.3±0.2 pM) were more potent than their corresponding monovalent FITC switches (FIG. 64B). The random FITC conjugate induced lytic activity (EC₅₀=1.8±0.0 pM) that was comparable to the site-specific EF switch, but significantly less than the site-specific AB switch (FIG. 64C). These results show that the site and stoichiometry of conjugation of FITC to anti-CD19 Fab affect the anti-FITC CAR-T activity. Non-conjugated CD19 Fabs failed to induce lysis of Nalm-6 cells, and no significant cytotoxic activity was observed with K562 (CD19-) cells for any site-specifically conjugated switch (FIG. 72A-D). In addition to lytic function, all FITC conjugates induced IFN-γ, TNF, and IL-2 release that correlated with the degree of cytotoxicity, with the AB-FITC switch yielding the highest cytokine induction (FIG. 73C-E). To confirm the specificity of the AB-FITC switch, competition assays were performed using excess anti-CD19 (IgG, clone FMC63) antibody or free fluorescein (FIG. 73F-G, raw data shown in Tables 37 and 38). With a 1000-fold excess of anti-CD19 antibody, lytic activity of anti-FITC CAR-T cells induced with 10 pM of anti-CD19 AB-FITC was significantly reduced (from 54.0±0.1% to 9.8±1.1%), whereas minimal changes were observed with an isotype control antibody. Similarly, excess fluorescein (10 uM) also decreased CAR-T cell cytotoxicity from 57.4±3.7% to 29.5±3.0% in the presence of anti-CD19 AB-FITC (10 pM). Overall, these results demonstrate that the ability to control the site and stoichiometry of FITC conjugation to the switch molecule significantly impacts CAR-T activity, in some cases the bivalent anti-CD19 AB-FITC switch inducing the most potent and selective in vitro cytotoxicity of anti-FITC CAR-T cells.

### Example 16

### Optimization of Anti-CD22 Switch

The feasibility of targeting other tumor antigens using the same anti-FITC CAR-T cells was determined. CD22 is another well-characterized B cell-associated tumor marker, which is found on most B-cell leukemias and lymphomas. To generate anti-CD22 switches, sequences of the variable region were obtained from the anti-CD22 antibody, clone M971, which has been previously incorporated into a CAR construct which showed in vivo efficacy in mouse xenograft models. Proximal and distal positions were selected similar to the CD19 switches to generate 4 monovalent (A, B, E, and F), as well as 2 bivalent (AB and EF) FITC conjugated switches using the same semi-synthetic approach described above. Interestingly, when the anti-CD22-FITC switches were compared with the anti-FITC CAR-T cells in cytotoxicity assays using Nalm-6 cells, it was found that FITC conjugated to distal positions (EC₅₀; E=0.6±0.1 nM and F=0.5±0.0 nM) was more cytotoxic to CD22⁺ cells in comparison to proximal FITC conjugates (EC₅₀; A=0.8±0.2 nM and B=1.8±0.4 nM) (FIG. 64E). These results are opposite to the findings with CD19 switches (FIG. 64B). However, as was the case with CD19, an enhancement in cytotoxicity was observed with bivalent switches (EC₅₀; EF=18±4 pM and AB=48±31 pM), but the improvement was more profound. A similar trend was observed in the cytotoxicity assays using CD22⁺ B cell lymphoma cells, Raji (FIG. 64E). This difference in CAR-T activity with different conjugation sites suggests that distinct geometries are required for each antigen-antibody interaction to realize optimal effector function. It was also observed that the anti-CD19 AB-FITC switch (EC₅₀; AB=1.2±0.2 pM) is approximately 20× more efficacious on Nalm-6 than the optimized anti-CD22 EF-FITC switch (EC₅₀; EF=20±1.0 pM) (FIG. 64F). This is due to lower CD22 expression levels on Nalm-6 cells (FIG. 71G). Indeed, when both optimized switches were compared in cytotoxicity assays using a CD22-high expressing Raji cells, similar cytotoxicity was observed (EC₅₀=1.8±0.1 pM and 3.7±0.1 pM, respectively) (FIG. 64G). Taken together, these results demonstrate that homogeneous, site-specific FITC-based switches can be readily optimized using a single CAR-T cell to enable highly effective geometries for CAR-T and tumor cell interactions regardless of antigen-antibody pairing.

### Example 17

### Evaluation of FITC-Conjugated Anti-CD22 Switches

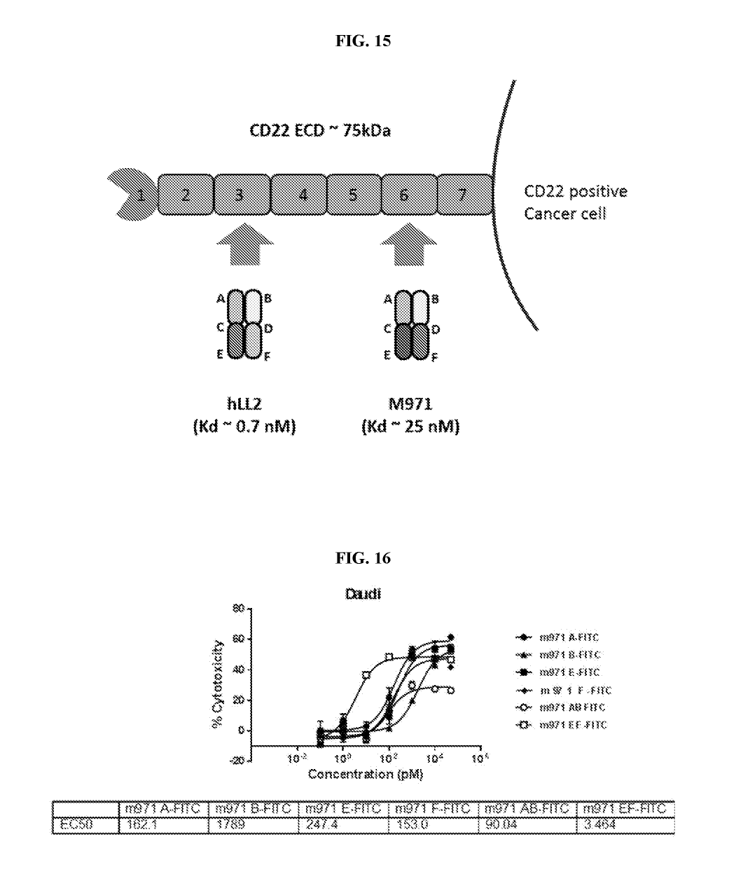

Two anti-CD22 switches were generated based on antibody clones, hLL2 and M971. hLL2 recognizes an epitope close to N-terminus of CD22 with high affinity (Kd ˜0.7 nM), whereas clone M971 binds a membrane proximal epitope of CD22 with lower affinity (Kd ˜25 nM) (see FIG. 15).

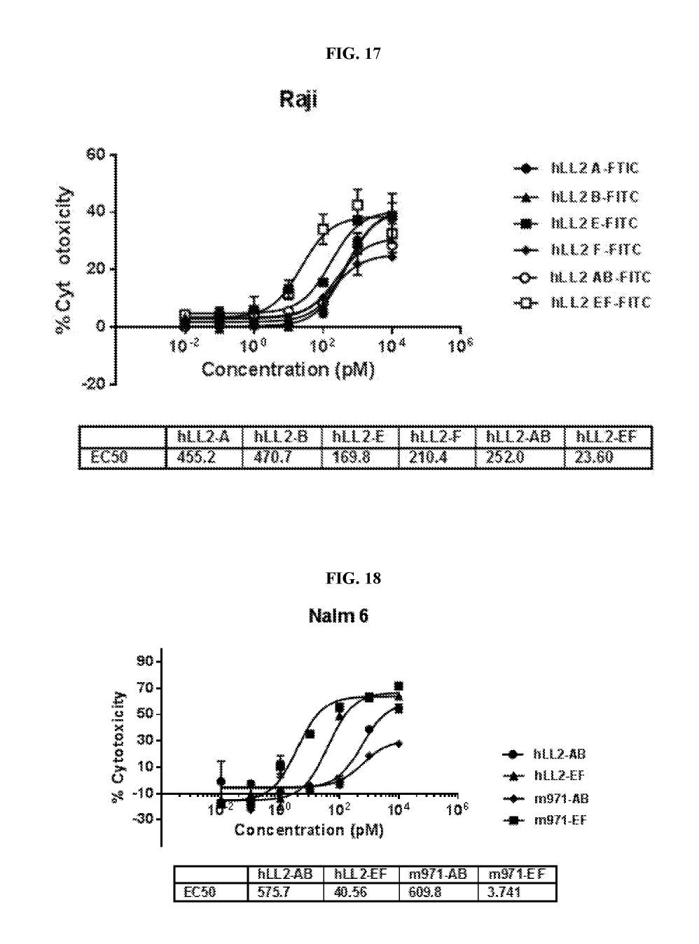

Cytotoxicity of anti-FITC CAR-T cells and switches with varying conjugation sites of FITC to hLL2 and M971 Fab were measured against CD22⁺ Daudi, Raji, and NALM6 cell lines, at E:T=5:1 for 24 hrs. Results are shown in FIGS. 16, 17, and 18.

In both hLL2 and M971, switches with dual conjugation sites further from the antigen binding site (i.e., EF-FITC from FIG. 15) had better efficacy than switches with conjugation sites close to the antigen binding site (i.e. AB-FITC from FIG. 15). While hLL2 has significantly higher (˜35 times) affinity than M971, the M971-EF-FITC switch demonstrated more efficient redirection of anti-FITC CAR-T cells than the hLL2-EF-FITC switch against Nalm6 cells, suggesting the distance between target and CAR-T cells may be more critical than the affinity of the switch for the optimal formation of pseudo-immunological synapse.

### Example 18

### CART 19 vs Anti-FITC sCAR T Cells: In Vitro and In Vivo Comparison

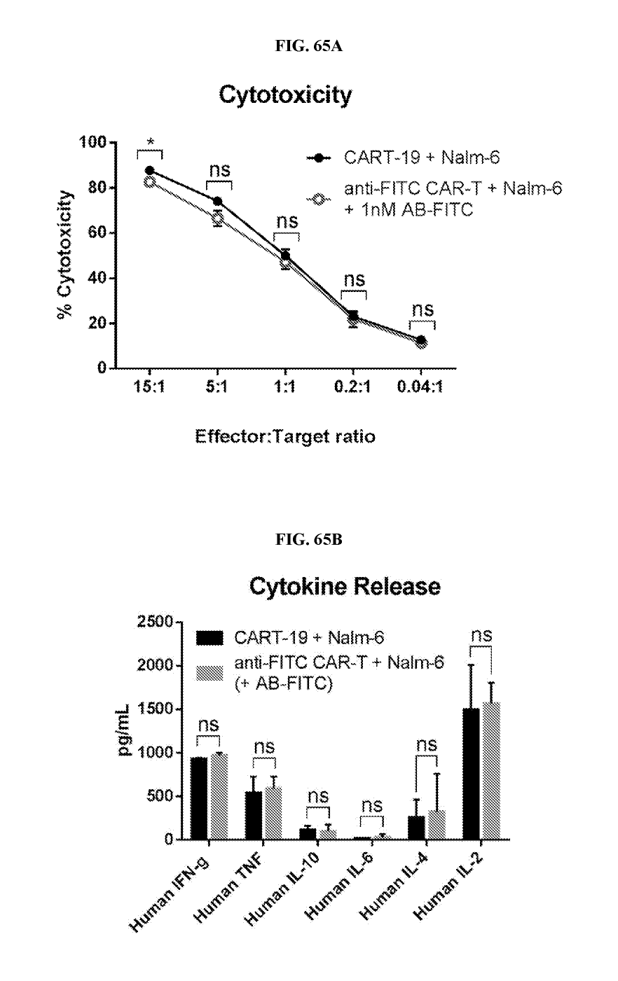

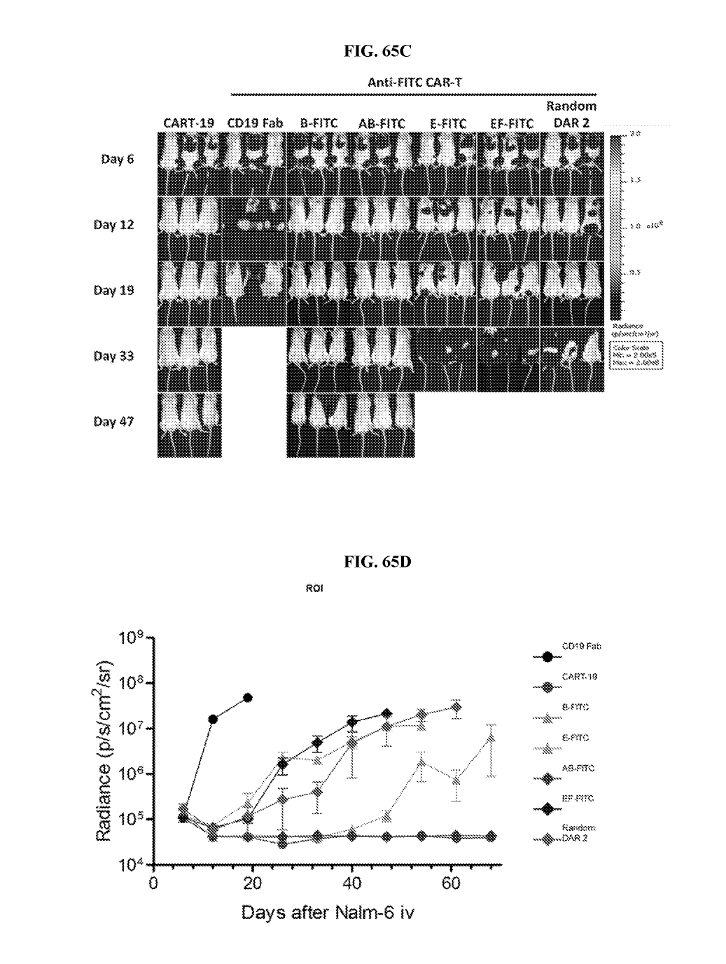

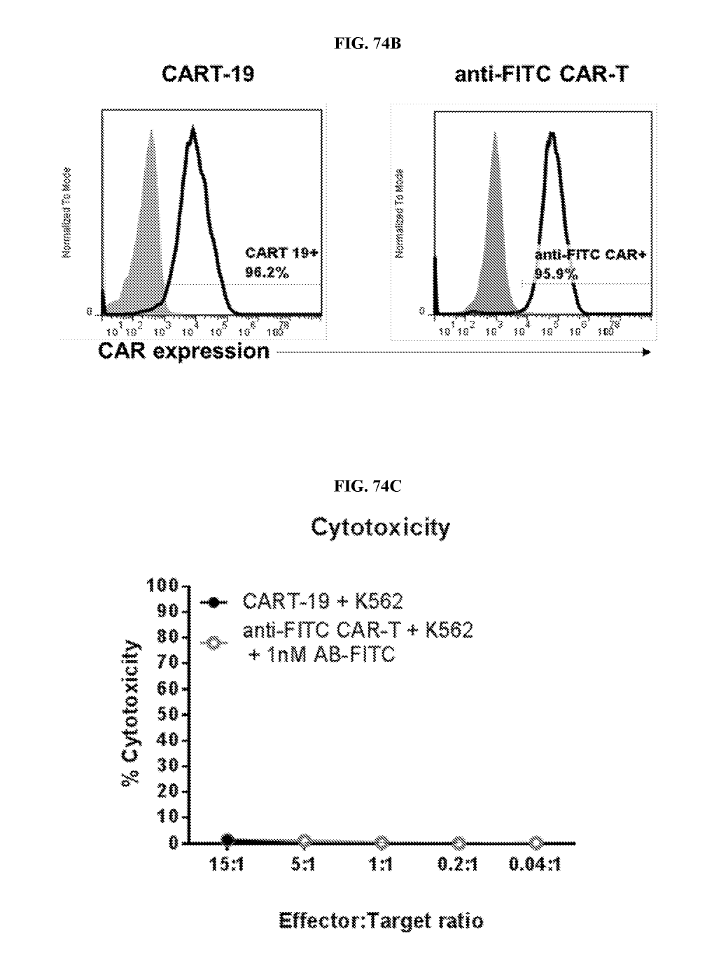

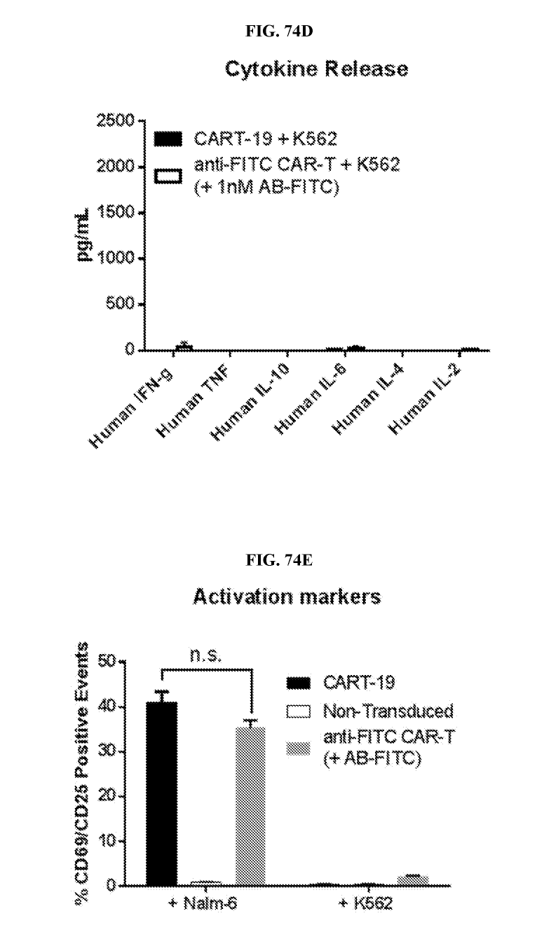

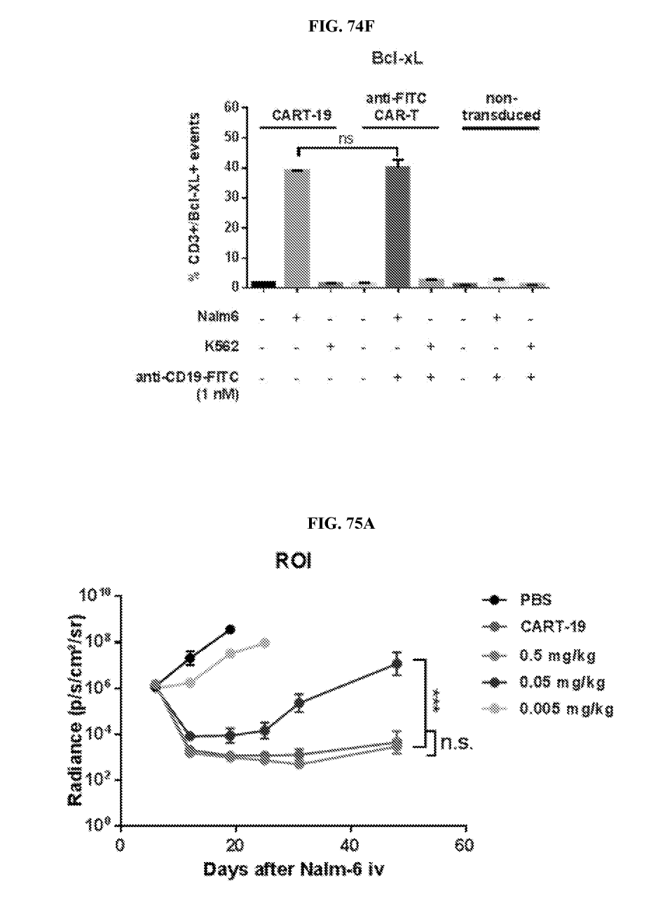

The relative activity of the sCAR-T cells compared with a conventional CAR was determined by comparing cytotoxicity and activation markers with a second generation CD19 specific CAR currently in clinical trials, which uses the same FMC63 anti-CD19 scFv (CART-19; FIG. 74A). Anti-FITC and anti-CD19 CAR lentiviral particles were generated and used to transduce T cells from the same donor (˜50-60% transduction efficiency, 7 days post-transduction). To minimize the effect of differential CAR expression, both CAR-T cells were affinity purified (>90% purity; FIG. 74B) and used for in vitro and in vivo efficacy studies. With 1 nM anti-CD19 AB-FITC, anti-FITC CAR-T cells lysed Nalm-6 cells to a similar extent as CART-19 in 24 hour cytotoxicity assays at varying effector to target (E:T) cell ratios (FIG. 65A, raw data shown in Table 24). Similarly, as shown in FIG. 74E and FIG. 65B (raw data for FIG. 65B shown in Table 25), comparable upregulation of activation markers (CD69 and CD25) and release of cytokines was observed. The induction of the anti-apoptotic marker Bcl-xL, which is a hallmark of 4-1BB costimulatory signaling was also assessed. As shown in FIG. 74F, an induction of Bcl-xL is observed in both CAR-T cells. Moreover, in all experiments, activity of both CAR-T cells was observed only in the presence of CD19-positive tumor cells (FIG. 74C-F). Overall, the in vitro findings suggest that anti-FITC CAR-T cells in conjunction with the optimized anti-CD19 AB-FITC switch are comparable to conventional CD19 targeting CAR-T cells (CART-19) in eliciting tumor-specific effector functions as well as inducing costimulatory signaling.

The CD19-targeting CAR-T cells were tested in vivo in a Nalm-6 xenograft model. Briefly, 0.5×10⁶ Nalm-6 cells transduced with luciferase were injected intravenously (IV) into female NSG mice. Seven days later, mice were infused with 40×10⁶ CAR-T cells IV and switch treatment was initiated with indicated anti-CD19 FITC conjugate(s) at 0.5 mg/kg (IV) or PBS (IV) every other day for a total of six doses (FIG. 65C). Tumor growth was monitored weekly by bioluminescence imaging. After three doses of anti-CD19 AB-FITC, mice infused with anti-FITC CAR-T cells cleared tumor to the same extent as CART-19, and both groups of mice remained tumor-free for greater than 60 days (FIG. 65D). In addition to the proximal bivalent anti-CD19 AB-FITC, selected monovalent (B and E) and bivalent (EF and random DAR 2) switches were evaluated. As shown in FIG. 65C and FIG. 65D, the in vivo activity of the switches correlated with the in vitro data but the differences were more pronounced: the anti-CD19 B-FITC initially exhibited anti-tumor efficacy, however, tumor relapse was observed on day 47 of the study; random and distal conjugates (E- and EF-FITC) delayed tumor growth but did not clear tumors. It is noteworthy that the B switch substantially outperformed bivalent EF and random switches, reaffirming the importance of the conjugation site on efficacy. Rapid tumor growth was observed in mice treated with PBS or non-conjugated anti-CD19 antibody, which confirms that the anti-tumor response is FITC switch-dependent. Importantly, this study demonstrates that these sCAR-T cells can achieve excellent in vivo anti-tumor activity that is comparable to the clinically validated CD19 targeting CAR-T (CART-19).

### Example 19

### Control of In Vivo Switchable CAR T Activity

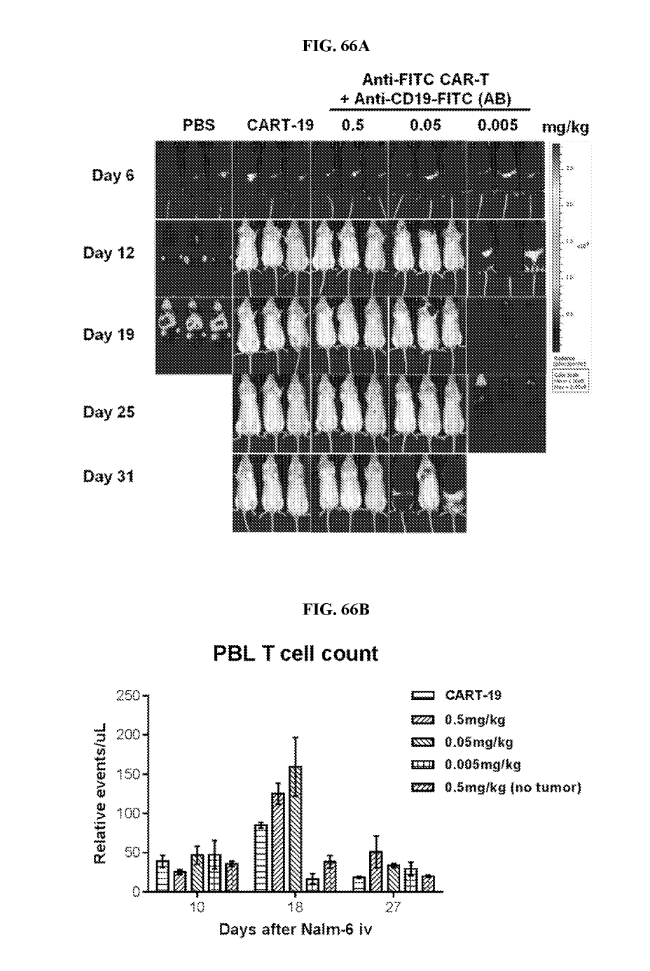

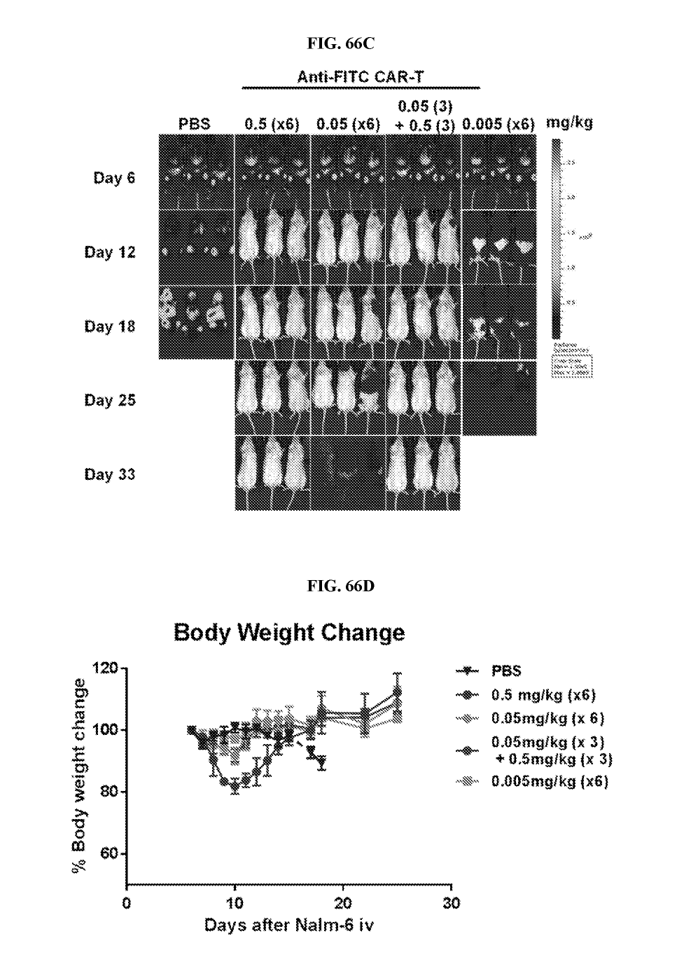

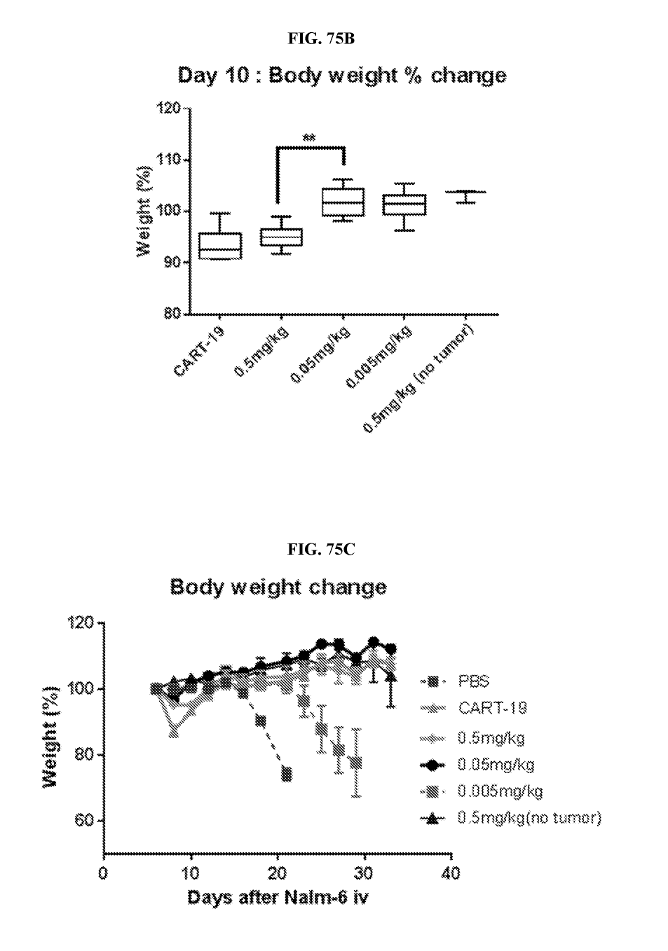

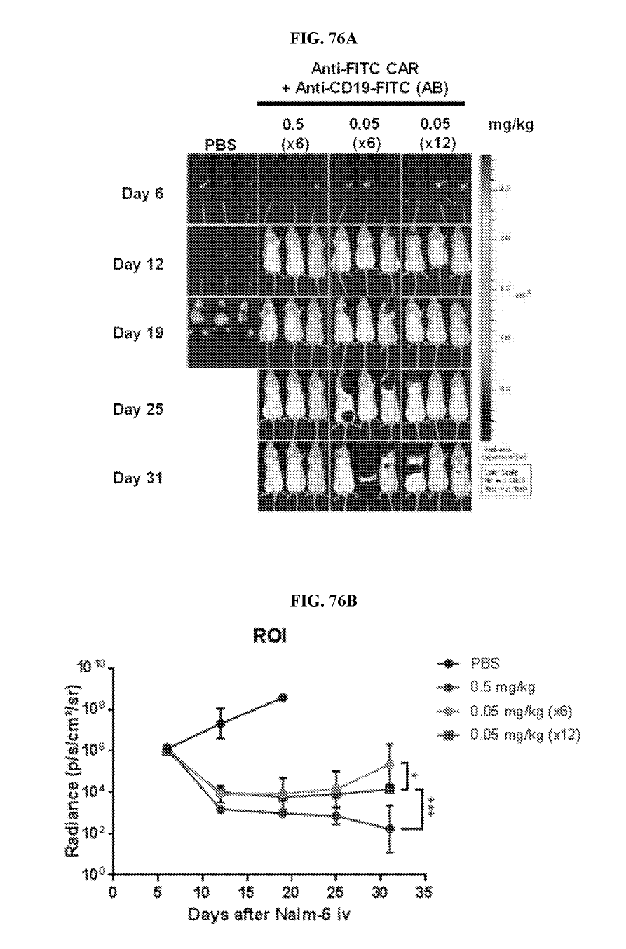

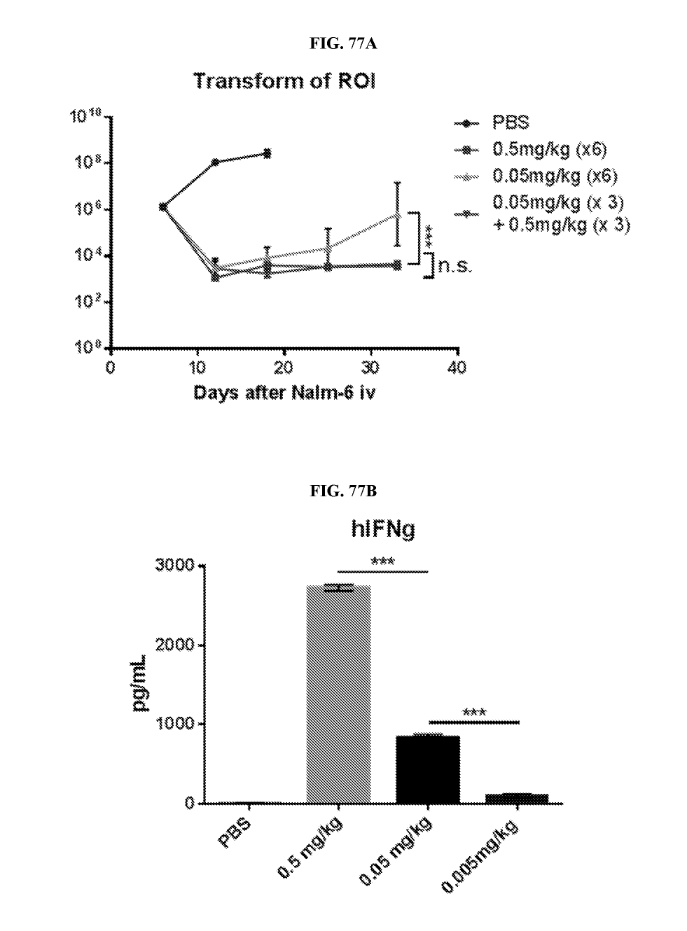

It was determined whether the in vivo activity of these sCAR-T cells can be regulated in a switch dose-dependent manner. Briefly, mice with established Nalm-6 tumor burden received the same number of anti-FITC CAR-T cells (40×10⁶) as described above, and anti-CD19 AB-FITC switches were injected at doses ranging from 0.005-0.5 mg/kg every other day over 12 days (day 7-day 17). Consistent with the previous study, mice treated with the effective dose (0.5 mg/kg) achieved rapid tumor clearance (FIG. 66A and FIG. 75A, raw data shown in Table 40). A reduction in tumor burden was initially observed with animals that received 0.05 mg/kg of switch, however, this group relapsed after treatment with 6 doses and succumbed to disease at day 48. When the 0.05 mg/kg treatment was extended up to 12 doses, tumor growth was stabilized but complete tumor clearance was not observed (FIG. 76A-B). Further decrease in switch dose (0.005 mg/kg) had minimal effect in this disease model. The expansion of anti-FITC CAR-T cells correlated with the observed dose-dependent anti-tumor activity (FIG. 66B, raw data shown in Table 26): after receiving six doses (day 18), animals treated with the highest dose (0.5 mg/kg) had a ˜7-fold increase in CD3⁺ cells compared to mice that received the lowest dose (0.005 mg/kg). The expansion of human T cells was tumor-dependent as demonstrated by the ˜4-fold higher T cell number observed in tumor bearing mice in comparison to disease-free mice. These results indicate that the in vivo sCAR-T cell activity and expansion can be controlled by switch dose. In these dose titration studies, mice that received CART-19 or anti-FITC sCAR-T cells with the anti-CD19 AB-FITC switch at 0.5 mg/kg dose exhibited significant body weight loss (≥10%) shortly after the initiation of treatment (FIG. 75B), which resolved within 4 days (FIG. 75C, raw data shown in Table 41). Conversely, mice treated with suboptimal switch doses (0.05 and 0.005 mg/kg) did not display similar signs of distress. This acute toxicity is likely related to the anti-tumor activity elicited by CAR-T cells, as the control group, healthy mice injected with anti-FITC CAR-T cells and 0.5 mg/kg of anti-CD19 AB-FITC, did not exhibit any signs of toxicity (FIG. 75B-C).

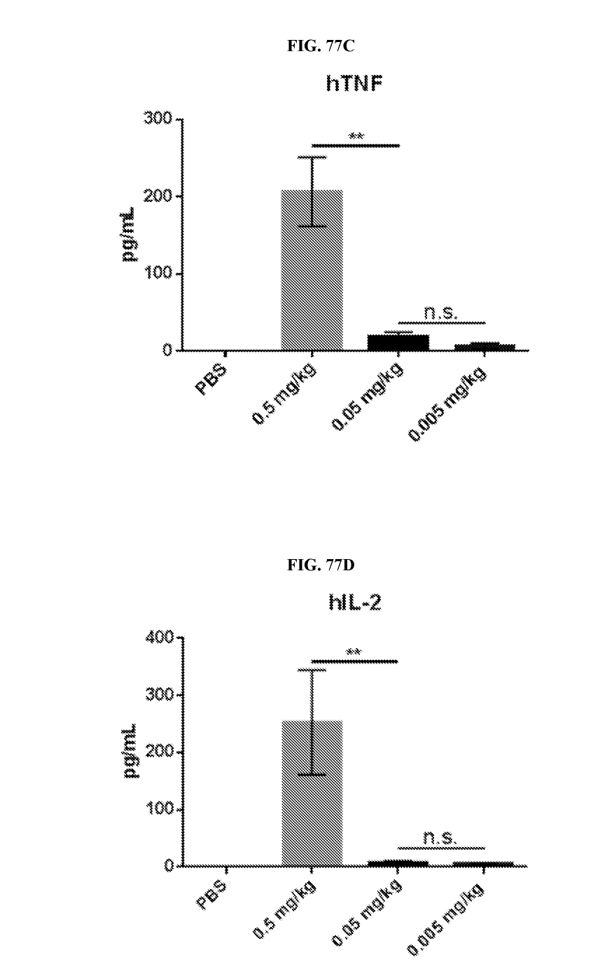

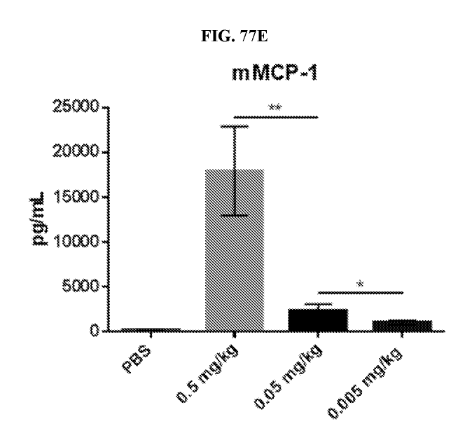

Adverse events such as severe CRS have been associated with the administration of CAR-T cells to patients with high tumor burden, which likely triggers massive antigen specific CAR-T cell expansion and activation. Therefore, it was determined whether the switch could be dose titrated to achieve a gradual tumor clearance to avoid the acute toxicity. In these dose-escalation studies, treatment of tumor bearing mice was initiated with the effective (0.5 mg/kg) or suboptimal (0.05 mg/kg) dose (FIG. 66C). Significant body weight loss was observed in mice treated with a starting dose at 0.5 mg/kg of anti-CD19 AB-FITC, whereas mice treated with 0.05 mg/kg did not lose weight to a similar extent (FIG. 66D, raw data shown in Table 27). Furthermore, evaluation of serum cytokines ˜24 hours after CAR-T cells and switch injections revealed a dose-dependent elevation of human (IFN γ, TNFα, and IL-2) and mouse (MCP-1) cytokines (FIG. 77B-E). After three doses, animals that received 0.05 mg/kg of AB-FITC were further divided into two groups: group (a) in which the switch dose was maintained at 0.05 mg/kg, or group (b) in which treatment was continued with an elevated switch dose (0.5 mg/kg). Given that a majority of the tumor burden was reduced with the first three suboptimal doses of 0.05 mg/kg, the subsequent increase in switch dose to 0.5 mg/kg did not result in significant weight loss (FIG. 66D and 77A, raw data shown in Table 43). More importantly, the dose escalation regimen achieved tumor clearance comparable to animals that were started with high dose switch (0.5 mg/kg, FIGS. 66C and 77A). Collectively, these results demonstrate that the activity of sCAR-T cells can be controlled by switch dosage to minimize treatment-related toxicities while retaining potent anti-tumor activity. This ability to control the temporal activation of the CAR-T cell response may be helpful clinically to ameliorate adverse events associated with the administration of CAR-T cells to patients with high tumor burden, such as severe CRS.

### Example 20

### Overcoming B Cell Aplasia by Switch Dose Termination

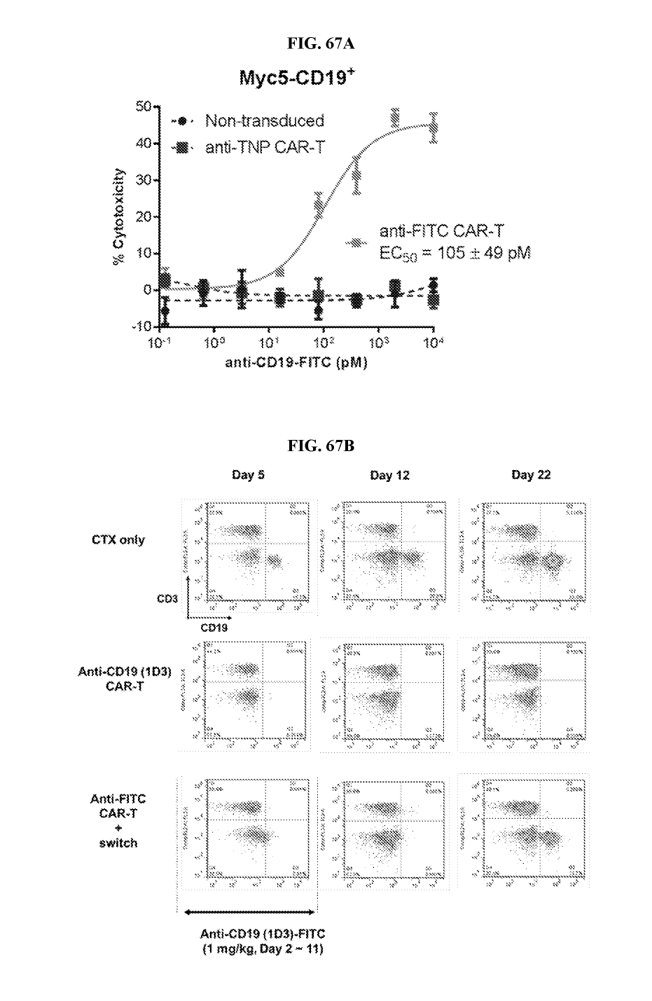

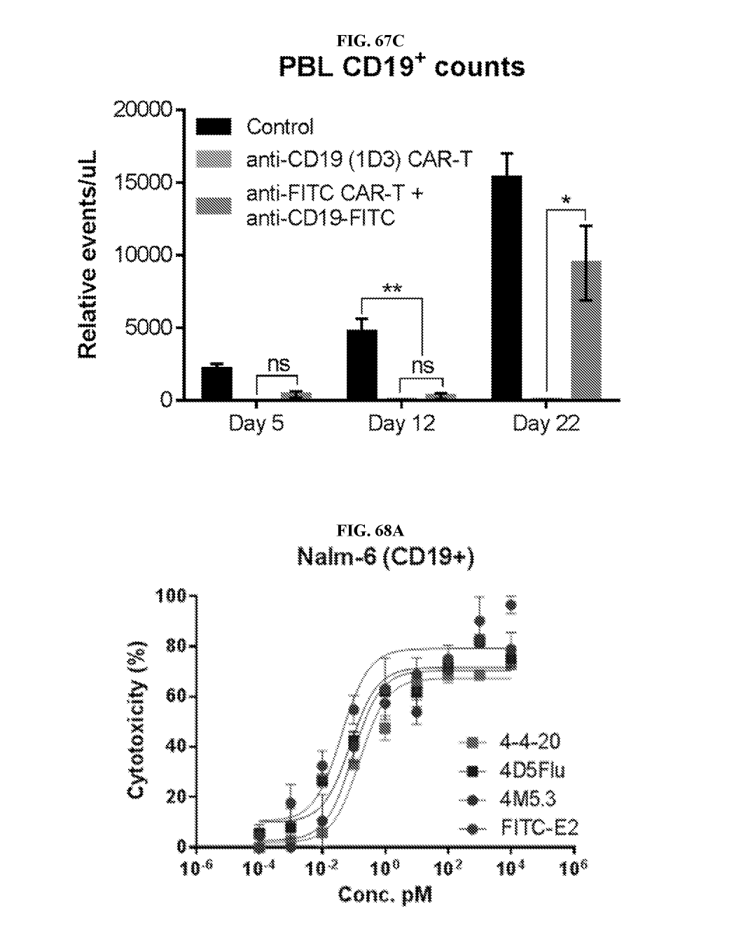

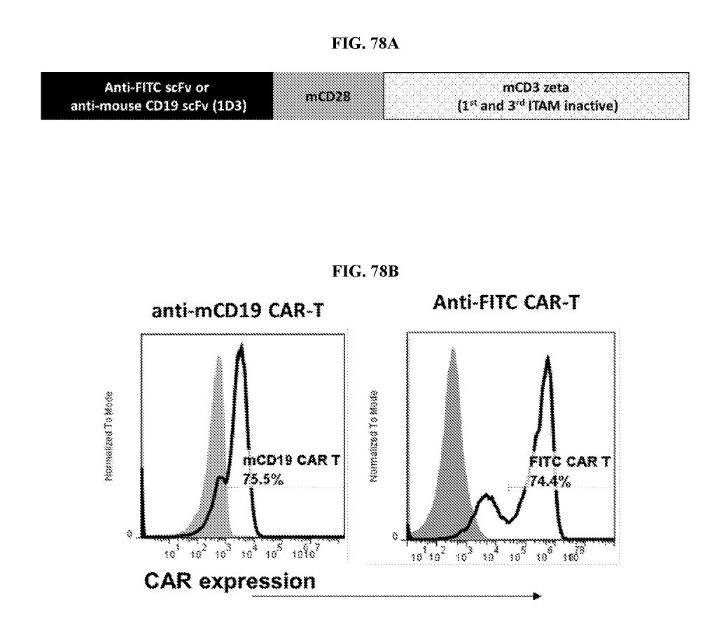

In addition to CRS, another major safety issue associated with current CART 19 therapy is the persistent ablation of normal B cells. Therefore, it was determined whether the CAR-T switch platform could be used to minimize this “on-target”-related adverse event. Towards this end, a second generation mouse surrogate anti-FITC CAR was generated based on the reported anti-mouse CD19 CAR vector, which encodes for anti-mouse CD19 scFv (clone 1D3) and mouse signaling domains (CD28 and CD3ζ) in a retroviral backbone (FIG. 78A). Murine anti-FITC sCAR-T cells were generated using splenocytes from immunocompetent C57BL/6 mice as previously described and tested for cytotoxicity against CD19⁻ Myc5 cells in the presence of an anti-mouse CD19 (1D3) FITC switch conjugated at the distal position of the light chain (Ser202). As shown in FIG. 67A, mouse anti-FITC sCAR-T cells induced switch-dependent target cell lysis (EC₅₀=105±49 pM). No activity was observed with non-transduced mouse T cells or with a CAR-T cell specific for 2,4,6-trinitrophenyl (TNP) group in the presence of murine FITC-anti-CD19 switch.

The activity of this CAR-T was determined in a surrogate B cell depletion model. In this model, C57BL/6 mice were preconditioned with cyclophosphamide (150 mg/kg) on day 1. The next day, 6×10⁶ of syngeneic anti-mouse CD19 or anti-FITC sCAR-T cells (˜75% transduction efficiency) were infused. Mice that had received anti-FITC sCAR-T cells were injected daily IV with anti-mouse CD19 FITC switch at 1 mg/kg (day 2-11). To assess the depletion of B cells, CD3⁺ and CD19⁺ cells in peripheral blood were monitored by flow cytometry (FIG. 67B-C). By day 5 of the study (after 3 injections of switch), peripheral B cell loss was observed in both CAR-T groups. On day 12 of the study, switch treatment was halted and animals were monitored for repopulation of CD19⁺ cells. In agreement with previous reports, a persistent loss of CD19⁺ cells was observed with the anti-mouse CD19 CAR (up to day 22). By contrast, there was repopulation of CD19⁺ cells in the peripheral blood of animals treated with the anti-FITC sCAR-T in adjunct with anti-CD19 FITC switch on day 22 (11 days after the last dose of switch) (FIG. 67B-C). This study demonstrates that a sCAR-T approach allows the CAR-T response to be “turned-off” by discontinuation of switch dosing once the desired efficacy is achieved, and can potentially prevent adverse effects associated with the persistent activity of CAR-T cells.

### Example 21

### Summary of FITC Switch sCAR-T Findings

Herein, is described a general approach to optimize hapten-based sCAR-Ts. Using a site-specific protein conjugation method, a panel of homogeneously FITC-labeled antibody switches was generated that mediate distinct spatial interactions between sCAR-T and cancer cells. This approach was applied to optimize switches to target Her2, CD19, CLL1 (all well studied and validated antigens for conventional CAR T therapies), CD22, CD33, and CD123 cell surface antigens. In vitro studies demonstrate that specific FITC conjugation sites and increasing valency of FITC conjugates results in increased sCAR-T cell activity against target cells, measured by sCAR-T cell activation markers and cytokine production, as well as levels of cytotoxicity induced their respective target cells. This indicates that switches can be customized for specific target antigens to achieve the maximal anti-tumor response.

More importantly, in vitro observations regarding site specificity for optimal target cell killing were confirmed in vivo. Optimal switches for targeting CD19, Her2, and CLL1 were determined through methods described herein and were applied in various murine xenograft models to assess the in vivo functionality of the sCAR-T cell and FITC-switch platform. In these models, the sCAR-T cell combined with the optimized FITC-switch resulted in tumor ablation comparable to responses seen with conventional CART cells.

These results, summarized in Table 16 demonstrate that specific switches can be optimized to a given target through modulating the distance of the conjugation sites from the antigen binding domain and altering the number of FITC conjugates in order to generate the optimal distances and geometries required for optimal pseudo-immune synapse formation and signaling. These optimization results suggest that the difference in optimal conjugation sites is likely a reflection of the need to create a pseudo-immunological synapse that is similar in distance to physiological TCR/MHC complexes, which is reportedly 130˜150 angstrom, while maintaining accessibility to anti-FITC scFv. Optimization also allows for the potential to induce in situ dimerization of the CAR. Indeed, in studies using peptide neo-epitope (PNE) grafted switches, anti-PNE CAR-T cell activity was improved when a mutation was introduced within the hinge region to enhance the dimerization of CAR via inter-chain disulfide bond. Importantly, randomly conjugated FITC switches demonstrated inferior in vitro and in vivo anti-tumor activity, highlighting the importance of site-specific conjugations. While in general, distance of the FITC conjugates relative to the antigen binding domain had marked effects on the activity of the resultant switch-sCAR-T cell complex, it is also possible that distances between the FITC conjugates themselves may be altered to increase the resultant complex activity, as the distance between conjugates in proximal and distal bivalent switches was not constant.

TABLE 16
Summary of anti-FITC sCAR-T cell and FITC-switch experimental data

| | | | | | | In vivo models |
|---|---|---|---|---|---|---|
| | | | | | Substantial | used to |
| | | | | | Cytotoxicity | confirm anti- |
| | | | | | observed in | tumor activity |
| | | | | | combination with | in combination |
| | | Effect of | Most effective | sCART | anti-FITC CAR-T | with anti-FITC |
| Target | Clone | Valency | conjugation sites | hinge | cells in cell lines: | sCAR-T cells |
| Her2 | Herceptin | Bivalent | Distal | CD8 | SKCR3, MDA 453, | HCC1954 |
| | | more robust | LS202X/HK136X | | MDA 431 | MDA 453, |
| | | | (E/F) | | MDA 468, BT20 | MDA 435 |
| | | | | | | MDA231, |
| | | | | | | subcutaneous in |
| | | | | | | flank or mammary |
| | | | | | | fat pad |
| CD19 | FMC63 | Bivalent | Proximal | CD8 | Nalm6 | Nalm6 |
| | | more robust | LG68/HS74 (A/B) | | | |
| CD22 | hLL2 | Bivalent | Distal | CD8 | Daudi, Raji, | |
| | | more robust | LG74/HS75 (E/F) | | Nalm6 | |
| | M971 | Bivalent | Distal | CD8 | | |
| | | more robust | LG68/HS78 (E/F) | | | |
| CLL1 | 1075.7 | Bivalent | Proximal | CD8 | U937, HL60 | U937 |
| | | more robust | LG69/HS75 (A/B) | | | subcutaneous |
| | | | | | | AML Model |
| CD33 | hM195 | Bivalent | Intermediate | CD8 | U937, THP-1, | Planned |
| | | more robust | LT113/HA117 (C/D) | | MOLM14 | |
| | Hp67.6 | Bivalent | Proximal | CD8 | | |
| | | more robust | LG72/HP75 (A/B) | | | |
| CD123 | 26292 | Bivalent | Distal | CD8 | MOLM13, | |
| | | more robust | LSA202/HK131 (E/F) | | KASUMI | |
| | 32716 | No effect | Proximal | CD8 | | Planned |
| | | | LR72/HS75 (A/B) | | | |

To establish whether the in vivo sCAR-T cell activity can be controlled with a switch, the approach was evaluated in Nalm-6 tumor xenograft models. In these studies, switch-mediated anti-tumor activity and proliferation of CAR-T cell was strictly switch dose dependent. More importantly, it was demonstrated that treatment related toxicities, such as body weight loss and elevated serum cytokines, were also switch-dose related. These preclinical studies demonstrate that controlling in vivo CAR-T activity via a switch dosing regimen may improve the safety profile while preserving the potent anti-tumor response.

Overcoming toxicities related to persistent CAR-T cell activity, such as B cell aplasia, was also demonstrated with the sCART approach. In immunocompetent mice, it was shown that CD19-targeting by CAR-T cells is reversible by simply terminating switch dosing. Furthermore, this demonstration of the reversibility of CAR-T cell activity in surrogate models may expand the potential application of the sCAR-T approach to other indications such as acute myeloid leukemia (AML) and solid tumors, to which the long-term persistence of CAR-T cells may pose a greater safety risk.

A general method for producing site-specifically conjugated antibody-FITC switches has been demonstrated that elicit potent anti-FITC CAR-T cell effector functions. Furthermore, it has been shown that the ability to chemically define specific conjugation sites significantly influenced the efficacy of anti-CD19 and anti-CD22 switch molecules. The versatility of this platform has been shown by targeting two different antigens with a single CAR. This aspect of this strategy should be useful in treating tumor escape variants or heterogeneous tumors expressing distinct tumor antigens, and also can simplify manufacturing of CAR-Ts for different indications (single CAR encoding vector). Importantly, using an optimized sCAR-T system for CD19, potent in vivo anti-tumor activity was achieved that is comparable to a clinically validated CAR-T therapy. In addition, it was demonstrated that this switchable approach may provide a way to prevent or manage major safety issues associated with current CD19 targeting CAR therapies, such as severe CRS and long term B-cell depletion. Finally, the ability to regulate in vivo activity as well as the specificity of engineered T cells with a soluble intermediate switch will allow for the safe application of this potent immune cell-based therapy to target other types of cancer including solid tumors as well as non-oncology indications.

### Example 22

### Methods of Making and Using CAR T Cell Switches Mouse Xenograft Cancer Models

Six to eight weeks old female NSG mice were intravenously inoculated with 0.5×10⁶ Nalm-6 cells transfected with luciferase and engraftment was confirmed by bioluminescence imaging. The next day, CAR-T cells were infused and treatment with indicated anti-CD19 (clone FMC63) FITC switches was initiated. In parallel, control groups (tumor only, CAR-T cells only, and tumor-bearing mice that received CART-19 T cells) were injected with PBS. Body weight was monitored daily, and tumor growth was monitored weekly by bioluminescence imaging.

The expansion of human T cells in the peripheral blood was monitored using weekly retro-orbital bleeds. Briefly, 40 μL of blood was incubated with PE-conjugated anti-CD3 (OKT3) conjugated at room temperature for 30 min. Next, red blood cells were lysed with 10× FACS Lysing Solution (BD Biosciences) according to manufacturer's instructions and remaining lymphocytes were washed with staining buffer. Liquid counting beads (BD Biosciences) were added prior to analysis on a BD Accuri C6, where 33 μL out of 330 μL was acquired from each sample. To enumerate the number of CD3-positive cells per microliter of blood, the following formula was used: Relative CD3⁻ events/uL=[Number of CD3⁺ events×Percentage of acquired volume]/[Volume of blood used for staining (uL)].

To determine whether the initial weight loss observed is associated with an elevated systemic cytokines and/or increased tumor lysis, serum from retro-orbital bleeds taken 24 hours after the initial switch treatment were used. Serum cytokines were quantified using BD CBA Human Th1/Th2 Kit II and Mouse Inflammation Kit according to product manuals. Uric acid and phosphate levels were also assessed using fluorescent-based assay kits (Abcam), but inconclusive results were obtained (data not shown).

sCAR-T Cell Engraftment

To facilitate the engraftment of CAR-T cells, six to eight weeks old C57BL/6 mice were preconditioned with 150 mg/kg of cyclophosphamide (Sigma) on day 1. The next day, 6×10⁶ syngeneic anti-FITC CAR-T cells and anti-CD19 (1D3) FITC switch (1 mg/kg) were sequentially administered by tail vein injections. Thereafter, switch molecules were injected daily at 1 mg/kg for a total of 10 doses (day 2-11). As a positive control, a separate cohort of mice received T cells transduced with the conventional anti-mouse CD19 (ID3) CAR. To assess the efficacy and specificity of mouse sCAR-T cells, CD3- and CD19-positive populations in the peripheral blood were monitored once a week for the duration of the study using PE-conjugated anti-mouse CD3 (2C11, Biolegend) and FITC-conjugated anti-mouse CD19 (6D5, Biolegend)—to evaluate the loss and repopulation of B cells during and after treatment, respectively. Unstained and single color controls were acquired and used for compensation.

### Synthesis of FITC CAR Switches

Synthesis of BCN-PEG₄-FITC (1) is shown below:

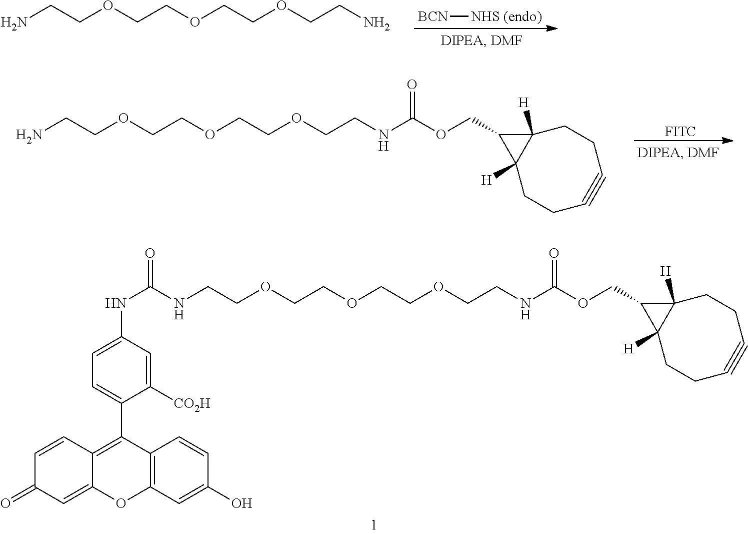

A solution of BCN-NHS (endo) (130 mg, 0.446 mmol) in anhydrous DMF (3 mL) was added dropwise to a solution of 1,11-diamino-3,6,9-trioxaundecane (515 mg, 2.68 mmol) and N,N-diisopropylethylamine (0.39 mL, 2.23 mmol) in DMF (5 mL). After stirring for 15 min, the reaction mixture was concentrated in vacuo. The remaining residue was dissolved in dichloromethane (100 mL) and washed with 1N NaOH (10 mL×2) and H₂O (10 mL) sequentially. The dichloromethane layer was dried over anhydrous sodium sulfate, filtered, and concentrated in vacuo.

The crude residue was dissolved in DMF (5 mL), followed by the addition of N,N-diisopropylethylamine (0.23 mL, 1.32 mmol). A solution of FITC (171 mg, 0.40 mmol) in DMF (4 mL) was added dropwise to the mixture over 5 min. After 1 hour of stirring, the reaction mixture was concentrated in vacuo and purified by column chromatography to yield the desired product (215 mg, 0.284 mmol, 64% yield for 2 steps).

¹H NMR (DMSO-d₆, 400 MHz) δ (ppm) 10.07 (s, br, 2H), 9.99 (s, br, 1H), 8.24 (s, 1H), 8.05 (s, br, 1H), 7.70 (d, 1H, J=8 Hz), 7.14 (d, 1H, J=8 Hz), 7.02 (s, br, 1H), 6.64-6.63 (m, 2H), 6.58-6.51 (m ,4H), 4.29-4.25 (m, 1H), 3.98 (d, 2H, J=8 Hz), 3.68-3.63 (m, 2H), 3.58-3.55 (m, 2H), 3.49-3.45 (m, 4H), 3.36-3.29 (m, 4H), 3.17-3.14 (m, 1H), 3.09-3.05 (m, 2H), 2.21-2.08 (m, 5H), 1.70-1.68 (m, 1H), 1.48-1.46 (m, 2H), 1.22-1.17 (m, 1H), 0.83-0.77 (m, 2H).

¹³C NMR (DMSO-d₆, 100 MHz) δ (ppm) 181.23, 169.33, 160.33, 157.26, 152.71, 129.85, 113.41, 110.56, 103.06, 99.81, 70.57, 70.37, 69.97, 67.53, 62.20, 41.27, 29.81, 29.42, 26.01, 21.01, 20.38, 18.49.

HRMS Calcd. for C₄₀H₄₄N₃O₁₁S ([M+H]⁻): 742.2970. Found: 742.2971.

Synthesis of FITC-PEG₄-NHS (2) is shown below:

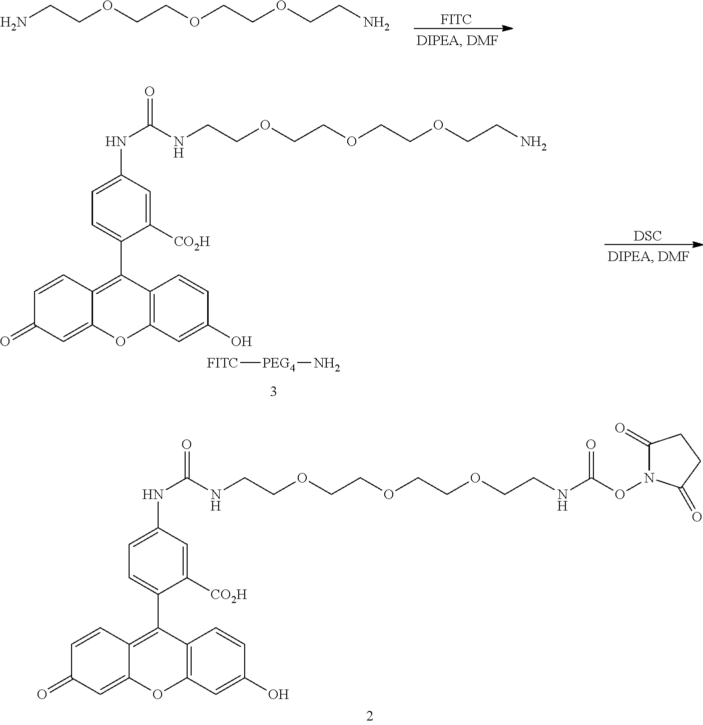

A solution of FITC (400 mg, 0.925 mmol) in anhydrous DMF (20 mL) was added dropwise to a solution of 1,11-diamino-3,6,9-trioxaundecane (1.0 g, 5.20 mmol) and N,N-diisopropylethylamine (1.0 mL, 5.73 mmol) in DMF (15 mL). After stirring for 30 min, the reaction mixture was concentrated in vacuo, and purified by preparative HPLC to yield FITC-PEG₄-NH₂ as TFA salt (compound 3, 574 mg, 0.826 mmol, 89%).

Compound 3 (TFA salt, 146 mg, 0.210 mmol) was dissolved in DMF (5 mL), followed by the addition of N,N-diisopropylethylamine (0.18 mL, 1.05 mmol). Next, N,N′-disuccinimidyl carbonate (81 mg, 0.315 mmol) was added to the reaction mixture. After stirring for 15 min, the reaction mixture was concentrated in vacuo, and purified by preparative HPLC to yield the desired product (2) (50.5 mg, 0.0699 mmol, 33%).

¹H NMR (DMSO-d₆, 400 MHz) δ (ppm) 10.06 (s, br, 1H), 8.35 (s, br, 1H), 8.28 (s, 1H), 7.74 (d, 1H, J=8 Hz), 7.18 (d, 1H, J=8 Hz), 6.76-6.75 (m, 2H), 6.68-6.55 (m, 4H), 3.71-3.67 (m, 2H), 3.62-3.60 (m, 2H), 3.58-3.52 (m, 8H), 3.47-3.44 (m, 2H), 3.23-3.17 (m, 2H), 2.75 (s, 4H).

¹³C NMR (DMSO-d₆, 100 MHz) δ (ppm) 182.32, 171.69, 169.33, 160.37, 152.71, 135.10, 129.86, 113.42, 110.55, 103.06, 70.60, 70.45, 70.40, 70.32, 70.20, 69.34, 41.84, 33.92, 26.09.

HRMS Calcd. for C₃₄H₃₅N₄O₁₃S ([M+H]⁻): 723.1967. Found: 723.1970.

### Expression and Generation of Bifunctional FITC Switch Molecules

Anti-human CD19 Fab (clone FMC63), anti-human CD22 Fab (clone M971), and anti-mouse CD19 Fab (clone 1D3) sequences were cloned into pBAD vectors and site-specific mutations to introduce TAG amber nonsense codon were generated using Quikchange Site-directed Mutagenesis Kit (Stratagene). Antibodies were expressed in Escherichia coli (E.coli) with an orthogonal Methanococcus jannaschii tRNA/aminoacyl-tRNA synthetase specific for p-azido phenylalanine (pAzF) and purified. Purity and incorporation of UAA was confirmed by SDS-PAGE gel and mass spectrometry (QTOF).

Mutant antibodies containing pAzF (≤1 mg/mL) were conjugated with 30-fold molar excess of BCN-PEG₄-FITC (1) in phosphate-buffered saline (PBS) pH 7.4 and incubated overnight at 37° C. The next day, completion of conjugation reaction was confirmed by QTOF, excess linkers were removed by size filtration (Amicon, 10K and 30K), and the size and purity of the final products were confirmed by SDS-PAGE gel.

For random anti-CD19 FITC conjugates, wildtype anti-CD19 Fab were expressed in E. coli and purified as above. After size and purity were confirmed, antibodies were incubated with 48-fold molar excess of FITC-PEG₄-NHS in PBS at 37° C. for 6 hours. Excess small molecules were removed by size filtration (Amicon, 10K and 30 K) and final product was analyzed on an Agilent Quadruple Time-of-Flight (QTOF) mass spectrometer and deconvoluted masses were obtained using Agilent Qualitative Analysis software. Random anti-CD19 FITC conjugates were also subject to CESI-MS analysis: Unmodified and random FITC labeled antibodies were prepared at 1 mg/mL using a 4-hour digestion protocol with RapiGest DTT, iodoacetamide, and trypsin, then diluted to 250 mg/mL in 125 mM ammonium acetate, pH 4. In parallel, intact antibodies were prepared at 1 mg/mL in 50 mM ammonium acetate, pH 4. CESI experiments were carried out on a SCIEX TripleTOF® 6600 system with a NanoSpray® III source and SCIEX CESI 8000 system. High resolution MS and MS/MS spectra were analyzed using SCIEX ProteinPilot™, PeakView®, and BioPharmaView™ softwares. Both MS strategies indicate that the random FITC conjugate has a drug-to-antibody ratio (DAR) of 2, where FITC conjugation was localized to the N-terminal glutamate of the heavy chain, and lysine residues at positions 31 and 145 within the light chain. To remove bacterial endotoxins, anti-CD19 FITC switch molecules were filtered with Mustang Q membranes (Pall) and confirmed endotoxin levels were <10 endotoxin units/mL using the Endosafe®-PTS system (Charles River).

### Cell Lines and Culturing Conditions

Leukemia and lymphoma cell lines (Nalm-6, Daudi, Raji, IM-9, and K562) were purchased from ATCC and maintained in RPMI 1640 media supplemented with 10% heat-inactivated fetal bovine serum (FBS, Hyclone) and 1 mM sodium pyruvate (Life Technologies). Virus producing cell lines, HEK293T and Platinum E (Plat E, Cell Biolabs), were maintained in DMEM media with the following additions: 10% FBS, 2 mM Glutamax, MEM non-essential amino acids, and 1 mM sodium pyruvate. Human PBMC and transduced CAR-T cells were cultured in AIM V media (Life Technologies) with added 5% human AB serum (Valley Biomedical). Mouse splenocytes and transduced CAR-T cells were cultured in RPMI 1640 fully supplemented with 10% FBS (Gemini Bioproducts), 5 mM HEPES, 1.5 mM L-glutamine, 50 μM 2-mercaptoethanol, and 0.05 mg/mL Gentamicin (Life Technologies). All media contained 100 units/mL of penicillin and 100 μg/mL of streptomycin. Unless specified, all media and supplements were purchased from Life Technologies.

### Binding Assays

Binding of anti-CD19 FITC conjugates was confirmed with Nalm-6 (CD19⁺) and anti-FITC CAR-T cells by flow cytometry. Briefly, cells were incubated with indicated switch antibodies at 4° C. for 30-60 min and washed twice with staining buffer (1% BSA in PBS). Primary antibodies were revealed with Alexa Fluor®647 conjugated anti-human IgG or anti-human kappa secondary antibodies. After several washes, samples were acquired on a BD LSR II or BD Accuri C6 and analyzed using FlowJo 7.6.2 software. In each study, cells were incubated with secondary antibody alone and the observed mean fluorescence intensity (MFI) was used to subtract for background and non-specific staining.

### Virus Production and Generation of CAR-T Cells

A gene cassette containing the human anti-FITC scFv, CD8α hinge and transmembrane region, and the cytoplasmic domains of 41BB and CD3ζ was synthesized by Genescript and cloned into the LV-vector. Lentivirus production and transduction of human T cells were performed. Briefly, HEK293FT cells were transfected with anti-FITC CAR plasmid and viral packaging vectors and 48 hours later, supernatants containing lentivirus were harvested or frozen at −80° C. until ready for use. PBMCs were isolated from normal healthy blood using Ficoll-Paque density gradient approach (GE healthcare), and activated with CD3/CD28 activation beads (Life Technologies) at 3:1 bead to cell ratio for 24 hours. Activated T cells were mixed with supernatant containing lentivirus in the presence of protamine sulfate (1 ug/ml), centrifuged at 1000×g for 90 min, and incubated overnight at 37° C. The next day, viral supernatant were replaced with fresh media containing recombinant human IL-2 (300 IU/mL; R&D systems). Transduced T cells were maintained at 0.125-2×10⁶ cells/mL in media containing IL-2, which was replenished every 2-3 days.

For mouse anti-FITC CAR-T cells, the anti-mouse CD19 (1D3) scFv within the MSGV1 1D3-28Z. 1-3 plasmid was replaced with the human anti-FITC scFv. The mouse version of anti-FITC CAR consists of the human anti-FITC scFv, murine CD28 (excluding the N-terminus of the extracellular domain) and the cytoplasmic domain of murine CD3ζ. Retrovirus supernatants were produced using Plat E cells and used directly to transduce activated mouse splenocytes (C57BL/6) following a spinoculation protocol using retronectin (Takara). Transduced mouse CAR-T cells were maintained at 0.5×10⁶ cells/mL in media containing recombinant human IL-2 (60 IU/mL). The original anti-mouse CD19 CAR and an irrelevant CAR, which recognizes 2,4,6-trinitrophenyl, were transduced in parallel and used as positive and negative controls, respectively.

Transduction efficiency was verified by flow cytometry using Alexa Fluor®647 conjugated anti-mouse or anti-human IgG F(ab)′2 antibodies (Jackson ImmunoResearch). Anti-FITC CAR expression was also confirmed using a FITC-labeled mouse IgG1 isotype control antibody (Biolegend). Non-transduced T cells labeled with F(ab)′2 antibodies served as background controls.

### Flow Cytometry-Based Cytotoxicity Assays

Target cells (1×10⁴ cells), pre-labeled with CellVue® Claret Far Red Fluorescent Cell linker (Sigma), were co-cultured with CAR-T cells at indicated E:T (effector-to-target) ratios in 96-well round bottom plates supplemented with different concentrations of switch molecules, and incubated at 37° C. For competition assays, in addition to switch molecules, cultures also consisted of excess amounts of fluorescein (Sigma) or anti-CD19 antibody (or isotype control; Millipore). For CART-19 and anti-FITC CAR-T cell comparison, pre-labeled target cells were incubated with effector cells in the presence of 1 nM anti-CD19 AB-FITC switch. After 24 hours, cells were labeled with 1:100 dilution of 7-AAD viability dye (BD Biosciences) in staining buffer, acquired on a BD Accuri C6 flow cytometer, and analyzed using FlowJo 7.6.2 software. Unstained and single color controls were acquired and compensation was done using FlowJo. The following formula was used to determine the percent cytotoxic activity: (values used represent percentage of Claret Red⁺/7AAD⁻ populations) % Cytotoxicity=100×[((Target cells+Effector cells only)−(Target cells+Effector cells+Switch))/(Target cells+Effector cells only)].

### Colorimetric-Based Cytotoxicity Assays

Co-cultures containing 1×10⁴ target cells (murine CD19 overexpressing Myc5 cells, and 1×10⁵ anti-FITC CAR-T cells with different concentrations of anti-CD19 (1D3) FITC switch were incubated at 37° C. for 6 hours. Cytotoxicity was measured using the Cytotox-96 Nonradioactive cytotoxicity assay kit (Promega), which quantifies the amount of lactate dehydrogenase (LDH) released from lysed cells into the supernatant. The percent lytic activity was calculated with the following formula: (values used represent absorbance at 490 nM) % Cytotoxicity=100×[((Target cells+Effector cells+Switch)−(Target cells+Effector cells only))/((Maximum target cell lysis)−(Target cells only))].

### CD19 and CD22 Expression Quantification

Cell surface CD19 and CD22 expression levels were quantified on indicated leukemia and lymphoma cell lines using PE-conjugated anti-CD19 (HIB19, BD Biosciences) and anti-CD22 (4JB128, ebioscience). To quantify the number of antigens per cell, the observed MFI from test samples were compared with a standard curve established with BD Quanti-Brite PE beads (bead MFI vs. number of PE molecules per bead) according to product manual.

### Enrichment of CAR-T Cells

To enrich for anti-CD19 and anti-FITC CAR expressing T cells, cells were pre-incubated at 4° C. for 30 min with biotin-conjugated anti-mouse or anti-human IgG F(ab)′2 antibodies (Jackson Immunoresearch), respectively. Labeled cells were incubated with anti-Biotin microbeads (Milltenyi) and separated according to manufacturer's instructions. Purity of CAR-T cells were confirmed by flow cytometry and verified once a week using Alexa Fluor®647 conjugated anti-mouse IgG F(ab)′2 or anti-human IgG F(ab)′2 antibodies as described above. Enriched cells were used for experimentation after 1-2 passages, with each passage taking place every 2-3 days.

### Activation and BcL-xL Upregulation Assay

Equal number (1×10⁵) of target and enriched CAR-T cells were co-cultured in the presence of 1 nM anti-CD19 AB-FITC switch in 96 well round bottom plates at 37° C. for 24 hours. The next day, cultures were labeled with APC-conjugated anti-CD3 (OKT3), PerCP/Cy5.5-conjugated CD25 (BC96) and PE-conjugated CD69 (FN50) antibodies (all purchased from Biolegend). To evaluate the upregulation of BcL-xl, cells were surface stained with APC-conjugated anti-CD3 (OKT3), then fix and permeabilized using Cyotfix/Cytoperm kit (BD Biosciences) prior to intracellular staining with anti-Bcl-xl antibody (7B2.5, Abcam). Appropriate isotype controls were included in each study to determine background and exclude non-specific staining. Unstained and single color controls were acquired and used for compensation.

### Cytokine Release Assays

Cytokines in cultured media from activation studies were quantified using BD CBA Human Th1/Th2 Kit II (BD Biosciences) according to manufacturer's protocol.

### Statistical Analysis

All graphs and statistics were conducted using the Graphpad Prism 6.0 software.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

TABLE 17
CAR-Nucleotide Sequence

| NAME | SEQ ID | SEQUENCE |
|---|---|---|
| LV- | 1 | caggtggcacttttcggggaaatgtgcgcggaacccctatttgtttatttttctaaatacattcaaatatgtatccg |
| EF1a-4- | | ctcatgagacaataaccctgataaatgcttcaataatattgaaaaaggaagagtatgagtattcaacatttccgt |
| 4-20- | | gtcgcccttattcccttttttgcggcattttgccttcctgtttttgctcacccagaaacgctggtgaaagtaaaaga |
| BBZ | | tgctgaagatcagttgggtgcacgagtgggttacatcgaactggatctcaacagcggtaagatccttgagagt |
| | | tttcgccccgaagaacgttttccaatgatgagcacttttaaagttctgctatgtggcgcggtattatcccgtattg |
| | | acgccgggcaagagcaactcggtcgccgcatacactattctcagaatgacttggttgagtactcaccagtca |
| | | cagaaaagcatcttacggatggcatgacagtaagagaattatgcagtgctgccataaccatgagtgataaca |
| | | ctgcggccaacttacttctgacaacgatcggaggaccgaaggagctaaccgcttttttgcacaacatggggg |
| | | atcatgtaactcgccttgatcgttgggaaccggagctgaatgaagccataccaaacgacgagcgtgacacc |
| | | acgatgcctgtagcaatggcaacaacgttgcgcaaactattaactggcgaactacttactctagcttcccggc |
| | | aacaattaatagactggatggaggcggataaagttgcaggaccacttctgcgctcggcccttccggctggct |
| | | ggtttattgctgataaatctggagccggtgagcgtgggtctcgcggtatcattgcagcactggggccagatg |
| | | gtaagccctcccgtatcgtagttatctacacgacggggagtcaggcaactatggatgaacgaaatagacaga |
| | | tcgctgagataggtgcctcactgattaagcattggtaactgtcagaccaagtttactcatatatactttagattgat |
| | | ttaaaacttcatttttaatttaaaaggatctaggtgaagatcctttttgataatctcatgaccaaaatcccttaacgt |
| | | gagttttcgttccactgagcgtcagaccccgtagaaaagatcaaaggatcttcttgagatcctttttttctgcgcg |
| | | taatctgctgcttgcaaacaaaaaaaccaccgctaccagcggtggtttgtttgccggatcaagagctaccaac |
| | | tctttttccgaaggtaactggcttcagcagagcgcagataccaaatactgtccttctagtgtagccgtagttagg |
| | | ccaccacttcaagaactctgtagcaccgcctacatacctcgctctgctaatcctgttaccagtggctgctgcca |
| | | gtggcgataagtcgtgtcttaccgggttggactcaagacgatagttaccggataaggcgcagcggtcgggc |
| | | tgaacggggggttcgtgcacacagcccagcttggagcgaacgacctacaccgaactgagatacctacagc |
| | | gtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgggtt |
| | | tcgccacctctgacttgagcgtcgatttttgtgatgctcgtcaggggggcggagcctatggaaaaacgccag |
| | | caacgcggcctttttacggttcctggccttttgctggccttttgctcacatgttctttcctgcgttatcccctgattct |
| | | gtggataaccgtattaccgcctttgagtgagctgataccgctcgccgcagccgaacgaccgagcgcagcga |
| | | gtcagtgagcgaggaagcggaagagcgcccaatacgcaaaccgcctctccccgcgcgttggccgattcat |
| | | taatgcagctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagcggata |
| | | acaatttcacacaggaaacagctatgaccatgattacgccaagcgcgcaattaaccctcactaaagggaaca |
| | | aaagctggagctgcaagcttaatgtagtcttatgcaatactcttgtagtcttgcaacatggtaacgatgagttag |
| | | caacatgccttacaaggagagaaaaagcaccgtgcatgccgattggtggaagtaaggtggtacgatcgtgc |
| | | cttattaggaaggcaacagacgggtctgacatggattggacgaaccactgaattgccgcattgcagagatatt |
| | | gtatttaagtgcctagctcgatacaataaacgggtctctctggttagaccagatctgagcctgggagctctctg |
| | | gctaactagggaacccactgcttaagcctcaataaagcttgccttgagtgcttcaagtagtgtgtgcccgtctg |
| | | ttgtgtgactctggtaactagagatccctcagacccttttagtcagtgtggaaaatctctagcagtggcgcccg |
| | | aacagggacctgaaagcgaaagggaaaccagagctctctcgacgcaggactcggcttgctgaagcgcgc |
| | | acggcaagaggcgaggggcggcgactggtgagtacgccaaaaattttgactagcggaggctagaaggag |
| | | agagatgggtgcgagagcgtcagtattaagcgggggagaattagatcgcgatgggaaaaaattcggttaag |
| | | gccagggggaaagaaaaaatataaattaaaacatatagtatgggcaagcagggagctagaacgattcgca |
| | | gttaatcctggcctgttagaaacatcagaaggctgtagacaaatactgggacagctacaaccatcccttcaga |
| | | caggatcagaagaacttagatcattatataatacagtagcaaccctctattgtgtgcatcaaaggatagagata |
| | | aaagacaccaaggaagctttagacaagatagaggaagagcaaaacaaaagtaagaccaccgcacagcaa |
| | | gcggccgctgatcttcagacctggaggaggagatatgagggacaattggagaagtgaattatataaatataa |
| | | agtagtaaaaattgaaccattaggagtagcacccaccaaggcaaagagaagagtggtgcagagagaaaaa |
| | | agagcagtgggaataggagctttgttccttgggttcttgggagcagcaggaagcactatgggcgcagcctc |
| | | aatgacgctgacggtacaggccagacaattattgtctggtatagtgcagcagcagaacaatttgctgagggc |
| | | tattgaggcgcaacagcatctgttgcaactcacagtctggggcatcaagcagctccaggcaagaatcctggc |
| | | tgtggaaagatacctaaaggatcaacagctcctggggatttggggttgctctggaaaactcatttgcaccact |
| | | gctgtgccttggaatgctagttggagtaataaatctctggaacagattggaatcacacgacctggatggagtg |
| | | ggacagagaaattaacaattacacaagcttaatacactccttaattgaagaatcgcaaaaccagcaagaaaa |
| | | gaatgaacaagaattattggaattagataaatgggcaagtttgtggaattggtttaacataacaaattggctgtg |
| | | gtatataaaattattcataatgatagtaggaggcttggtaggtttaagaatagtttttgctgtactttctatagtgaat |
| | | agagttaggcagggatattcaccattatcgtttcagacccacctcccaaccccgaggggacccgacaggcc |
| | | cgaaggaatagaagaagaaggtggagagagagacagagacagatccattcgattagtgaacggatctcga |
| | | cggttaacttttaaaagaaaaggggggattggggggtacagtgcaggggaaagaatagtagacataatagc |
| | | aacagacatacaaactaaagaattacaaaaacaaattacaaaaattcaaaattttatcgagctttgcaaagatg |
| | | gataaagttttaaacagagaggaatctttgcagctaatggaccttctaggtcttgaaaggagtgcctcgtgagg |
| | | ctccggtgcccgtcagtgggcagagcgcacatcgcccacagtccccgagaagttggggggaggggtcgg |
| | | caattgaaccggtgcctagagaaggtggcgcggggtaaactgggaaagtgatgtcgtgtactggctccgcc |
| | | tttttcccgagggtgggggagaaccgtatataagtgcagtagtcgccgtgaacgttctttttcgcaacgggttt |
| | | gccgccagaacacaggtaagtgccgtgtgtggttcccgcgggcctggcctctttacgggttatggcccttgc |
| | | gtgccttgaattacttccacctggctgcagtacgtgattcttgatcccgagcttcgggttggaagtgggtggga |
| | | gagttcgaggccttgcgcttaaggagccccttcgcctcgtgcttgagttgaggcctggcctgggcgctgggg |
| | | ccgccgcgtgcgaatctggtggcaccttcgcgcctgtctcgctgctttcgataagtctctagccatttaaaatttt |
| | | tgatgacctgctgcgacgctttttttctggcaagatagtcttgtaaatgcgggccaagatctgcacactggtattt |
| | | cggtttttggggccgcgggcggcgacggggcccgtgcgtcccagcgcacatgttcggcgaggcggggc |
| | | ctgcgagcgcggccaccgagaatcggacgggggtagtctcaagctggccggcctgctctggtgcctggc |
| | | ctcgcgccgccgtgtatcgccccgccctgggcggcaaggctggcccggtcggcaccagttgcgtgagcg |
| | | gaaagatggccgcttcccggccctgctgcagggagctcaaaatggaggacgcggcgctcgggagagcg |
| | | ggcgggtgagtcacccacacaaaggaaaagggcctttccgtcctcagccgtcgcttcatgtgactccacgg |
| | | agtaccgggcgccgtccaggcacctcgattagttctcgagcttttggagtacgtcgtctttaggttgggggga |
| | | ggggttttatgcgatggagtttccccacactgagtgggtggagactgaagttaggccagcttggcacttgatg |
| | | taattctccttggaatttgccctttttgagtttggatcttggttcattctcaagcctcagacagtggttcaaagtttttt |
| | | tcttccatttcaggtgtcgtgaggaattcggtaccgcggccgcccggggatccatggccttaccagtgaccg |
| | | ccttgctcctgccgctggccttgctgctccacgccgccaggccggatgttgttatgacccagaccccgctgtc |
| | | cctgcccgtttccctgggtgaccaggcgagtatctcttgccgttcctctcagtccctggttcacagtcagggta |
| | | acacctatctgcgctggtatctgcaaaaaccaggccagagccctaaagtgctgatttataaggtttcaaatcg |
| | | gtttagcggcgtcccggatcgcttctctgggagtggatcagggaccgactttacactgaaaattagccgcgtg |
| | | gaagcagaggatctgggcgtgtacttttgcagccagtccactcatgtgccgtggaccttcggcggtgggaca |
| | | aaactggaaattaagcgtgcaggaggcggtgggagcggaggcggtgggtccggaggcggtgggtctgg |
| | | aggcggtgggagtggaggcggtgggtcaggcggtggtgggagcgaagtgaaactggatgagacagga |
| | | ggaggtctggttcagccaggtcggcccatgaagctgtcctgtgtggcctctggctttaccttctccgactattg |
| | | gatgaactgggtccgtcagtctccggaaaaaggtctggagtgggtggcgcagattcggaacaagccctaca |
| | | actacgaaacttactactctgatagtgttaaaggccgcttcaccatcagtcgtgatgactcaaaaagcagcgtt |
| | | tacctgcaaatgaacaatctgcgtgtcgaggacatgggcatctattactgcacaggctcctactatgggatgg |
| | | attattgggggcaggggacttcagttacagtttcctcaaccacgacgccagcgccgcgaccaccaacaccg |
| | | gcgcccaccatcgcgtcgcagcccctgtccctgcgcccagaggcgtgccggccagcggcggggggcgc |
| | | agtgcacacgagggggctggacttcgcctgtgatatctacatctgggcgcccttggccgggacttgtggggt |
| | | ccttctcctgtcactggttatcaccctttactgcaaacggggcagaaagaaactcctgtatatattcaaacaacc |
| | | atttatgagaccagtacaaactactcaagaggaagatggctgtagctgccgatttccagaagaagaagaagg |
| | | aggatgtgaactgagagtgaagttcagcaggagcgcagacgcccccgcgtacaagcagggccagaacc |
| | | agctctataacgagctcaatctaggacgaagagaggagtacgatgttttggacaagagacgtggccgggac |
| | | cctgagatggggggaaagccgagaaggaagaaccctcaggaaggcctgtacaatgaactgcagaaagat |
| | | aagatggcggaggcctacagtgagattgggatgaaaggcgagcgccggaggggcaaggggcacgatg |
| | | gcctttaccagggtctcagtacagccaccaaggacacctacgacgcccttcacatgcaggccctgccccct |
| | | cgctaagtcgacaatcaacctctggattacaaaatttgtgaaagattgactggtattcttaactatgttgctcctttt |
| | | acgctatgtggatacgctgctttaatgcctttgtatcatgctattgcttcccgtatggctttcattttctcctccttgta |
| | | taaatcctggttgctgtctctttatgaggagttgtggcccgttgtcaggcaacgtggcgtggtgtgcactgtgttt |
| | | gctgacgcaacccccactggttggggcattgccaccacctgtcagctcctttccgggactttcgctttccccct |
| | | ccctattgccacggcggaactcatcgccgcctgccttgcccgctgctggacaggggctcggctgttgggca |
| | | ctgacaattccgtggtgttgtcggggaagctgacgtcctttccatggctgctcgcctgtgttgccacctggattc |
| | | tgcgcgggacgtccttctgctacgtcccttcggccctcaatccagcggaccttccttcccgcggcctgctgcc |
| | | ggctctgcggcctcttccgcgtcttcgccttcgccctcagacgagtcggatctccctttgggccgcctccccg |
| | | cctggaattcgagctcggtacctttaagaccaatgacttacaaggcagctgtagatcttagccactttttaaaag |
| | | aaaaggggggactggaagggctaattcactcccaacgaagacaagatctgctttttgcttgtactgggtctct |
| | | ctggttagaccagatctgagcctgggagctctctggctaactagggaacccactgcttaagcctcaataaag |
| | | cttgccttgagtgcttcaagtagtgtgtgcccgtctgttgtgtgactctggtaactagagatccctcagacccttt |
| | | tagtcagtgtggaaaatctctagcagtagtagttcatgtcatcttattattcagtatttataacttgcaaagaaatg |
| | | aatatcagagagtgagaggaacttgtttattgcagcttataatggttacaaataaagcaatagcatcacaaattt |
| | | cacaaataaagcatttttttcactgcattctagttgtggtttgtccaaactcatcaatgtatcttatcatgtctggctc |
| | | tagctatcccgcccctaactccgcccagttccgcccattctccgccccatggctgactaattttttttatttatgca |
| | | gaggccgaggccgcctcggcctctgagctattccagaagtagtgaggaggcttttttggaggcctaggctttt |
| | | gcgtcgagacgtacccaattcgccctatagtgagtcgtattacgcgcgctcactggccgtcgttttacaacgtc |
| | | gtgactgggaaaaccctggcgttacccaacttaatcgccttgcagcacatccccctttcgccagctggcgtaa |
| | | tagcgaagaggcccgcaccgatcgcccttcccaacagttgcgcagcctgaatggcgaatggcgcgacgc |
| | | gccctgtagcggcgcattaagcgcggcgggtgtggtggttacgcgcagcgtgaccgctacacttgccagc |
| | | gccctagcgcccgctcctttcgctttcttcccttcctttctcgccacgttcgccggctttccccgtcaagctctaa |
| | | atcgggggctccctttagggttccgatttagtgctttacggcacctcgaccccaaaaaacttgattagggtgat |
| | | ggttcacgtagtgggccatcgccctgatagacggtttttcgccctttgacgttggagtccacgttctttaatagt |
| | | ggactcttgttccaaactggaacaacactcaaccctatctcggtctattcttttgatttataagggattttgccgatt |
| | | tcggcctattggttaaaaaatgagctgatttaacaaaaatttaacgcgaattttaacaaaatattaacgtttacaat |
| | | ttcc |
| LV- | 2 | caggtggcacttttcggggaaatgtgcgcggaacccctatttgtttatttttctaaatacattcaaatatgtatccg |
| EF1a- | | ctcatgagacaataaccctgataaatgcttcaataatattgaaaaaggaagagtatgagtattcaacatttccgt |
| 4D5F1u- | | gtcgcccttattcccttttttgcggcattttgccttcctgtttttgctcacccagaaacgctggtgaaagtaaaaga |
| BBZ | | tgctgaagatcagttgggtgcacgagtgggttacatcgaactggatctcaacagcggtaagatccttgagagt |
| | | tttcgccccgaagaacgttttccaatgatgagcacttttaaagttctgctatgtggcgcggtattatcccgtattg |
| | | acgccgggcaagagcaactcggtcgccgcatacactattctcagaatgacttggttgagtactcaccagtca |
| | | cagaaaagcatcttacggatggcatgacagtaagagaattatgcagtgctgccataaccatgagtgataaca |
| | | ctgcggccaacttacttctgacaacgatcggaggaccgaaggagctaaccgcttttttgcacaacatggggg |
| | | atcatgtaactcgccttgatcgttgggaaccggagctgaatgaagccataccaaacgacgagcgtgacacc |
| | | acgatgcctgtagcaatggcaacaacgttgcgcaaactattaactggcgaactacttactctagcttcccggc |
| | | aacaattaatagactggatggaggcggataaagttgcaggaccacttctgcgctcggcccttccggctggct |
| | | ggtttattgctgataaatctggagccggtgagcgtgggtctcgcggtatcattgcagcactggggccagatg |
| | | gtaagccctcccgtatcgtagttatctacacgacggggagtcaggcaactatggatgaacgaaatagacaga |
| | | tcgctgagataggtgcctcactgattaagcattggtaactgtcagaccaagtttactcatatatactttagattgat |
| | | ttaaaacttcatttttaatttaaaaggatctaggtgaagatcctttttgataatctcatgaccaaaatcccttaacgt |
| | | gagttttcgttccactgagcgtcagaccccgtagaaaagatcaaaggatcttcttgagatcctttttttctgcgcg |
| | | taatctgctgcttgcaaacaaaaaaaccaccgctaccagcggtggtttgtttgccggatcaagagctaccaac |
| | | tctttttccgaaggtaactggcttcagcagagcgcagataccaaatactgtccttctagtgtagccgtagttagg |
| | | ccaccacttcaagaactctgtagcaccgcctacatacctcgctctgctaatcctgttaccagtggctgctgcca |
| | | gtggcgataagtcgtgtcttaccgggttggactcaagacgatagttaccggataaggcgcagcggtcgggc |
| | | tgaacggggggttcgtgcacacagcccagcttggagcgaacgacctacaccgaactgagatacctacagc |
| | | gtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgggtt |
| | | tcgccacctctgacttgagcgtcgatttttgtgatgctcgtcaggggggcggagcctatggaaaaacgccag |
| | | caacgcggcctttttacggttcctggccttttgctggccttttgctcacatgttctttcctgcgttatcccctgattct |
| | | gtggataaccgtattaccgcctttgagtgagctgataccgctcgccgcagccgaacgaccgagcgcagcga |
| | | gtcagtgagcgaggaagcggaagagcgcccaatacgcaaaccgcctctccccgcgcgttggccgattcat |
| | | taatgcagctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagcggata |
| | | acaatttcacacaggaaacagctatgaccatgattacgccaagcgcgcaattaaccctcactaaagggaaca |
| | | aaagctggagctgcaagcttaatgtagtcttatgcaatactcttgtagtcttgcaacatggtaacgatgagttag |
| | | caacatgccttacaaggagagaaaaagcaccgtgcatgccgattggtggaagtaaggtggtacgatcgtgc |
| | | cttattaggaaggcaacagacgggtctgacatggattggacgaaccactgaattgccgcattgcagagatatt |
| | | gtatttaagtgcctagctcgatacaataaacgggtctctctggttagaccagatctgagcctgggagctctctg |
| | | gctaactagggaacccactgcttaagcctcaataaagcttgccttgagtgcttcaagtagtgtgtgcccgtctg |
| | | ttgtgtgactctggtaactagagatccctcagacccttttagtcagtgtggaaaatctctagcagtggcgcccg |
| | | aacagggacctgaaagcgaaagggaaaccagagctctctcgacgcaggactcggcttgctgaagcgcgc |
| | | acggcaagaggcgaggggcggcgactggtgagtacgccaaaaattttgactagcggaggctagaaggag |
| | | agagatgggtgcgagagcgtcagtattaagcgggggagaattagatcgcgatgggaaaaaattcggttaag |
| | | gccagggggaaagaaaaaatataaattaaaacatatagtatgggcaagcagggagctagaacgattcgca |
| | | gttaatcctggcctgttagaaacatcagaaggctgtagacaaatactgggacagctacaaccatcccttcaga |
| | | caggatcagaagaacttagatcattatataatacagtagcaaccctctattgtgtgcatcaaaggatagagata |
| | | aaagacaccaaggaagctttagacaagatagaggaagagcaaaacaaaagtaagaccaccgcacagcaa |
| | | gcggccgctgatcttcagacctggaggaggagatatgagggacaattggagaagtgaattatataaatataa |
| | | agtagtaaaaattgaaccattaggagtagcacccaccaaggcaaagagaagagtggtgcagagagaaaaa |
| | | agagcagtgggaataggagctttgttccttgggttcttgggagcagcaggaagcactatgggcgcagcctc |
| | | aatgacgctgacggtacaggccagacaattattgtctggtatagtgcagcagcagaacaatttgctgagggc |
| | | tattgaggcgcaacagcatctgttgcaactcacagtctggggcatcaagcagctccaggcaagaatcctggc |
| | | tgtggaaagatacctaaaggatcaacagctcctggggatttggggttgctctggaaaactcatttgcaccact |
| | | gctgtgccttggaatgctagttggagtaataaatctctggaacagattggaatcacacgacctggatggagtg |
| | | ggacagagaaattaacaattacacaagcttaatacactccttaattgaagaatcgcaaaaccagcaagaaaa |
| | | gaatgaacaagaattattggaattagataaatgggcaagtttgtggaattggtttaacataacaaattggctgtg |
| | | gtatataaaattattcataatgatagtaggaggcttggtaggtttaagaatagtttttgctgtactttctatagtgaat |
| | | agagttaggcagggatattcaccattatcgtttcagacccacctcccaaccccgaggggacccgacaggcc |
| | | cgaaggaatagaagaagaaggtggagagagagacagagacagatccattcgattagtgaacggatctcga |
| | | cggttaacttttaaaagaaaaggggggattggggggtacagtgcaggggaaagaatagtagacataatagc |
| | | aacagacatacaaactaaagaattacaaaaacaaattacaaaaattcaaaattttatcgagctttgcaaagatg |
| | | gataaagttttaaacagagaggaatctttgcagctaatggaccttctaggtcttgaaaggagtgcctcgtgagg |
| | | ctccggtgcccgtcagtgggcagagcgcacatcgcccacagtccccgagaagttggggggaggggtcgg |
| | | caattgaaccggtgcctagagaaggtggcgcggggtaaactgggaaagtgatgtcgtgtactggctccgcc |
| | | tttttcccgagggtgggggagaaccgtatataagtgcagtagtcgccgtgaacgttctttttcgcaacgggttt |
| | | gccgccagaacacaggtaagtgccgtgtgtggttcccgcgggcctggcctctttacgggttatggcccttgc |
| | | gtgccttgaattacttccacctggctgcagtacgtgattcttgatcccgagcttcgggttggaagtgggtggga |
| | | gagttcgaggccttgcgcttaaggagccccttcgcctcgtgcttgagttgaggcctggcctgggcgctgggg |
| | | ccgccgcgtgcgaatctggtggcaccttcgcgcctgtctcgctgctttcgataagtctctagccatttaaaatttt |
| | | tgatgacctgctgcgacgctttttttctggcaagatagtcttgtaaatgcgggccaagatctgcacactggtattt |
| | | cggtttttggggccgcgggcggcgacggggcccgtgcgtcccagcgcacatgttcggcgaggcggggc |
| | | ctgcgagcgcggccaccgagaatcggacgggggtagtctcaagctggccggcctgctctggtgcctggc |
| | | ctcgcgccgccgtgtatcgccccgccctgggcggcaaggctggcccggtcggcaccagttgcgtgagcg |
| | | gaaagatggccgcttcccggccctgctgcagggagctcaaaatggaggacgcggcgctcgggagagcg |
| | | ggcgggtgagtcacccacacaaaggaaaagggcctttccgtcctcagccgtcgcttcatgtgactccacgg |
| | | agtaccgggcgccgtccaggcacctcgattagttctcgagcttttggagtacgtcgtctttaggttgggggga |
| | | ggggttttatgcgatggagtttccccacactgagtgggtggagactgaagttaggccagcttggcacttgatg |
| | | taattctccttggaatttgccctttttgagtttggatcttggttcattctcaagcctcagacagtggttcaaagtttttt |
| | | tcttccatttcaggtgtcgtgaggaattcggtaccgcggccgcccggggatccatggccttaccagtgaccg |
| | | ccttgctcctgccgctggccttgctgctccacgccgccaggccggacattcagatgactcagagcccgtctt |
| | | ccctgtccgcttcagtgggtgaccgtgttaccattacctgccgtgcttctcagagcctggtgcactcccagggt |
| | | aacacctatctgcggtggtatcagcagaaaccgggcaaggcccccaaagttctgatttataaggtctctaatc |
| | | ggtttagtggcgttccgtcacgcttctcaggtagcgggtccggaacagattttaccctgacaattagcagcctg |
| | | caaccggaagacttcgcaacctattactgccagcagtctacccatgtgccctggacatttggccagggtacta |
| | | aggttgagctgaaacgtgctggaggtggaggaagcggaggtggaggcagcggcggtgggggatctggc |
| | | ggtgggggaagtggcggtgggggatcaggcggtgggggaagcgaagtccagctggtggagagtggag |
| | | gtggactggtgcagccaggaggcagcctgcgtctgtcctgtgccgcgtctggctttaccttcagcgattactg |
| | | gatgaactgggtccgtcaggcaccaggaaagggcctggaatgggtggcgcagattcggaacaagccttac |
| | | aactacgagacttactacgcggatagcgtgaagggtcgcttcaccatcagtcgtgacacatcaaagaacact |
| | | gtttacctgcaaatgaacagcctgcgtgcagaagataccgctgtctattactgcacaggctcttattacggcat |
| | | ggactactggggccagggtactctggtgaccgtttctagtaccacgacgccagcgccgcgaccaccaaca |
| | | ccggcgcccaccatcgcgtcgcagcccctgtccctgcgcccagaggcgtgccggccagcggcgggggg |
| | | cgcagtgcacacgagggggctggacttcgcctgtgatatctacatctgggcgcccttggccgggacttgtg |
| | | gggtccttctcctgtcactggttatcaccctttactgcaaacggggcagaaagaaactcctgtatatattcaaac |
| | | aaccatttatgagaccagtacaaactactcaagaggaagatggctgtagctgccgatttccagaagaagaag |
| | | aaggaggatgtgaactgagagtgaagttcagcaggagcgcagacgcccccgcgtacaagcagggccag |
| | | aaccagctctataacgagctcaatctaggacgaagagaggagtacgatgttttggacaagagacgtggccg |
| | | ggaccctgagatggggggaaagccgagaaggaagaaccctcaggaaggcctgtacaatgaactgcaga |
| | | aagataagatggcggaggcctacagtgagattgggatgaaaggcgagcgccggaggggcaaggggcac |
| | | gatggcctttaccagggtctcagtacagccaccaaggacacctacgacgcccttcacatgcaggccctgcc |
| | | ccctcgctaagtcgacaatcaacctctggattacaaaatttgtgaaagattgactggtattcttaactatgttgct |
| | | ccttttacgctatgtggatacgctgctttaatgcctttgtatcatgctattgcttcccgtatggctttcattttctcctc |
| | | cttgtataaatcctggttgctgtctctttatgaggagttgtggcccgttgtcaggcaacgtggcgtggtgtgcac |
| | | tgtgtttgctgacgcaacccccactggttggggcattgccaccacctgtcagctcctttccgggactttcgcttt |
| | | ccccctccctattgccacggcggaactcatcgccgcctgccttgcccgctgctggacaggggctcggctgtt |
| | | gggcactgacaattccgtggtgttgtcggggaagctgacgtcctttccatggctgctcgcctgtgttgccacct |
| | | ggattctgcgcgggacgtccttctgctacgtcccttcggccctcaatccagcggaccttccttcccgcggcct |
| | | gctgccggctctgcggcctcttccgcgtcttcgccttcgccctcagacgagtcggatctccctttgggccgcc |
| | | tccccgcctggaattcgagctcggtacctttaagaccaatgacttacaaggcagctgtagatcttagccactttt |
| | | taaaagaaaaggggggactggaagggctaattcactcccaacgaagacaagatctgctttttgcttgtactgg |
| | | gtctctctggttagaccagatctgagcctgggagctctctggctaactagggaacccactgcttaagcctcaat |
| | | aaagcttgccttgagtgcttcaagtagtgtgtgcccgtctgttgtgtgactctggtaactagagatccctcagac |
| | | ccttttagtcagtgtggaaaatctctagcagtagtagttcatgtcatcttattattcagtatttataacttgcaaaga |
| | | aatgaatatcagagagtgagaggaacttgtttattgcagcttataatggttacaaataaagcaatagcatcaca |
| | | aatttcacaaataaagcatttttttcactgcattctagttgtggtttgtccaaactcatcaatgtatcttatcatgtctg |
| | | gctctagctatcccgcccctaactccgcccagttccgcccattctccgccccatggctgactaattttttttattta |
| | | tgcagaggccgaggccgcctcggcctctgagctattccagaagtagtgaggaggcttttttggaggcctag |
| | | gcttttgcgtcgagacgtacccaattcgccctatagtgagtcgtattacgcgcgctcactggccgtcgttttaca |
| | | acgtcgtgactgggaaaaccctggcgttacccaacttaatcgccttgcagcacatccccctttcgccagctgg |
| | | cgtaatagcgaagaggcccgcaccgatcgcccttcccaacagttgcgcagcctgaatggcgaatggcgcg |
| | | acgcgccctgtagcggcgcattaagcgcggcgggtgtggtggttacgcgcagcgtgaccgctacacttgc |
| | | cagcgccctagcgcccgctcctttcgctttcttcccttcctttctcgccacgttcgccggctttccccgtcaagct |
| | | ctaaatcgggggctccctttagggttccgatttagtgctttacggcacctcgaccccaaaaaacttgattaggg |
| | | tgatggttcacgtagtgggccatcgccctgatagacggtttttcgccctttgacgttggagtccacgttctttaat |
| | | agtggactcttgttccaaactggaacaacactcaaccctatctcggtctattcttttgatttataagggattttgcc |
| | | gatttcggcctattggttaaaaaatgagctgatttaacaaaaatttaacgcgaattttaacaaaatattaacgttta |
| | | caatttcc |
| LV- | 3 | caggtggcacttttcggggaaatgtgcgcggaacccctatttgtttatttttctaaatacattcaaatatgtatccg |
| EF1a- | | ctcatgagacaataaccctgataaatgcttcaataatattgaaaaaggaagagtatgagtattcaacatttccgt |
| 4M5.3- | | gtcgcccttattcccttttttgcggcattttgccttcctgtttttgctcacccagaaacgctggtgaaagtaaaaga |
| BBZ | | tgctgaagatcagttgggtgcacgagtgggttacatcgaactggatctcaacagcggtaagatccttgagagt |
| | | tttcgccccgaagaacgttttccaatgatgagcacttttaaagttctgctatgtggcgcggtattatcccgtattg |
| | | acgccgggcaagagcaactcggtcgccgcatacactattctcagaatgacttggttgagtactcaccagtca |
| | | cagaaaagcatcttacggatggcatgacagtaagagaattatgcagtgctgccataaccatgagtgataaca |
| | | ctgcggccaacttacttctgacaacgatcggaggaccgaaggagctaaccgcttttttgcacaacatggggg |
| | | atcatgtaactcgccttgatcgttgggaaccggagctgaatgaagccataccaaacgacgagcgtgacacc |
| | | acgatgcctgtagcaatggcaacaacgttgcgcaaactattaactggcgaactacttactctagcttcccggc |
| | | aacaattaatagactggatggaggcggataaagttgcaggaccacttctgcgctcggcccttccggctggct |
| | | ggtttattgctgataaatctggagccggtgagcgtgggtctcgcggtatcattgcagcactggggccagatg |
| | | gtaagccctcccgtatcgtagttatctacacgacggggagtcaggcaactatggatgaacgaaatagacaga |
| | | tcgctgagataggtgcctcactgattaagcattggtaactgtcagaccaagtttactcatatatactttagattgat |
| | | ttaaaacttcatttttaatttaaaaggatctaggtgaagatcctttttgataatctcatgaccaaaatcccttaacgt |
| | | gagttttcgttccactgagcgtcagaccccgtagaaaagatcaaaggatcttcttgagatcctttttttctgcgcg |
| | | taatctgctgcttgcaaacaaaaaaaccaccgctaccagcggtggtttgtttgccggatcaagagctaccaac |
| | | tctttttccgaaggtaactggcttcagcagagcgcagataccaaatactgtccttctagtgtagccgtagttagg |
| | | ccaccacttcaagaactctgtagcaccgcctacatacctcgctctgctaatcctgttaccagtggctgctgcca |
| | | gtggcgataagtcgtgtcttaccgggttggactcaagacgatagttaccggataaggcgcagcggtcgggc |
| | | tgaacggggggttcgtgcacacagcccagcttggagcgaacgacctacaccgaactgagatacctacagc |
| | | gtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgggtt |
| | | tcgccacctctgacttgagcgtcgatttttgtgatgctcgtcaggggggcggagcctatggaaaaacgccag |
| | | caacgcggcctttttacggttcctggccttttgctggccttttgctcacatgttctttcctgcgttatcccctgattct |
| | | gtggataaccgtattaccgcctttgagtgagctgataccgctcgccgcagccgaacgaccgagcgcagcga |
| | | gtcagtgagcgaggaagcggaagagcgcccaatacgcaaaccgcctctccccgcgcgttggccgattcat |
| | | taatgcagctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagcggata |
| | | acaatttcacacaggaaacagctatgaccatgattacgccaagcgcgcaattaaccctcactaaagggaaca |
| | | aaagctggagctgcaagcttaatgtagtcttatgcaatactcttgtagtcttgcaacatggtaacgatgagttag |
| | | caacatgccttacaaggagagaaaaagcaccgtgcatgccgattggtggaagtaaggtggtacgatcgtgc |
| | | cttattaggaaggcaacagacgggtctgacatggattggacgaaccactgaattgccgcattgcagagatatt |
| | | gtatttaagtgcctagctcgatacaataaacgggtctctctggttagaccagatctgagcctgggagctctctg |
| | | gctaactagggaacccactgcttaagcctcaataaagcttgccttgagtgcttcaagtagtgtgtgcccgtctg |
| | | ttgtgtgactctggtaactagagatccctcagacccttttagtcagtgtggaaaatctctagcagtggcgcccg |
| | | aacagggacctgaaagcgaaagggaaaccagagctctctcgacgcaggactcggcttgctgaagcgcgc |
| | | acggcaagaggcgaggggcggcgactggtgagtacgccaaaaattttgactagcggaggctagaaggag |
| | | agagatgggtgcgagagcgtcagtattaagcgggggagaattagatcgcgatgggaaaaaattcggttaag |
| | | gccagggggaaagaaaaaatataaattaaaacatatagtatgggcaagcagggagctagaacgattcgca |
| | | gttaatcctggcctgttagaaacatcagaaggctgtagacaaatactgggacagctacaaccatcccttcaga |
| | | caggatcagaagaacttagatcattatataatacagtagcaaccctctattgtgtgcatcaaaggatagagata |
| | | aaagacaccaaggaagctttagacaagatagaggaagagcaaaacaaaagtaagaccaccgcacagcaa |
| | | gcggccgctgatcttcagacctggaggaggagatatgagggacaattggagaagtgaattatataaatataa |
| | | agtagtaaaaattgaaccattaggagtagcacccaccaaggcaaagagaagagtggtgcagagagaaaaa |
| | | agagcagtgggaataggagctttgttccttgggttcttgggagcagcaggaagcactatgggcgcagcctc |
| | | aatgacgctgacggtacaggccagacaattattgtctggtatagtgcagcagcagaacaatttgctgagggc |
| | | tattgaggcgcaacagcatctgttgcaactcacagtctggggcatcaagcagctccaggcaagaatcctggc |
| | | tgtggaaagatacctaaaggatcaacagctcctggggatttggggttgctctggaaaactcatttgcaccact |
| | | gctgtgccttggaatgctagttggagtaataaatctctggaacagattggaatcacacgacctggatggagtg |
| | | ggacagagaaattaacaattacacaagcttaatacactccttaattgaagaatcgcaaaaccagcaagaaaa |
| | | gaatgaacaagaattattggaattagataaatgggcaagtttgtggaattggtttaacataacaaattggctgtg |
| | | gtatataaaattattcataatgatagtaggaggcttggtaggtttaagaatagtttttgctgtactttctatagtgaat |
| | | agagttaggcagggatattcaccattatcgtttcagacccacctcccaaccccgaggggacccgacaggcc |
| | | cgaaggaatagaagaagaaggtggagagagagacagagacagatccattcgattagtgaacggatctcga |
| | | cggttaacttttaaaagaaaaggggggattggggggtacagtgcaggggaaagaatagtagacataatagc |
| | | aacagacatacaaactaaagaattacaaaaacaaattacaaaaattcaaaattttatcgagctttgcaaagatg |
| | | gataaagttttaaacagagaggaatctttgcagctaatggaccttctaggtcttgaaaggagtgcctcgtgagg |
| | | ctccggtgcccgtcagtgggcagagcgcacatcgcccacagtccccgagaagttggggggaggggtcgg |
| | | caattgaaccggtgcctagagaaggtggcgcggggtaaactgggaaagtgatgtcgtgtactggctccgcc |
| | | tttttcccgagggtgggggagaaccgtatataagtgcagtagtcgccgtgaacgttctttttcgcaacgggttt |
| | | gccgccagaacacaggtaagtgccgtgtgtggttcccgcgggcctggcctctttacgggttatggcccttgc |
| | | gtgccttgaattacttccacctggctgcagtacgtgattcttgatcccgagcttcgggttggaagtgggtggga |
| | | gagttcgaggccttgcgcttaaggagccccttcgcctcgtgcttgagttgaggcctggcctgggcgctgggg |
| | | ccgccgcgtgcgaatctggtggcaccttcgcgcctgtctcgctgctttcgataagtctctagccatttaaaatttt |
| | | tgatgacctgctgcgacgctttttttctggcaagatagtcttgtaaatgcgggccaagatctgcacactggtattt |
| | | cggtttttggggccgcgggcggcgacggggcccgtgcgtcccagcgcacatgttcggcgaggcggggc |
| | | ctgcgagcgcggccaccgagaatcggacgggggtagtctcaagctggccggcctgctctggtgcctggc |
| | | ctcgcgccgccgtgtatcgccccgccctgggcggcaaggctggcccggtcggcaccagttgcgtgagcg |
| | | gaaagatggccgcttcccggccctgctgcagggagctcaaaatggaggacgcggcgctcgggagagcg |
| | | ggcgggtgagtcacccacacaaaggaaaagggcctttccgtcctcagccgtcgcttcatgtgactccacgg |
| | | agtaccgggcgccgtccaggcacctcgattagttctcgagcttttggagtacgtcgtctttaggttgggggga |
| | | ggggttttatgcgatggagtttccccacactgagtgggtggagactgaagttaggccagcttggcacttgatg |
| | | taattctccttggaatttgccctttttgagtttggatcttggttcattctcaagcctcagacagtggttcaaagtttttt |
| | | tcttccatttcaggtgtcgtgaggaattcggtaccgcggccgcccggggatccatggccttaccagtgaccg |
| | | ccttgctcctgccgctggccttgctgctccacgccgccaggccggatgttgttatgacacagaccccgctga |
| | | gtctgcccgtctccctgggcgaccaggcctccatttcttgccgtagttcacagtccctggtgcactctaacgga |
| | | aatacctatctgcgctggtacctgcagaaaccgggccagagccccaaagtcctgatctataaggtgtccaac |
| | | cgggtgtctggtgttccggatcgcttttcagggagcggcagcggcaccgacttcacactgaagatcaatcgt |
| | | gttgaagcagaggatctgggcgtctacttttgctctcagagtacacatgtgccatggactttcggcggtggga |
| | | ccaaactggaaattaagagctccgcagatgacgctaaaaaggacgccgcgaaaaaggatgacgccaaaa |
| | | aggatgacgcgaaaaaggatggaggcgtcaaactggacgagaccggtgggggactggtgcagccgggc |
| | | ggtgcaatgaagctgagttgtgtgacttcaggttttaccttcgggcactattggatgaactgggttcgtcagagt |
| | | ccagaaaaaggtctggagtgggtcgctcagtttcggaacaagccttacaactacgaaacatactactcagat |
| | | agcgttaaagggcgcttcactattagccgtgatgactccaagtctagtgtgtacctgcagatgaacaatctgc |
| | | gggttgaggacacaggcatctattactgcacaggggcgtcctatggtatggagtatctggggcagggaaca |
| | | agcgtcaccgtctcaaccacgacgccagcgccgcgaccaccaacaccggcgcccaccatcgcgtcgcag |
| | | cccctgtccctgcgcccagaggcgtgccggccagcggcggggggcgcagtgcacacgagggggctgg |
| | | acttcgcctgtgatatctacatctgggcgcccttggccgggacttgtggggtccttctcctgtcactggttatca |
| | | ccctttactgcaaacggggcagaaagaaactcctgtatatattcaaacaaccatttatgagaccagtacaaact |
| | | actcaagaggaagatggctgtagctgccgatttccagaagaagaagaaggaggatgtgaactgagagtga |
| | | agttcagcaggagcgcagacgcccccgcgtacaagcagggccagaaccagctctataacgagctcaatct |
| | | aggacgaagagaggagtacgatgttttggacaagagacgtggccgggaccctgagatggggggaaagc |
| | | cgagaaggaagaaccctcaggaaggcctgtacaatgaactgcagaaagataagatggcggaggcctaca |
| | | gtgagattgggatgaaaggcgagcgccggaggggcaaggggcacgatggcctttaccagggtctcagta |
| | | cagccaccaaggacacctacgacgcccttcacatgcaggccctgccccctcgctaagtcgacaatcaacct |
| | | ctggattacaaaatttgtgaaagattgactggtattcttaactatgttgctccttttacgctatgtggatacgctgct |
| | | ttaatgcctttgtatcatgctattgcttcccgtatggctttcattttctcctccttgtataaatcctggttgctgtctcttt |
| | | atgaggagttgtggcccgttgtcaggcaacgtggcgtggtgtgcactgtgtttgctgacgcaacccccactg |
| | | gttggggcattgccaccacctgtcagctcctttccgggactttcgctttccccctccctattgccacggcggaa |
| | | ctcatcgccgcctgccttgcccgctgctggacaggggctcggctgttgggcactgacaattccgtggtgttgt |
| | | cggggaagctgacgtcctttccatggctgctcgcctgtgttgccacctggattctgcgcgggacgtccttctg |
| | | ctacgtcccttcggccctcaatccagcggaccttccttcccgcggcctgctgccggctctgcggcctcttccg |
| | | cgtcttcgccttcgccctcagacgagtcggatctccctttgggccgcctccccgcctggaattcgagctcggt |
| | | acctttaagaccaatgacttacaaggcagctgtagatcttagccactttttaaaagaaaaggggggactggaa |
| | | gggctaattcactcccaacgaagacaagatctgctttttgcttgtactgggtctctctggttagaccagatctga |
| | | gcctgggagctctctggctaactagggaacccactgcttaagcctcaataaagcttgccttgagtgcttcaagt |
| | | agtgtgtgcccgtctgttgtgtgactctggtaactagagatccctcagacccttttagtcagtgtggaaaatctct |
| | | agcagtagtagttcatgtcatcttattattcagtatttataacttgcaaagaaatgaatatcagagagtgagagga |
| | | acttgtttattgcagcttataatggttacaaataaagcaatagcatcacaaatttcacaaataaagcatttttttcac |
| | | tgcattctagttgtggtttgtccaaactcatcaatgtatcttatcatgtctggctctagctatcccgcccctaactcc |
| | | gcccagttccgcccattctccgccccatggctgactaattttttttatttatgcagaggccgaggccgcctcgg |
| | | cctctgagctattccagaagtagtgaggaggcttttttggaggcctaggcttttgcgtcgagacgtacccaatt |
| | | cgccctatagtgagtcgtattacgcgcgctcactggccgtcgttttacaacgtcgtgactgggaaaaccctgg |
| | | cgttacccaacttaatcgccttgcagcacatccccctttcgccagctggcgtaatagcgaagaggcccgcac |
| | | cgatcgcccttcccaacagttgcgcagcctgaatggcgaatggcgcgacgcgccctgtagcggcgcattaa |
| | | gcgcggcgggtgtggtggttacgcgcagcgtgaccgctacacttgccagcgccctagcgcccgctcctttc |
| | | gctttcttcccttcctttctcgccacgttcgccggctttccccgtcaagctctaaatcgggggctccctttagggt |
| | | tccgatttagtgctttacggcacctcgaccccaaaaaacttgattagggtgatggttcacgtagtgggccatcg |
| | | ccctgatagacggtttttcgccctttgacgttggagtccacgttctttaatagtggactcttgttccaaactggaa |
| | | caacactcaaccctatctcggtctattcttttgatttataagggattttgccgatttcggcctattggttaaaaaatg |
| | | agctgatttaacaaaaatttaacgcgaattttaacaaaatattaacgtttacaatttcc |
| LV- | 4 | caggtggcacttttcggggaaatgtgcgcggaacccctatttgtttatttttctaaatacattcaaatatgtatccg |
| EF1a- | | ctcatgagacaataaccctgataaatgcttcaataatattgaaaaaggaagagtatgagtattcaacatttccgt |
| FITC(E2)- | | gtcgcccttattcccttttttgcggcattttgccttcctgtttttgctcacccagaaacgctggtgaaagtaaaaga |
| BBZ | | tgctgaagatcagttgggtgcacgagtgggttacatcgaactggatctcaacagcggtaagatccttgagagt |
| | | tttcgccccgaagaacgttttccaatgatgagcacttttaaagttctgctatgtggcgcggtattatcccgtattg |
| | | acgccgggcaagagcaactcggtcgccgcatacactattctcagaatgacttggttgagtactcaccagtca |
| | | cagaaaagcatcttacggatggcatgacagtaagagaattatgcagtgctgccataaccatgagtgataaca |
| | | ctgcggccaacttacttctgacaacgatcggaggaccgaaggagctaaccgcttttttgcacaacatggggg |
| | | atcatgtaactcgccttgatcgttgggaaccggagctgaatgaagccataccaaacgacgagcgtgacacc |
| | | acgatgcctgtagcaatggcaacaacgttgcgcaaactattaactggcgaactacttactctagcttcccggc |
| | | aacaattaatagactggatggaggcggataaagttgcaggaccacttctgcgctcggcccttccggctggct |
| | | ggtttattgctgataaatctggagccggtgagcgtgggtctcgcggtatcattgcagcactggggccagatg |
| | | gtaagccctcccgtatcgtagttatctacacgacggggagtcaggcaactatggatgaacgaaatagacaga |
| | | tcgctgagataggtgcctcactgattaagcattggtaactgtcagaccaagtttactcatatatactttagattgat |
| | | ttaaaacttcatttttaatttaaaaggatctaggtgaagatcctttttgataatctcatgaccaaaatcccttaacgt |
| | | gagttttcgttccactgagcgtcagaccccgtagaaaagatcaaaggatcttcttgagatcctttttttctgcgcg |
| | | taatctgctgcttgcaaacaaaaaaaccaccgctaccagcggtggtttgtttgccggatcaagagctaccaac |
| | | tctttttccgaaggtaactggcttcagcagagcgcagataccaaatactgtccttctagtgtagccgtagttagg |
| | | ccaccacttcaagaactctgtagcaccgcctacatacctcgctctgctaatcctgttaccagtggctgctgcca |
| | | gtggcgataagtcgtgtcttaccgggttggactcaagacgatagttaccggataaggcgcagcggtcgggc |
| | | tgaacggggggttcgtgcacacagcccagcttggagcgaacgacctacaccgaactgagatacctacagc |
| | | gtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgggtt |
| | | tcgccacctctgacttgagcgtcgatttttgtgatgctcgtcaggggggcggagcctatggaaaaacgccag |
| | | caacgcggcctttttacggttcctggccttttgctggccttttgctcacatgttctttcctgcgttatcccctgattct |
| | | gtggataaccgtattaccgcctttgagtgagctgataccgctcgccgcagccgaacgaccgagcgcagcga |
| | | gtcagtgagcgaggaagcggaagagcgcccaatacgcaaaccgcctctccccgcgcgttggccgattcat |
| | | taatgcagctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagcggata |
| | | acaatttcacacaggaaacagctatgaccatgattacgccaagcgcgcaattaaccctcactaaagggaaca |
| | | aaagctggagctgcaagcttaatgtagtcttatgcaatactcttgtagtcttgcaacatggtaacgatgagttag |
| | | caacatgccttacaaggagagaaaaagcaccgtgcatgccgattggtggaagtaaggtggtacgatcgtgc |
| | | cttattaggaaggcaacagacgggtctgacatggattggacgaaccactgaattgccgcattgcagagatatt |
| | | gtatttaagtgcctagctcgatacaataaacgggtctctctggttagaccagatctgagcctgggagctctctg |
| | | gctaactagggaacccactgcttaagcctcaataaagcttgccttgagtgcttcaagtagtgtgtgcccgtctg |
| | | ttgtgtgactctggtaactagagatccctcagacccttttagtcagtgtggaaaatctctagcagtggcgcccg |
| | | aacagggacctgaaagcgaaagggaaaccagagctctctcgacgcaggactcggcttgctgaagcgcgc |
| | | acggcaagaggcgaggggcggcgactggtgagtacgccaaaaattttgactagcggaggctagaaggag |
| | | agagatgggtgcgagagcgtcagtattaagcgggggagaattagatcgcgatgggaaaaaattcggttaag |
| | | gccagggggaaagaaaaaatataaattaaaacatatagtatgggcaagcagggagctagaacgattcgca |
| | | gttaatcctggcctgttagaaacatcagaaggctgtagacaaatactgggacagctacaaccatcccttcaga |
| | | caggatcagaagaacttagatcattatataatacagtagcaaccctctattgtgtgcatcaaaggatagagata |
| | | aaagacaccaaggaagctttagacaagatagaggaagagcaaaacaaaagtaagaccaccgcacagcaa |
| | | gcggccgctgatcttcagacctggaggaggagatatgagggacaattggagaagtgaattatataaatataa |
| | | agtagtaaaaattgaaccattaggagtagcacccaccaaggcaaagagaagagtggtgcagagagaaaaa |
| | | agagcagtgggaataggagctttgttccttgggttcttgggagcagcaggaagcactatgggcgcagcctc |
| | | aatgacgctgacggtacaggccagacaattattgtctggtatagtgcagcagcagaacaatttgctgagggc |
| | | tattgaggcgcaacagcatctgttgcaactcacagtctggggcatcaagcagctccaggcaagaatcctggc |
| | | tgtggaaagatacctaaaggatcaacagctcctggggatttggggttgctctggaaaactcatttgcaccact |
| | | gctgtgccttggaatgctagttggagtaataaatctctggaacagattggaatcacacgacctggatggagtg |
| | | ggacagagaaattaacaattacacaagcttaatacactccttaattgaagaatcgcaaaaccagcaagaaaa |
| | | gaatgaacaagaattattggaattagataaatgggcaagtttgtggaattggtttaacataacaaattggctgtg |
| | | gtatataaaattattcataatgatagtaggaggcttggtaggtttaagaatagtttttgctgtactttctatagtgaat |
| | | agagttaggcagggatattcaccattatcgtttcagacccacctcccaaccccgaggggacccgacaggcc |
| | | cgaaggaatagaagaagaaggtggagagagagacagagacagatccattcgattagtgaacggatctcga |
| | | cggttaacttttaaaagaaaaggggggattggggggtacagtgcaggggaaagaatagtagacataatagc |
| | | aacagacatacaaactaaagaattacaaaaacaaattacaaaaattcaaaattttatcgagctttgcaaagatg |
| | | gataaagttttaaacagagaggaatctttgcagctaatggaccttctaggtcttgaaaggagtgcctCGTG |
| | | AGGCTCCGGTGCCCGTCAGTGGGCAGAGCGCACATCGCCCACAG |
| | | TCCCCGAGAAGTTGGGGGGAGGGGTCGGCAATTGAACCGGTGCC |
| | | TAGAGAAGGTGGCGCGGGGTAAACTGGGAAAGTGATGTCGTGTA |
| | | CTGGCTCCGCCTTTTTCCCGAGGGTGGGGGAGAACCGTATATAAG |
| | | TGCAGTAGTCGCCGTGAACGTTCTTTTTCGCAACGGGTTTGCCGC |
| | | CAGAACACAGGTAAGTGCCGTGTGTGGTTCCCGCGGGCCTGGCC |
| | | TCTTTACGGGTTATGGCCCTTGCGTGCCTTGAATTACTTCCACCTG |
| | | GCTGCAGTACGTGATTCTTGATCCCGAGCTTCGGGTTGGAAGTGG |
| | | GTGGGAGAGTTCGAGGCCTTGCGCTTAAGGAGCCCCTTCGCCTCG |
| | | TGCTTGAGTTGAGGCCTGGCCTGGGCGCTGGGGCCGCCGCGTGC |
| | | GAATCTGGTGGCACCTTCGCGCCTGTCTCGCTGCTTTCGATAAGT |
| | | CTCTAGCCATTTAAAATTTTTGATGACCTGCTGCGACGCTTTTTTT |
| | | CTGGCAAGATAGTCTTGTAAATGCGGGCCAAGATCTGCACACTG |
| | | GTATTTCGGTTTTTGGGGCCGCGGGCGGCGACGGGGCCCGTGCGT |
| | | CCCAGCGCACATGTTCGGCGAGGCGGGGCCTGCGAGCGCGGCCA |
| | | CCGAGAATCGGACGGGGGTAGTCTCAAGCTGGCCGGCCTGCTCT |
| | | GGTGCCTGGCCTCGCGCCGCCGTGTATCGCCCCGCCCTGGGCGGC |
| | | AAGGCTGGCCCGGTCGGCACCAGTTGCGTGAGCGGAAAGATGGC |
| | | CGCTTCCCGGCCCTGCTGCAGGGAGCTCAAAATGGAGGACGCGG |
| | | CGCTCGGGAGAGCGGGCGGGTGAGTCACCCACACAAAGGAAAA |
| | | GGGCCTTTCCGTCCTCAGCCGTCGCTTCATGTGACTCCACGGAGT |
| | | ACCGGGCGCCGTCCAGGCACCTCGATTAGTTCTCGAGCTTTTGGA |
| | | GTACGTCGTCTTTAGGTTGGGGGGAGGGGTTTTATGCGATGGAGT |
| | | TTCCCCACACTGAGTGGGTGGAGACTGAAGTTAGGCCAGCTTGG |
| | | CACTTGATGTAATTCTCCTTGGAATTTGCCCTTTTTGAGTTTGGAT |
| | | CTTGGTTCATTCTCAAGCCTCAGACAGTGGTTCAAAGTTTTTTTCT |
| | | TCCATTTCAGGTGTCGTGAggaattcggtaccgcggccgcccggggatccatggccttac |
| | | cagtgaccgccttgctcctgccgctggccttgctgctccacgccgccaggccgCAGGTTCAGCT |
| | | GGTTGAGAGCGGAGGCAATCTGGTTCAGCCCGGTGGTAGTCTGC |
| | | GTCTGTCTTGTGCGGCGTCAGGGTTCACTTTCGGTAGTTTTTCAAT |
| | | GAGCTGGGTCCGTCAGGCACCAGGCGGTGGGCTGGAATGGGTGG |
| | | CAGGTCTGTCTGCACGTAGCTCCCTGACCCACTATGCAGATAGTG |
| | | TTAAAGGGCGGTTCACAATTTCACGCGACAACGCTAAGAATAGC |
| | | GTCTACCTGCAAATGAACTCCCTGCGGGTCGAGGATACCGCAGT |
| | | GTATTACTGCGCTCGCCGTTCTTATGACTCTAGTGGATACTGGGG |
| | | CCATTTTTATAGCTACATGGATGTGTGGGGACAGGGCACTCTGGT |
| | | GACCGTTTCCGGAGGCGGTGGGTCTGGAGGCGGTGGGAGTGGAG |
| | | GCGGTGGGTCAAGCGTTCTGACCCAGCCGTCCTCTGTCAGCGCCG |
| | | CGCCAGGCCAGAAAGTGACAATTTCCTGTTCTGGAAGTACTTCAA |
| | | ACATCGGCAACAATTATGTTTCCTGGTATCAGCAGCACCCGGGCA |
| | | AAGCGCCCAAGCTGATGATTTATGATGTGTCTAAACGTCCAAGTG |
| | | GTGTTCCTGACCGGTTCAGCGGTTCCAAGTCTGGGAATAGTGCCT |
| | | CACTGGACATCTCAGGCCTGCAAAGCGAAGATGAGGCGGACTAT |
| | | TACTGCGCAGCTTGGGATGACAGCCTGTCCGAATTTCTGTTCGGC |
| | | ACCGGGACAAAGCTGACCGTGCTGGGCaccacgacgccagcgccgcgaccacc |
| | | aacaccggcgcccaccatcgcgtcgcagcccctgtccctgcgcccagaggcgtgccggccagcggcgg |
| | | ggggcgcagtgcacacgagggggctggacttcgcctgtgatatctacatctgggcgcccttggccgggact |
| | | tgtggggtccttctcctgtcactggttatcaccctttactgcaaacggggcagaaagaaactcctgtatatattc |
| | | aaacaaccatttatgagaccagtacaaactactcaagaggaagatggctgtagctgccgatttccagaagaa |
| | | gaagaaggaggatgtgaactgagagtgaagttcagcaggagcgcagacgcccccgcgtacaagcaggg |
| | | ccagaaccagctctataacgagctcaatctaggacgaagagaggagtacgatgttttggacaagagacgtg |
| | | gccgggaccctgagatggggggaaagccgagaaggaagaaccctcaggaaggcctgtacaatgaactg |
| | | cagaaagataagatggcggaggcctacagtgagattgggatgaaaggcgagcgccggaggggcaaggg |
| | | gcacgatggcctttaccagggtctcagtacagccaccaaggacacctacgacgcccttcacatgcaggccc |
| | | tgccccctcgctaagtcgacaatcaacctctggattacaaaatttgtgaaagattgactggtattcttaactatgt |
| | | tgctccttttacgctatgtggatacgctgctttaatgcctttgtatcatgctattgcttcccgtatggctttcattttct |
| | | cctccttgtataaatcctggttgctgtctctttatgaggagttgtggcccgttgtcaggcaacgtggcgtggtgt |
| | | gcactgtgtttgctgacgcaacccccactggttggggcattgccaccacctgtcagctcctttccgggactttc |
| | | gctttccccctccctattgccacggcggaactcatcgccgcctgccttgcccgctgctggacaggggctcgg |
| | | ctgttgggcactgacaattccgtggtgttgtcggggaagctgacgtcctttccatggctgctcgcctgtgttgc |
| | | cacctggattctgcgcgggacgtccttctgctacgtcccttcggccctcaatccagcggaccttccttcccgc |
| | | ggcctgctgccggctctgcggcctcttccgcgtcttcgccttcgccctcagacgagtcggatctccctttggg |
| | | ccgcctccccgcctggaattcgagctcggtacctttaagaccaatgacttacaaggcagctgtagatcttagc |
| | | cactttttaaaagaaaaggggggactggaagggctaattcactcccaacgaagacaagatctgctttttgcttg |
| | | tactgggtctctctggttagaccagatctgagcctgggagctctctggctaactagggaacccactgcttaag |
| | | cctcaataaagcttgccttgagtgcttcaagtagtgtgtgcccgtctgttgtgtgactctggtaactagagatcc |
| | | ctcagacccttttagtcagtgtggaaaatctctagcagtagtagttcatgtcatcttattattcagtatttataacttg |
| | | caaagaaatgaatatcagagagtgagaggaacttgtttattgcagcttataatggttacaaataaagcaatagc |
| | | atcacaaatttcacaaataaagcatttttttcactgcattctagttgtggtttgtccaaactcatcaatgtatcttatc |
| | | atgtctggctctagctatcccgcccctaactccgcccagttccgcccattctccgccccatggctgactaatttt |
| | | ttttatttatgcagaggccgaggccgcctcggcctctgagctattccagaagtagtgaggaggcttttttggag |
| | | gcctaggcttttgcgtcgagacgtacccaattcgccctatagtgagtcgtattacgcgcgctcactggccgtc |
| | | gttttacaacgtcgtgactgggaaaaccctggcgttacccaacttaatcgccttgcagcacatccccctttcgc |
| | | cagctggcgtaatagcgaagaggcccgcaccgatcgcccttcccaacagttgcgcagcctgaatggcgaa |
| | | tggcgcgacgcgccctgtagcggcgcattaagcgcggcgggtgtggtggttacgcgcagcgtgaccgcta |
| | | cacttgccagcgccctagcgcccgctcctttcgctttcttcccttcctttctcgccacgttcgccggctttccccg |
| | | tcaagctctaaatcgggggctccctttagggttccgatttagtgctttacggcacctcgaccccaaaaaacttg |
| | | attagggtgatggttcacgtagtgggccatcgccctgatagacggtttttcgccctttgacgttggagtccacg |
| | | ttctttaatagtggactcttgttccaaactggaacaacactcaaccctatctcggtctattcttttgatttataaggg |
| | | attttgccgatttcggcctattggttaaaaaatgagctgatttaacaaaaatttaacgcgaattttaacaaaatatt |
| | | aacgtttacaatttcc |

TABLE 18
CAR-EC switch target interacting domain (antibodies)-Nucleotide Sequence

| NAME | SEQ ID | SEQUENCE |
|---|---|---|
| pBAD- | 5 | aagaaaccaattgtccatattgcatcagacattgccgtcactgcgtcttttactggctcttctcgctaaccaaacc |
| CD19wt | | ggtaaccctgattatttgcacggagtcacactttgctatgccatagcatttttatccataagattagcggatccta |
| | | cctgacgctttttatcgcaactctctactgtttctccatacccgtttttttgggctagaaataattttgtttaactttaa |
| | | gaaggagaatacatcaactagtacgcaagttcacgtaaaaagggtatctagaggttgaggtgattttatgaaa |
| | | aagaatatcgcatttcttcttgctagcatgttcgttttttctattgctacaaacgcatacgctgacatccagatgac |
| | | acagactacatcctccctgtctgcctctctgggagacagagtcaccatcagttgcagggcaagtcaggacatt |
| | | agtaaatatttaaattggtatcagcagaaaccagatggaactgttaaactcctgatctaccatacatcaagatta |
| | | cactcaggagtcccatcaaggttcagtggcagtgggtctggaacagattattctctcaccattagcaacctgg |
| | | agcaagaagatattgccacttacttttgccaacagggtaatacgcttccgtacacgttcggaggggggacca |
| | | agcttgagatcaaacgaactgtggctgcaccatctgtcttcatcttcccgccatctgatgagcagttgaaatctg |
| | | gaactgcctctgtcgtgtgcctgctgaataacttctatcccagagaggccaaagtacagtggaaggtggataa |
| | | cgccctccaatcgggtaactcccaggagagtgtcacagagcaggacagcaaggacagcacctacagcct |
| | | cagcagcaccctgacgctgagcaaagcagactacgagaaacacaaagtctacgcctgcgaagtcacccat |
| | | cagggcctgtcctcgcccgtcacaaagagcttcaacaggggagagtgttaagctggggatcctctagaggt |
| | | tgaggtgattttatgaaaaagaatatcgcatttcttcttgcatctatgttcgttttttctattgctacaaacgcgtacg |
| | | ctgaggtgaaactgcaggagtcaggacctggcctggtggcgccctcacagagcctgtccgtcacatgcact |
| | | gtctcaggggtctcattacccgactatggtgtaagctggattcgccagcctccacgaaagggtctggagtgg |
| | | ctgggagtaatatggggtagtgaaaccacatactataattcagctctcaaatccagactgaccatcatcaagg |
| | | acaactccaagagccaagttttcttaaaaatgaacagtctgcaaactgatgacacagccatttactactgtgcc |
| | | aaacattattactacggtggtagctatgctatggactactggggccaaggaacctcagtcaccgtctcctcag |
| | | cctccaccaagggcccatcggtcttccccctggcaccctcctccaagagcacctctgggggcacagcggc |
| | | cctgggctgcctggtcaaggactacttccccgaaccggtgacggtgtcgtggaactcaggcgccctgacca |
| | | gcggcgtgcacaccttcccggctgtcctacagtcctcaggactctactccctcagcagcgtggtgactgtgc |
| | | cctctagcagcttgggcacccagacctacatctgcaacgtgaatcacaagcccagcaacaccaaggtggac |
| | | aagaaagttgagcccaaatcttgtgacaaaactcacacataataagtcgaccgatgcccttgagagccttcaa |
| | | cccagtcagctccttccggtgggcgcggggcatgactatcgtcgccgcacttatgactgtcttctttatcatgc |
| | | aactcgtaggacaggtgccaaacggtctccagcttggctgttttggcggatgagagaagattttcagcctgat |
| | | acagattaaatcagaacgcagaagcggtctgataaaacagaatttgcctggcggcagtagcgcggtggtcc |
| | | cacctgaccccatgccgaactcagaagtgaaacgccgtagcgccgatggtagtgtggggtctccccatgcg |
| | | agagtagggaactgccaggcatcaaataaaacgaaaggctcagtcgaaagactgggcctttcgttttatctgt |
| | | tgtttgtcggtgaacgctctcctgagtaggacaaatccgccgggagcggatttgaacgttgcgaagcaacgg |
| | | cccggagggtggcgggcaggacgcccgccataaactgccaggcatcaaattaagcagaaggccatcctg |
| | | acggatggcctttttgcgtttctacaaactctttttgtttatttttctaaatacattcaaatatgtatccgctcatgaga |
| | | caataaccctgataaatgcttcaataatattgaaaaaggaagagtatgagtattcaacatttccgtgtcgccctt |
| | | attcccttttttgcggcattttgccttcctgtttttgctcacccagaaacgctggtgaaagtaaaagatgctgaag |
| | | atcagttgggtgcacgagtgggttacatcgaactggatctcaacagcggtaagatccttgagagttttcgccc |
| | | cgaagaacgttttccaatgatgagcacttttaaagttctgctatgtggcgcggtattatcccgtgttgacgccgg |
| | | gcaagagcaactcggtcgccgcatacactattctcagaatgacttggttgagtactcaccagtcacagaaaa |
| | | gcatcttacggatggcatgacagtaagagaattatgcagtgctgccataaccatgagtgataacactgcggc |
| | | caacttacttctgacaacgatcggaggaccgaaggagctaaccgcttttttgcacaacatgggggatcatgta |
| | | actcgccttgatcgttgggaaccggagctgaatgaagccataccaaacgacgagcgtgacaccacgatgc |
| | | ctgtagcaatggcaacaacgttgcgcaaactattaactggcgaactacttactctagcttcccggcaacaatta |
| | | atagactggatggaggcggataaagttgcaggaccacttctgcgctcggcccttccggctggctggtttattg |
| | | ctgataaatctggagccggtgagcgtgggtctcgcggtatcattgcagcactggggccagatggtaagccct |
| | | cccgtatcgtagttatctacacgacggggagtcaggcaactatggatgaacgaaatagacagatcgctgag |
| | | ataggtgcctcactgattaagcattggtaactgtcagaccaagtttactcatatatactttagattgatttaaaactt |
| | | catttttaatttaaaaggatctaggtgaagatcctttttgataatctcatgaccaaaatcccttaacgtgagttttcg |
| | | ttccactgagcgtcagaccccgtagaaaagatcaaaggatcttcttgagatcctttttttctgcgcgtaatctgct |
| | | gcttgcaaacaaaaaaaccaccgctaccagcggtggtttgtttgccggatcaagagctaccaactctttttccg |
| | | aaggtaactggcttcagcagagcgcagataccaaatactgtccttctagtgtagccgtagttaggccaccact |
| | | tcaagaactctgtagcaccgcctacatacctcgctctgctaatcctgttaccagtggctgctgccagtggcgat |
| | | aagtcgtgtcttaccgggttggactcaagacgatagttaccggataaggcgcagcggtcgggctgaacggg |
| | | gggttcgtgcacacagcccagcttggagcgaacgacctacaccgaactgagatacctacagcgtgagctat |
| | | gagaaagcgccacgcttcccgaagggagaaaggcggacaggtatccggtaagcggcagggtcggaaca |
| | | ggagagcgcacgagggagcttccagggggaaacgcctggtatctttatagtcctgtcgggtttcgccacctc |
| | | tgacttgagcgtcgatttttgtgatgctcgtcaggggggcggagcctatggaaaaacgccagcaacgcggc |
| | | ctttttacggttcctggccttttgctggccttttgctcacatgttctttcctgcgttatcccctgattctgtggataacc |
| | | gtattaccgcctttgagtgagctgataccgctcgccgcagccgaacgaccgagcgcagcgagtcagtgag |
| | | cgaggaagcggaagagcgcctgatgcggtattttctccttacgcatctgtgcggtatttcacaccgcatatggt |
| | | gcactctcagtacaatctgctctgatgccgcatagttaagccagtatacactccgctatcgctacgtgactggg |
| | | tcatggctgcgccccgacacccgccaacacccgctgacgcgccctgacgggcttgtctgctcccggcatcc |
| | | gcttacagacaagctgtgaccgtctccgggagctgcatgtgtcagaggttttcaccgtcatcaccgaaacgc |
| | | gcgaggcagcagatcaattcgcgcgcgaaggcgaagcggcatgcataatgtgcctgtcaaatggacgaa |
| | | gcagggattctgcaaaccctatgctactccgtcaagccgtcaattgtctgattcgttaccaattatgacaacttg |
| | | acggctacatcattcactttttcttcacaaccggcacggaactcgctcgggctggccccggtgcattttttaaat |
| | | acccgcgagaaatagagttgatcgtcaaaaccaacattgcgaccgacggtggcgataggcatccgggtgg |
| | | tgctcaaaagcagcttcgcctggctgatacgttggtcctcgcgccagcttaagacgctaatccctaactgctg |
| | | gcggaaaagatgtgacagacgcgacggcgacaagcaaacatgctgtgcgacgctggcgatatcaaaattg |
| | | ctgtctgccaggtgatcgctgatgtactgacaagcctcgcgtacccgattatccatcggtggatggagcgact |
| | | cgttaatcgcttccatgcgccgcagtaacaattgctcaagcagatttatcgccagcagctccgaatagcgccc |
| | | ttccccttgcccggcgttaatgatttgcccaaacaggtcgctgaaatgcggctggtgcgcttcatccgggcga |
| | | aagaaccccgtattggcaaatattgacggccagttaagccattcatgccagtaggcgcgcggacgaaagta |
| | | aacccactggtgataccattcgcgagcctccggatgacgaccgtagtgatgaatctctcctggcgggaaca |
| | | gcaaaatatcacccggtcggcaaacaaattctcgtccctgatttttcaccaccccctgaccgcgaatggtgag |
| | | attgagaatataacctttcattcccagcggtcggtcgataaaaaaatcgagataaccgttggcctcaatcggc |
| | | gttaaacccgccaccagatgggcattaaacgagtatcccggcagcaggggatcattttgcgcttcagccata |
| | | cttttcatactcccgccattcagag |
| pBAD- | 6 | aagaaaccaattgtccatattgcatcagacattgccgtcactgcgtcttttactggctcttctcgctaaccaaacc |
| CD19 | | ggtaaccctgattatttgcacggagtcacactttgctatgccatagcatttttatccataagattagcggatccta |
| LS202Xmt | | cctgacgctttttatcgcaactctctactgtttctccatacccgtttttttgggctagaaataattttgtttaactttaa |
| | | gaaggagaatacatcaactagtacgcaagttcacgtaaaaagggtatctagaggttgaggtgattttatgaaa |
| | | aagaatatcgcatttcttcttgctagcatgttcgttttttctattgctacaaacgcatacgctgacatccagatgac |
| | | acagactacatcctccctgtctgcctctctgggagacagagtcaccatcagttgcagggcaagtcaggacatt |
| | | agtaaatatttaaattggtatcagcagaaaccagatggaactgttaaactcctgatctaccatacatcaagatta |
| | | cactcaggagtcccatcaaggttcagtggcagtgggtctggaacagattattctctcaccattagcaacctgg |
| | | agcaagaagatattgccacttacttttgccaacagggtaatacgcttccgtacacgttcggaggggggacca |
| | | agcttgagatcaaacgaactgtggctgcaccatctgtcttcatcttcccgccatctgatgagcagttgaaatctg |
| | | gaactgcctctgtcgtgtgcctgctgaataacttctatcccagagaggccaaagtacagtggaaggtggataa |
| | | cgccctccaatcgggtaactcccaggagagtgtcacagagcaggacagcaaggacagcacctacagcct |
| | | cagcagcaccctgacgctgagcaaagcagactacgagaaacacaaagtctacgcctgcgaagtcacccat |
| | | cagggcctgTAGtcgcccgtcacaaagagcttcaacaggggagagtgttaagctggggatcctctagag |
| | | gttgaggtgattttatgaaaaagaatatcgcatttcttcttgcatctatgttcgttttttctattgctacaaacgcgta |
| | | cgctgaggtgaaactgcaggagtcaggacctggcctggtggcgccctcacagagcctgtccgtcacatgc |
| | | actgtctcaggggtctcattacccgactatggtgtaagctggattcgccagcctccacgaaagggtctggagt |
| | | ggctgggagtaatatggggtagtgaaaccacatactataattcagctctcaaatccagactgaccatcatcaa |
| | | ggacaactccaagagccaagttttcttaaaaatgaacagtctgcaaactgatgacacagccatttactactgtg |
| | | ccaaacattattactacggtggtagctatgctatggactactggggccaaggaacctcagtcaccgtctcctc |
| | | agcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgac |
| | | cagcggcgtgcacaccttcccggctgtcctacagtcctcaggactctactccctcagcagcgtggtgactgt |
| | | gccctctagcagcttgggcacccagacctacatctgcaacgtgaatcacaagcccagcaacaccaaggtg |
| | | gacaagaaagttgagcccaaatcttgtgacaaaactcacacataataagtcgaccgatgcccttgagagcctt |
| | | caacccagtcagctccttccggtgggcgcggggcatgactatcgtcgccgcacttatgactgtcttctttatca |
| | | tgcaactcgtaggacaggtgccaaacggtctccagcttggctgttttggcggatgagagaagattttcagcct |
| | | gatacagattaaatcagaacgcagaagcggtctgataaaacagaatttgcctggcggcagtagcgcggtgg |
| | | tcccacctgaccccatgccgaactcagaagtgaaacgccgtagcgccgatggtagtgtggggtctccccat |
| | | gcgagagtagggaactgccaggcatcaaataaaacgaaaggctcagtcgaaagactgggcctttcgttttat |
| | | ctgttgtttgtcggtgaacgctctcctgagtaggacaaatccgccgggagcggatttgaacgttgcgaagcaa |
| | | cggcccggagggtggcgggcaggacgcccgccataaactgccaggcatcaaattaagcagaaggccat |
| | | cctgacggatggcctttttgcgtttctacaaactctttttgtttatttttctaaatacattcaaatatgtatccgctcat |
| | | gagacaataaccctgataaatgcttcaataatattgaaaaaggaagagtatgagtattcaacatttccgtgtcg |
| | | cccttattcccttttttgcggcattttgccttcctgtttttgctcacccagaaacgctggtgaaagtaaaagatgct |
| | | gaagatcagttgggtgcacgagtgggttacatcgaactggatctcaacagcggtaagatccttgagagttttc |
| | | gccccgaagaacgttttccaatgatgagcacttttaaagttctgctatgtggcgcggtattatcccgtgttgacg |
| | | ccgggcaagagcaactcggtcgccgcatacactattctcagaatgacttggttgagtactcaccagtcacag |
| | | aaaagcatcttacggatggcatgacagtaagagaattatgcagtgctgccataaccatgagtgataacactgc |
| | | ggccaacttacttctgacaacgatcggaggaccgaaggagctaaccgcttttttgcacaacatgggggatca |
| | | tgtaactcgccttgatcgttgggaaccggagctgaatgaagccataccaaacgacgagcgtgacaccacga |
| | | tgcctgtagcaatggcaacaacgttgcgcaaactattaactggcgaactacttactctagcttcccggcaaca |
| | | attaatagactggatggaggcggataaagttgcaggaccacttctgcgctcggcccttccggctggctggttt |
| | | attgctgataaatctggagccggtgagcgtgggtctcgcggtatcattgcagcactggggccagatggtaag |
| | | ccctcccgtatcgtagttatctacacgacggggagtcaggcaactatggatgaacgaaatagacagatcgct |
| | | gagataggtgcctcactgattaagcattggtaactgtcagaccaagtttactcatatatactttagattgatttaaa |
| | | acttcatttttaatttaaaaggatctaggtgaagatcctttttgataatctcatgaccaaaatcccttaacgtgagttt |
| | | tcgttccactgagcgtcagaccccgtagaaaagatcaaaggatcttcttgagatcctttttttctgcgcgtaatct |
| | | gctgcttgcaaacaaaaaaaccaccgctaccagcggtggtttgtttgccggatcaagagctaccaactcttttt |
| | | ccgaaggtaactggcttcagcagagcgcagataccaaatactgtccttctagtgtagccgtagttaggccac |
| | | cacttcaagaactctgtagcaccgcctacatacctcgctctgctaatcctgttaccagtggctgctgccagtgg |
| | | cgataagtcgtgtcttaccgggttggactcaagacgatagttaccggataaggcgcagcggtcgggctgaa |
| | | cggggggttcgtgcacacagcccagcttggagcgaacgacctacaccgaactgagatacctacagcgtga |
| | | gctatgagaaagcgccacgcttcccgaagggagaaaggcggacaggtatccggtaagcggcagggtcg |
| | | gaacaggagagcgcacgagggagcttccagggggaaacgcctggtatctttatagtcctgtcgggtttcgc |
| | | cacctctgacttgagcgtcgatttttgtgatgctcgtcaggggggcggagcctatggaaaaacgccagcaac |
| | | gcggcctttttacggttcctggccttttgctggccttttgctcacatgttctttcctgcgttatcccctgattctgtgg |
| | | ataaccgtattaccgcctttgagtgagctgataccgctcgccgcagccgaacgaccgagcgcagcgagtca |
| | | gtgagcgaggaagcggaagagcgcctgatgcggtattttctccttacgcatctgtgcggtatttcacaccgca |
| | | tatggtgcactctcagtacaatctgctctgatgccgcatagttaagccagtatacactccgctatcgctacgtga |
| | | ctgggtcatggctgcgccccgacacccgccaacacccgctgacgcgccctgacgggcttgtctgctcccg |
| | | gcatccgcttacagacaagctgtgaccgtctccgggagctgcatgtgtcagaggttttcaccgtcatcaccga |
| | | aacgcgcgaggcagcagatcaattcgcgcgcgaaggcgaagcggcatgcataatgtgcctgtcaaatgga |
| | | cgaagcagggattctgcaaaccctatgctactccgtcaagccgtcaattgtctgattcgttaccaattatgaca |
| | | acttgacggctacatcattcactttttcttcacaaccggcacggaactcgctcgggctggccccggtgcattttt |
| | | taaatacccgcgagaaatagagttgatcgtcaaaaccaacattgcgaccgacggtggcgataggcatccgg |
| | | gtggtgctcaaaagcagcttcgcctggctgatacgttggtcctcgcgccagcttaagacgctaatccctaact |
| | | gctggcggaaaagatgtgacagacgcgacggcgacaagcaaacatgctgtgcgacgctggcgatatcaa |
| | | aattgctgtctgccaggtgatcgctgatgtactgacaagcctcgcgtacccgattatccatcggtggatggag |
| | | cgactcgttaatcgcttccatgcgccgcagtaacaattgctcaagcagatttatcgccagcagctccgaatag |
| | | cgcccttccccttgcccggcgttaatgatttgcccaaacaggtcgctgaaatgcggctggtgcgcttcatccg |
| | | ggcgaaagaaccccgtattggcaaatattgacggccagttaagccattcatgccagtaggcgcgcggacga |
| | | aagtaaacccactggtgataccattcgcgagcctccggatgacgaccgtagtgatgaatctctcctggcggg |
| | | aacagcaaaatatcacccggtcggcaaacaaattctcgtccctgatttttcaccaccccctgaccgcgaatgg |
| | | tgagattgagaatataacctttcattcccagcggtcggtcgataaaaaaatcgagataaccgttggcctcaatc |
| | | ggcgttaaacccgccaccagatgggcattaaacgagtatcccggcagcaggggatcattttgcgcttcagc |
| | | catacttttcatactcccgccattcagag |
| pBAD- | 7 | aagaaaccaattgtccatattgcatcagacattgccgtcactgcgtcttttactggctcttctcgctaaccaaacc |
| CD19 | | ggtaaccctgattatttgcacggagtcacactttgctatgccatagcatttttatccataagattagcggatccta |
| HK136Xmt | | cctgacgctttttatcgcaactctctactgtttctccatacccgtttttttgggctagaaataattttgtttaactttaa |
| | | gaaggagaatacatcaactagtacgcaagttcacgtaaaaagggtatctagaggttgaggtgattttatgaaa |
| | | aagaatatcgcatttcttcttgctagcatgttcgttttttctattgctacaaacgcatacgctgacatccagatgac |
| | | acagactacatcctccctgtctgcctctctgggagacagagtcaccatcagttgcagggcaagtcaggacatt |
| | | agtaaatatttaaattggtatcagcagaaaccagatggaactgttaaactcctgatctaccatacatcaagatta |
| | | cactcaggagtcccatcaaggttcagtggcagtgggtctggaacagattattctctcaccattagcaacctgg |
| | | agcaagaagatattgccacttacttttgccaacagggtaatacgcttccgtacacgttcggaggggggacca |
| | | agcttgagatcaaacgaactgtggctgcaccatctgtcttcatcttcccgccatctgatgagcagttgaaatctg |
| | | gaactgcctctgtcgtgtgcctgctgaataacttctatcccagagaggccaaagtacagtggaaggtggataa |
| | | cgccctccaatcgggtaactcccaggagagtgtcacagagcaggacagcaaggacagcacctacagcct |
| | | cagcagcaccctgacgctgagcaaagcagactacgagaaacacaaagtctacgcctgcgaagtcacccat |
| | | cagggcctgtcctcgcccgtcacaaagagcttcaacaggggagagtgttaagctggggatcctctagaggt |
| | | tgaggtgattttatgaaaaagaatatcgcatttcttcttgcatctatgttcgttttttctattgctacaaacgcgtacg |
| | | ctgaggtgaaactgcaggagtcaggacctggcctggtggcgccctcacagagcctgtccgtcacatgcact |
| | | gtctcaggggtctcattacccgactatggtgtaagctggattcgccagcctccacgaaagggtctggagtgg |
| | | ctgggagtaatatggggtagtgaaaccacatactataattcagctctcaaatccagactgaccatcatcaagg |
| | | acaactccaagagccaagttttcttaaaaatgaacagtctgcaaactgatgacacagccatttactactgtgcc |
| | | aaacattattactacggtggtagctatgctatggactactggggccaaggaacctcagtcaccgtctcctcag |
| | | cctccaccaagggcccatcggtcttccccctggcaccctcctccTAGagcacctctgggggcacagcgg |
| | | ccctgggctgcctggtcaaggactacttccccgaaccggtgacggtgtcgtggaactcaggcgccctgacc |
| | | agcggcgtgcacaccttcccggctgtcctacagtcctcaggactctactccctcagcagcgtggtgactgtg |
| | | ccctctagcagcttgggcacccagacctacatctgcaacgtgaatcacaagcccagcaacaccaaggtgga |
| | | caagaaagttgagcccaaatcttgtgacaaaactcacacataataagtcgaccgatgcccttgagagccttca |
| | | acccagtcagctccttccggtgggcgcggggcatgactatcgtcgccgcacttatgactgtcttctttatcatg |
| | | caactcgtaggacaggtgccaaacggtctccagcttggctgttttggcggatgagagaagattttcagcctga |
| | | tacagattaaatcagaacgcagaagcggtctgataaaacagaatttgcctggcggcagtagcgcggtggtc |
| | | ccacctgaccccatgccgaactcagaagtgaaacgccgtagcgccgatggtagtgtggggtctccccatgc |
| | | gagagtagggaactgccaggcatcaaataaaacgaaaggctcagtcgaaagactgggcctttcgttttatct |
| | | gttgtttgtcggtgaacgctctcctgagtaggacaaatccgccgggagcggatttgaacgttgcgaagcaac |
| | | ggcccggagggtggcgggcaggacgcccgccataaactgccaggcatcaaattaagcagaaggccatc |
| | | ctgacggatggcctttttgcgtttctacaaactctttttgtttatttttctaaatacattcaaatatgtatccgctcatg |
| | | agacaataaccctgataaatgcttcaataatattgaaaaaggaagagtatgagtattcaacatttccgtgtcgcc |
| | | cttattcccttttttgcggcattttgccttcctgtttttgctcacccagaaacgctggtgaaagtaaaagatgctga |
| | | agatcagttgggtgcacgagtgggttacatcgaactggatctcaacagcggtaagatccttgagagttttcgc |
| | | cccgaagaacgttttccaatgatgagcacttttaaagttctgctatgtggcgcggtattatcccgtgttgacgcc |
| | | gggcaagagcaactcggtcgccgcatacactattctcagaatgacttggttgagtactcaccagtcacagaa |
| | | aagcatcttacggatggcatgacagtaagagaattatgcagtgctgccataaccatgagtgataacactgcg |
| | | gccaacttacttctgacaacgatcggaggaccgaaggagctaaccgcttttttgcacaacatgggggatcat |
| | | gtaactcgccttgatcgttgggaaccggagctgaatgaagccataccaaacgacgagcgtgacaccacgat |
| | | gcctgtagcaatggcaacaacgttgcgcaaactattaactggcgaactacttactctagcttcccggcaacaa |
| | | ttaatagactggatggaggcggataaagttgcaggaccacttctgcgctcggcccttccggctggctggttta |
| | | ttgctgataaatctggagccggtgagcgtgggtctcgcggtatcattgcagcactggggccagatggtaagc |
| | | cctcccgtatcgtagttatctacacgacggggagtcaggcaactatggatgaacgaaatagacagatcgctg |
| | | agataggtgcctcactgattaagcattggtaactgtcagaccaagtttactcatatatactttagattgatttaaaa |
| | | cttcatttttaatttaaaaggatctaggtgaagatcctttttgataatctcatgaccaaaatcccttaacgtgagtttt |
| | | cgttccactgagcgtcagaccccgtagaaaagatcaaaggatcttcttgagatcctttttttctgcgcgtaatct |
| | | gctgcttgcaaacaaaaaaaccaccgctaccagcggtggtttgtttgccggatcaagagctaccaactcttttt |
| | | ccgaaggtaactggcttcagcagagcgcagataccaaatactgtccttctagtgtagccgtagttaggccac |
| | | cacttcaagaactctgtagcaccgcctacatacctcgctctgctaatcctgttaccagtggctgctgccagtgg |
| | | cgataagtcgtgtcttaccgggttggactcaagacgatagttaccggataaggcgcagcggtcgggctgaa |
| | | cggggggttcgtgcacacagcccagcttggagcgaacgacctacaccgaactgagatacctacagcgtga |
| | | gctatgagaaagcgccacgcttcccgaagggagaaaggcggacaggtatccggtaagcggcagggtcg |
| | | gaacaggagagcgcacgagggagcttccagggggaaacgcctggtatctttatagtcctgtcgggtttcgc |
| | | cacctctgacttgagcgtcgatttttgtgatgctcgtcaggggggcggagcctatggaaaaacgccagcaac |
| | | gcggcctttttacggttcctggccttttgctggccttttgctcacatgttctttcctgcgttatcccctgattctgtgg |
| | | ataaccgtattaccgcctttgagtgagctgataccgctcgccgcagccgaacgaccgagcgcagcgagtca |
| | | gtgagcgaggaagcggaagagcgcctgatgcggtattttctccttacgcatctgtgcggtatttcacaccgca |
| | | tatggtgcactctcagtacaatctgctctgatgccgcatagttaagccagtatacactccgctatcgctacgtga |
| | | ctgggtcatggctgcgccccgacacccgccaacacccgctgacgcgccctgacgggcttgtctgctcccg |
| | | gcatccgcttacagacaagctgtgaccgtctccgggagctgcatgtgtcagaggttttcaccgtcatcaccga |
| | | aacgcgcgaggcagcagatcaattcgcgcgcgaaggcgaagcggcatgcataatgtgcctgtcaaatgga |
| | | cgaagcagggattctgcaaaccctatgctactccgtcaagccgtcaattgtctgattcgttaccaattatgaca |
| | | acttgacggctacatcattcactttttcttcacaaccggcacggaactcgctcgggctggccccggtgcattttt |
| | | taaatacccgcgagaaatagagttgatcgtcaaaaccaacattgcgaccgacggtggcgataggcatccgg |
| | | gtggtgctcaaaagcagcttcgcctggctgatacgttggtcctcgcgccagcttaagacgctaatccctaact |
| | | gctggcggaaaagatgtgacagacgcgacggcgacaagcaaacatgctgtgcgacgctggcgatatcaa |
| | | aattgctgtctgccaggtgatcgctgatgtactgacaagcctcgcgtacccgattatccatcggtggatggag |
| | | cgactcgttaatcgcttccatgcgccgcagtaacaattgctcaagcagatttatcgccagcagctccgaatag |
| | | cgcccttccccttgcccggcgttaatgatttgcccaaacaggtcgctgaaatgcggctggtgcgcttcatccg |
| | | ggcgaaagaaccccgtattggcaaatattgacggccagttaagccattcatgccagtaggcgcgcggacga |
| | | aagtaaacccactggtgataccattcgcgagcctccggatgacgaccgtagtgatgaatctctcctggcggg |
| | | aacagcaaaatatcacccggtcggcaaacaaattctcgtccctgatttttcaccaccccctgaccgcgaatgg |
| | | tgagattgagaatataacctttcattcccagcggtcggtcgataaaaaaatcgagataaccgttggcctcaatc |
| | | ggcgttaaacccgccaccagatgggcattaaacgagtatcccggcagcaggggatcattttgcgcttcagc |
| | | catacttttcatactcccgccattcagag |
| pBAD- | 8 | aagaaaccaattgtccatattgcatcagacattgccgtcactgcgtcttttactggctcttctcgctaaccaaacc |
| CD19 | | ggtaaccctgattatttgcacggagtcacactttgctatgccatagcatttttatccataagattagcggatccta |
| LS202/HK136Xmt | | cctgacgctttttatcgcaactctctactgtttctccatacccgtttttttgggctagaaataattttgtttaactttaa |
| | | gaaggagaatacatcaactagtacgcaagttcacgtaaaaagggtatctagaggttgaggtgattttatgaaa |
| | | aagaatatcgcatttcttcttgctagcatgttcgttttttctattgctacaaacgcatacgctgacatccagatgac |
| | | acagactacatcctccctgtctgcctctctgggagacagagtcaccatcagttgcagggcaagtcaggacatt |
| | | agtaaatatttaaattggtatcagcagaaaccagatggaactgttaaactcctgatctaccatacatcaagatta |
| | | cactcaggagtcccatcaaggttcagtggcagtgggtctggaacagattattctctcaccattagcaacctgg |
| | | agcaagaagatattgccacttacttttgccaacagggtaatacgcttccgtacacgttcggaggggggacca |
| | | agcttgagatcaaacgaactgtggctgcaccatctgtcttcatcttcccgccatctgatgagcagttgaaatctg |
| | | gaactgcctctgtcgtgtgcctgctgaataacttctatcccagagaggccaaagtacagtggaaggtggataa |
| | | cgccctccaatcgggtaactcccaggagagtgtcacagagcaggacagcaaggacagcacctacagcct |
| | | cagcagcaccctgacgctgagcaaagcagactacgagaaacacaaagtctacgcctgcgaagtcacccat |
| | | cagggcctgTAGtcgcccgtcacaaagagcttcaacaggggagagtgttaagctggggatcctctagag |
| | | gttgaggtgattttatgaaaaagaatatcgcatttcttcttgcatctatgttcgttttttctattgctacaaacgcgta |
| | | cgctgaggtgaaactgcaggagtcaggacctggcctggtggcgccctcacagagcctgtccgtcacatgc |
| | | actgtctcaggggtctcattacccgactatggtgtaagctggattcgccagcctccacgaaagggtctggagt |
| | | ggctgggagtaatatggggtagtgaaaccacatactataattcagctctcaaatccagactgaccatcatcaa |
| | | ggacaactccaagagccaagttttcttaaaaatgaacagtctgcaaactgatgacacagccatttactactgtg |
| | | ccaaacattattactacggtggtagctatgctatggactactggggccaaggaacctcagtcaccgtctcctc |
| | | agcctccaccaagggcccatcggtcttccccctggcaccctcctccTAGagcacctctgggggcacagc |
| | | ggccctgggctgcctggtcaaggactacttccccgaaccggtgacggtgtcgtggaactcaggcgccctga |
| | | ccagcggcgtgcacaccttcccggctgtcctacagtcctcaggactctactccctcagcagcgtggtgactg |
| | | tgccctctagcagcttgggcacccagacctacatctgcaacgtgaatcacaagcccagcaacaccaaggtg |
| | | gacaagaaagttgagcccaaatcttgtgacaaaactcacacataataagtcgaccgatgcccttgagagcctt |
| | | caacccagtcagctccttccggtgggcgcggggcatgactatcgtcgccgcacttatgactgtcttctttatca |
| | | tgcaactcgtaggacaggtgccaaacggtctccagcttggctgttttggcggatgagagaagattttcagcct |
| | | gatacagattaaatcagaacgcagaagcggtctgataaaacagaatttgcctggcggcagtagcgcggtgg |
| | | tcccacctgaccccatgccgaactcagaagtgaaacgccgtagcgccgatggtagtgtggggtctccccat |
| | | gcgagagtagggaactgccaggcatcaaataaaacgaaaggctcagtcgaaagactgggcctttcgttttat |
| | | ctgttgtttgtcggtgaacgctctcctgagtaggacaaatccgccgggagcggatttgaacgttgcgaagcaa |
| | | cggcccggagggtggcgggcaggacgcccgccataaactgccaggcatcaaattaagcagaaggccat |
| | | cctgacggatggcctttttgcgtttctacaaactctttttgtttatttttctaaatacattcaaatatgtatccgctcat |
| | | gagacaataaccctgataaatgcttcaataatattgaaaaaggaagagtatgagtattcaacatttccgtgtcg |
| | | cccttattcccttttttgcggcattttgccttcctgtttttgctcacccagaaacgctggtgaaagtaaaagatgct |
| | | gaagatcagttgggtgcacgagtgggttacatcgaactggatctcaacagcggtaagatccttgagagttttc |
| | | gccccgaagaacgttttccaatgatgagcacttttaaagttctgctatgtggcgcggtattatcccgtgttgacg |
| | | ccgggcaagagcaactcggtcgccgcatacactattctcagaatgacttggttgagtactcaccagtcacag |
| | | aaaagcatcttacggatggcatgacagtaagagaattatgcagtgctgccataaccatgagtgataacactgc |
| | | ggccaacttacttctgacaacgatcggaggaccgaaggagctaaccgcttttttgcacaacatgggggatca |
| | | tgtaactcgccttgatcgttgggaaccggagctgaatgaagccataccaaacgacgagcgtgacaccacga |
| | | tgcctgtagcaatggcaacaacgttgcgcaaactattaactggcgaactacttactctagcttcccggcaaca |
| | | attaatagactggatggaggcggataaagttgcaggaccacttctgcgctcggcccttccggctggctggttt |
| | | attgctgataaatctggagccggtgagcgtgggtctcgcggtatcattgcagcactggggccagatggtaag |
| | | ccctcccgtatcgtagttatctacacgacggggagtcaggcaactatggatgaacgaaatagacagatcgct |
| | | gagataggtgcctcactgattaagcattggtaactgtcagaccaagtttactcatatatactttagattgatttaaa |
| | | acttcatttttaatttaaaaggatctaggtgaagatcctttttgataatctcatgaccaaaatcccttaacgtgagttt |
| | | tcgttccactgagcgtcagaccccgtagaaaagatcaaaggatcttcttgagatcctttttttctgcgcgtaatct |
| | | gctgcttgcaaacaaaaaaaccaccgctaccagcggtggtttgtttgccggatcaagagctaccaactcttttt |
| | | ccgaaggtaactggcttcagcagagcgcagataccaaatactgtccttctagtgtagccgtagttaggccac |
| | | cacttcaagaactctgtagcaccgcctacatacctcgctctgctaatcctgttaccagtggctgctgccagtgg |
| | | cgataagtcgtgtcttaccgggttggactcaagacgatagttaccggataaggcgcagcggtcgggctgaa |
| | | cggggggttcgtgcacacagcccagcttggagcgaacgacctacaccgaactgagatacctacagcgtga |
| | | gctatgagaaagcgccacgcttcccgaagggagaaaggcggacaggtatccggtaagcggcagggtcg |
| | | gaacaggagagcgcacgagggagcttccagggggaaacgcctggtatctttatagtcctgtcgggtttcgc |
| | | cacctctgacttgagcgtcgatttttgtgatgctcgtcaggggggcggagcctatggaaaaacgccagcaac |
| | | gcggcctttttacggttcctggccttttgctggccttttgctcacatgttctttcctgcgttatcccctgattctgtgg |
| | | ataaccgtattaccgcctttgagtgagctgataccgctcgccgcagccgaacgaccgagcgcagcgagtca |
| | | gtgagcgaggaagcggaagagcgcctgatgcggtattttctccttacgcatctgtgcggtatttcacaccgca |
| | | tatggtgcactctcagtacaatctgctctgatgccgcatagttaagccagtatacactccgctatcgctacgtga |
| | | ctgggtcatggctgcgccccgacacccgccaacacccgctgacgcgccctgacgggcttgtctgctcccg |
| | | gcatccgcttacagacaagctgtgaccgtctccgggagctgcatgtgtcagaggttttcaccgtcatcaccga |
| | | aacgcgcgaggcagcagatcaattcgcgcgcgaaggcgaagcggcatgcataatgtgcctgtcaaatgga |
| | | cgaagcagggattctgcaaaccctatgctactccgtcaagccgtcaattgtctgattcgttaccaattatgaca |
| | | acttgacggctacatcattcactttttcttcacaaccggcacggaactcgctcgggctggccccggtgcattttt |
| | | taaatacccgcgagaaatagagttgatcgtcaaaaccaacattgcgaccgacggtggcgataggcatccgg |
| | | gtggtgctcaaaagcagcttcgcctggctgatacgttggtcctcgcgccagcttaagacgctaatccctaact |
| | | gctggcggaaaagatgtgacagacgcgacggcgacaagcaaacatgctgtgcgacgctggcgatatcaa |
| | | aattgctgtctgccaggtgatcgctgatgtactgacaagcctcgcgtacccgattatccatcggtggatggag |
| | | cgactcgttaatcgcttccatgcgccgcagtaacaattgctcaagcagatttatcgccagcagctccgaatag |
| | | cgcccttccccttgcccggcgttaatgatttgcccaaacaggtcgctgaaatgcggctggtgcgcttcatccg |
| | | ggcgaaagaaccccgtattggcaaatattgacggccagttaagccattcatgccagtaggcgcgcggacga |
| | | aagtaaacccactggtgataccattcgcgagcctccggatgacgaccgtagtgatgaatctctcctggcggg |
| | | aacagcaaaatatcacccggtcggcaaacaaattctcgtccctgatttttcaccaccccctgaccgcgaatgg |
| | | tgagattgagaatataacctttcattcccagcggtcggtcgataaaaaaatcgagataaccgttggcctcaatc |
| | | ggcgttaaacccgccaccagatgggcattaaacgagtatcccggcagcaggggatcattttgcgcttcagc |
| | | catacttttcatactcccgccattcagag |
| LV- | 9 | caggtggcacttttcggggaaatgtgcgcggaacccctatttgtttatttttctaaatacattcaaatatgtatccg |
| EF1a- | | ctcatgagacaataaccctgataaatgcttcaataatattgaaaaaggaagagtatgagtattcaacatttccgt |
| CD19(FMC63)- | | gtcgcccttattcccttttttgcggcattttgccttcctgtttttgctcacccagaaacgctggtgaaagtaaaaga |
| BBZ | | tgctgaagatcagttgggtgcacgagtgggttacatcgaactggatctcaacagcggtaagatccttgagagt |
| | | tttcgccccgaagaacgttttccaatgatgagcacttttaaagttctgctatgtggcgcggtattatcccgtattg |
| | | acgccgggcaagagcaactcggtcgccgcatacactattctcagaatgacttggttgagtactcaccagtca |
| | | cagaaaagcatcttacggatggcatgacagtaagagaattatgcagtgctgccataaccatgagtgataaca |
| | | ctgcggccaacttacttctgacaacgatcggaggaccgaaggagctaaccgcttttttgcacaacatggggg |
| | | atcatgtaactcgccttgatcgttgggaaccggagctgaatgaagccataccaaacgacgagcgtgacacc |
| | | acgatgcctgtagcaatggcaacaacgttgcgcaaactattaactggcgaactacttactctagcttcccggc |
| | | aacaattaatagactggatggaggcggataaagttgcaggaccacttctgcgctcggcccttccggctggct |
| | | ggtttattgctgataaatctggagccggtgagcgtgggtctcgcggtatcattgcagcactggggccagatg |
| | | gtaagccctcccgtatcgtagttatctacacgacggggagtcaggcaactatggatgaacgaaatagacaga |
| | | tcgctgagataggtgcctcactgattaagcattggtaactgtcagaccaagtttactcatatatactttagattgat |
| | | ttaaaacttcatttttaatttaaaaggatctaggtgaagatcctttttgataatctcatgaccaaaatcccttaacgt |
| | | gagttttcgttccactgagcgtcagaccccgtagaaaagatcaaaggatcttcttgagatcctttttttctgcgcg |
| | | taatctgctgcttgcaaacaaaaaaaccaccgctaccagcggtggtttgtttgccggatcaagagctaccaac |
| | | tctttttccgaaggtaactggcttcagcagagcgcagataccaaatactgtccttctagtgtagccgtagttagg |
| | | ccaccacttcaagaactctgtagcaccgcctacatacctcgctctgctaatcctgttaccagtggctgctgcca |
| | | gtggcgataagtcgtgtcttaccgggttggactcaagacgatagttaccggataaggcgcagcggtcgggc |
| | | tgaacggggggttcgtgcacacagcccagcttggagcgaacgacctacaccgaactgagatacctacagc |
| | | gtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgggtt |
| | | tcgccacctctgacttgagcgtcgatttttgtgatgctcgtcaggggggcggagcctatggaaaaacgccag |
| | | caacgcggcctttttacggttcctggccttttgctggccttttgctcacatgttctttcctgcgttatcccctgattct |
| | | gtggataaccgtattaccgcctttgagtgagctgataccgctcgccgcagccgaacgaccgagcgcagcga |
| | | gtcagtgagcgaggaagcggaagagcgcccaatacgcaaaccgcctctccccgcgcgttggccgattcat |
| | | taatgcagctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagcggata |
| | | acaatttcacacaggaaacagctatgaccatgattacgccaagcgcgcaattaaccctcactaaagggaaca |
| | | aaagctggagctgcaagcttaatgtagtcttatgcaatactcttgtagtcttgcaacatggtaacgatgagttag |
| | | caacatgccttacaaggagagaaaaagcaccgtgcatgccgattggtggaagtaaggtggtacgatcgtgc |
| | | cttattaggaaggcaacagacgggtctgacatggattggacgaaccactgaattgccgcattgcagagatatt |
| | | gtatttaagtgcctagctcgatacaataaacgggtctctctggttagaccagatctgagcctgggagctctctg |
| | | gctaactagggaacccactgcttaagcctcaataaagcttgccttgagtgcttcaagtagtgtgtgcccgtctg |
| | | ttgtgtgactctggtaactagagatccctcagacccttttagtcagtgtggaaaatctctagcagtggcgcccg |
| | | aacagggacctgaaagcgaaagggaaaccagagctctctcgacgcaggactcggcttgctgaagcgcgc |
| | | acggcaagaggcgaggggcggcgactggtgagtacgccaaaaattttgactagcggaggctagaaggag |
| | | agagatgggtgcgagagcgtcagtattaagcgggggagaattagatcgcgatgggaaaaaattcggttaag |
| | | gccagggggaaagaaaaaatataaattaaaacatatagtatgggcaagcagggagctagaacgattcgca |
| | | gttaatcctggcctgttagaaacatcagaaggctgtagacaaatactgggacagctacaaccatcccttcaga |
| | | caggatcagaagaacttagatcattatataatacagtagcaaccctctattgtgtgcatcaaaggatagagata |
| | | aaagacaccaaggaagctttagacaagatagaggaagagcaaaacaaaagtaagaccaccgcacagcaa |
| | | gcggccgctgatcttcagacctggaggaggagatatgagggacaattggagaagtgaattatataaatataa |
| | | agtagtaaaaattgaaccattaggagtagcacccaccaaggcaaagagaagagtggtgcagagagaaaaa |
| | | agagcagtgggaataggagctttgttccttgggttcttgggagcagcaggaagcactatgggcgcagcctc |
| | | aatgacgctgacggtacaggccagacaattattgtctggtatagtgcagcagcagaacaatttgctgagggc |
| | | tattgaggcgcaacagcatctgttgcaactcacagtctggggcatcaagcagctccaggcaagaatcctggc |
| | | tgtggaaagatacctaaaggatcaacagctcctggggatttggggttgctctggaaaactcatttgcaccact |
| | | gctgtgccttggaatgctagttggagtaataaatctctggaacagattggaatcacacgacctggatggagtg |
| | | ggacagagaaattaacaattacacaagcttaatacactccttaattgaagaatcgcaaaaccagcaagaaaa |
| | | gaatgaacaagaattattggaattagataaatgggcaagtttgtggaattggtttaacataacaaattggctgtg |
| | | gtatataaaattattcataatgatagtaggaggcttggtaggtttaagaatagtttttgctgtactttctatagtgaat |
| | | agagttaggcagggatattcaccattatcgtttcagacccacctcccaaccccgaggggacccgacaggcc |
| | | cgaaggaatagaagaagaaggtggagagagagacagagacagatccattcgattagtgaacggatctcga |
| | | cggttaacttttaaaagaaaaggggggattggggggtacagtgcaggggaaagaatagtagacataatagc |
| | | aacagacatacaaactaaagaattacaaaaacaaattacaaaaattcaaaattttatcgagctttgcaaagatg |
| | | gataaagttttaaacagagaggaatctttgcagctaatggaccttctaggtcttgaaaggagtgcctCGTG |
| | | AGGCTCCGGTGCCCGTCAGTGGGCAGAGCGCACATCGCCCACAG |
| | | TCCCCGAGAAGTTGGGGGGAGGGGTCGGCAATTGAACCGGTGCC |
| | | TAGAGAAGGTGGCGCGGGGTAAACTGGGAAAGTGATGTCGTGTA |
| | | CTGGCTCCGCCTTTTTCCCGAGGGTGGGGGAGAACCGTATATAAG |
| | | TGCAGTAGTCGCCGTGAACGTTCTTTTTCGCAACGGGTTTGCCGC |
| | | CAGAACACAGGTAAGTGCCGTGTGTGGTTCCCGCGGGCCTGGCC |
| | | TCTTTACGGGTTATGGCCCTTGCGTGCCTTGAATTACTTCCACCTG |
| | | GCTGCAGTACGTGATTCTTGATCCCGAGCTTCGGGTTGGAAGTGG |
| | | GTGGGAGAGTTCGAGGCCTTGCGCTTAAGGAGCCCCTTCGCCTCG |
| | | TGCTTGAGTTGAGGCCTGGCCTGGGCGCTGGGGCCGCCGCGTGC |
| | | GAATCTGGTGGCACCTTCGCGCCTGTCTCGCTGCTTTCGATAAGT |
| | | CTCTAGCCATTTAAAATTTTTGATGACCTGCTGCGACGCTTTTTTT |
| | | CTGGCAAGATAGTCTTGTAAATGCGGGCCAAGATCTGCACACTG |
| | | GTATTTCGGTTTTTGGGGCCGCGGGCGGCGACGGGGCCCGTGCGT |
| | | CCCAGCGCACATGTTCGGCGAGGCGGGGCCTGCGAGCGCGGCCA |
| | | CCGAGAATCGGACGGGGGTAGTCTCAAGCTGGCCGGCCTGCTCT |
| | | GGTGCCTGGCCTCGCGCCGCCGTGTATCGCCCCGCCCTGGGCGGC |
| | | AAGGCTGGCCCGGTCGGCACCAGTTGCGTGAGCGGAAAGATGGC |
| | | CGCTTCCCGGCCCTGCTGCAGGGAGCTCAAAATGGAGGACGCGG |
| | | CGCTCGGGAGAGCGGGCGGGTGAGTCACCCACACAAAGGAAAA |
| | | GGGCCTTTCCGTCCTCAGCCGTCGCTTCATGTGACTCCACGGAGT |
| | | ACCGGGCGCCGTCCAGGCACCTCGATTAGTTCTCGAGCTTTTGGA |
| | | GTACGTCGTCTTTAGGTTGGGGGGAGGGGTTTTATGCGATGGAGT |
| | | TTCCCCACACTGAGTGGGTGGAGACTGAAGTTAGGCCAGCTTGG |
| | | CACTTGATGTAATTCTCCTTGGAATTTGCCCTTTTTGAGTTTGGAT |
| | | CTTGGTTCATTCTCAAGCCTCAGACAGTGGTTCAAAGTTTTTTTCT |
| | | TCCATTTCAGGTGTCGTGAggaattcggtaccgcggccgcccggggatccatggccttac |
| | | cagtgaccgccttgctcctgccgctggccttgctgctccacgccgccaggccggacatccagatgacacag |
| | | actacatcctccctgtctgcctctctgggagacagagtcaccatcagttgcagggcaagtcaggacattagta |
| | | aatatttaaattggtatcagcagaaaccagatggaactgttaaactcctgatctaccatacatcaagattacact |
| | | caggagtcccatcaaggttcagtggcagtgggtctggaacagattattctctcaccattagcaacctggagca |
| | | agaagatattgccacttacttttgccaacagggtaatacgcttccgtacacgttcggaggggggaccaagctg |
| | | gagatcacaggtggcggtggctcgggcggtggtgggtcgggtggcggcggatctgaggtgaaactgcag |
| | | gagtcaggacctggcctggtggcgccctcacagagcctgtccgtcacatgcactgtctcaggggtctcatta |
| | | cccgactatggtgtaagctggattcgccagcctccacgaaagggtctggagtggctgggagtaatatgggg |
| | | tagtgaaaccacatactataattcagctctcaaatccagactgaccatcatcaaggacaactccaagagccaa |
| | | gttttcttaaaaatgaacagtctgcaaactgatgacacagccatttactactgtgccaaacattattactacggtg |
| | | gtagctatgctatggactactggggccaaggaacctcagtcaccgtctcctcaaccacgacgccagcgccg |
| | | cgaccaccaacaccggcgcccaccatcgcgtcgcagcccctgtccctgcgcccagaggcgtgccggcca |
| | | gcggcggggggcgcagtgcacacgagggggctggacttcgcctgtgatatctacatctgggcgcccttgg |
| | | ccgggacttgtggggtccttctcctgtcactggttatcaccctttactgcaaacggggcagaaagaaactcct |
| | | gtatatattcaaacaaccatttatgagaccagtacaaactactcaagaggaagatggctgtagctgccgatttc |
| | | cagaagaagaagaaggaggatgtgaactgagagtgaagttcagcaggagcgcagacgcccccgcgtac |
| | | aagcagggccagaaccagctctataacgagctcaatctaggacgaagagaggagtacgatgttttggacaa |
| | | gagacgtggccgggaccctgagatggggggaaagccgagaaggaagaaccctcaggaaggcctgtaca |
| | | atgaactgcagaaagataagatggcggaggcctacagtgagattgggatgaaaggcgagcgccggaggg |
| | | gcaaggggcacgatggcctttaccagggtctcagtacagccaccaaggacacctacgacgcccttcacatg |
| | | caggccctgccccctcgctaagtcgacaatcaacctctggattacaaaatttgtgaaagattgactggtattctt |
| | | aactatgttgctccttttacgctatgtggatacgctgctttaatgcctttgtatcatgctattgcttcccgtatggcttt |
| | | cattttctcctccttgtataaatcctggttgctgtctctttatgaggagttgtggcccgttgtcaggcaacgtggcg |
| | | tggtgtgcactgtgtttgctgacgcaacccccactggttggggcattgccaccacctgtcagctcctttccggg |
| | | actttcgctttccccctccctattgccacggcggaactcatcgccgcctgccttgcccgctgctggacagggg |
| | | ctcggctgttgggcactgacaattccgtggtgttgtcggggaagctgacgtcctttccatggctgctcgcctgt |
| | | gttgccacctggattctgcgcgggacgtccttctgctacgtcccttcggccctcaatccagcggaccttccttc |
| | | ccgcggcctgctgccggctctgcggcctcttccgcgtcttcgccttcgccctcagacgagtcggatctccctt |
| | | tgggccgcctccccgcctggaattcgagctcggtacctttaagaccaatgacttacaaggcagctgtagatct |
| | | tagccactttttaaaagaaaaggggggactggaagggctaattcactcccaacgaagacaagatctgcttttt |
| | | gcttgtactgggtctctctggttagaccagatctgagcctgggagctctctggctaactagggaacccactgct |
| | | taagcctcaataaagcttgccttgagtgcttcaagtagtgtgtgcccgtctgttgtgtgactctggtaactagag |
| | | atccctcagacccttttagtcagtgtggaaaatctctagcagtagtagttcatgtcatcttattattcagtatttata |
| | | acttgcaaagaaatgaatatcagagagtgagaggaacttgtttattgcagcttataatggttacaaataaagca |
| | | atagcatcacaaatttcacaaataaagcatttttttcactgcattctagttgtggtttgtccaaactcatcaatgtat |
| | | cttatcatgtctggctctagctatcccgcccctaactccgcccagttccgcccattctccgccccatggctgact |
| | | aattttttttatttatgcagaggccgaggccgcctcggcctctgagctattccagaagtagtgaggaggctttttt |
| | | ggaggcctaggcttttgcgtcgagacgtacccaattcgccctatagtgagtcgtattacgcgcgctcactggc |
| | | cgtcgttttacaacgtcgtgactgggaaaaccctggcgttacccaacttaatcgccttgcagcacatccccctt |
| | | tcgccagctggcgtaatagcgaagaggcccgcaccgatcgcccttcccaacagttgcgcagcctgaatgg |
| | | cgaatggcgcgacgcgccctgtagcggcgcattaagcgcggcgggtgtggtggttacgcgcagcgtgac |
| | | cgctacacttgccagcgccctagcgcccgctcctttcgctttcttcccttcctttctcgccacgttcgccggcttt |
| | | ccccgtcaagctctaaatcgggggctccctttagggttccgatttagtgctttacggcacctcgaccccaaaa |
| | | aacttgattagggtgatggttcacgtagtgggccatcgccctgatagacggtttttcgccctttgacgttggagt |
| | | ccacgttctttaatagtggactcttgttccaaactggaacaacactcaaccctatctcggtctattcttttgatttat |
| | | aagggattttgccgatttcggcctattggttaaaaaatgagctgatttaacaaaaatttaacgcgaattttaacaa |
| | | aatattaacgtttacaatttcc |

TABLE 19
CAR-EC switch target interacting domains
(antibodies, variable regions)-Amino Acid Sequence

| NAME | SEQ ID | SEQUENCE |
|---|---|---|
| Light chain of | 10 | DIQMTQSPSSLSASVGDRVTITCKASQDVGIAVAWYQQ |
| wildtype anti-CS1 | | KPGKVPKLLIYWASTRHTGVPDRFSGSGSGTDFTLTISSL |
| antibody | | QPEDVATYYCQQYSSYPYTFGQGTKLEIK |
| Heavy chain of | 11 | EVQLVESGGGLVQPGGSLRLSCAASGFDFSRYWMSWV |
| wildtype anti-CS1 | | RQAPGKGLEWIGEINPDSSTINYAPSLKDKFIISRDNAKN |
| antibody | | SLYLQMNSLRAEDTAVYYCARPDGNYWYFDVWGQGT |
| | | LVTVSS |
| anti-Her2-Fab light | 12 | DIQMTQSPSSLSASVGDRVTITCRASQDVNTAVAWYQQ |
| chain | | KPGKAPKLLIYSASFLYSGVPSRFSGSRSGTDFTLTISSLQ |
| | | PEDFATYYCQQHYTTPPTFGQGTKLEIK |
| Anti-Her2-Fab heavy | 13 | QLQESGPGLVKPSQTLSLTCTVSGYSISSDFAWNWIRQP |
| chain | | PGKGLEWMGYISYSGNTRYQPSLKSRITISRDTSKNQFF |
| | | LKLNSVTAADTATYYCVTAGRGFPYWGQGTLVTVSS |
| Light chain of anti- | 14 | DIQMTQSPSSLSASVGDRVTITCRANQGISNNLNWYQQ |
| BCMA antibody | | KPGKAPKPLIYYTSNLQSGVPSRFSGSGSGTDYTLTISSL |
| (BCMA98) | | QPEDFATYYCQQFTSLPYTFGQGTKLEIK |
| Heavy chain of anti- | 15 | EVQLVESGGGLVQPGGSLRLSCAASGFTFSNFDMAWVR |
| BCMA antibody | | QAPGKGLVWVSSITTGGGDTYYADSVKGRFTISRDNAK |
| (BCMA98) | | STLYLQMDSLRSEDTAVYYCVRHGYYDGYEILFDYWG |
| | | QGTLVTVSS |
| anti-CD19-Fab Light | 16 | DIQMTQTTSSLSASLGDRVTISCRASQDISKYLNWYQQK |
| Chain | | PDGTVKLLIYHTSRLHSGVPSRFSGSGSGTDYSLTISNLE |
| | | QEDIATYFCQQGNTLPYTFGGGTKLEIK |
| anti-CD19-Fab Heavy | 17 | EVKLQESGPGLVAPSQSLSVTCTVSGVSLPDYGVSWIRQ |
| Chain | | PPRKGLEWLGVIWGSETTYYNSALKSRLTIIKDNSKSQV |
| | | FLKMNSLQTDDTAIYYCAKHYYYGGSYAMDYWGQGT |
| | | SVTVSS |
| anti-CLL1-Fab Light | 18 | ENVLTQSPAIMSASPGEKVTMTCRASSNVISSYVHWYQ |
| Chain (clone 1075.7) | | QRSGASPKLWIYSTSNLASGVPARFSGSGSGTSYSLTISS |
| | | VEAEDAATYYCQQYSGYPLTFGAGTKLELK |
| anti-CLL1-Fab Heavy | 19 | DIQLQESGPGLVKPSQSLSLTCSVTGYSITSAYYWNWIR |
| Chain (clone 1075.7) | | QFPGNKLEWMGYISYDGRNNYNPSLKNRISITRDTSKN |
| | | QFFLKLNSVTTEDTATYYCAKEGDYDVGNYYAMDYW |
| | | GQGTSVTVSS |
| anti-CD33-Fab Light | 20 | DIQLTQSPSTLSASVGDRVTITCRASESLDNYGIRFLTWF |
| Chain (clone HP67.6) | | QQKPGKAPKLLMYAASNQGSGVPSRFSGSGSGTEFTLTI |
| | | SSLQPDDFATYYCQQTKEVPWSFGQGTKVEVK |
| anti-CD33-Fab | 21 | EVQLVQSGAEVKKPGSSVKVSCKASGYTITDSNIHWVR |
| Heavy Chain (clone | | QAPGQSLEWIGYIYPYNGGTDYNQKFKNRATLTVDNPT |
| HP67.6) | | NTAYMELSSLRSEDTAFYYCVNGNPWLAYWGQGTLVT |
| | | VSS |
| anti-CD33-Fab Light | 22 | DIQMTQSPSSLSASVGDRVTITCRASESVDNYGISFMNW |
| Chain (clone hM195) | | FQQKPGKAPKLLIYAASNQGSGVPSRFSGSGSGTDFTLN |
| | | ISSLQPDDFATYYCQQSKEVPWTFGQGTKVEIK |
| anti-CD33-Fab | 23 | QVQLVQSGAEVKKPGSSVKVSCKASGYTFTDYNMHWV |
| Heavy Chain (clone | | RQAPGQGLEWIGYIYPYNGGTGYNQKFKSKATITADES |
| hM195) | | TNTAYMELSSLRSEDTAVYYCARGRPAMDYWGQGTLV |
| | | TVSS |
| anti-CD123-Fab | 24 | DIVLTQSPASLAVSLGQRATISCRASESVDNYGNTFMH |
| Light Chain (clone | | WYQQKPGQPPKLLIYRASNLESGIPARFSGSGSRTDFTL |
| 32716) | | TINPVEADDVATYYCQQSNEDPPTFGAGTKLELK |
| anti-CD123-Fab | 25 | QIQLVQSGPELKKPGETVKISCKASGYIFTNYGMNWVK |
| Heavy Chain (clone | | QAPGKSFKWMGWINTYTGESTYSADFKGRFAFSLETSA |
| 32716) | | STAYLHINDLKNEDTATYFCARSGGYDPMDYWGQGTS |
| | | VTVSS |
| anti-CD123-Fab | 26 | DVQITQSPSYLAASPGETITINCRASKSISKDLAWYQEKP |
| Light Chain (clone | | GKTNKLLIYSGSTLQSGIPSRFSGSGSGTDFTLTISSLEPE |
| 26292) | | DFAMYYCQQHNKYPYTFGGGTKLEIK |
| anti-CD123-Fab | 27 | QVQLQQPGAELVRPGASVKLSCKASGYTFTSYWMNWV |
| Heavy Chain (clone | | KQRPDQGLEWIGRIDPYDSETHYNQKFKDKAILTVDKS |
| 26292) | | SSTAYMQLSSLTSEDSAVYYCARGNWDDYWGQGTTLT |
| | | VSS |
| Anti-CD22-Fab light | 28 | DIQMTQSPSSLSASVGDRVTITCRASQTIWSYLNWYQQR |
| Chain (clone M971) | | PGKAPNLLIYAASSLQSGVPSRFSGRGSGTDFTLTISSLQ |
| | | AEDFATYYCQQSYSIPQTFGQGTKLEIK |
| Anti-CD22-Fab | 29 | QVQLQQSGPGLVKPSQTLSLTCAISGDSVSSNSAAWNWI |
| heavy Chain (clone | | RQSPSRGLEWLGRTYYRSKWYNDYAVSVKSRITINPDT |
| M971) | | SKNQFSLQLNSVTPEDTAVYYCAREVTGDLEDAFDIWG |
| | | QGTMVTVSS |
| Anti-CD22-Fab light | 30 | DIQLTQSPSSLSASVGDRVTMSCKSSQSVLYSANHKNYL |
| Chain (clone hLL2) | | AWYQQKPGKAPKLLIYVVASTRESGVPSRFSGSGSGTDF |
| | | TFTISSLQPEDIATYYCHQYLSSWTEGGGTKLEIK |
| Anti-CD22-Fab | 31 | QVQLVQSGAEVKKPGSSVKVSCKASGYTFTSYWLHWV |
| Heavy Chain (clone | | RQAPGQGLEWIGYINPRNDYTEYNQNFKDKATITADES |
| hLL2) | | TNTAYMELSSLRSEDTAFYFCARRDITTFYWGQGTTVT |
| | | VSS |

TABLE 20
Soluble T cell receptors-Nucleotide Sequence

| | SEQ | |
|---|---|---|
| | ID | |
| NAME | NO | SEQUENCE |
| NYESO1(1G4- | 32 | tccggatatagttcctcctttcagcaaaaaacccctcaagacccgtttagaggccccaaggggttatgcta |
| 113)-TCRb- | | gttattgctcagcggtggcagcagccaactcagcttcctttcgggctttgttagcagccggatctcagtggt |
| WT | | ggtggtggtggtgctcgagTTAGTCAGCACGACCCCACGCTTCTGCACTGA |
| | | CAATTTGCGTAACCGGTTTTGCACGGTCCTGGGTCCATTCATCA |
| | | TTTTCGCTCAGGCCGTAGAATTGAACCTGACAGCGAAAATGGTT |
| | | ACGCGGGTCCTGCCAGAAGGTGGCAGACACGCGCAGACGGGA |
| | | ACTCAGGGCATAACGGCTGTCATTCAGCGCCGGTTGTTCTTTCA |
| | | GCGGCTGCGGATCGGTACACACACCCGAGTGGACTTCTTTGCC |
| | | GTTCACCCACCATGACAGTTCGACATGGTCCGGATAAAAGCCG |
| | | GTGGCCAGGCACACCAGCGTCGCTTTCTGGGTATGGGAGATTTC |
| | | AGCTTCACTCGGTTCGAAAACTGCCACTTCCGGCGGAAAGACA |
| | | TTTTTCAGATCTTCCAGAACCGTCAGACGAGAACCTTCGCCGAA |
| | | AAACAGTTCACCGGTGTTGCCCAGGTAAGAGCTTGCGCAGAAA |
| | | TAAACAGACGTTTGGCTCGGTGCTGCGCTCAGCAGACGCAGCG |
| | | GAAAGTCTTCAATGGTCGAACGTGAAACATTGTAACCGTTCGG |
| | | CACTTCGCCTTGATCCGTGGTCTGGATGGCGACCGAATAGTGAA |
| | | TCAGACGCAGACCCATACCCGGGTCCTGACGGTACCATGACAT |
| | | ATATTCATGGTTCATATCTTGGGCACATTGCAGCGTCATGGATT |
| | | GGCCCGTTTTCAGGACTTGGAATTTCGGCGTTTGGGTCACACCA |
| | | GCGTTCATatgtatatctccttcttaaagttaaacaaaattatttctagaggggaattgttatccgctca |
| | | caattcccctatagtgagtcgtattaatttcgcgggatcgagatctcgatcctctacgccggacgcatcgtg |
| | | gccggcatcaccggcgccacaggtgcggttgctggcgcctatatcgccgacatcaccgatggggaaga |
| | | tcgggctcgccacttcgggctcatgagcgcttgtttcggcgtgggtatggtggcaggccccgtggccgg |
| | | gggactgttgggcgccatctccttgcatgcaccattccttgcggcggcggtgctcaacggcctcaaccta |
| | | ctactgggctgcttcctaatgcaggagtcgcataagggagagcgtcgagatcccggacaccatcgaatg |
| | | gcgcaaaacctttcgcggtatggcatgatagcgcccggaagagagtcaattcagggtggtgaatgtgaa |
| | | accagtaacgttatacgatgtcgcagagtatgccggtgtctcttatcagaccgtttcccgcgtggtgaacca |
| | | ggccagccacgtttctgcgaaaacgcgggaaaaagtggaagcggcgatggcggagctgaattacattc |
| | | ccaaccgcgtggcacaacaactggcgggcaaacagtcgttgctgattggcgttgccacctccagtctgg |
| | | ccctgcacgcgccgtcgcaaattgtcgcggcgattaaatctcgcgccgatcaactgggtgccagcgtggt |
| | | ggtgtcgatggtagaacgaagcggcgtcgaagcctgtaaagcggcggtgcacaatcttctcgcgcaac |
| | | gcgtcagtgggctgatcattaactatccgctggatgaccaggatgccattgctgtggaagctgcctgcact |
| | | aatgttccggcgttatttcttgatgtctctgaccagacacccatcaacagtattattttctcccatgaagacggt |
| | | acgcgactgggcgtggagcatctggtcgcattgggtcaccagcaaatcgcgctgttagcgggcccatta |
| | | agttctgtctcggcgcgtctgcgtctggctggctggcataaatatctcactcgcaatcaaattcagccgata |
| | | gcggaacgggaaggcgactggagtgccatgtccggttttcaacaaaccatgcaaatgctgaatgagggc |
| | | atcgttcccactgcgatgctggttgccaacgatcagatggcgctgggcgcaatgcgcgccattaccgagt |
| | | ccgggctgcgcgttggtgcggatatctcggtagtgggatacgacgataccgaagacagctcatgttatat |
| | | cccgccgttaaccaccatcaaacaggattttcgcctgctggggcaaaccagcgtggaccgcttgctgcaa |
| | | ctctctcagggccaggcggtgaagggcaatcagctgttgcccgtctcactggtgaaaagaaaaaccacc |
| | | ctggcgcccaatacgcaaaccgcctctccccgcgcgttggccgattcattaatgcagctggcacgacag |
| | | gtttcccgactggaaagcgggcagtgagcgcaacgcaattaatgtaagttagctcactcattaggcaccg |
| | | ggatctcgaccgatgcccttgagagccttcaacccagtcagctccttccggtgggcgcggggcatgacta |
| | | tcgtcgccgcacttatgactgtcttctttatcatgcaactcgtaggacaggtgccggcagcgctctgggtca |
| | | ttttcggcgaggaccgctttcgctggagcgcgacgatgatcggcctgtcgcttgcggtattcggaatcttg |
| | | cacgccctcgctcaagccttcgtcactggtcccgccaccaaacgtttcggcgagaagcaggccattatcg |
| | | ccggcatggcggccccacgggtgcgcatgatcgtgctcctgtcgttgaggacccggctaggctggcgg |
| | | ggttgccttactggttagcagaatgaatcaccgatacgcgagcgaacgtgaagcgactgctgctgcaaaa |
| | | cgtctgcgacctgagcaacaacatgaatggtcttcggtttccgtgtttcgtaaagtctggaaacgcggaagt |
| | | cagcgccctgcaccattatgttccggatctgcatcgcaggatgctgctggctaccctgtggaacacctaca |
| | | tctgtattaacgaagcgctggcattgaccctgagtgatttttctctggtcccgccgcatccataccgccagtt |
| | | gtttaccctcacaacgttccagtaaccgggcatgttcatcatcagtaacccgtatcgtgagcatcctctctcg |
| | | tttcatcggtatcattacccccatgaacagaaatcccccttacacggaggcatcagtgaccaaacaggaaa |
| | | aaaccgcccttaacatggcccgctttatcagaagccagacattaacgcttctggagaaactcaacgagct |
| | | ggacgcggatgaacaggcagacatctgtgaatcgcttcacgaccacgctgatgagctttaccgcagctg |
| | | cctcgcgcgtttcggtgatgacggtgaaaacctctgacacatgcagctcccggagacggtcacagcttgt |
| | | ctgtaagcggatgccgggagcagacaagcccgtcagggcgcgtcagcgggtgttggcgggtgtcggg |
| | | gcgcagccatgacccagtcacgtagcgatagcggagtgtatactggcttaactatgcggcatcagagca |
| | | gattgtactgagagtgcaccatatatgcggtgtgaaataccgcacagatgcgtaaggagaaaataccgca |
| | | tcaggcgctcttccgcttcctcgctcactgactcgctgcgctcggtcgttcggctgcggcgagcggtatca |
| | | gctcactcaaaggcggtaatacggttatccacagaatcaggggataacgcaggaaagaacatgtgagca |
| | | aaaggccagcaaaaggccaggaaccgtaaaaaggccgcgttgctggcgtttttccataggctccgcccc |
| | | cctgacgagcatcacaaaaatcgacgctcaagtcagaggtggcgaaacccgacaggactataaagata |
| | | ccaggcgtttccccctggaagctccctcgtgcgctctcctgttccgaccctgccgcttaccggatacctgtc |
| | | cgcctttctcccttcgggaagcgtggcgctttctcatagctcacgctgtaggtatctcagttcggtgtaggtc |
| | | gttcgctccaagctgggctgtgtgcacgaaccccccgttcagcccgaccgctgcgccttatccggtaact |
| | | atcgtcttgagtccaacccggtaagacacgacttatcgccactggcagcagccactggtaacaggattag |
| | | cagagcgaggtatgtaggcggtgctacagagttcttgaagtggtggcctaactacggctacactagaagg |
| | | acagtatttggtatctgcgctctgctgaagccagttaccttcggaaaaagagttggtagctcttgatccggc |
| | | aaacaaaccaccgctggtagcggtggtttttttgtttgcaagcagcagattacgcgcagaaaaaaaggatc |
| | | tcaagaagatcctttgatcttttctacggggtctgacgctcagtggaacgaaaactcacgttaagggattttg |
| | | gtcatgagattatcaaaaaggatcttcacctagatcatttaaattaaaaatgaagattaaatcaatctaaagt |
| | | atatatgagtaaacttggtctgacagttaccaatgcttaatcagtgaggcacctatctcagcgatctgtctattt |
| | | cgttcatccatagttgcctgactccccgtcgtgtagataactacgatacgggagggcttaccatctggcccc |
| | | agtgctgcaatgataccgcgagacccacgctcaccggctccagatttatcagcaataaaccagccagcc |
| | | ggaagggccgagcgcagaagtggtcctgcaactttatccgcctccatccagtctattaattgttgccggga |
| | | agctagagtaagtagttcgccagttaatagtttgcgcaacgttgttgccattgctgcaggcatcgtggtgtca |
| | | cgctcgtcgtttggtatggcttcattcagctccggttcccaacgatcaaggcgagttacatgatcccccatgt |
| | | tgtgcaaaaaagcggttagctccttcggtcctccgatcgttgtcagaagtaagttggccgcagtgttatcac |
| | | tcatggttatggcagcactgcataattctcttactgtcatgccatccgtaagatgcttttctgtgactggtgagt |
| | | actcaaccaagtcattctgagaatagtgtatgcggcgaccgagttgctcttgcccggcgtcaatacgggat |
| | | aataccgcgccacatagcagaactttaaaagtgctcatcattggaaaacgttcttcggggcgaaaactctc |
| | | aaggatcttaccgctgttgagatccagttcgatgtaacccactcgtgcacccaactgatcttcagcatctttta |
| | | ctttcaccagcgtttctgggtgagcaaaaacaggaaggcaaaatgccgcaaaaaagggaataagggcg |
| | | acacggaaatgttgaatactcatactcttcattttcaatattattgaagcatttatcagggttattgtctcatgag |
| | | cggatacatatttgaatgtatttagaaaaataaacaaataggggttccgcgcacatttccccgaaaagtgcc |
| | | acctgaaattgtaaacgttaatattttgttaaaattcgcgttaaatttttgttaaatcagctcattttttaaccaata |
| | | ggccgaaatcggcaaaatcccttataaatcaaaagaatagaccgagatagggttgagtgagttccagttt |
| | | ggaacaagagtccactattaaagaacgtggactccaacgtcaaagggcgaaaaaccgtctatcagggc |
| | | gatggcccactacgtgaaccatcaccctaatcaagttattggggtcgaggtgccgtaaagcactaaatcg |
| | | gaaccctaaagggagcccccgatttagagcttgacggggaaagccggcgaacgtggcgagaaaggaa |
| | | gggaagaaagcgaaaggagcgggcgctagggcgctggcaagtgtagcggtcacgctgcgcgtaacc |
| | | accacacccgccgcgcttaatgcgccgctacagggcgcgtcccattcgcca |
| NYESO1(1G4- | 33 | atccggatatagttcctcctttcagcaaaaaacccctcaagacccgtttagaggccccaaggggttatgcta |
| 113)-TCRa- | | gttattgctcagcggtggcagcagccaactcagcttcctttcgggctttgttagcagccggatctcagtggt |
| WT | | ggtggtggtggtgctcgagTTAAGAGCTTTCCGGGCTCGGGAAAAACGTA |
| | | TCTTCCGGGATAATACTATTGTTGAATGCGTTGGCGCACGCAAA |
| | | GTCAGATTTATTGCTCCAAGCAACTGCCGAGTTTGATTTGAAAT |
| | | CCATAGAGCGCATGTCCAGAACACATTTATCGGTAATATACAC |
| | | GTCGGAATCTTTGGATTGACTGACATTCGTCTGACTGTCGAAAT |
| | | CGGTAAACAGGCACACGCTTTTGTCCGATGATTTGCTATCACGC |
| | | AGTTGGTACACCGCCGGGTCCGGGTTCTGGATATACGGATGGA |
| | | CAATCAGGCTGGTACCGCGGCCAAACGTCGGGATATAGGTACC |
| | | ATCCAGCAGCGGACGCACCGCACACAGGTACGTAGCGGAGTCA |
| | | CCCGGCTGACTTGCTGCAATATACAGGGTGGAACTACCAGAGC |
| | | TTTTATCCAGCGATGCATTCAGGCGGCCTGAGGTTTGTTCACGC |
| | | TGCCACGGCGTGATCAGCAGCAGGCTGGTCAGACCTTTGCCCG |
| | | GATCTTGACGAAACCACTGCAGGTTGTAAATGGCGCTATCCGT |
| | | AAAGGAGCAATTCAGCACCAGATTTTCACCTTCCGGGACCGAC |
| | | AGAGCAGCCGGGATTTGGGTAACTTCTTGTTTCATatgtatatctccttctt |
| | | aaagttaaacaaaattatttctagaggggaattgttatccgctcacaattcccctatagtgagtcgtattaattt |
| | | cgcgggatcgagatctcgatcctctacgccggacgcatcgtggccggcatcaccggcgccacaggtgc |
| | | ggttgctggcgcctatatcgccgacatcaccgatggggaagatcgggctcgccacttcgggctcatgag |
| | | cgcttgtttcggcgtgggtatggtggcaggccccgtggccgggggactgagggcgccatctccttgcat |
| | | gcaccattccttgcggcggcggtgctcaacggcctcaacctactactgggctgcttcctaatgcaggagtc |
| | | gcataagggagagcgtcgagatcccggacaccatcgaatggcgcaaaacctttcgcggtatggcatgat |
| | | agcgcccggaagagagtcaattcagggtggtgaatgtgaaaccagtaacgttatacgatgtcgcagagt |
| | | atgccggtgtctcttatcagaccgtttcccgcgtggtgaaccaggccagccacgtttctgcgaaaacgcg |
| | | ggaaaaagtggaagcggcgatggcggagctgaattacattcccaaccgcgtggcacaacaactggcg |
| | | ggcaaacagtcgttgctgattggcgttgccacctccagtctggccctgcacgcgccgtcgcaaattgtcg |
| | | cggcgattaaatctcgcgccgatcaactgggtgccagcgtggtggtgtcgatggtagaacgaagcggcg |
| | | tcgaagcctgtaaagcggcggtgcacaatcttctcgcgcaacgcgtcagtgggctgatcattaactatccg |
| | | ctggatgaccaggatgccattgctgtggaagctgcctgcactaatgttccggcgttatttcttgatgtctctg |
| | | accagacacccatcaacagtattattactcccatgaagacggtacgcgactgggcgtggagcatctggtc |
| | | gcattgggtcaccagcaaatcgcgctgttagcgggcccattaagttctgtctcggcgcgtctgcgtctggc |
| | | tggctggcataaatatctcactcgcaatcaaattcagccgatagcggaacgggaaggcgactggagtgc |
| | | catgtccggattcaacaaaccatgcaaatgctgaatgagggcatcgttcccactgcgatgctggttgccaa |
| | | cgatcagatggcgctgggcgcaatgcgcgccattaccgagtccgggctgcgcgttggtgcggatatctc |
| | | ggtagtgggatacgacgataccgaagacagctcatgttatatcccgccgttaaccaccatcaaacaggatt |
| | | ttcgcctgctggggcaaaccagcgtggaccgcttgctgcaactctctcagggccaggcggtgaagggc |
| | | aatcagctgttgcccgtctcactggtgaaaagaaaaaccaccctggcgcccaatacgcaaaccgcctctc |
| | | cccgcgcgttggccgattcattaatgcagctggcacgacaggtttcccgactggaaagcgggcagtgag |
| | | cgcaacgcaattaatgtaagttagctcactcattaggcaccgggatctcgaccgatgcccttgagagcctt |
| | | caacccagtcagctccttccggtgggcgcggggcatgactatcgtcgccgcacttatgactgtcttctttat |
| | | catgcaactcgtaggacaggtgccggcagcgctctgggtcattttcggcgaggaccgctttcgctggagc |
| | | gcgacgatgatcggcctgtcgcttgcggtattcggaatcttgcacgccctcgctcaagccttcgtcactggt |
| | | cccgccaccaaacgtttcggcgagaagcaggccattatcgccggcatggcggccccacgggtgcgcat |
| | | gatcgtgctcctgtcgttgaggacccggctaggctggcggggttgccttactggttagcagaatgaatcac |
| | | cgatacgcgagcgaacgtgaagcgactgctgctgcaaaacgtctgcgacctgagcaacaacatgaatg |
| | | gtcttcggtttccgtgtttcgtaaagtctggaaacgcggaagtcagcgccctgcaccattatgttccggatct |
| | | gcatcgcaggatgctgctggctaccctgtggaacacctacatctgtattaacgaagcgctggcattgaccc |
| | | tgagtgatttttctctggtcccgccgcatccataccgccagttgtttaccctcacaacgttccagtaaccggg |
| | | catgttcatcatcagtaacccgtatcgtgagcatcctctctcgtttcatcggtatcattacccccatgaacaga |
| | | aatcccccttacacggaggcatcagtgaccaaacaggaaaaaaccgcccttaacatggcccgctttatca |
| | | gaagccagacattaacgcttctggagaaactcaacgagctggacgcggatgaacaggcagacatctgtg |
| | | aatcgcttcacgaccacgctgatgagctttaccgcagctgcctcgcgcgtttcggtgatgacggtgaaaac |
| | | ctctgacacatgcagctcccggagacggtcacagcttgtctgtaagcggatgccgggagcagacaagc |
| | | ccgtcagggcgcgtcagcgggtgttggcgggtgtcggggcgcagccatgacccagtcacgtagcgata |
| | | gcggagtgtatactggcttaactatgcggcatcagagcagattgtactgagagtgcaccatatatgcggtg |
| | | tgaaataccgcacagatgcgtaaggagaaaataccgcatcaggcgctcttccgcttcctcgctcactgact |
| | | cgctgcgctcggtcgttcggctgcggcgagcggtatcagctcactcaaaggcggtaatacggttatccac |
| | | agaatcaggggataacgcaggaaagaacatgtgagcaaaaggccagcaaaaggccaggaaccgtaa |
| | | aaaggccgcgttgctggcgtttttccataggctccgcccccctgacgagcatcacaaaaatcgacgctca |
| | | agtcagaggtggcgaaacccgacaggactataaagataccaggcgtttccccctggaagctccctcgtg |
| | | cgctctcctgttccgaccctgccgcttaccggatacctgtccgcctttctcccttcgggaagcgtggcgcttt |
| | | ctcatagctcacgctgtaggtatctcagttcggtgtaggtcgttcgctccaagctgggctgtgtgcacgaac |
| | | cccccgttcagcccgaccgctgcgccttatccggtaactatcgtcttgagtccaacccggtaagacacga |
| | | cttatcgccactggcagcagccactggtaacaggattagcagagcgaggtatgtaggcggtgctacaga |
| | | gttcttgaagtggtggcctaactacggctacactagaaggacagtatttggtatctgcgctctgctgaagcc |
| | | agttaccttcggaaaaagagttggtagctcttgatccggcaaacaaaccaccgctggtagcggtggtttttt |
| | | tgtttgcaagcagcagattacgcgcagaaaaaaaggatctcaagaagatcctttgatcttttctacggggtc |
| | | tgacgctcagtggaacgaaaactcacgttaagggattttggtcatgagattatcaaaaaggatcttcaccta |
| | | gatccttttaaattaaaaatgaagttttaaatcaatctaaagtatatatgagtaaacttggtctgacagttacca |
| | | atgcttaatcagtgaggcacctatctcagcgatctgtctatttcgttcatccatagttgcctgactccccgtcgt |
| | | gtagataactacgatacgggagggcttaccatctggccccagtgctgcaatgataccgcgagacccacg |
| | | ctcaccggctccagatttatcagcaataaaccagccagccggaagggccgagcgcagaagtggtcctg |
| | | caactttatccgcctccatccagtctattaattgttgccgggaagctagagtaagtagttcgccagttaatagt |
| | | ttgcgcaacgttgttgccattgctgcaggcatcgtggtgtcacgctcgtcgtttggtatggcttcattcagctc |
| | | cggttcccaacgatcaaggcgagttacatgatcccccatgttgtgcaaaaaagcggttagctccttcggtc |
| | | ctccgatcgttgtcagaagtaagttggccgcagtgttatcactcatggttatggcagcactgcataattctctt |
| | | actgtcatgccatccgtaagatgcttttctgtgactggtgagtactcaaccaagtcattctgagaatagtgtat |
| | | gcggcgaccgagttgctcttgcccggcgtcaatacgggataataccgcgccacatagcagaactttaaaa |
| | | gtgctcatcattggaaaacgttcttcggggcgaaaactctcaaggatcttaccgctgttgagatccagttcg |
| | | atgtaacccactcgtgcacccaactgatcttcagcatcttttactttcaccagcgtttctgggtgagcaaaaa |
| | | caggaaggcaaaatgccgcaaaaaagggaataagggcgacacggaaatgttgaatactcatactcttcc |
| | | tttttcaatattattgaagcatttatcagggttattgtctcatgagcggatacatatttgaatgtatttagaaaaat |
| | | aaacaaataggggttccgcgcacatttccccgaaaagtgccacctgaaattgtaaacgttaatattttgttaa |
| | | aattcgcgttaaatttttgttaaatcagctcattttttaaccaataggccgaaatcggcaaaatcccttataaatc |
| | | aaaagaatagaccgagatagggttgagtgttgttccagtttggaacaagagtccactattaaagaacgtgg |
| | | actccaacgtcaaagggcgaaaaaccgtctatcagggcgatggcccactacgtgaaccatcaccctaat |
| | | caagttttttggggtcgaggtgccgtaaagcactaaatcggaaccctaaagggagcccccgatttagagc |
| | | ttgacggggaaagccggcgaacgtggcgagaaaggaagggaagaaagcgaaaggagcgggcgcta |
| | | gggcgctggcaagtgtagcggtcacgctgcgcgtaaccaccacacccgccgcgcttaatgcgccgcta |
| | | cagggcgcgtcccattcgcca |

TABLE 21
CAR Hinge-Amino Acid Sequence

| | SEQ ID | |
|---|---|---|
| NAME | NO | SEQUENCE |
| CD8 hinge peptide | 34 | TTTPAPRPPTPAPTIASQPLSL |
| sequence | | RPEACRPAAGGAVHTRGLDFACD |
| no hinge peptide | 35 | D |
| sequence | | |
| IgG4 hinge peptide | 36 | ESKYGPPCPSCP |
| sequence | | |
| IgG4 m hinge | 37 | ESKYGPPCPPCP |
| peptide sequence | | |

TABLE 22
Mouse anti-CD19 Antibody

| | SEQ | |
|---|---|---|
| | ID | |
| NAME | NO | SEQUENCE |
| mCD19(1D3)- | 38 | ggatctgcgatcgctccggtgcccgtcagtgggcagagcgcacatcgcccacagtccccgagaagttggggggagg |
| LC-WT | | ggtcggcaattgaacgggtgcctagagaaggtggcgcggggtaaactgggaaagtgatgtcgtgtactggctccgcct |
| (wild type | | ttacccgagggtgggggagaaccgtatataagtgcagtagtcgccgtgaacgttctttttcgcaacgggtttgccgccag |
| light | | aacacagctgaagcttcgaggggctcgcatctctccttcacgcgcccgccgccctacctgaggccgccatccacgccg |
| chain | | gagagtcgcgttctgccgcctcccgcctgtggtgcctcctgaactgcgtccgccgtctaggtaagataaagctcaggtc |
| sequence) | | gagaccgggcctttgtccggcgctcccttggagcctacctagactcagccggctctccacgctttgcctgaccctgcttg |
| | | ctcaactctacgtctagatcgtatctgactgcgccgttacagatccaagctgtgaccggcgcctacctgagatcaccgg |
| | | cgaaggagggccaccatgtacaggatgcaactcctgtcttgcattgcactaagtcttgcacttgtcacgaattcgGAC |
| | | ATTCAGCTGACCCAGTCTCCAGCTTCCCTGTCTACATCTCTGGGAGAAAC |
| | | TGTCACCATCCAATGTCAAGCAAGTGAGGACATTTACAGTGGTTTAGCG |
| | | TGGTATCAGCAGAAGCCAGGGAAATCTCCTCAGCTCCTGATCTATGGTG |
| | | CAAGTGACTTACAAGACGGCGTCCCATCACGATTCAGTGGCAGTGGATC |
| | | TGGCACACAGTATTCTCTCAAGATCACCAGCATGCAAACTGAAGATGAA |
| | | GGGGTTTATTTCTGTCAACAGGGTTTAACGTATCCTCGGACGTTCGGTGG |
| | | CGGCACCAAGCTGGAAATCaaacgaactgtggctgcaccatctgtcttcatcttcccgccatctgatga |
| | | gcagttgaaatctggaactgcctctgtcgtgtgcctgctgaataacttctatcccagagaggccaaagtacagtggaaggt |
| | | ggataacgccctccaatcgggtaactcccaggagagtgtcacagagcaggacagcGGCGGAGGCGGGA |
| | | GCaattatcatcttgaaaatgaggtcgctcgtctcaagaaactcGGTGGCGGAGGCAGCgacagcaccta |
| | | cagcctcagcagcaccctgacgcttagcaaagcagactacgagaaacacaaagtctacgcctgcgaagtcacccatca |
| | | gggcctgtcctcgcccgtcacaaagagcttcaacaggggagagtgttgataagtgctagctggccagacatgataagat |
| | | acattgatgagtaggacaaaccacaactagaatgcagtgaaaaaaatgctaatagtgaaatttgtgatgctattgctaatt |
| | | tgtaaccattataagctgcaataaacaagttaacaacaacaattgcattcattttatgtttcaggttcagggggaggtgtggg |
| | | aggttattaaagcaagtaaaacctctacaaatgtggtatggaattaattctaaaatacagcatagcaaaacataacctccaa |
| | | atcaagcctctacttgaatccttactgagggatgaataaggcataggcatcaggggctgttgccaatgtgcattagctgat |
| | | gcagcctcaccttctttcatggagtttaagatatagtgtattttcccaaggtttgaactagctcttcatttctttatgttttaaatgc |
| | | actgacctcccacattccctattagtaaaatattcagaaataataaaatacatcattgcaatgaaaataaatgattttattagg |
| | | cagaatccagatgctcaaggcccttcataatatcccccagtaagtagaggacttagggaacaaaggaacctttaataga |
| | | aattggacagcaagaaagcgagcttctagcttatcctcagtcctgctcctctgccacaaagtgcacgcagttgccggccg |
| | | ggtcgcgcagggcgaactcccgcccccacggctgctcgccgatctcggtcatggccggcccggaggcgtcccggaa |
| | | gttcgtggacacgacctccgaccactcggcgtacagctcgtccaggccgcgcacccacacccaggccagggtgttgtc |
| | | cggcaccacctggtcctggaccgcgctgatgaacagggtcacgtcgtcccggaccacaccggcgaagtcgtcctcca |
| | | cgaagtcccgggagaacccgagccggtcggtccagaactcgaccgctccggcgacgtcgcgcgcggtgagcaccg |
| | | gaacggcactggtcaacttggccatgatggctcctcctgtcaggagaggaaagagaagaaggttagtacaattgctata |
| | | gtgagttgtattatactatgcagatatactatgccaatgattaattgtcaaactagggctgcagggttcatagtgccacttac |
| | | ctgcactgccccatctcctgcccaccctttcccaggcatagacagtcagtgacttaccaaactcacaggagggagaagg |
| | | cagaagcttgagacagacccgcgggaccgccgaactgcgaggggacgtggctagggcggcttatttatggtgcgcc |
| | | ggccctcggaggcagggcgctcggggaggcctagcggccaatctgcggtggcaggaggcggggccgaaggccgt |
| | | gcctgaccaatccggagcacataggagtctcagccccccgccccaaagcaaggggaagtcacgcgcctgtagcgcc |
| | | agcgtgttgtgaaatgggggcttgggggggttggggccctgactagtcaaaacaaactcccattgacgtcaatggggtg |
| | | gagacttggaaatccccgtgagtcaaaccgctatccacgcccattgatgtactgccaaaaccgcatcatcatggtaatag |
| | | cgatgactaatacgtagatgtactgccaagtaggaaagtcccataaggtcatgtactgggcataatgccaggcgggcca |
| | | tttaccgtcattgacgtcaatagggggcgtacttggcatatgatacacttgatgtactgccaagtgggcagtttaccgtaaat |
| | | actccacccattgacgtcaatggaaagtccctattggcgttactatgggaacatacgtcattattgacgtcaatgggcggg |
| | | ggtcgttgggcggtcagccaggcgggccatttaccgtaagttatgtaacgcctgcaggttaattaagaacatgtgagcaa |
| | | aaggccagcaaaaggccaggaaccgtaaaaaggccgcgttgctggcgtttttccataggctccgcccccctgacgagc |
| | | atcacaaaaatcgacgctcaagtcagaggtggcgaaacccgacaggactataaagataccaggcgtttccccctggaa |
| | | gctccctcgtgcgctctcctgttccgaccctgccgcttaccggatacctgtccgcctttctcccttcgggaagcgtggcgct |
| | | ttctcatagctcacgctgtaggtatctcagttcggtgtaggtcgttcgctccaagctgggctgtgtgcacgaaccccccgtt |
| | | cagcccgaccgctgcgccttatccggtaactatcgtcttgagtccaacccggtaagacacgacttatcgccactggcagc |
| | | agccactggtaacaggattagcagagcgaggtatgtaggcggtgctacagagttcttgaagtggtggcctaactacggc |
| | | tacactagaagaacagtatttggtatctgcgctctgctgaagccagttaccttcggaaaaagagttggtagctcttgatccg |
| | | gcaaacaaaccaccgctggtagcggtggtttttttgtttgcaagcagcagattacgcgcagaaaaaaaggatctcaagaa |
| | | gatcctttgatcttttctacggggtctgacgctcagtggaacgaaaactcacgttaagggattttggtcatggctagttaatta |
| | | acatttaaatcagcggccgcaataaaatatattattttcattacatctgtgtgaggttttttgtgtgaatcgtaactaacatacg |
| | | ctctccatcaaaacaaaacgaaacaaaacaaactagcaaaataggctgtccccagtgcaagtgcaggtgccagaacatt |
| | | tctctatcgaa |
| mCD19(1D3)- | 39 | ggatctgcgatcgctccggtgcccgtcagtgggcagagcgcacatcgcccacagtccccgagaagttggggggagg |
| HC-WT | | ggtcggcaattgaacgggtgcctagagaaggtggcgcggggtaaactgggaaagtgatgtcgtgtactggctccgcct |
| Fab (wild | | ttttcccgagggtgggggagaaccgtatataagtgcagtagtcgccgtgaacgttctttttcgcaacgggtttgccgccag |
| type | | aacacagctgaagcttcgaggggctcgcatctctccttcacgcgcccgccgccctacctgaggccgccatccacgccg |
| heavy | | gttgagtcgcgttctgccgcctcccgcctgtggtgcctcctgaactgcgtccgccgtctaggtaagtttaaagctcaggtc |
| chain | | gagaccgggcctttgtccggcgctcccttggagcctacctagactcagccggctctccacgctttgcctgaccctgcttg |
| sequence) | | ctcaactctacgtattgtttcgttttctgttctgcgccgttacagatccaagctgtgaccggcgcctacctgagatcaccgg |
| | | cgaaggagggccaccatgtacaggatgcaactcctgtcttgcattgcactaagtcttgcacttgtcacgaattcgGAC |
| | | ATTCAGCTGACCCAGTCTCCAGCTTCCCTGTCTACATCTCTGGGAGAAAC |
| | | TGTCACCATCCAATGTCAAGCAAGTGAGGACATTTACAGTGGTTTAGCG |
| | | TGGTATCAGCAGAAGCCAGGGAAATCTCCTCAGCTCCTGATCTATGGTG |
| | | CAAGTGACTTACAAGACGGCGTCCCATCACGATTCAGTGGCAGTGGATC |
| | | TGGCACACAGTATTCTCTCAAGATCACCAGCATGCAAACTGAAGATGAA |
| | | GGGGTTTATTTCTGTCAACAGGGTTTAACGTATCCTCGGACGTTCGGTGG |
| | | CGGCACCAAGCTgGAaATCaaacgaactgtggctgcaccatctgtcttcatcttcccgccatctgatgag |
| | | cagttgaaatctggaactgcctctgtcgtgtgcctgctgaataacttctatcccagagaggccaaagtacagtggaaggtg |
| | | gataacgccctccaatcgggtaactcccaggagagtgtcacagagcaggacagcaaggacagcacctacagcctcag |
| | | cagcaccctgacgctgagcaaagcagactacgagaaacacaaagtctacgcctgcgaagtcacccatcagggcctgt |
| | | cctcgcccgtcacaaagagcttcaacaggggagagtgttgataagtgctagctggccagacatgataagatacattgat |
| | | gagtttggacaaaccacaactagaatgcagtgaaaaaaatgctttatttgtgaaatttgtgatgctattgctttatttgtaacca |
| | | ttataagctgcaataaacaagttaacaacaacaattgcattcattttatgtttcaggttcagggggaggtgtgggaggtttttt |
| | | aaagcaagtaaaacctctacaaatgtggtatggaattaattctaaaatacagcatagcaaaactttaacctccaaatcaagc |
| | | ctctacttgaatccttttctgagggatgaataaggcataggcatcaggggctgttgccaatgtgcattagctgtttgcagcct |
| | | caccttctttcatggagtttaagatatagtgtattttcccaaggtttgaactagctcttcatttattatgttttaaatgcactgacc |
| | | tcccacattccctttttagtaaaatattcagaaataatttaaatacatcattgcaatgaaaataaatgttttttattaggcagaatc |
| | | cagatgctcaaggcccttcataatatcccccagtttagtagttggacttagggaacaaaggaacctttaatagaaattggac |
| | | agcaagaaagcgagcttctagcttatcctcagtcctgctcctctgccacaaagtgcacgcagttgccggccgggtcgcg |
| | | cagggcgaactcccgcccccacggctgctcgccgatctcggtcatggccggcccggaggcgtcccggaagttcgtgg |
| | | acacgacctccgaccactcggcgtacagctcgtccaggccgcgcacccacacccaggccagggtgttgtccggcacc |
| | | acctggtcctggaccgcgctgatgaacagggtcacgtcgtcccggaccacaccggcgaagtcgtcctccacgaagtcc |
| | | cgggagaacccgagccggtcggtccagaactcgaccgctccggcgacgtcgcgcgcggtgagcaccggaacggca |
| | | ctggtcaacttggccatgatggctcctcctgtcaggagaggaaagagaagaaggttagtacaattgctatagtgagttgta |
| | | ttatactatgcagatatactatgccaatgattaattgtcaaactagggctgcagggttcatagtgccacttttcctgcactgcc |
| | | ccatctcctgcccaccctttcccaggcatagacagtcagtgacttaccaaactcacaggagggagaaggcagaagcttg |
| | | agacagacccgcgggaccgccgaactgcgaggggacgtggctagggcggcttcttttatggtgcgccggccctcgga |
| | | ggcagggcgctcggggaggcctagcggccaatctgcggtggcaggaggcggggccgaaggccgtgcctgaccaat |
| | | ccggagcacataggagtctcagccccccgccccaaagcaaggggaagtcacgcgcctgtagcgccagcgtgttgtga |
| | | aatgggggcttgggggggttggggccctgactagtcaaaacaaactcccattgacgtcaatggggtggagacttggaa |
| | | atccccgtgagtcaaaccgctatccacgcccattgatgtactgccaaaaccgcatcatcatggtaatagcgatgactaata |
| | | cgtagatgtactgccaagtaggaaagtcccataaggtcatgtactgggcataatgccaggcgggccatttaccgtcattg |
| | | acgtcaatagggggcgtacttggcatatgatacacttgatgtactgccaagtgggcagtttaccgtaaatactccacccatt |
| | | gacgtcaatggaaagtccctattggcgttactatgggaacatacgtcattattgacgtcaatgggcgggggtcgttgggc |
| | | ggtcagccaggcgggccatttaccgtaagttatgtaacgcctgcaggttaattaagaacatgtgagcaaaaggccagca |
| | | aaaggccaggaaccgtaaaaaggccgcgttgctggcgtttttccataggctccgcccccctgacgagcatcacaaaaat |
| | | cgacgctcaagtcagaggtggcgaaacccgacaggactataaagataccaggcgtttccccctggaagctccctcgtg |
| | | cgctctcctgttccgaccctgccgcttaccggatacctgtccgcctttctcccttcgggaagcgtggcgattctcatagctc |
| | | acgctgtaggtatctcagttcggtgtaggtcgttcgctccaagctgggctgtgtgcacgaaccccccgttcagcccgacc |
| | | gctgcgccttatccggtaactatcgtcttgagtccaacccggtaagacacgacttatcgccactggcagcagccactggt |
| | | aacaggattagcagagcgaggtatgtaggcggtgctacagagttcttgaagtggtggcctaactacggctacactagaa |
| | | gaacagtatttggtatctgcgctctgctgaagccagttaccttcggaaaaagagttggtagctcttgatccggcaaacaaa |
| | | ccaccgctggtagcggtggtttttttgtttgcaagcagcagattacgcgcagaaaaaaaggatctcaagaagatcattga |
| | | tcttttctacggggtctgacgctcagtggaacgaaaactcacgttaagggattttggtcatggctagttaattaacatttaaat |
| | | cagcggccgcaataaaatatctttattttcattacatctgtgtgttggttttttgtgtgaatcgtaactaacatacgctctccatc |
| | | aaaacaaaacgaaacaaaacaaactagcaaaataggctgtccccagtgcaagtgcaggtgccagaacatttctctatcg |
| | | aa |

TABLE 23
CAR-EC switch target interacting domains (antibodies) with
FITC-Amino Acid Sequence

| | SEQ | | | |
|---|---|---|---|---|
| Name | ID | Light chain | SEQ ID | Heavy chain |
| anti-Her2 | 40 | DIQMTQSPSSLSASVGDRVT | 41 | EVQLVESGGGLVQPGGSLR |
| Fab wild | | ITCRASQDVNTAVAWYQQ | | LSCAASGFNIKDTYIHWVRQ |
| type | | KPGKAPKLLIYSASFLYSGV | | APGKGLEWVARIYPTNGYT |
| | | PSRFSGSRSGTDFTLTISSLQ | | RYADSVKGRFTISADTSKNT |
| | | PEDFATYYCQQHYTTPPTF | | AYLQMNSLRAEDTAVYYCS |
| | | GQGTKLEIKRTVAAPSVFIF | | RWGGDGFYAMDYWGQGT |
| | | PPSDEQLKSGTASVVCLLN | | LVTVSSASTKGPSVFPLAPSS |
| | | NFYPREAKVQWKVDNALQ | | KSTSGGTAALGCLVKDYFP |
| | | SGNSQESVTEQDSKDSTYSL | | EPVTVSWNSGALTSGVHTF |
| | | SSTLTLSKADYEKHKVYAC | | PAVLQSSGLYSLSSVVTVPS |
| | | EVTHQGLSSPVTKSFNRGEC | | SSLGTQTYICNVNHKPSNTK |
| | | | | VDKKVEPKSCDKTHT |
| anti-Her2 | 42 | DIQMTQSPSSLSASVGDRVT | 41 | EVQLVESGGGLVQPGGSLR |
| Fab | | ITCRASQDVNTAVAWYQQ | | LSCAASGFNIKDTYIHWVRQ |
| (LG68X) | | KPGKAPKLLIYSASFLYSGV | | APGKGLEWVARIYPTNGYT |
| | | PSRFSGSRSXTDFTLTISSLQ | | RYADSVKGRFTISADTSKNT |
| | | PEDFATYYCQQHYTTPPTF | | AYLQMNSLRAEDTAVYYCS |
| | | GQGTKLEIKRTVAAPSVFIF | | RWGGDGFYAMDYWGQGT |
| | | PPSDEQLKSGTASVVCLLN | | LVTVSSASTKGPSVFPLAPSS |
| | | NFYPREAKVQWKVDNALQ | | KSTSGGTAALGCLVKDYFP |
| | | SGNSQESVTEQDSKDSTYSL | | EPVTVSWNSGALTSGVHTF |
| | | SSTLTLSKADYEKHKVYAC | | PAVLQSSGLYSLSSVVTVPS |
| | | EVTHQGLSSPVTKSFNRGEC | | SSLGTQTYICNVNHKPSNTK |
| | | | | VDKKVEPKSCDKTHT |
| anti-Her2 | 43 | DIQMTQSPSSLSASVGDRVT | 41 | EVQLVESGGGLVQPGGSLR |
| Fab | | ITCRASQDVNTAVAWYQQ | | LSCAASGFNIKDTYIHWVRQ |
| (LS202X) | | KPGKAPKLLIYSASFLYSGV | | APGKGLEWVARIYPTNGYT |
| | | PSRFSGSRSGTDFTLTISSLQ | | RYADSVKGRFTISADTSKNT |
| | | PEDFATYYCQQHYTTPPTF | | AYLQMNSLRAEDTAVYYCS |
| | | GQGTKLEIKRTVAAPSVFIF | | RWGGDGFYAMDYWGQGT |
| | | PPSDEQLKSGTASVVCLLN | | LVTVSSASTKGPSVFPLAPSS |
| | | NFYPREAKVQWKVDNALQ | | KSTSGGTAALGCLVKDYFP |
| | | SGNSQESVTEQDSKDSTYSL | | EPVTVSWNSGALTSGVHTF |
| | | SSTLTLSKADYEKHKVYAC | | PAVLQSSGLYSLSSVVTVPS |
| | | EVTHQGLXSPVTKSFNRGEC | | SSLGTQTYICNVNHKPSNTK |
| | | | | VDKKVEPKSCDKTHT |
| anti-Her2 | 40 | DIQMTQSPSSLSASVGDRVT | 44 | EVQLVESGGGLVQPGGSLR |
| Fab | | ITCRASQDVNTAVAWYQQ | | LSCAASGFNIKDTYIHWVRQ |
| (HS75X) | | KPGKAPKLLIYSASFLYSGV | | APGKGLEWVARIYPTNGYT |
| | | PSRFSGSRSGTDFTLTISSLQ | | RYADSVKGRFTISADTXKN |
| | | PEDFATYYCQQHYTTPPTF | | TAYLQMNSLRAEDTAVYYC |
| | | GQGTKLEIKRTVAAPSVFIF | | SRWGGDGFYAMDYWGQG |
| | | PPSDEQLKSGTASVVCLLN | | TLVTVSSASTKGPSVFPLAP |
| | | NFYPREAKVQWKVDNALQ | | SSKSTSGGTAALGCLVKDY |
| | | SGNSQESVTEQDSKDSTYSL | | FPEPVTVSWNSGALTSGVH |
| | | SSTLTLSKADYEKHKVYAC | | TFPAVLQSSGLYSLSSVVTV |
| | | EVTHQGLSSPVTKSFNRGEC | | PSSSLGTQTYICNVNHKPSN |
| | | | | TKVDKKVEPKSCDKTHT |
| anti-Her2 | 40 | DIQMTQSPSSLSASVGDRVT | 45 | EVQLVESGGGLVQPGGSLR |
| Fab | | ITCRASQDVNTAVAWYQQ | | LSCAASGFNIKDTYIHWVRQ |
| (HK136X) | | KPGKAPKLLIYSASFLYSGV | | APGKGLEWVARIYPTNGYT |
| | | PSRFSGSRSGTDFTLTISSLQ | | RYADSVKGRFTISADTSKNT |
| | | PEDFATYYCQQHYTTPPTF | | AYLQMNSLRAEDTAVYYCS |
| | | GQGTKLEIKRTVAAPSVFIF | | RWGGDGFYAMDYWGQGT |
| | | PPSDEQLKSGTASVVCLLN | | LVTVSSASTKGPSVFPLAPSS |
| | | NFYPREAKVQWKVDNALQ | | XSTSGGTAALGCLVKDYFP |
| | | SGNSQESVTEQDSKDSTYSL | | EPVTVSWNSGALTSGVHTF |
| | | SSTLTLSKADYEKHKVYAC | | PAVLQSSGLYSLSSVVTVPS |
| | | EVTHQGLSSPVTKSFNRGEC | | SSLGTQTYICNVNHKPSNTK |
| | | | | VDKKVEPKSCDKTHT |
| anti-Her2 | 42 | DIQMTQSPSSLSASVGDRVT | 44 | EVQLVESGGGLVQPGGSLR |
| Fab | | ITCRASQDVNTAVAWYQQ | | LSCAASGFNIKDTYIHWVRQ |
| (LG68XHS75X) | | KPGKAPKLLIYSASFLYSGV | | APGKGLEWVARIYPTNGYT |
| | | PSRFSGSRSXTDFTLTISSLQ | | RYADSVKGRFTISADTXKN |
| | | PEDFATYYCQQHYTTPPTF | | TAYLQMNSLRAEDTAVYYC |
| | | GQGTKLEIKRTVAAPSVFIF | | SRWGGDGFYAMDYWGQG |
| | | PPSDEQLKSGTASVVCLLN | | TLVTVSSASTKGPSVFPLAP |
| | | NFYPREAKVQWKVDNALQ | | SSKSTSGGTAALGCLVKDY |
| | | SGNSQESVTEQDSKDSTYSL | | FPEPVTVSWNSGALTSGVH |
| | | SSTLTLSKADYEKHKVYAC | | TFPAVLQSSGLYSLSSVVTV |
| | | EVTHQGLSSPVTKSFNRGEC | | PSSSLGTQTYICNVNHKPSN |
| | | | | TKVDKKVEPKSCDKTHT |
| anti-Her2 | 43 | DIQMTQSPSSLSASVGDRVT | 45 | EVQLVESGGGLVQPGGSLR |
| Fab | | ITCRASQDVNTAVAWYQQ | | LSCAASGFNIKDTYIHWVRQ |
| (LS202XHK136X) | | KPGKAPKLLIYSASFLYSGV | | APGKGLEWVARIYPTNGYT |
| | | PSRFSGSRSGTDFTLTISSLQ | | RYADSVKGRFTISADTSKNT |
| | | PEDFATYYCQQHYTTPPTF | | AYLQMNSLRAEDTAVYYCS |
| | | GQGTKLEIKRTVAAPSVFIF | | RWGGDGFYAMDYWGQGT |
| | | PPSDEQLKSGTASVVCLLN | | LVTVSSASTKGPSVFPLAPSS |
| | | NFYPREAKVQWKVDNALQ | | XSTSGGTAALGCLVKDYFP |
| | | SGNSQESVTEQDSKDSTYSL | | EPVTVSWNSGALTSGVHTF |
| | | SSTLTLSKADYEKHKVYAC | | PAVLQSSGLYSLSSVVTVPS |
| | | EVTHQGLXSPVTKSFNRGEC | | SSLGTQTYICNVNHKPSNTK |
| | | | | VDKKVEPKSCDKTHT |

TABLE 24
Raw Data from FIG. 65A

| | |
|---|---|
| | FITC-E2 CAR + |
| CART19 + | Nalm-6 + |
| Nalm-6 | 1 nM AB-FITC |

| | | | | |
|---|---|---|---|---|
| 15:01 | 87.286 | 88.264 | 82.518 | 82.885 |
| 5:01 | 75.151 | 73.282 | 68.994 | 64.156 |
| 1:01 | 51.987 | 48.013 | 44.979 | 49.582 |
| 0.2:1 | 21.663 | 24.538 | 19.507 | 24.435 |
| 0.04:1 | 12.621 | 13.030 | 11.804 | 10.986 |

TABLE 25
Raw Data from FIG. 65B

| | anti-FITC CAR + Nalm6 | | | CART19 + Nalm-6 | | |
|---|---|---|---|---|---|---|
| Human IFN-g | 1006.37 | 959.1 | 938.35 | 915.26 | 924.3 | 950.53 |

TABLE 26
Raw Data from FIG. 66B

| Days | | | | | | | | | | | | | | | |
|---|---|---|---|---|---|---|---|---|---|---|---|---|---|---|---|
| after | | | | | | | | | | | | | | | |
| Nalm- | | | | | | | | | | | | | | | |
| 6 iv | Nalm-6, CART-19 | | | Nalm-6, E2, 0.5 mg/kg | | | Nalm-6, E2, 0.05 mg/kg | | | Nalm-6, E2, 0.005 mg/kg | | | No Nalm-6, E2, 0.5 mg/kg | | |
| 10 | 25.5 | 51.75 | 40.5 | 24.25 | 31 | 21.75 | 48 | 26.75 | 66.25 | 50 | 15 | 77.25 | 34 | 31.25 | 42 |
| 18 | 79.5 | 91.25 | 85 | 117.75 | 107 | 151.25 | 190 | 85.5 | 203.25 | 29.75 | 9.25 | 11 | 44.25 | 48.5 | 22.5 |
| 27 | 16.5 | 21 | 19.25 | 27.5 | 34.5 | 91.25 | 38.75 | 29.75 | 32.75 | 12.25 | 39.25 | 36.75 | 20.75 | 22.25 | 18.25 |

TABLE 27
Raw Data from FIG. 66D

| Days | | | | | | | | | | | | | | | |
|---|---|---|---|---|---|---|---|---|---|---|---|---|---|---|---|
| after | | | | | | | | | | | | | | | |
| Nalm- | | | | | | | | | | | | | | | |
| 6 iv | PBS | | | | | | 0.5 mg/kg (×6) | | | | | | 0.05 mg/kg (×6) | | |
| 6 | 100 | | | | | | 100 | | | | | | 100 | | |
| 7 | 93.8 | 95.3 | 94.6 | 95.8 | 98.5 | 96.2 | 98.3 | 99.4 | 97.3 | 96.4 | 97.9 | 97.6 | 99.5 | 96.8 | 100.0 |
| 8 | 96.7 | 96.9 | 97.3 | 99.4 | 100.5 | 97.8 | 92.1 | 90.1 | 90.8 | 96.9 | 91.8 | 80.9 | 92.6 | 90.9 | 101.2 |
| 9 | 95.2 | 96.9 | 98.4 | 98.8 | 103.4 | 97.8 | 83.1 | 82.9 | 83.7 | 84.5 | 85.1 | 81.3 | 92.6 | 94.1 | 98.8 |
| 10 | 98.6 | 100.0 | 101.1 | 101.2 | 103.4 | 100.5 | 80.8 | 81.2 | 79.9 | 86.6 | 82.5 | 80.4 | 89.9 | 94.6 | 95.2 |
| 11 | 96.2 | 97.9 | 100.0 | 99.4 | 101.0 | 102.7 | 81.9 | 81.8 | 84.8 | 86.1 | 86.6 | 81.8 | 98.4 | 103.2 | 103.0 |
| 12 | 100.5 | 99.0 | 102.2 | 101.2 | 99.5 | 101.6 | 83.1 | 81.8 | 85.3 | 92.8 | 91.2 | 85.2 | 101.1 | 107.5 | 107.3 |
| 13 | 97.6 | 97.9 | 99.5 | 97.6 | 98.0 | 97.8 | 87.6 | 81.8 | 90.8 | 94.8 | 95.4 | 90.9 | 103.7 | 105.4 | 107.3 |
| 14 | 94.3 | 96.4 | 98.4 | 94.0 | 98.5 | 98.9 | 93.8 | 90.6 | 96.7 | 97.9 | 95.4 | 94.3 | 103.7 | 105.4 | 106.1 |
| 15 | 95.2 | 100.0 | 100.0 | 97.6 | 98.0 | 98.4 | 96.0 | 96.7 | 95.1 | 102.1 | 97.4 | 97.6 | 107.9 | 107.0 | 106.7 |
| 17 | 89.5 | 92.7 | 92.9 | 94.0 | 95.1 | 92.5 | 101.1 | 102.8 | 103.8 | 97.4 | 96.4 | 98.6 | 104.2 | 103.2 | 101.2 |
| 18 | 85.6 | 89.6 | 90.8 | 89.8 | 92.1 | 88.2 | 109.0 | 107.7 | 109.2 | 105.2 | 110.3 | 92.3 | 111.6 | 109.1 | 107.9 |
| 22 | | | | | | | 105.6 | 115.5 | 109.8 | 102.1 | 97.4 | 101.9 | 106.9 | 98.9 | 103.0 |
| 25 | | | | | | | 109.0 | 123.8 | 114.7 | 108.8 | 107.7 | 109.6 | 111.1 | 108.1 | 107.9 |
| Days | | | | | | | | | | | | | | | |
| after | | | | | | | | | | | | | | | |
| Nalm- | | | | | | | | | | | | | | | |
| 6 iv | 0.05 mg/kg (×6) | | | 0.05 mg/kg (×3) + 0.5 mg/kg (×3) | | | | | | | 0.005 mg/kg (×6) | | | | |
| 6 | | | | 100 | | | | | | 100 | | | | | |
| 7 | 97.5 | 95.1 | 99.5 | 96.5 | 95.1 | 98.5 | 93.4 | 94.8 | 96.9 | 95.0 | 96.3 | 95.2 | 95.8 | 96.7 | 95.8 |
| 8 | 94.1 | 96.0 | 94.6 | 96.0 | 89.8 | 96.6 | 94.9 | 95.9 | 96.4 | 97.8 | 99.5 | 101.6 | 96.3 | 100.9 | 98.6 |
| 9 | 94.6 | 93.3 | 92.4 | 95.5 | 90.8 | 96.1 | 93.4 | 94.3 | 95.8 | 95.5 | 98.9 | 96.8 | 94.9 | 105.2 | 98.1 |
| 10 | 91.6 | 90.6 | 88.0 | 94.0 | 88.8 | 91.7 | 91.3 | 90.7 | 94.3 | 97.8 | 95.8 | 97.4 | 94.4 | 103.3 | 94.4 |
| 11 | 97.0 | 93.7 | 92.9 | 98.0 | 93.2 | 96.6 | 95.9 | 94.8 | 96.9 | 97.2 | 96.3 | 94.7 | 98.1 | 107.1 | 95.3 |
| 12 | 103.5 | 98.7 | 99.5 | 102.0 | 98.1 | 102.4 | 97.4 | 99.0 | 104.7 | 101.1 | 100.0 | 102.1 | 99.1 | 108.5 | 98.6 |
| 13 | 102.5 | 97.3 | 101.1 | 101.5 | 98.5 | 101.9 | 96.4 | 99.0 | 101.0 | 101.1 | 96.8 | 101.6 | 96.7 | 108.5 | 98.1 |
| 14 | 103.0 | 97.8 | 103.8 | 102.0 | 100.0 | 102.4 | 96.4 | 101.0 | 105.7 | 105.6 | 92.6 | 101.6 | 96.7 | 109.5 | 97.7 |
| 15 | 100.5 | 98.7 | 101.6 | 102.0 | 100.5 | 104.4 | 98.0 | 101.0 | 104.7 | 98.9 | 100.5 | 103.2 | 98.1 | 107.1 | 99.5 |
| 17 | 98.5 | 97.8 | 104.3 | 101.0 | 97.6 | 104.4 | 96.4 | 100.5 | 103.1 | 97.2 | 96.8 | 102.1 | 96.7 | 107.1 | 97.7 |
| 18 | 101.5 | 102.7 | 109.8 | 105.5 | 101.5 | 103.9 | 100.5 | 105.2 | 107.3 | 101.7 | 102.1 | 106.3 | 100.0 | 111.4 | 102.3 |
| 22 | 99.0 | 97.3 | 107.1 | 104.5 | 101.0 | 108.7 | 102.6 | 103.1 | 105.2 | 98.3 | 97.4 | 103.2 | 96.7 | 107.6 | 100.0 |
| 25 | 104.0 | 102.7 | 117.9 | 110.0 | 105.8 | 115.0 | 106.6 | 109.8 | 106.3 | 104.5 | 98.9 | 104.8 | 102.3 | 106.6 | 105.5 |

TABLE 28
Raw Data from FIG. 67C

| | | | | | | | | | | | | | aFITC CAR + aCD19 FITC | | | | |
|---|---|---|---|---|---|---|---|---|---|---|---|---|---|---|---|---|---|
| | Control | | | | | | aCD19 (1D3) CAR | | | | | | (1 mg/kg) | | | | |
| Day 22 | 11897.5 | 13827.5 | 10285 | 16392.5 | 20480 | 19250 | 90 | 37.5 | 70 | 52.5 | 57.5 | 27.5 | 6647.5 | 4540 | 5087.5 | 13657.5 | 17355 |

TABLE 29
Raw Data from FIG. 68A

| Conc. | | | | | | | | |
|---|---|---|---|---|---|---|---|---|
| pM | Apr. 4, 2020 | | 4D5Flu | | 4M5.3 | | FITC-E2 | |
| 1.00E−04 | 0.000 | 0.000 | 4.994 | 6.287 | 9.004 | 0.000 | 0.000 | 0.000 |
| 1.00E−03 | 0.000 | 4.966 | 0.000 | 15.629 | 0.000 | 0.000 | 10.277 | 25.172 |
| 0.01 | 0.526 | 11.175 | 24.008 | 29.461 | 21.009 | 0.000 | 26.680 | 38.448 |
| 0.1 | 32.848 | 33.275 | 39.347 | 45.749 | 46.405 | 33.925 | 49.012 | 60.517 |
| 1 | 52.337 | 42.711 | 60.115 | 63.952 | 58.166 | 56.613 | 50.988 | 75.345 |
| 10 | 64.908 | 66.054 | 56.082 | 67.725 | 58.872 | 48.925 | 62.055 | 75.517 |
| 100 | 71.740 | 65.557 | 74.328 | 69.341 | 70.488 | 71.290 | 69.565 | 80.517 |
| 1000 | 67.798 | 69.530 | 79.513 | 84.790 | 81.959 | 83.226 | 80.632 | 99.828 |
| 10000 | 74.119 | 71.517 | 73.560 | 76.946 | 85.653 | 72.043 | 93.083 | 100.000 |

TABLE 30
Raw Data from FIG. 68B

| Conc. | | | | | | | | |
|---|---|---|---|---|---|---|---|---|
| pM | Apr. 4, 2020 | | 4D5Flu | | 4M5.3 | | FITC-E2 | |
| 0.0001 | 0.000 | 13.884 | 0.000 | 0.000 | 0.000 | 2.005 | 3.264 | 17.048 |
| 0.001 | 0.000 | 0.000 | 0.000 | 13.947 | 0.000 | 9.167 | 0.000 | 19.593 |
| 0.01 | 0.000 | 12.015 | 1.901 | 8.367 | 0.000 | 1.146 | 17.363 | 0.000 |
| 0.1 | 14.754 | 10.947 | 10.924 | 10.423 | 25.411 | 11.172 | 15.013 | 25.700 |
| 1 | 36.703 | 37.791 | 39.378 | 32.930 | 28.460 | 27.500 | 47.389 | 43.187 |
| 10 | 59.008 | 46.723 | 63.173 | 68.080 | 67.105 | 58.385 | 64.621 | 70.115 |
| 100 | 57.890 | 43.786 | 69.357 | 61.225 | 60.832 | 65.833 | 60.744 | 67.595 |
| 1000 | 63.763 | 55.801 | 72.450 | 63.589 | 73.378 | 71.536 | 70.437 | 74.943 |
| 10000 | 68.644 | 63.819 | 69.137 | 66.805 | 64.168 | 55.174 | 72.689 | 75.420 |

TABLE 31
Raw Data from FIG. 68C

| Conc. | | | | | | | | |
|---|---|---|---|---|---|---|---|---|
| pM | Apr. 4, 2020 | | 4D5Flu | | 4M5.3 | | FITC-E2 | |
| 0.0001 | 0.000 | 0.000 | 10.698 | 0.000 | 0.000 | 0.000 | 0.000 | 0.000 |
| 0.001 | 8.730 | 10.013 | 0.000 | 9.011 | 7.344 | 0.000 | 3.614 | 0.000 |
| 0.01 | 18.380 | 17.523 | 0.315 | 5.904 | 6.113 | 0.468 | 9.200 | 3.623 |
| 0.1 | 22.196 | 20.052 | 39.960 | 9.633 | 10.608 | 19.784 | 9.528 | 16.257 |
| 1 | 68.730 | 35.403 | 30.521 | 67.034 | 39.169 | 66.115 | 42.628 | 64.281 |
| 10 | 63.657 | 56.860 | 57.580 | 60.282 | 61.016 | 63.561 | 62.198 | 63.713 |
| 100 | 85.119 | 79.057 | 88.730 | 92.288 | 82.426 | 87.770 | 88.955 | 80.060 |
| 1000 | 80.946 | 81.130 | 78.661 | 87.938 | 92.018 | 88.849 | 80.824 | 92.575 |
| 10000 | 81.852 | 81.905 | 78.799 | 79.237 | 80.386 | 85.683 | 78.853 | 86.737 |

TABLE 32
Raw Data from FIG. 68D

| Conc. | | | | | | | | |
|---|---|---|---|---|---|---|---|---|
| pM | Apr. 4, 2020 | | 4D5Flu | | 4M5.3 | | FITC-E2 | |
| 0.0001 | 0.000 | 0.000 | 0.000 | 0.000 | 0.000 | 8.535 | 0.000 | 3.871 |
| 0.001 | 0.000 | 0.000 | 0.000 | 0.000 | 0.000 | 0.000 | 0.577 | 9.785 |
| 0.01 | 0.000 | 10.801 | 0.000 | 2.910 | 0.000 | 8.280 | 0.000 | 0.000 |
| 0.1 | 0.000 | 8.007 | 3.030 | 1.552 | 1.233 | 6.242 | 0.000 | 8.280 |
| 1 | 0.000 | 0.000 | 0.489 | 0.000 | 0.000 | 0.000 | 0.000 | 5.591 |
| 10 | 0.000 | 4.190 | 2.346 | | 0.000 | 5.732 | 0.000 | 7.957 |
| 100 | 0.000 | 0.372 | 7.234 | 6.499 | 4.247 | 0.000 | 0.000 | 11.613 |
| 1000 | 12.063 | 7.742 | 8.147 | 4.462 | 11.872 | 0.000 | 0.000 | 11.541 |
| 10000 | 7.007 | 11.541 | 7.398 | 16.416 | 18.447 | 0.000 | 0.000 | 16.416 |

TABLE 33
Raw Data from FIG. 72A

| Conc. | Non- | | Random | | | | | | | | | |
|---|---|---|---|---|---|---|---|---|---|---|---|---|
| (pM) | conjugated | | (DAR2) | | B-FITC | | AB-FITC | | E-FITC | | EF-FITC | |
| 0.0001 | 0.000 | 1.779 | 0.000 | 0.000 | 0.000 | 0.000 | 0.000 | 4.928 | 0.000 | 0.000 | 0.854 | 0.000 |
| 0.001 | 0.731 | 7.100 | 0.000 | 0.000 | 0.000 | 0.000 | 0.000 | 0.000 | 0.000 | 0.000 | 4.820 | 0.000 |
| 0.01 | 0.000 | 8.146 | 0.000 | 5.695 | 0.000 | 3.994 | 0.000 | 19.710 | 2.482 | 0.000 | 6.711 | 4.462 |
| 0.1 | 0.000 | 1.958 | 0.000 | 3.498 | 10.000 | 12.229 | 20.000 | 6.860 | 16.045 | 10.963 | 4.149 | 14.000 |
| 1 | 0.000 | 4.933 | 21.001 | 27.982 | 27.821 | 36.425 | 60.465 | 71.594 | 25.861 | 14.542 | 30.506 | 27.270 |
| 10 | 3.728 | 2.556 | 63.181 | 67.803 | 60.550 | 72.793 | 61.334 | 43.478 | 65.548 | 57.696 | 56.541 | 55.298 |
| 100 | 4.605 | 8.924 | 64.739 | 64.126 | 56.651 | 77.207 | 77.295 | 61.353 | 70.216 | 75.165 | 57.035 | 57.463 |
| 1000 | 0.000 | 6.697 | 66.407 | 70.673 | 67.775 | 75.109 | 63.647 | 69.662 | 75.821 | 70.000 | 79.012 | 81.439 |
| 10000 | 11.696 | 1.868 | 68.966 | 63.049 | 71.904 | 62.880 | 78.849 | 61.643 | 69.736 | 59.868 | 81.879 | 74.555 |

TABLE 34
Raw Data from FIG. 72B

| Conc. | Non- | | Random | | | | | | | | | |
|---|---|---|---|---|---|---|---|---|---|---|---|---|
| (pM) | conjugated | | (DAR 2) | | B-FITC | | AB-FITC | | E-FITC | | EF-FITC | |
| 0.0001 | 0.000 | 0.000 | 0.000 | 0.000 | 0.000 | 2.992 | 8.496 | 5.424 | 0.000 | 4.724 | 0.000 | 0.000 |
| 0.001 | 0.000 | 2.727 | 5.460 | 0.000 | 2.502 | 2.043 | 5.220 | 2.763 | 0.000 | 7.615 | 0.000 | 4.846 |
| 0.01 | 2.041 | 0.000 | 0.000 | 5.187 | 0.000 | 4.378 | 15.424 | 18.973 | 0.000 | 6.346 | 0.000 | 7.985 |
| 0.1 | 0.000 | 0.000 | 5.869 | 12.899 | 1.930 | 3.211 | 21.021 | 22.386 | 0.072 | 5.570 | 6.688 | 12.012 |
| 1 | 3.842 | 0.000 | 22.659 | 15.834 | 6.718 | 16.710 | 52.688 | 49.481 | 7.340 | 11.563 | 9.009 | 16.448 |
| 10 | 4.562 | 2.093 | 67.157 | 63.335 | 61.181 | 61.148 | 65.315 | 68.727 | 41.020 | 42.023 | 36.718 | 41.632 |
| 100 | 3.241 | 1.395 | 82.513 | 82.718 | 69.758 | 71.218 | 84.970 | 81.490 | 77.578 | 79.675 | 87.018 | 86.745 |
| 1000 | 10.504 | 2.600 | 83.128 | 86.199 | 74.761 | 72.240 | 84.424 | 80.125 | 84.919 | 88.207 | 92.273 | 93.433 |
| 10000 | 2.581 | 0.000 | 83.264 | 85.039 | 76.976 | 81.069 | 77.122 | 80.056 | 82.976 | 81.861 | 85.312 | 88.042 |

TABLE 35
Raw Data from FIG. 72C

| Conc. | Non- | | Random | | | | | | | | | |
|---|---|---|---|---|---|---|---|---|---|---|---|---|
| (pM) | conjugated | | (DAR 2) | | B-FITC | | AB-FITC | | E-FITC | | EF-FITC | |
| 0.0001 | 0.000 | 1.122 | 1.290 | 7.188 | 0.000 | 1.416 | 1.813 | 0.000 | 0.000 | 0.000 | 7.006 | 0.000 |
| 0.001 | 0.000 | 5.244 | 1.919 | 0.000 | 0.000 | 0.000 | 2.461 | 9.730 | 9.156 | 2.414 | 0.000 | 12.145 |
| 0.01 | 0.000 | 0.000 | 0.000 | 1.786 | 8.645 | 3.223 | 10.816 | 4.420 | 0.000 | 0.000 | 2.224 | 4.665 |
| 0.1 | 2.657 | 0.000 | 0.000 | 0.000 | 13.201 | 19.821 | 43.187 | 33.453 | 0.000 | 4.969 | 11.010 | 11.468 |
| 1 | 1.994 | 0.000 | 32.603 | 23.514 | 31.425 | 50.931 | 58.549 | 76.348 | 31.070 | 17.612 | 35.454 | 26.978 |
| 10 | 1.854 | 9.939 | 54.250 | 68.177 | 73.715 | 64.307 | 67.811 | 64.439 | 74.588 | 72.476 | 73.799 | 74.613 |
| 100 | 4.451 | 12.134 | 73.838 | 59.211 | 65.421 | 69.834 | 76.684 | 76.440 | 75.926 | 72.800 | 74.177 | 80.181 |
| 1000 | 2.181 | 5.610 | 68.598 | 82.139 | 79.673 | 67.602 | 74.547 | 69.147 | 71.502 | 75.065 | 80.160 | 72.334 |
| 10000 | 1.373 | 1.829 | 84.530 | 73.904 | 84.930 | 69.090 | 77.979 | 76.071 | 78.601 | 74.094 | 87.433 | 73.732 |

TABLE 36
Raw Data from FIG. 72D

| | | | | | | | | | | | LS202/ | |
|---|---|---|---|---|---|---|---|---|---|---|---|---|
| Conc. | Non- | | Random | | HS74 | | LG68/HS74 | | LS202 | | HK136 | |
| (pM) | conjugated | | (DAR 2) | | (B-FITC) | | (AB-FITC) | | (E-FITC) | | (EF-FITC) | |
| 0.0001 | 1.252 | 0.000 | 5.305 | 3.184 | 3.304 | 0.598 | 0.000 | 0.688 | 0.000 | 0.000 | 0.000 | 0.000 |
| 0.001 | 0.000 | 0.000 | 7.393 | 1.453 | 0.000 | 3.368 | 0.000 | 4.405 | 0.000 | 0.000 | 0.000 | 0.000 |
| 0.01 | 3.984 | 0.000 | 10.045 | 7.821 | 1.872 | 1.630 | 0.000 | 5.299 | 0.000 | 0.000 | 0.000 | 5.747 |
| 0.1 | 5.862 | 0.000 | 3.330 | 4.302 | 3.029 | 0.109 | 0.000 | −0.069 | 5.681 | 0.000 | 0.000 | 4.693 |
| 1 | 6.318 | 0.169 | 9.763 | 6.927 | 0.000 | 2.553 | 0.000 | 0.000 | 0.174 | 4.485 | 0.000 | 0.670 |
| 10 | 1.594 | 0.000 | 7.393 | 3.464 | 1.156 | 0.760 | 0.000 | −0.344 | 4.406 | 0.000 | 0.000 | 0.287 |
| 100 | 0.000 | 0.000 | 11.005 | 12.123 | 2.203 | 0.054 | 0.000 | 0.688 | 2.145 | 0.000 | 2.860 | 0.000 |
| 1000 | 1.821 | 0.000 | 20.316 | 18.045 | 2.808 | 9.180 | 0.000 | 10.530 | 7.304 | 0.000 | 0.000 | 0.000 |
| 10000 | 4.667 | 8.723 | 22.009 | 17.151 | 6.222 | 6.029 | 3.957 | 11.425 | 2.261 | 9.260 | 5.720 | 0.000 |

TABLE 37
Raw Data from FIG. 73F

| | Non- | | anti-FITC | | Non- | | anti-FITC | |
|---|---|---|---|---|---|---|---|---|
| Conc. | transduced + | | CAR + | | transduced + | | CAR + | |
| (pM) | aCD19 (IgG) | | aCD19 (IgG) | | isotype (IgG) | | isotype (IgG) | |
| 10000 | 0.760 | −0.760 | 9.003 | 10.574 | −1.501 | −2.309 | 44.431 | 43.009 |
| 1000 | −0.994 | −0.058 | 25.680 | 28.943 | −0.693 | −1.617 | 45.735 | 41.588 |
| 100 | 1.110 | 0.292 | 45.015 | 42.719 | −2.540 | −3.464 | 50.000 | 44.668 |
| 10 | 0.643 | 1.227 | 41.390 | 46.344 | 1.155 | 1.039 | 49.645 | 49.882 |
| 1 | 1.110 | 4.033 | 49.366 | 48.520 | 5.196 | −1.501 | 48.341 | 48.223 |
| 0 | 3.916 | 1.578 | 54.683 | 53.474 | −1.386 | 0.577 | 54.621 | 51.422 |

TABLE 38
Raw Data from FIG. 73G

| | | |
|---|---|---|
| Fluorescein | | |
| Concentration | | |
| (nM) | Non-transduced | anti-FITC CAR |

| | | | | |
|---|---|---|---|---|
| 10000 | −3.188 | −1.299 | 27.391 | 31.641 |
| 1000 | 0.826 | 0.118 | 50.413 | 50.885 |
| 100 | −0.354 | −0.236 | 55.726 | 60.213 |
| 10 | −0.118 | 0.945 | 56.198 | 55.962 |
| 1 | 2.479 | 2.125 | 57.143 | 58.087 |
| 0 | −0.826 | 2.243 | 54.782 | 59.976 |

TABLE 39
Raw Data from FIG. 73H

| | |
|---|---|
| CD19 | CD22 |

| | | | | | |
|---|---|---|---|---|---|
| 25856.31 | 25856.31 | 26131.4 | 23386.52 | 23797.37 | 23318.07 |

TABLE 40
Raw Data from FIG. 75A

| Days after | No CAR-T | | | | | | CART-19 | | | |
|---|---|---|---|---|---|---|---|---|---|---|
| 6 | 1.00E+06 | 1.00E+06 | 1.00E+06 | 1.00E+06 | 1.00E+06 | 1.00E+06 | 1.00E+06 | 1.00E+06 | 2.00E+06 | 2.00E+06 |
| 12 | 1.00E+08 | 7.00E+07 | 8.00E+07 | 5.00E+06 | 5.00E+06 | 4.00E+06 | 3.00E+03 | 3.00E+03 | 3.00E+03 | 2.00E+03 |
| 19 | 5.00E+08 | 4.00E+08 | 5.00E+08 | 4.00E+08 | 3.00E+08 | 2.00E+08 | 2.00E+03 | 9.00E+02 | 1.00E+03 | 7.00E+02 |
| 25 | | | | | | | 7.00E+02 | 2.00E+03 | 6.00E+02 | 1.00E+03 |
| 31 | | | | | | | 6.00E+02 | 2.00E+04 | 4.00E+02 | 1.00E+03 |
| 48 | | | | | | | 2.00E+03 | 2.00E+05 | 9.00E+03 | −3.00E+00 |

| | | | | | | | | | FITC-E2 CART, | |
|---|---|---|---|---|---|---|---|---|---|---|
| Days after | CART-19 | | FITC-E2 CART, 0.5 mg/kg | | | | | | 0.05 mg/kg | |
| 6 | 2.00E+06 | 2.00E+06 | 2.00E+06 | 1.00E+06 | 1.00E+06 | 2.00E+06 | 1.00E+06 | 2.00E+06 | 1.00E+06 | 1.00E+06 |
| 12 | 1.00E+03 | 1.00E+03 | 2.00E+03 | 2.00E+03 | 1.00E+03 | 9.00E+02 | 1.00E+03 | 3.00E+03 | 1.00E+04 | 4.00E+04 |
| 19 | 2.00E+03 | 1.00E+03 | 1.00E+03 | 8.00E+02 | 9.00E+02 | 7.00E+02 | 1.00E+03 | 1.00E+03 | 2.00E+03 | 4.00E+04 |
| 25 | 2.00E+03 | 1.00E+03 | 4.00E+02 | 3.00E+02 | 4.00E+03 | 7.00E+02 | 4.00E+02 | 1.00E+03 | 5.00E+03 | 6.00E+03 |
| 31 | 1.00E+03 | 8.00E+02 | 1.00E+02 | −1.00E+02 | 5.00E+02 | | | | | |
| 48 | 1.00E+03 | 3.00E+02 | 4.00E+04 | 1.00E+03 | 2.00E+04 | | | | | |

| Days after | FITC-E2 CART, 0.05 mg/kg | | | | FITC-E2 CART, 0.005 mg/kg | | | | | |
|---|---|---|---|---|---|---|---|---|---|---|
| 6 | 1.00E+06 | 1.00E+06 | 1.00E+06 | 1.00E+06 | 1.00E+06 | 6.00E+05 | 9.00E+05 | 1.00E+06 | 1.00E+06 | 2.00E+06 |
| 12 | 6.00E+03 | 3.00E+03 | 3.00E+03 | 1.00E+04 | 1.00E+06 | 3.00E+06 | 6.00E+05 | 4.00E+06 | 2.00E+06 | 1.00E+06 |
| 19 | 2.00E+03 | 4.00E+04 | 1.00E+03 | 5.00E+04 | 1.00E+07 | 6.00E+07 | 1.00E+07 | 8.00E+07 | 5.00E+07 | 3.00E+07 |
| 25 | 5.00E+03 | 4.00E+05 | 2.00E+03 | 7.00E+04 | 1.00E+08 | 4.00E+07 | 4.00E+07 | 1.00E+08 | 1.00E+08 | 2.00E+08 |
| Days after | No CAR-T | | | | | | CART-19 | | | |
| 31 | 5.00E+02 | 8.00E+02 | 1.00E+03 | 3.00E+04 | 2.00E+06 | 6.00E+04 | 3.00E+06 | 2.00E+04 | 9.00E+05 | |
| 48 | 4.00E+02 | 1.00E+03 | 1.00E+03 | 6.00E+07 | 5.00E+05 | 2.00E+08 | 6.00E+05 | 3.00E+06 | 3.00E+08 | |

TABLE 41
Raw Data from FIG. 75C

| Days | | | | | | | | | | | | |
|---|---|---|---|---|---|---|---|---|---|---|---|---|
| after | | | | | | | | | | | | |
| Nalm- | | | | | | | | | | | | |
| 6 iv | Nalm-6, No CAR-T | | | | | | Nalm-6, CART-19 | | | | | |
| 6 | 100.0 | 100.0 | 100.0 | 100.0 | 100.0 | 100.0 | 100.0 | 100.0 | 100.0 | 100.0 | 100.0 | 100.0 |
| 8 | 98.3 | 98.1 | 98.6 | 100.0 | 99.5 | 103.7 | 86.6 | 91.0 | 86.7 | 88.6 | 90.9 | 79.4 |
| 10 | 100.6 | 100.5 | 99.5 | 97.2 | 98.9 | 104.2 | 94.3 | 91.4 | 93.6 | 90.7 | 90.9 | 99.5 |
| 12 | 100.6 | 98.6 | 98.6 | 99.1 | 100.0 | 103.2 | 91.9 | 95.7 | 98.7 | 99.5 | 100.0 | 105.6 |
| 14 | 103.9 | 99.5 | 101.0 | 99.1 | 102.2 | 105.8 | 101.2 | 95.7 | 100.9 | 101.0 | 102.5 | 108.9 |
| 16 | 100.0 | 96.2 | 98.6 | 96.3 | 98.9 | 102.6 | 98.0 | 97.0 | 99.6 | 101.6 | 100.5 | 107.0 |
| 18 | 89.9 | 86.7 | 87.0 | 91.7 | 93.0 | 93.7 | 98.8 | 97.4 | 98.3 | 103.1 | 101.5 | 109.8 |
| 21 | 76.4 | 65.7 | 72.5 | 75.1 | 76.3 | 78.4 | 100.8 | 97.8 | 100.0 | 105.7 | 100.0 | 109.8 |
| 23 | | | | | | | 102.4 | 100.9 | 101.3 | 108.3 | 101.0 | 112.6 |
| 25 | | | | | | | 107.7 | 102.2 | 106.9 | 110.9 | 103.6 | 117.8 |
| 27 | | | | | | | 102.8 | 103.9 | 107.7 | 113.0 | 104.6 | 116.8 |
| 29 | | | | | | | 103.6 | 103.4 | 102.6 | 105.2 | 99.0 | 113.6 |
| 31 | | | | | | | 104.0 | 105.2 | 110.3 | 111.4 | 104.1 | 115.4 |
| 33 | | | | | | | 104.0 | 107.8 | 107.3 | 109.3 | 103.6 | 113.6 |
| Days | | | | | | | | | | | | |
| after | | | | | | | | | | | | |
| Nalm- | | | | | | | | | | | | |
| 6 iv | Nalm-6, E2 0.5 mg/kg | | | | | | Nalm-6, E2, 0.05 mg/kg | | | | | |
| 6 | 100.0 | 100.0 | 100.0 | 100.0 | 100.0 | 100.0 | 100.0 | 100.0 | 100.0 | 100.0 | 100.0 | 100.0 |
| 8 | 95.1 | 98.5 | 96.2 | 95.6 | 96.7 | 89.3 | 93.8 | 94.8 | 97.3 | 98.1 | 97.0 | 99.5 |
| 10 | 95.5 | 99.0 | 95.8 | 94.0 | 94.4 | 91.8 | 98.1 | 99.6 | 100.5 | 103.8 | 103.0 | 106.2 |
| 12 | 97.3 | 102.5 | 101.4 | 101.6 | 99.5 | 98.5 | 102.4 | 102.4 | 102.7 | 106.6 | 105.0 | 104.8 |
| 14 | 99.6 | 109.6 | 103.3 | 108.2 | 101.4 | 101.5 | 104.7 | 104.8 | 108.2 | 104.7 | 104.5 | 104.3 |
| 16 | 96.9 | 111.2 | 102.3 | 104.4 | 99.5 | 99.0 | 106.2 | 103.2 | 106.0 | 105.2 | 102.5 | 106.7 |
| 18 | 99.6 | 112.7 | 99.5 | 104.9 | 99.1 | 104.4 | 104.7 | 103.6 | 108.7 | 110.0 | 105.5 | 107.7 |
| 21 | 96.8 | 118.3 | 98.6 | 105.5 | 100.0 | 103.4 | 110.0 | 104.0 | 109.3 | 110.9 | 105.0 | 111.5 |
| 23 | 99.1 | 117.8 | 101.4 | 106.0 | 101.9 | 106.8 | 111.4 | 106.8 | 110.4 | 114.2 | 106.5 | 111.0 |
| 25 | 100.9 | 120.8 | 105.6 | 109.3 | 104.7 | 102.4 | 115.2 | 108.8 | 112.6 | 116.6 | 111.4 | 117.2 |
| 27 | 100.9 | 119.8 | 103.8 | 109.3 | 92.1 | 106.8 | 112.8 | 108.0 | 113.1 | 117.1 | 111.4 | 118.2 |
| 29 | 96.8 | 114.7 | 104.7 | 103.8 | 102.3 | 101.0 | 110.4 | 104.0 | 111.5 | 111.8 | 107.5 | 112.0 |
| 31 | 104.5 | 119.8 | 107.0 | 110.4 | 108.4 | 105.3 | 114.2 | 110.0 | 114.2 | 115.2 | 113.9 | 118.2 |
| 33 | 103.2 | 115.7 | 103.8 | 109.3 | 108.4 | 105.3 | 111.4 | 105.6 | 115.8 | 113.3 | 111.9 | 114.4 |

| Days after | | | | | | | No Nalm-6, E2, | | |
|---|---|---|---|---|---|---|---|---|---|
| Nalm-6 iv | Nalm-6, E2, 0.005 mg/kg | | | | | | 0.5 mg/kg | | |
| 6 | 100.0 | 100.0 | 100.0 | 100.0 | 100.0 | 100.0 | 100.0 | 100.0 | 100.0 |
| 8 | 101.4 | 99.6 | 100.5 | 98.2 | 96.4 | 103.2 | 102.6 | 101.2 | 102.8 |
| 10 | 102.3 | 100.4 | 102.0 | 100.9 | 96.4 | 105.3 | 103.9 | 101.7 | 103.8 |
| 12 | 104.5 | 104.0 | 105.1 | 104.4 | 96.8 | 105.8 | 98.3 | 102.1 | 103.3 |
| 14 | 106.8 | 107.5 | 106.1 | 104.0 | 99.6 | 108.9 | 101.7 | 103.7 | 104.7 |
| 16 | 101.4 | 104.0 | 103.1 | 104.4 | 97.6 | 108.4 | 97.8 | 106.6 | 105.7 |
| 18 | 101.4 | 100.4 | 102.6 | 103.5 | 94.4 | 105.8 | 99.1 | 111.2 | 107.1 |
| 21 | 101.8 | 98.7 | 103.1 | 102.6 | 91.2 | 108.9 | 100.4 | 112.4 | 109.0 |
| 23 | 101.8 | 85.8 | 105.6 | 96.9 | 79.1 | 108.4 | 105.2 | 113.2 | 109.0 |
| 25 | 90.0 | 72.1 | 107.7 | 81.5 | 67.5 | 107.9 | 103.5 | 109.5 | 109.0 |
| 27 | 75.5 | 65.5 | 104.6 | 73.1 | | 88.4 | 105.6 | 113.2 | 113.3 |
| 29 | | | 87.8 | 67.4 | | | 106.9 | 107.9 | 109.5 |
| 31 | | | | | | | 95.7 | 113.2 | 116.1 |
| 33 | | | | | | | 85.3 | 112.0 | 114.7 |

TABLE 42
Raw Data from FIG. 76B

| Days after Nalm-6 | | | | | | |
|---|---|---|---|---|---|---|
| iv | PBS | | | | | |
| 6 | 1.00E+06 | 1.00E+06 | 1.00E+06 | 1.00E+06 | 1.00E+06 | 1.00E+06 |
| 12 | 1.00E+08 | 7.00E+07 | 8.00E+07 | 5.00E+06 | 5.00E+06 | 4.00E+06 |
| 19 | 5.00E+08 | 4.00E+08 | 5.00E+08 | 4.00E+08 | 3.00E+08 | 2.00E+08 |
| 25 | | | | | | |
| 31 | | | | | | |
| Days after Nalm-6 | | | | | | |
| iv | FITC-E2 CART, 0.5 mg/kg | | | | | |
| 6 | 2.00E+06 | 1.00E+06 | 1.00E+06 | 2.00E+06 | 1.00E+06 | 2.00E+06 |
| 12 | 2.00E+03 | 2.00E+03 | 1.00E+03 | 9.00E+02 | 1.00E+03 | 3.00E+03 |
| 19 | 1.00E+03 | 8.00E+02 | 9.00E+02 | 7.00E+02 | 1.00E+03 | 1.00E+03 |
| 25 | 4.00E+02 | 3.00E+02 | 4.00E+03 | 7.00E+02 | 4.00E+02 | 1.00E+03 |
| 31 | 1.00E+02 | 1.00E+00 | 5.00E+02 | 5.00E+02 | 8.00E+02 | 1.00E+03 |
| Days after Nalm-6 | | | | | | |
| iv | FITC-E2 CART, 0.05 mg/kg (×6) | | | | | |
| 6 | 1.00E+06 | 1.00E+06 | 1.00E+06 | 1.00E+06 | 1.00E+06 | 1.00E+06 |
| 12 | 1.00E+04 | 4.00E+04 | 6.00E+03 | 3.00E+03 | 3.00E+03 | 1.00E+04 |
| 19 | 2.00E+03 | 4.00E+04 | 2.00E+03 | 4.00E+04 | 1.00E+03 | 5.00E+04 |
| 25 | 5.00E+03 | 6.00E+03 | 5.00E+03 | 4.00E+05 | 2.00E+03 | 7.00E+04 |
| 31 | 3.00E+04 | 2.00E+06 | 6.00E+04 | 3.00E+06 | 2.00E+04 | 9.00E+05 |
| Days after Nalm-6 | | | | | | |
| iv | FITC-E2 CART, 0.05 mg/kg (×12) | | | | | |
| 6 | 1.00E+06 | 9.00E+05 | 6.00E+05 | 1.00E+06 | 1.00E+06 | 6.00E+05 |
| 12 | 1.00E+04 | 3.00E+03 | 2.00E+03 | 7.00E+04 | 9.00E+04 | 3.00E+03 |
| 19 | 1.00E+03 | 2.00E+03 | 2.00E+04 | 5.00E+03 | 1.00E+05 | 3.00E+03 |
| 25 | 4.00E+04 | 1.00E+03 | 5.00E+03 | 5.00E+03 | 7.00E+04 | 5.00E+03 |
| 31 | 1.00E+05 | 5.00E+02 | 6.00E+03 | 1.00E+04 | 3.00E+05 | 5.00E+03 |

TABLE 43
Raw Data from FIG. 77A

| Days after Nalm-6 | | | | | | |
|---|---|---|---|---|---|---|
| iv | PBS | | | | | |
| 6.00E+00 | 1.00E+06 | 2.00E+06 | 1.00E+06 | 2.00E+06 | 1.00E+06 | 2.00E+06 |
| 1.00E+01 | 1.00E+08 | 1.00E+08 | 2.00E+08 | 9.00E+07 | 8.00E+07 | 1.00E+08 |
| 2.00E+01 | 2.00E+08 | 2.00E+08 | 3.00E+08 | 5.00E+08 | 2.00E+08 | 4.00E+08 |
| 3.00E+01 | | | | | | |
| 3.00E+01 | | | | | | |
| Days after Nalm-6 | | | | | | |
| iv | 0.5 mg/kg (×6) | | | | | |
| 6.00E+00 | 2.00E+06 | 1.00E+06 | 1.00E+06 | 2.00E+06 | 1.00E+06 | 1.00E+06 |
| 1.00E+01 | 2.00E+03 | 1.00E+03 | 1.00E+03 | 8.00E+02 | 9.00E+02 | 1.00E+03 |
| 2.00E+01 | 4.00E+03 | 4.00E+03 | 4.00E+03 | 4.00E+03 | 4.00E+03 | 4.00E+03 |
| 3.00E+01 | 3.00E+03 | 3.00E+03 | 3.00E+03 | 4.00E+03 | 4.00E+03 | 4.00E+03 |
| 3.00E+01 | 3.00E+03 | 3.00E+03 | 3.00E+03 | 5.00E+03 | 4.00E+03 | 4.00E+03 |
| Days after Nalm-6 | | | | | | |
| iv | 0.05 mg/kg (×6) | | | | | |
| 6.00E+00 | 1.00E+06 | 1.00E+06 | 2.00E+06 | 1.00E+06 | 1.00E+06 | 1.00E+06 |
| 1.00E+01 | 3.00E+03 | 2.00E+03 | 1.00E+03 | 6.00E+03 | 5.00E+03 | 5.00E+03 |
| 2.00E+01 | 8.00E+03 | 4.00E+03 | 4.00E+03 | 6.00E+03 | 7.00E+03 | 7.00E+04 |
| 3.00E+01 | 7.00E+04 | 3.00E+03 | 4.00E+03 | 6.00E+03 | 4.00E+04 | 5.00E+05 |
| 3.00E+01 | 3.00E+06 | 6.00E+04 | 1.00E+04 | 2.00E+05 | 2.00E+06 | 7.00E+07 |
| Days after Nalm-6 | | | | | | |
| iv | 0.05 mg/kg (×3) + 0.5 mg/kg (×3) | | | | | |
| 6.00E+00 | 2.00E+06 | 1.00E+06 | 1.00E+06 | 9.00E+05 | 1.00E+06 | 1.00E+06 |
| 1.00E+01 | 1.00E+03 | 2.00E+03 | 2.00E+04 | 8.00E+02 | 4.00E+03 | 4.00E+03 |
| 2.00E+01 | 2.00E+03 | 1.00E+03 | 1.00E+03 | 3.00E+03 | 2.00E+03 | 2.00E+03 |
| 3.00E+01 | 3.00E+03 | 3.00E+03 | 3.00E+03 | 4.00E+03 | 4.00E+03 | 4.00E+03 |
| 3.00E+01 | 4.00E+03 | 4.00E+03 | 4.00E+03 | 5.00E+03 | 5.00E+03 | 5.00E+03 |

TABLE 45
CAR Amino Acid Sequences

| | SEQ | |
|---|---|---|
| Name | ID | Sequence |
| CAR-Her2 (CD8 | 48 | DIQMTQSPSSLSASVGDRVTITCRASQDVNTAVAWYQQKPGKAP |
| hinge) | | KLLIYSASFLYSGVPSRFSGSRSGTDFTLTISSLQPEDFATYYCQQ |
| | | HYTTPPTFGQGTKVEIKRTGGGGSGGGGSGGGGSEVQLVESGGG |
| | | LVQPGGSLRLSCAASGFNIKDTYIHWVRQAPGKGLEWVARIYPT |
| | | NGYTRYADSVKGRFTISADTSKNTAYLQMNSLRAEDTAVYYCS |
| | | RWGGDGFYAMDYWGQGTLVTVSSAAATTTPAPRPPTPAPTIAS |
| | | QPLSLRPEACRPAAGGAVHTRGLDFACDIYIWAPLAGTCGVLLL |
| | | SLVITLYCKRGRKKLLYIFKQPFMRPVQTTQEEDGCSCRFPEEEE |
| | | GGCELRVKFSRSADAPAYKQGQNQLYNELNLGRREEYDVLDKR |
| | | RGRDPEMGGKPRRKNPQEGLYNELQKDKMAEAYSEIGMKGERR |
| | | RGKGHDGLYQGLSTATKDTYDALHMQALPPR (SEQ ID NO: 48) |
| CAR-Her2 | 49 | DIQMTQSPSSLSASVGDRVTITCRASQDVNTAVAWYQQKPGKAP |
| (IgG4m hinge) | | KLLIYSASFLYSGVPSRFSGSRSGTDFTLTISSLQPEDFATYYCQQ |
| | | HYTTPPTFGQGTKVEIKRTGGGGSGGGGSGGGGSEVQLVESGGG |
| | | LVQPGGSLRLSCAASGFNIKDTYIHWVRQAPGKGLEWVARIYPT |
| | | NGYTRYADSVKGRFTISADTSKNTAYLQMNSLRAEDTAVYYCS |
| | | RWGGDGFYAMDYWGQGTLVTVSSAAAESKYGPPCPPCPDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| | | (SEQ ID NO: 49) |
| CAR-FITC (CD8 | 50 | QVQLVESGGNLVQPGGSLRLSCAASGFTFGSFSMSWVRQAPGG |
| hinge) | | GLEWVAGLSARSSLTHYADSVKGRFTISRDNAKNSVYLQMNSL |
| | | RVEDTAVYYCARRSYDSSGYWGHFYSYMDVWGQGTLVTVSGG |
| | | GGSGGGGSGGGGSSVLTQPSSVSAAPGQKVTISCSGSTSNIGNNY |
| | | VSWYQQHPGKAPKLMIYDVSKRPSGVPDRFSGSKSGNSASLDIS |
| | | GLQSEDEADYYCAAWDDSLSEFLFGTGTKLTVLGTTTPAPRPPTP |
| | | APTIASQPLSLRPEACRPAAGGAVHTRGLDFACDIYIWAPLAGTC |
| | | GVLLLSLVITLYCKRGRKKLLYIFKQPFMRPVQTTQEEDGCSCRF |
| | | PEEEEGGCELRVKFSRSADAPAYKQGQNQLYNELNLGRREEYD |
| | | VLDKRRGRDPEMGGKPRRKNPQEGLYNELQKDKMAEAYSEIG |
| | | MKGERRRGKGHDGLYQGLSTATKDTYDALHMQALPPR (SEQ ID |
| | | NO: 50) |
| CAR-FITC | 51 | QVQLVESGGNLVQPGGSLRLSCAASGFTFGSFSMSWVRQAPGG |
| (IgG4m hinge) | | GLEWVAGLSARSSLTHYADSVKGRFTISRDNAKNSVYLQMNSL |
| | | RVEDTAVYYCARRSYDSSGYWGHFYSYMDVWGQGTLVTVSGG |
| | | GGSGGGGSGGGGSSVLTQPSSVSAAPGQKVTISCSGSTSNIGNNY |
| | | VSWYQQHPGKAPKLMIYDVSKRPSGVPDRFSGSKSGNSASLDIS |
| | | GLQSEDEADYYCAAWDDSLSEFLFGTGTKLTVLGESKYGPPCPP |
| | | CPDIYIWAPLAGTCGVLLLSLVITLYCKRGRKKLLYIFKQPFMRP |
| | | VQTTQEEDGCSCRFPEEEEGGCELRVKFSRSADAPAYKQGQNQL |
| | | YNELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR (SEQ ID NO: 51) |

TABLE 46
CAR-EC switch target interacting domain
(antibodies, variable regions)-Amino Acid Sequence

| NAME | SEQ ID | SEQUENCE |
|---|---|---|
| Fab | 52 | ASTKGPSVFPLAPSSKSTSGGTAALGCLVKDYFPEPV |
| Heavy | | TVSWNSGALTSGVHTFPAVLQSSGLYSLSSVVTVPSS |
| Chain | | SLGTQTYICNVNEIKPSNTKVDKKVEPKSCDKTHT |
| Fab | 53 | RTVAAPSVFIFPPSDEQLKSGTASVVCLLNNFYPREA |
| Light | | KVQWKVDNALQSGNSQESVTEQDSKDSTYSLSSTLTL |
| Chain | | SKADYEKHKVYACEVTHQGLSSPVTKSFNRGEC |

What is claimed is:

1. A chimeric antigen receptor comprising an extracellular domain, a transmembrane domain, and an intracellular signaling domain, wherein the extracellular domain comprises:
(a) a region that interacts with a chimeric antigen receptor switch; and
(b) a hinge domain.

2. The chimeric antigen receptor of claim 1, wherein the hinge domain is about 10 amino acids long.

3. The chimeric antigen receptor of claim 1, wherein the hinge domain is about 45 amino acids long.

4. The chimeric antigen receptor of any one of claims 1-3, wherein the hinge domain is flexible.

5. The chimeric antigen receptor of any one of claims 1-3, wherein the hinge domain is rigid.

6. The chimeric antigen receptor of any one of claims 1-5, wherein a first cysteine of the first chimeric antigen receptor and a second cysteine of a second chimeric antigen receptor form a disulfide bond, resulting in multimerization of the chimeric antigen receptor and the second chimeric antigen receptor.

7. The chimeric antigen receptor of any one of claims 1-6, wherein the hinge domain has a sequence selected from SEQ ID NOS: 34-37.

8. The chimeric antigen receptor of any one of claims 1-6, wherein the hinge domain has a sequence that is at least about 50% homologous to a sequence selected from SEQ ID NOS: 34-37.

9. The chimeric antigen receptor of any one of claims 1-8, wherein the extracellular domain comprises an antibody or antibody fragment that binds a hapten of the chimeric antigen receptor switch, wherein the chimeric antigen receptor switch comprises a target interacting domain.

10. The chimeric antigen receptor of claim 9, wherein the hapten is FITC or a derivative thereof.

11. The chimeric antigen receptor of any one of claims 1-10, wherein the hinge domain comprises a peptide derived from a protein selected from a CD8, an IgG, portions thereof, and combinations thereof.

12. A soluble T cell receptor switch comprising:
(a) a chimeric antigen receptor interacting domain; and
(b) a soluble T cell receptor or portion thereof.

13. The soluble T cell receptor switch of claim 12, wherein the chimeric antigen receptor interacting domain is linked or conjugated to a terminus of a domain of the soluble T cell receptor.

14. The soluble T cell receptor switch of claim 12 wherein the chimeric antigen receptor interacting domain is linked or conjugated into an internal site of a domain of the soluble T cell receptor.

15. The soluble T cell receptor switch of claim 13 or 14, wherein the domain of the soluble T cell receptor is selected from an alpha chain, a beta chain, a gamma chain, a delta chain, an epsilon chain and a zeta chain.

16. The soluble T cell receptor switch of claim 12, further comprising a linker, wherein the linker links the chimeric antigen receptor interacting domain to the soluble T cell receptor or portion thereof.

17. The soluble T cell receptor switch of claim 16, wherein the linker is selected from a linker depicted in FIGS. 19-22 and 51, 52, 54 and 55.

18. The soluble T cell receptor switch of any one of claims 12-17, wherein the chimeric antigen receptor interacting domain comprises a hapten.

19. The soluble T cell receptor switch of claim 18, wherein the hapten is FITC or a derivative thereof.

20. The soluble T cell receptor switch of any one of claims 12-19, wherein the chimeric antigen receptor interacting domain does not comprise a peptide.

21. The soluble T cell receptor switch of any one of claims 12-19, wherein the soluble T cell receptor comprises an unnatural amino acid.

22. The soluble T cell receptor switch of claim 21, wherein the chimeric antigen receptor interacting domain is linked or conjugated to the unnatural amino acid.

23. A chimeric antigen receptor switch comprising:
(a) a chimeric antigen receptor interacting domain; and
(b) a target interacting domain.

24. The chimeric antigen receptor switch of claim 23, wherein the target interacting domain comprises an antibody or antibody fragment.

25. The chimeric antigen receptor of claim 24, wherein the chimeric antigen receptor interacting domain is connected to a chain of the targeting antibody or antibody fragment and is selected from a light chain, a heavy chain, and a portion thereof.

26. The chimeric antigen receptor switch of claim 24, wherein the antibody or antibody fragment is selected from an anti-CS1 antibody, an anti Her2 antibody, an anti-BCMA antibody, an anti-CD19 antibody, an anti-CD22 antibody, an anti-CLL1 antibody, an anti-CD33 antibody, an anti-CD123 antibody, and fragments thereof.

27. The chimeric antigen receptor switch of any one of claims 24 to 26, wherein the antibody fragment is a Fab.

28. The chimeric antigen receptor switch of any one of claims 24 to 26, wherein the antibody fragment is a variable region of the targeting antibody.

29. The chimeric antigen receptor switch of claim 25, wherein the heavy chain has a sequence selected from SEQ ID NOS: 11, 13, 15, 17, 19, 21, 23, 25, 27, 29 and 31 and optionally SEQ ID NO: 52.

30. The chimeric antigen receptor switch of claim 25, wherein the heavy chain has a sequence that is at least about 50% homologous to a sequence selected from SEQ ID NOS: 11, 13, 15, 17, 19, 21, 23, 25, 27, 29 and 31 and optionally SEQ ID NO: 52.

31. The chimeric antigen receptor switch of claim 25, wherein the light chain has a sequence selected from SEQ ID NOS: 10, 12, 14, 16, 18, 20, 22, 24, 26, 28 and 30 and optionally SEQ ID NO: 53.

32. The chimeric antigen receptor switch of claim 25, wherein the light chain has a sequence that is at least about 50% homologous to a sequence selected from SEQ ID NOS: 10, 12, 14, 16, 18, 20, 22, 24, 26, 28 and 30 and optionally SEQ ID NO: 53.

33. The chimeric antigen receptor switch of any one of claims 23-32, wherein the chimeric antigen receptor interacting domain is a small molecule.

34. The chimeric antigen receptor switch of any one of claims 23-32, wherein the chimeric antigen receptor interacting domain is a hapten.

35. The chimeric antigen receptor switch of any one of claims 23-32, wherein the chimeric antigen receptor interacting domain is selected from FITC, biotin, and dinitrophenol.

36. The chimeric antigen receptor of any one of claims 23-32, wherein the chimeric antigen receptor interacting domain comprises FITC.

37. The chimeric antigen receptor switch of claim 23, further comprising a linker, wherein the linker connects the chimeric antigen receptor interacting domain and the target interacting domain.

38. The chimeric antigen receptor switch of claim 23, wherein the target interacting domain comprises an unnatural amino acid.

39. The chimeric antigen receptor switch of claim 38, wherein the chimeric antigen receptor interacting domain and the target interacting domain are connected or linked by the unnatural amino acid.

40. The chimeric antigen receptor switch of claim 39, wherein the target interacting domain comprises an antibody or antibody fragment.

41. The chimeric antigen receptor switch of claim 39, wherein the chimeric antigen receptor interacting domain is connected to a chain of the targeting antibody or antibody fragment and is selected from a light chain, a heavy chain, and a portion thereof.

42. The chimeric antigen receptor switch of claim 41, wherein the targeting antibody or antibody fragment is an anti-CLL1 antibody or antibody fragment comprising a variable light chain of SEQ ID NO. 18 and optionally a constant light chain of SEQ ID NO: 53 and a variable heavy chain of SEQ ID NO. 19 and optionally a constant heavy chain of SEQ ID NO: 52, and the unnatural amino acid is located at a site selected from Glycine 69, Alanine 110, and Serine 203 of a light chain of the anti-CLL1 antibody or antibody fragment, and Serine 75, Alanine 124, Lysine 139 of a heavy chain of the anti-CLL1 antibody or antibody fragment.

43. The chimeric antigen receptor switch of claim 41, wherein the targeting antibody or antibody fragment is an anti-CD33 antibody or antibody fragment comprising a variable light chain of SEQ ID NO. 20 and optionally a constant light chain of SEQ ID NO: 53 and a variable heavy chain of SEQ ID NO. 21 and optionally a constant heavy chain of SEQ ID NO: 52, and the unnatural amino acid is located at a site selected from Glycine 72, Threonine 113 and Serine 206 of a light chain of the anti-CD33 antibody or antibody fragment, and Proline 75, Alanine 117 and Lysine 132 of a heavy chain of the anti-CD33 antibody or antibody fragment.

44. The chimeric antigen receptor switch of claim 41, wherein the targeting antibody or antibody fragment is an anti-CD33 antibody or antibody fragment comprising a variable light chain of SEQ ID NO. 22 and optionally a constant light chain of SEQ ID NO: 53 and a variable heavy chain of SEQ ID NO. 23 and optionally a constant heavy chain of SEQ ID NO: 52, and the unnatural amino acid is located at a site selected from Glycine 72, Threonine 113 and Serine 206 of a light chain of the anti-CD33 antibody or antibody fragment, and Serine 75, Alanine 117 and Lysine 132 of a heavy chain of the anti-CD33 antibody or antibody fragment.

45. The chimeric antigen receptor switch of claim 41, wherein the targeting antibody or antibody fragment is an anti-CD19 antibody or antibody fragment comprising a variable light chain of SEQ ID NO. 16 and optionally a constant light chain of SEQ ID NO: 53 and a variable heavy chain of SEQ ID NO. 17 and optionally a constant heavy chain of SEQ ID NO: 52, and the unnatural amino acid is located at a site selected from Glycine 68, Threonine 109 and Serine 202 of a light chain of the anti-CD19 antibody or antibody fragment, and Serine 74, Alanine 121, Lysine 136 of a heavy chain of the anti-CD19 antibody or antibody fragment.

46. The chimeric antigen receptor switch of claim 41, wherein the targeting antibody or antibody fragment is an anti-CD22 antibody or antibody fragment comprising a variable light chain of SEQ ID NO. 30 and optionally a constant light chain of SEQ ID NO: 53 and a variable heavy chain of SEQ ID NO. 31 and optionally a constant heavy chain of SEQ ID NO: 52, and the unnatural amino acid is located at a site selected from Glycine 74, Threonine 114 and Serine 207 of a light chain of the anti-CD22 antibody or antibody fragment, and Serine 75, Alanine 117, Lysine 132 of a heavy chain of the anti-CD22 antibody or antibody fragment.

47. The chimeric antigen receptor switch of claim 41, wherein the targeting antibody or antibody fragment is an anti-CD22 antibody or antibody fragment comprising a variable light chain of SEQ ID NO. 28 and optionally SEQ ID NO: 53 a constant light chain of and a variable heavy chain of SEQ ID NO. 29 and optionally a constant heavy chain of SEQ ID NO: 52, and the unnatural amino acid is located at a site selected from Glycine 68, Threonine 109 and Serine 202 of a light chain of the anti-CD22 antibody or antibody fragment, and Serine 78, Alanine 125, Lysine 140 of a heavy chain of the anti-CD22 antibody or antibody fragment.

48. The chimeric antigen receptor switch of claim 41, wherein the targeting antibody or antibody fragment is an anti-Her2 antibody or antibody fragment comprising a variable light chain of SEQ ID NO. 12 and optionally SEQ ID NO: 53 a constant light chain of and a variable heavy chain of SEQ ID NO. 13 and optionally a constant heavy chain of SEQ ID NO: 52, and the unnatural amino acid is located at a site selected from Glycine 68, Threonine 109 and Serine 202 of a light chain of the anti-Her2 antibody or antibody fragment, and Serine 75, Alanine 121, Lysine 136 of a heavy chain of the anti-Her2 antibody or antibody fragment.

49. The chimeric antigen receptor switch of claim 41, wherein the targeting antibody or antibody fragment is an anti-CD123 antibody or antibody fragment comprising a variable light chain of SEQ ID NO. 26 and optionally SEQ ID NO: 53 a constant light chain of and a variable heavy chain of SEQ ID NO. 27 and optionally a constant heavy chain of SEQ ID NO: 52, and the unnatural amino acid is located at a site selected from Glycine 68, Threonine 109 and Serine 202 of a light chain of the anti-CD123 antibody or antibody fragment, and Serine 75, Alanine 116, Lysine 131 of a heavy chain of the anti-CD123 antibody or antibody fragment.

50. The chimeric antigen receptor switch of claim 41, wherein the targeting antibody or antibody fragment is an anti-CD123 antibody or antibody fragment comprising a variable light chain of SEQ ID NO. 24 and optionally SEQ ID NO: 53 a constant light chain of and a variable heavy chain of SEQ ID NO. 25 and optionally a constant heavy chain of SEQ ID NO: 52, and the unnatural amino acid is located at a site selected from Arginine 72, Threonine 113 and Serine 206 of a light chain of the anti-CD123 antibody or antibody fragment, and Serine 75, Alanine 119, Lysine 134 of a heavy chain of the anti-CD123 antibody or antibody fragment.

51. A pharmaceutical composition comprising the chimeric antigen receptor switch of any one of claims 23-50.

52. A pharmaceutical composition comprising one or more of the chimeric antigen receptor switches in any one of claims 23-50.

53. A method of treating a disease or condition in a subject in need thereof, comprising administering a first chimeric antigen receptor switch selected from any one of claim 23-50 or 109 and a chimeric antigen receptor effector cell, wherein the chimeric antigen receptor switch and/or chimeric antigen receptor effector cell is administered by a method selected from intraperitoneal injection and intravenous injection.

54. The method of claim 53, comprising administering the chimeric antigen receptor switch and/or the chimeric antigen receptor effector cell multiple times.

55. The method of claim 53 or 54, wherein the disease or condition is AML, ALL, CLL, multiple myeloma, breast cancer, neuroblastoma, pancreatic cancer, endometrial cancer, ovarian cancer, or colon cancer.

56. The method of any one of claims 53-54, comprising administering the first chimeric antigen receptor switch comprising a first targeting antibody or antibody fragment and a second chimeric antigen receptor switch comprising a second targeting antibody or antibody fragment, wherein the first targeting antibody or antibody fragment binds a first antigen and the second targeting antibody or antibody fragment binds a second antigen, wherein the first antigen and the second antigen are different.

57. The method of claim 56, wherein the first and/or second antigen is selected from CD19, CD22, Her2, CLL1, CD33, BMCA, CD123, CS1, EGFR, EGFRvIII, CD20, or CEA.

58. A method of selecting an optimal switchable chimeric antigen receptor platform, comprising:
(a) attaching a first chimeric antigen receptor interacting domain to a first site of a target interacting domain that binds a first cell surface molecule on a first target cell to produce a first switch;
(b) attaching a second chimeric antigen receptor interacting domain to a second site of a second target interacting domain that binds a second cell surface molecule on a second target cell to produce a second switch;
(c) contacting the first target cell with the first switch and a first chimeric antigen receptor effector cell expressing a first chimeric antigen receptor;
(d) contacting the second target cell with the second switch and a second chimeric antigen receptor effector cell expressing a second chimeric antigen receptor; and
(e) comparing a first cytotoxic effect of the first switch and the first chimeric antigen receptor effector cell on the first target cell to a second cytotoxic effect of the second switch and the second chimeric antigen receptor effector cell on the second target cell; and
(f) selecting the first switch and first chimeric antigen receptor effector cell or the second switch and the second chimeric antigen receptor effector cell as the optimal switchable chimeric antigen receptor platform based on comparing the first cytotoxic effect to the second cytotoxic effect.

59. The method of claim 58, wherein the first chimeric antigen receptor interacting domain and the second chimeric antigen receptor interacting domain are the same.

60. The method of claim 58 wherein the first chimeric antigen receptor interacting domain and the second chimeric antigen receptor interacting domain are comprised of FITC.

61. The method of any one of claims 58 to 60, wherein the first target interacting domain and the second target interacting domain are the same.

62. The method of any one of claims 58-60, wherein the first site and the second site are different.

63. The method of any one of claims 58-60, wherein the first site and the second site are the same.

64. The method of any one of claims 58-63, wherein the first and/or second target interacting domain comprises a peptide or protein.

65. The method of claim 63, wherein the first site and/or second site is selected from an N terminus of the peptide or protein, a C terminus of the peptide or protein, and an internal site of the peptide or protein.

66. The method of any one of claims 58-64, wherein the first and/or second targeting moiety comprises an antibody or antibody fragment.

67. The method of claim 66, wherein the first site and/or second site is selected from an N terminus of the antibody or antibody fragment, a C terminus of the antibody or antibody fragment, and an internal site of the antibody or antibody fragment.

68. The method of claim 66, wherein the first site and/or second site is selected from a light chain of the antibody or antibody fragment and a heavy chain of the antibody or antibody fragment.

69. The method of claim 66, wherein the first site and/or second site is selected from a variable region of the antibody or antibody fragment and a constant region of the antibody or antibody fragment.

70. The method of claim 66, wherein the first site and/or second site is selected from a VL domain, a CL domain, a VH domain, a CH1 domain, a CH2 domain, a CH3 domain, and a hinge domain of the antibody or antibody fragment.

71. The method of any one of claims 58-70, wherein the attaching the first/second chimeric antigen receptor interacting domain comprises a method selected from fusing, grafting, conjugating and linking.

72. The method of any one of claims 58-70, further comprising attaching a first linker to the first site, wherein the first linker links the first chimeric antigen receptor interacting domain to the first target interacting domain.

73. The method of claim 72, further comprising attaching a second linker to the second site wherein the second linker links the second chimeric antigen receptor interacting domain to the second target interacting domain.

74. The method of claim 73, wherein the first linker and the second linker are the same.

75. The method of claim 73, wherein the first linker and the second linker are different.

76. The method of claim 75, wherein the first linker and the second linker differ by a feature selected from flexibility, length, chemistry, and combinations thereof.

77. The method of any one of claims 58-76, wherein the first chimeric antigen receptor and the second chimeric antigen receptor are the same.

78. The method of any one of claims 58-76, wherein the first chimeric antigen receptor and the second chimeric antigen receptor are different.

79. The method of claim 78, wherein the first chimeric antigen receptor and the second chimeric antigen receptor differ by a domain selected from an extracellular domain, a transmembrane domain, an intracellular domain and a hinge domain.

80. The method of claim 78, wherein a first hinge domain of the first chimeric antigen receptor and a second hinge domain of the second chimeric antigen receptor differ by a feature selected from flexibility, length, amino acid sequence and combinations thereof.

81. The method of any one of claims 58-80, further comprising incorporating one or more additional chimeric antigen receptor interacting domains to the first and/or second target interacting domain to produce a first multivalent switch and/or a second multivalent switch.

82. The method of any one of claims 58-80, further comprising incorporating a cysteine residue into the first chimeric antigen receptor and/or the second chimeric antigen receptor in order to multimerize the first chimeric antigen receptor and/or the second chimeric antigen receptor through a disulfide bond.

83. The method of any one of claims 58-82, wherein contacting the first target cell and/or contacting the second target cell occurs in vitro.

84. The method of any one of claims 58-82, wherein contacting the first target cell and/or contacting the second target cell occurs in vivo.

85. The method of claim 84, wherein comparing the first cytotoxic effect to the second cytotoxic effect comprises comparing a feature selected from viability of target cells, viability of off-target cells, tumor burden, and health of an in vivo model.

86. An optimized chimeric antigen receptor-effector cell platform, comprising:
(a) a chimeric antigen receptor-effector cell switch comprising a chimeric antigen receptor interacting domain and a targeting interacting domain; and
(b) a chimeric antigen receptor-effector cell that expresses a chimeric antigen receptor, wherein the chimeric antigen receptor-effector cell platform is derived by a method selected from claims 58-85.

87. The optimized chimeric antigen receptor-effector cell platform of claim 86, wherein the chimeric antigen receptor effector cell is derived from a T cell.

88. The optimized chimeric antigen receptor-effector cell platform of claim 86 or 87, wherein the targeting interacting domain is selected from a protein, a peptide, an antibody, an antibody fragment, a small molecule, and a soluble T cell receptor or portion thereof.

89. The optimized chimeric antigen receptor-effector cell platform of claim 86 or 87, wherein the targeting interacting domain comprises an antibody or antibody fragment that binds a cell surface molecule selected from CD19, CD22, Her2, CLL1, CD33, BMCA, CD123, CS1, EGFR, EGFRvIII, CD20, or CEA.

90. The optimized chimeric antigen receptor-effector cell platform of claim 86 or 87, wherein the target interacting domain comprises a sequence selected from SEQ ID NOS: 10-31 and optionally SEQ ID NOS: 52 and 53.

91. The optimized chimeric antigen receptor-effector cell platform of claim 84 or 85, wherein the target interacting domain comprises a sequence at least about 50% homologous to a sequence selected from SEQ ID NOS: 10-31 and optionally SEQ ID NOS: 52 and 53.

92. The optimized chimeric antigen receptor-effector cell platform of any one of claims 86-91, wherein the chimeric antigen receptor-interacting domain comprises a small molecule.

93. The optimized chimeric antigen receptor-effector cell platform of any one of claims 86-91, wherein the chimeric antigen receptor-interacting domain comprises a hapten.

94. The optimized chimeric antigen receptor-effector cell platform of any one of claims 86-91, wherein the chimeric antigen receptor-interacting domain is selected from FITC, biotin, and dinitrophenol.

95. The optimized chimeric antigen receptor-effector cell platform of any one of claims 86-91, wherein the chimeric antigen receptor-interacting domain comprises FITC.

96. A pharmaceutical composition comprising the soluble T cell receptor switch of any one of claims 12-22.

97. A method of treating a disease or condition in a subject in need thereof, comprising administering the soluble T cell receptor switch of claims 12-22 and a chimeric antigen receptor effector cell, wherein the soluble T cell receptor switch and/or chimeric antigen receptor effector cell is administered by a method selected from intraperitoneal injection and intravenous injection.

98. A method of treating a disease in a subject comprising administering a chimeric antigen receptor effector cell and a chimeric antigen receptor switch to the subject.

99. The method of claim 98, wherein the disease is cancer.

100. The method of claim 99, wherein the cancer is selected from AML, ALL, CLL, multiple myeloma, breast cancer, neuroblastoma, pancreatic cancer, endometrial cancer, ovarian cancer, or colon cancer.

101. The method of claim 98, wherein the chimeric antigen receptor effector cell is administered to the subject prior to administration of the chimeric antigen receptor switch.

102. The method of claim 97 or claim 98, wherein the chimeric antigen receptor switch comprises the soluble T cell receptor switch of any one of claims 12-22 or the chimeric antigen receptor switch of any one of claim 23-50 or 109.

103. The method of any one of claims 97 to 102, wherein the chimeric antigen receptor switch is administered in an amount titrated in the subject to optimize the treatment of the cancer.

104. The method of claim 101, wherein the chimeric antigen receptor switch is administered at an initial dose, wherein the initial dose is the smallest dose necessary to treat the cancer.

105. The method of claim 101 or 102, wherein the chimeric antigen receptor switch is administered at a second dose after about 1, 2, 3, 4, 5, 6, 7, 8, or more weeks of treatment at the initial dose, wherein the second dose is larger than the initial dose.

106. The method of any one of claims 97-105, wherein administration of the chimeric antigen receptor switch is terminated after one or more of a) adverse effects or b) elimination of the cancer.

107. The method of claim 106, wherein adverse effects comprise one or more of tumor lysis syndrome or cytokine release syndrome.

108. The method of claim 106, wherein administration of the chimeric antigen receptor switch is continued after recurrence of the cancer.

109. A chimeric antigen receptor switch comprising:
(a) a first chimeric antigen receptor interacting domain;
(b) a second chimeric antigen receptor interacting domain; and
(c) a target interacting domain.


SEQUENCE LISTING (53 sequences)

SEQ ID NO: 1 - DNA, 8892 bp, Artificial Sequene
  Made in lab - LV-EF1a-4-4-20-BBZ chimerica antigen receptor polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcggtaccgcggccgcccggggatccatggccttaccagtgaccgccttgctcctgccgctggccttgctgctccacgccgccaggccggatgttgttatgacccagaccccgctgtccctgcccgtttccctgggtgaccaggcgagtatctcttgccgttcctctcagtccctggttcacagtcagggtaacacctatctgcgctggtatctgcaaaaaccaggccagagccctaaagtgctgatttataaggtttcaaatcggtttagcggcgtcccggatcgcttctctgggagtggatcagggaccgactttacactgaaaattagccgcgtggaagcagaggatctgggcgtgtacttttgcagccagtccactcatgtgccgtggaccttcggcggtgggacaaaactggaaattaagcgtgcaggaggcggtgggagcggaggcggtgggtccggaggcggtgggtctggaggcggtgggagtggaggcggtgggtcaggcggtggtgggagcgaagtgaaactggatgagacaggaggaggtctggttcagccaggtcggcccatgaagctgtcctgtgtggcctctggctttaccttctccgactattggatgaactgggtccgtcagtctccggaaaaaggtctggagtgggtggcgcagattcggaacaagccctacaactacgaaacttactactctgatagtgttaaaggccgcttcaccatcagtcgtgatgactcaaaaagcagcgtttacctgcaaatgaacaatctgcgtgtcgaggacatgggcatctattactgcacaggctcctactatgggatggattattgggggcaggggacttcagttacagtttcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 2 - DNA, 8892 bp, Artificial Sequence
  Made in lab - LV-EF1a-4D5Flu-BBZ chimeric antigen receptor polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcggtaccgcggccgcccggggatccatggccttaccagtgaccgccttgctcctgccgctggccttgctgctccacgccgccaggccggacattcagatgactcagagcccgtcttccctgtccgcttcagtgggtgaccgtgttaccattacctgccgtgcttctcagagcctggtgcactcccagggtaacacctatctgcggtggtatcagcagaaaccgggcaaggcccccaaagttctgatttataaggtctctaatcggtttagtggcgttccgtcacgcttctcaggtagcgggtccggaacagattttaccctgacaattagcagcctgcaaccggaagacttcgcaacctattactgccagcagtctacccatgtgccctggacatttggccagggtactaaggttgagctgaaacgtgctggaggtggaggaagcggaggtggaggcagcggcggtgggggatctggcggtgggggaagtggcggtgggggatcaggcggtgggggaagcgaagtccagctggtggagagtggaggtggactggtgcagccaggaggcagcctgcgtctgtcctgtgccgcgtctggctttaccttcagcgattactggatgaactgggtccgtcaggcaccaggaaagggcctggaatgggtggcgcagattcggaacaagccttacaactacgagacttactacgcggatagcgtgaagggtcgcttcaccatcagtcgtgacacatcaaagaacactgtttacctgcaaatgaacagcctgcgtgcagaagataccgctgtctattactgcacaggctcttattacggcatggactactggggccagggtactctggtgaccgtttctagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 3 - DNA, 8868 bp, Artificial Sequence
  Made in lab - LV-EF1a-4M5.3-BBZ chimeric antigen receptor polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcggtaccgcggccgcccggggatccatggccttaccagtgaccgccttgctcctgccgctggccttgctgctccacgccgccaggccggatgttgttatgacacagaccccgctgagtctgcccgtctccctgggcgaccaggcctccatttcttgccgtagttcacagtccctggtgcactctaacggaaatacctatctgcgctggtacctgcagaaaccgggccagagccccaaagtcctgatctataaggtgtccaaccgggtgtctggtgttccggatcgcttttcagggagcggcagcggcaccgacttcacactgaagatcaatcgtgttgaagcagaggatctgggcgtctacttttgctctcagagtacacatgtgccatggactttcggcggtgggaccaaactggaaattaagagctccgcagatgacgctaaaaaggacgccgcgaaaaaggatgacgccaaaaaggatgacgcgaaaaaggatggaggcgtcaaactggacgagaccggtgggggactggtgcagccgggcggtgcaatgaagctgagttgtgtgacttcaggttttaccttcgggcactattggatgaactgggttcgtcagagtccagaaaaaggtctggagtgggtcgctcagtttcggaacaagccttacaactacgaaacatactactcagatagcgttaaagggcgcttcactattagccgtgatgactccaagtctagtgtgtacctgcagatgaacaatctgcgggttgaggacacaggcatctattactgcacaggggcgtcctatggtatggagtatctggggcagggaacaagcgtcaccgtctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 4 - DNA, 8859 bp, Artificial Sequence
  Made in lab - LV-EF1a-FITC(E2)-BBZ chimeric antigen receptor polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcggtaccgcggccgcccggggatccatggccttaccagtgaccgccttgctcctgccgctggccttgctgctccacgccgccaggccgcaggttcagctggttgagagcggaggcaatctggttcagcccggtggtagtctgcgtctgtcttgtgcggcgtcagggttcactttcggtagtttttcaatgagctgggtccgtcaggcaccaggcggtgggctggaatgggtggcaggtctgtctgcacgtagctccctgacccactatgcagatagtgttaaagggcggttcacaatttcacgcgacaacgctaagaatagcgtctacctgcaaatgaactccctgcgggtcgaggataccgcagtgtattactgcgctcgccgttcttatgactctagtggatactggggccatttttatagctacatggatgtgtggggacagggcactctggtgaccgtttccggaggcggtgggtctggaggcggtgggagtggaggcggtgggtcaagcgttctgacccagccgtcctctgtcagcgccgcgccaggccagaaagtgacaatttcctgttctggaagtacttcaaacatcggcaacaattatgtttcctggtatcagcagcacccgggcaaagcgcccaagctgatgatttatgatgtgtctaaacgtccaagtggtgttcctgaccggttcagcggttccaagtctgggaatagtgcctcactggacatctcaggcctgcaaagcgaagatgaggcggactattactgcgcagcttgggatgacagcctgtccgaatttctgttcggcaccgggacaaagctgaccgtgctgggcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 5 - DNA, 5574 bp, Artificial Sequence
  Made in Lab - pBAD-CD19wt CAR-EC switch target interacting domain polynucleotide
aagaaaccaattgtccatattgcatcagacattgccgtcactgcgtcttttactggctcttctcgctaaccaaaccggtaaccctgattatttgcacggagtcacactttgctatgccatagcatttttatccataagattagcggatcctacctgacgctttttatcgcaactctctactgtttctccatacccgtttttttgggctagaaataattttgtttaactttaagaaggagaatacatcaactagtacgcaagttcacgtaaaaagggtatctagaggttgaggtgattttatgaaaaagaatatcgcatttcttcttgctagcatgttcgttttttctattgctacaaacgcatacgctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgttaagctggggatcctctagaggttgaggtgattttatgaaaaagaatatcgcatttcttcttgcatctatgttcgttttttctattgctacaaacgcgtacgctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacataataagtcgaccgatgcccttgagagccttcaacccagtcagctccttccggtgggcgcggggcatgactatcgtcgccgcacttatgactgtcttctttatcatgcaactcgtaggacaggtgccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 6 - DNA, 5574 bp, Artificial Sequence
  Made in Lab - pBAD-CD19 LS202X mt CAR-EC switch target interacting domain polynucleotide
aagaaaccaattgtccatattgcatcagacattgccgtcactgcgtcttttactggctcttctcgctaaccaaaccggtaaccctgattatttgcacggagtcacactttgctatgccatagcatttttatccataagattagcggatcctacctgacgctttttatcgcaactctctactgtttctccatacccgtttttttgggctagaaataattttgtttaactttaagaaggagaatacatcaactagtacgcaagttcacgtaaaaagggtatctagaggttgaggtgattttatgaaaaagaatatcgcatttcttcttgctagcatgttcgttttttctattgctacaaacgcatacgctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtagtcgcccgtcacaaagagcttcaacaggggagagtgttaagctggggatcctctagaggttgaggtgattttatgaaaaagaatatcgcatttcttcttgcatctatgttcgttttttctattgctacaaacgcgtacgctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacataataagtcgaccgatgcccttgagagccttcaacccagtcagctccttccggtgggcgcggggcatgactatcgtcgccgcacttatgactgtcttctttatcatgcaactcgtaggacaggtgccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 7 - DNA, 5574 bp, Artificial Sequence
  Made in Lab - pBAD-CD19 HK136X mt CAR-EC switch target interacting domain polynucleotide
aagaaaccaattgtccatattgcatcagacattgccgtcactgcgtcttttactggctcttctcgctaaccaaaccggtaaccctgattatttgcacggagtcacactttgctatgccatagcatttttatccataagattagcggatcctacctgacgctttttatcgcaactctctactgtttctccatacccgtttttttgggctagaaataattttgtttaactttaagaaggagaatacatcaactagtacgcaagttcacgtaaaaagggtatctagaggttgaggtgattttatgaaaaagaatatcgcatttcttcttgctagcatgttcgttttttctattgctacaaacgcatacgctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgttaagctggggatcctctagaggttgaggtgattttatgaaaaagaatatcgcatttcttcttgcatctatgttcgttttttctattgctacaaacgcgtacgctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagcctccaccaagggcccatcggtcttccccctggcaccctcctcctagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacataataagtcgaccgatgcccttgagagccttcaacccagtcagctccttccggtgggcgcggggcatgactatcgtcgccgcacttatgactgtcttctttatcatgcaactcgtaggacaggtgccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 8 - DNA, 5574 bp, Artificial Sequence
  Made in Lab - pBAD-CD19 LS202/HK136X mt CAR-EC switch target interacting domain polynucleotide
aagaaaccaattgtccatattgcatcagacattgccgtcactgcgtcttttactggctcttctcgctaaccaaaccggtaaccctgattatttgcacggagtcacactttgctatgccatagcatttttatccataagattagcggatcctacctgacgctttttatcgcaactctctactgtttctccatacccgtttttttgggctagaaataattttgtttaactttaagaaggagaatacatcaactagtacgcaagttcacgtaaaaagggtatctagaggttgaggtgattttatgaaaaagaatatcgcatttcttcttgctagcatgttcgttttttctattgctacaaacgcatacgctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtagtcgcccgtcacaaagagcttcaacaggggagagtgttaagctggggatcctctagaggttgaggtgattttatgaaaaagaatatcgcatttcttcttgcatctatgttcgttttttctattgctacaaacgcgtacgctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagcctccaccaagggcccatcggtcttccccctggcaccctcctcctagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacataataagtcgaccgatgcccttgagagccttcaacccagtcagctccttccggtgggcgcggggcatgactatcgtcgccgcacttatgactgtcttctttatcatgcaactcgtaggacaggtgccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 9 - DNA, 8832 bp, Artificial Sequence
  Made in Lab - LV-EF1a-CD19(FMC63)-BBZ CAR-EC switch target interacting domain polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcggtaccgcggccgcccggggatccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 10 - protein, 107 aa, Artificial Sequence
  Made in Lab - Light chain of wildtype anti-CS1 antibody
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKLEIK

SEQ ID NO: 11 - protein, 119 aa, Artificial Sequence
  Made in Lab - Heavy chain of wildtype anti-CS1 antibody
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 12 - protein, 107 aa, Artificial Sequence
  Made in Lab - anti-Her2-Fab Variable light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIK

SEQ ID NO: 13 - protein, 114 aa, Artificial Sequence
  Made in Lab - Anti-Her2-Fab Variable heavy chain
QLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 14 - protein, 107 aa, Artificial Sequence
  Made in Lab - Light chain of anti-BCMA antibody (BCMA98)
DIQMTQSPSSLSASVGDRVTITCRANQGISNNLNWYQQKPGKAPKPLIYYTSNLQSGVPSRFSGSGSGTDYTLTISSLQPEDFATYYCQQFTSLPYTFGQGTKLEIK

SEQ ID NO: 15 - protein, 121 aa, Artificial Sequence
  Made in Lab - Heavy chain of anti-BCMA antibody (BCMA98)
EVQLVESGGGLVQPGGSLRLSCAASGFTFSNFDMAWVRQAPGKGLVWVSSITTGGGDTYYADSVKGRFTISRDNAKSTLYLQMDSLRSEDTAVYYCVRHGYYDGYHLFDYWGQGTLVTVSS

SEQ ID NO: 16 - protein, 214 aa, Artificial Sequence
  Made in Lab - anti-CD19-Fab Light Chain
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 228 aa, Artificial Sequence
  Made in Lab - anti-CD19-Fab Heavy Chain
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 18 - protein, 108 aa, Artificial Sequence
  Made in Lab - anti-CLL1-Fab Variable Light Chain (clone 1075.7)
ENVLTQSPAIMSASPGEKVTMTCRASSNVISSYVHWYQQRSGASPKLWIYSTSNLASGVPARFSGSGSGTSYSLTISSVEAEDAATYYCQQYSGYPLTFGAGTKLELK

SEQ ID NO: 19 - protein, 123 aa, Artificial Sequence
  Made in Lab - anti-CLL1-Fab Variable Heavy Chain (clone 1075.7)
DIQLQESGPGLVKPSQSLSLTCSVTGYSITSAYYWNWIRQFPGNKLEWMGYISYDGRNNYNPSLKNRISITRDTSKNQFFLKLNSVTTEDTATYYCAKEGDYDVGNYYAMDYWGQGTSVTVSS

SEQ ID NO: 20 - protein, 111 aa, Artificial Sequence [sequence omitted]

SEQ ID NO: 21 - protein, 116 aa, Artificial Sequence [sequence omitted]

SEQ ID NO: 22 - protein, 111 aa, Artificial Sequence
  Made in Lab - anti-CD33-Fab Variable Light Chain (clone hM195)
DIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIK

SEQ ID NO: 23 - protein, 116 aa, Artificial Sequence
  Made in Lab - anti- CD33-Fab Variable Heavy Chain (clone hM195)
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 24 - protein, 111 aa, Artificial Sequence
  Made in Lab - anti-CD123-Fab Variable Light Chain (clone 32716)
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 25 - protein, 118 aa, Artificial Sequence
  Made in Lab - anti- CD123-Fab Variable Heavy Chain (clone 32716)
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 26 - protein, 107 aa, Artificial Sequence
  Made in Lab - anti- CD123-Fab Variable Light Chain (clone 26292)
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 27 - protein, 115 aa, Artificial Sequence
  Made in Lab - anti- CD123-Fab Variable Heavy Chain (clone 26292)
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 28 - protein, 106 aa, Artificial Sequence
  Made in Lab - Anti- CD22- Fab Variable Heavy Chain (clone M971)
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEI

SEQ ID NO: 29 - protein, 125 aa, Artificial Sequence
  Made in lab - Anti- CD22- Fab Variable Light Chain (clone M971)
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSA

SEQ ID NO: 30 - protein, 112 aa, Artificial Sequence [sequence omitted]

SEQ ID NO: 31 - protein, 116 aa, Artificial Sequence [sequence omitted]

SEQ ID NO: 32 - DNA, 6102 bp, Artificial Sequence
  Made in lab - NYESO1(1G4-113)-TCRb-WT
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttagtcagcacgaccccacgcttctgcactgacaatttgcgtaaccggttttgcacggtcctgggtccattcatcattttcgctcaggccgtagaattgaacctgacagcgaaaatggttacgcgggtcctgccagaaggtggcagacacgcgcagacgggaactcagggcataacggctgtcattcagcgccggttgttctttcagcggctgcggatcggtacacacacccgagtggacttctttgccgttcacccaccatgacagttcgacatggtccggataaaagccggtggccaggcacaccagcgtcgctttctgggtatgggagatttcagcttcactcggttcgaaaactgccacttccggcggaaagacatttttcagatcttccagaaccgtcagacgagaaccttcgccgaaaaacagttcaccggtgttgcccaggtaagagcttgcgcagaaataaacagacgtttggctcggtgctgcgctcagcagacgcagcggaaagtcttcaatggtcgaacgtgaaacattgtaaccgttcggcacttcgccttgatccgtggtctggatggcgaccgaatagtgaatcagacgcagacccatacccgggtcctgacggtaccatgacatatattcatggttcatatcttgggcacattgcagcgtcatggattggcccgttttcaggacttggaatttcggcgtttgggtcacaccagcgttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 33 - DNA, 5997 bp, Artificial Sequence
  Made in Lab - NYESO1(1G4-113)-TCRa-WT
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttaagagctttccgggctcgggaaaaacgtatcttccgggataatactattgttgaatgcgttggcgcacgcaaagtcagatttattgctccaagcaactgccgagtttgatttgaaatccatagagcgcatgtccagaacacatttatcggtaatatacacgtcggaatctttggattgactgacattcgtctgactgtcgaaatcggtaaacaggcacacgcttttgtccgatgatttgctatcacgcagttggtacaccgccgggtccgggttctggatatacggatggacaatcaggctggtaccgcggccaaacgtcgggatataggtaccatccagcagcggacgcaccgcacacaggtacgtagcggagtcacccggctgacttgctgcaatatacagggtggaactaccagagcttttatccagcgatgcattcaggcggcctgaggtttgttcacgctgccacggcgtgatcagcagcaggctggtcagacctttgcccggatcttgacgaaaccactgcaggttgtaaatggcgctatccgtaaaggagcaattcagcaccagattttcaccttccgggaccgacagagcagccgggatttgggtaacttcttgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 34 - protein, 45 aa, Artificial Sequence
  Hinge sequence - CD8 hinge peptide
TTTPAPRPPTPAPTIASQPLSLRPEACRPAAGGAVHTRGLDFACD

SEQ ID NO: 35 - skipped

SEQ ID NO: 36 - protein, 12 aa, Artificial Sequence
  Hinge sequence - IgG4 hinge peptide
ESKYGPPCPSCP

SEQ ID NO: 37 - protein, 12 aa, Artificial Sequence
  Hinge sequence - IgG4 m hinge peptide
ESKYGPPCPPCP

SEQ ID NO: 38 - DNA, 4206 bp, Artificial Sequence
  Made in Lab - mCD19(1D3)-LC-WT (wild type light chain sequence)
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggacattcagctgacccagtctccagcttccctgtctacatctctgggagaaactgtcaccatccaatgtcaagcaagtgaggacatttacagtggtttagcgtggtatcagcagaagccagggaaatctcctcagctcctgatctatggtgcaagtgacttacaagacggcgtcccatcacgattcagtggcagtggatctggcacacagtattctctcaagatcaccagcatgcaaactgaagatgaaggggtttatttctgtcaacagggtttaacgtatcctcggacgttcggtggcggcaccaagctggaaatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaggcgggagcaattatcatcttgaaaatgaggtcgctcgtctcaagaaactcggtggcggaggcagcgacagcacctacagcctcagcagcaccctgacgcttagcaaagcagactacgagaaacacaaagtctacgcctgcgaagtcacccatcagggcctgtcctcgcccgtcacaaagagcttcaacaggggagagtgttgataagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 39 - DNA, 4137 bp, Artificial Sequence
  Made in Lab - mCD19(1D3)-HC-WT Fab (wild type heavy chain sequence)
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggacattcagctgacccagtctccagcttccctgtctacatctctgggagaaactgtcaccatccaatgtcaagcaagtgaggacatttacagtggtttagcgtggtatcagcagaagccagggaaatctcctcagctcctgatctatggtgcaagtgacttacaagacggcgtcccatcacgattcagtggcagtggatctggcacacagtattctctcaagatcaccagcatgcaaactgaagatgaaggggtttatttctgtcaacagggtttaacgtatcctcggacgttcggtggcggcaccaagctggaaatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgttgataagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 40 - protein, 214 aa, Artificial Sequence
  Made in Lab - anti-Her2 Fab wild type light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 228 aa, Artificial Sequence
  Made in lab - anti-Her2 Fab wild type heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 42 - protein, 214 aa, Artificial Sequence
  Made in Lab - anti-Her2 Fab (G68X) Light chain
  MOD_RES (68)..(68): Xaa = p-acetylphenylalanine (pAcF)
DIQMTQSPSSLSASVGDRVTITCRASQDVNTAVAWYQQKPGKAPKLLIYSASFLYSGVPSRFSGSRSXTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 214 aa, Artificial Sequence
  Made in Lab - anti-Her2 Fab (S202X) light chain
  MOD_RES (202)..(202): Xaa = p-acetylphenylalanine (pAcF)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLXSPVTKSFNRGEC

SEQ ID NO: 44 - protein, 228 aa, Artificial Sequence
  Made in Lab - anti-Her2 Fab (S75X) heavy chain
  MOD_RES (75)..(75): Xaa = p-acetylphenylalanine (pAcF)
EVQLVESGGGLVQPGGSLRLSCAASGFNIKDTYIHWVRQAPGKGLEWVARIYPTNGYTRYADSVKGRFTISADTXKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 45 - protein, 228 aa, Artificial Sequence
  Made in Lab - anti-Her2 Fab (K136X) heavy chain
  MOD_RES (136)..(136): Xaa = p-acetylphenylalanine (pAcF)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSXSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 46-47 - skipped (2 entries)

SEQ ID NO: 48 - protein, 470 aa, Artificial Sequence
  Made in Lab - CAR-Her2 (CD8 hinge)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 438 aa, Artificial Sequence
  Made in Lab - CAR-Her2 (IgG4m hinge)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAAESKYGPPCPPCPDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 474 aa, Artificial Sequence
  Made in Lab - CAR-FITC (CD8 hinge)
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSGGGGSGGGGSGGGGSSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 442 aa, Artificial Sequence
  Made in Lab - CAR-FITC (IgG4m hinge)
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSGGGGSGGGGSGGGGSSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGESKYGPPCPPCPDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 108 aa, Artificial Sequence
  Fab Heavy Chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 53 - protein, 107 aa, Artificial Sequence [sequence omitted]